(19) United States
(12) Patent Application Publication
(10) Pub. No.: US 2012/0214798 A1
(43) Pub. Date: Aug. 23, 2012

(54) Novel Ethanediamone Hepcidine Antagonists

(75) Inventors: Franz Dürrenberger, Dornach (CH); Susanna Burckhardt, Zurich (CH); Wilm Buhr, Konstanz (DE); Peter O. Geisser, St. Gallen (CH); Felix Funk, Winterthur (CH); Vincent A. Corden, Stanford in the Vale (GB); Stephen M. Courtney, Stanford in the Vale (GB); Tara Davenport, Southmoor (GB); Stefan Jaeger, Hamburg (DE); Mark P. Ridgill, Horsham (GB); Mark Slack, Hamburg (DE); Christopher J. Yarnold, Didcot Oxon (GB); Wei Tsung Yau, Didcot Oxon (GB)

(73) Assignee: VIFOR (INTERNATIONAL) AG, St. Gallen (CH)

(21) Appl. No.: 13/394,201

(22) PCT Filed: Sep. 6, 2010
(86) PCT No.: PCT/EP2010/063001

§ 371 Date: May 3, 2012

(30) Foreign Application Priority Data
Sep. 7, 2009 (EP) 09169614.6

(51) Int. Cl. A61K 31/497; A61K 31/5377; A61P 3/02; A61K 31/445; A61K 31/551; A61K 31/496; A61K 31/4545

(52) U.S. Cl. 514/218; 514/252.11; 514/253.11; 514/253.01; 514/237.2; 514/316; 514/254.1; 514/235.8; 514/331

(57) ABSTRACT

The present invention relates to novel hepcidin antagonists of formula (I), pharmaceutical compositions comprising them and the use thereof as medicaments, in particular for treatment of disorders in iron metabolism, such as, in particular, iron deficiency diseases and anaemias, in particular anaemias in connection with chronic inflammatory diseases (ACD: anaemia of chronic disease and AI: anaemia of inflammation).

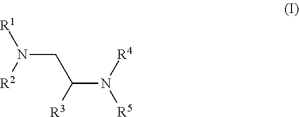

### INTRODUCTION

The invention relates to novel hepcidin antagonists of the general formula (I), pharmaceutical compositions comprising these and their use for treatment of iron metabolism disorders, in particular of anaemias in connection with chronic inflammatory diseases (anaemia of chronic disease (ACD) and anaemia of inflammation (AI)) or of iron deficiency symptoms and iron deficiency anaemias.

### BACKGROUND

Iron is an essential trace element for almost all organisms and in this context is relevant in particular for growth and blood formation. The balance of iron metabolism in this context is primarily regulated at the level of recovery of iron from haemoglobin from ageing erythrocytes and duodenal absorption of iron bonded in food. The iron released is absorbed via the intestine, in particular by way of specific transport systems (DMT-1, ferroportin, transferrin, transferrin receptors), transported into the blood stream and passed on by this means into the corresponding tissue and organs.

The element iron is of great importance in the human body inter alia for oxygen transport, oxygen uptake, cell functions, such as mitochondrial electron transport, and finally for energy metabolism in total.

The body of a human contains on average 4 to 5 g of iron, this being present in enzymes, in haemoglobin and myoglobin and as depot or reserve iron in the form of ferritin and haemosiderin.

About half of this iron, approx. 2 g, is present as haem iron bonded in the haemoglobin of red blood corpuscles. Since these erythrocytes have only a limited life (75-150 days), new ones must constantly be formed and old ones eliminated (over 2 million new erythrocytes are formed per second). This high regeneration capacity is achieved by macrophages, in that these absorb the ageing erythrocytes by phagocytosis, lyse them and in this way can recycle the iron contained in them for the iron metabolism. The amount of iron required daily for erythropoiesis of approx. 25 mg is thus mostly provided.

The daily iron requirement of an adult human is between 0.5 and 1.5 mg per day, and for infants and women in pregnancy the iron requirement is 2 to 5 mg per day. Daily iron losses, e.g. by exfoliation of skin cells and epithelial cells, is comparatively low, but increased iron losses occur, for example, in women during menstrual bleeding. Blood losses generally can considerably reduce iron metabolism, since about 1 mg of iron is lost per 2 ml of blood. The normal daily iron loss of approx. 1 mg is conventionally replaced again by an adult, healthy human via the daily food intake. Iron metabolism is regulated via absorption, the absorption rate of the iron present in food being between 6 and 12%, and in the event of iron deficiency the absorption rate is up to 25%. The absorption rate is regulated by the organism as a function of iron requirement and the size of the iron store. In this context, the human organism uses both divalent and trivalent iron ions. Iron(III) compounds are conventionally dissolved in the stomach at a sufficiently acid pH and are thus made available for absorption. Absorption of the iron takes place in the upper small intestine by mucosa cells. In this context, for absorption trivalent non-haem iron is first reduced to Fe²⁺ e.g. by ferrireductase (duodenal cytochrome b at the membrane) in the membrane of intestinal cells, so that it can then be transported by the transport protein DMT! (divalent metal transporter 1) into the intestinal cells. On the other hand, haem iron enters into the enterocytes unchanged via the cell membrane. In the enterocytes, iron is either stored as depot iron in ferritin or released into the blood by the transport protein ferroportin, bonded to transferrin. Hepcidin plays a central role in this operation, since it is the essential regulation factor of iron uptake. The divalent iron transported into the blood by the ferroportin is converted into trivalent iron by oxidases (ceruloplasmin, hephaestin), which is then transported to the relevant places in the organism by means of transferrin (see for example: “Balancing acts: molecular control of mammalian iron metabolism”. M. W. Hentze, Cell 117,2004,285-297.)

The regulation of the iron level in this context is controlled or regulated by hepcidin.

Hepcidin is a peptide hormone which is produced in the liver. The prevailing active form has 25 amino acids (see for example: “Hepcidin, a key regulator of iron metabolism and mediator of anemia of inflammation”. T. Ganz Blood 102,2003,783-8), although two forms shortened at the amino end, hepcidin-22 and hepcidin-20, have been found. Hepcidin acts on iron uptake via the intestine, via the placenta and on the release of iron from the reticuloendothelial system. In the body, hepcidin is synthesized from so-called pro-hepcidin in the liver, pro-hepcidin being coded by the so-called HAMP gene. If the organism is adequately supplied with iron and oxygen, increased hepcidin is formed. In the mucosa cells of the small intestine and in the macrophages, hepcidin binds to ferroportin, by means of which iron is conventionally transported out of the cell interior into the blood.

The transport protein ferroportin is a membrane transport protein comprising 571 amino acids which is formed and located in the liver, spleen, kidneys, heart, intestine and placenta. In particular, in this context ferroportin is located in the basolateral membrane of intestinal epithelial cells. The ferroportin bound in this way effects export of iron into the blood here. In this context, ferroportin very probably transports iron as Fe²⁺. If hepcidin is bound to ferroportin, ferroportin is transported into the cell interior and degraded, as a result of which the release of iron from the cells is then almost completely blocked. If the ferroportin is inactivated via hepcidin, the iron stored in the mucosa cells therefore cannot be transported away, and the iron is lost with the natural exfoliation of cells via the stool. As a result, absorption of iron in the intestine is reduced by hepcidin. On the other hand, if the iron content in the serum is lowered, hepcidin production in the hepatocytes of the liver is reduced, so that less hepcidin is released and therefore less ferroportin is inactivated, as a result of which an increased amount of iron can be transported into the serum.

Ferroportin is moreover located to a high degree in the reticuloendothelial system (RES), to which the macrophages also belong.

Hepcidin plays an important role here in the event of impaired iron metabolism in the context of chronic inflammations, since interleukin-6 in particular is increased with such inflammations, which leads to an increase in the hepcidin level. Increased hepcidin is bound to the ferroportin of the macrophages by this means, as a result of which release of iron is blocked here, which in the end then leads to an inflammation-related anaemia (ACD or AI).

Since the organism of mammals cannot actively excrete iron, iron metabolism is essentially controlled via cellular release of iron from macrophages, hepatocytes and enterocytes by way of hepcidin.

Hepcidin thus plays an important role in functional anaemia. In this case, in spite of a full iron store, the iron requirement of bone marrow for erythropoiesis is not met sufficiently. The reason for this is assumed to be an increased hepcidin concentration, which in particular limits the transport of iron from the macrophages by blocking the ferroportin and thus greatly reduces the release of iron recycled by phagocytosis.

In the event of a disturbance in the hepcidin regulation mechanism, a direct effect thus manifests itself on iron metabolism in the organism. For example, if hepcidin expression is prevented, for example by a genetic defect, this leads directly to an overloading of iron, which is known as the iron storage disease haemochromatosis.

On the other hand, overexpression of hepcidin, for example due to inflammation processes, for example with chronic inflammations, results directly in reduced serum iron levels. In pathological cases this can lead to a reduced content of haemoglobin, reduced erythrocyte production and therefore to an anaemia.

The duration of use of chemotherapeutics in carcinoma treatments can be significantly reduced by an existing anaemia, since the state of reduced formation of red blood corpuscles caused by the chemotherapeutics employed is intensified still further by an existing anaemia.

Further symptoms of anaemias include tiredness, pallor and reduced attention capacities. The clinical symptoms of anaemia include low serum iron contents (hypoferraemia), low haemoglobin contents, low haematocrit level and a reduced number of red blood corpuscles, reduced reticulocytes and increased values of soluble transferrin receptors.

Iron deficiency symptoms or iron anaemias are conventionally treated by supplying iron. In this context, substitution with iron takes place either by the oral route or by intravenous administration of iron. Erythropoietin and other erythropoiesis-stimulating substances can moreover also be employed in the treatment of anaemias to give a boost to the formation of red blood corpuscles.

Anaemias which are caused by chronic diseases, e.g. chronic inflammatory diseases, can be treated only inadequately with such conventional treatment methods. Cytokines, such as in particular inflammatory cytokine, in particular play a particular role in anaemias which are based on chronic inflammation processes. An overexpression of hepcidin occurs in particular with such chronic inflammatory diseases and is known to lead to a reduced availability of iron for the formation of the red blood corpuscles.

From this emerges the need for an effective treatment method for hepcidin-mediated or -imparted anaemias, in particular those which cannot be treated with conventional iron substitution, such as those anaemias which are caused by chronic inflammatory diseases (ACD and AI).

Anaemia is to be attributed inter alia to those chronic inflammatory diseases mentioned, and to malnutrition or low-iron diets or unbalanced, low-iron eating habits. Anaemias moreover occur due to reduced or poor absorption of iron, for example due to gastrectomies or diseases such as Crohn's disease. An iron deficiency can also occur as a result of an increased blood loss, e.g. due to an injury, heavy menstrual bleeding or blood donation. An increased iron requirement in the growth phase of adolescents and children and in pregnant women is also known. Since an iron deficiency leads not only to a reduced formation of red blood corpuscles but therefore also to a poor supply of oxygen to the organism, which can lead to the above-mentioned symptoms, such as tiredness, pallor and lack of concentration and also precisely in adolescents to long-term negative effects on cognitive development, a particularly effective therapy in addition to the known conventional substitution therapy is also of particular interest for this sector.

Compounds which bind to hepcidin or to ferroportin and therefore inhibit the binding of hepcidin to ferroportin and therefore in turn prevent the inactivation of ferroportin by hepcidin, or compounds which, although hepcidin is bound to ferroportin, prevent the internalization of the hepcidin-ferroportin complex, and in this manner prevent the inactivation of the ferroportin by the hepcidin, can be called in general terms hepcidin antagonists.

By using such hepcidin antagonists, there is moreover also generally the possibility, for example by inhibiting hepcidin expression or by blocking the hepcidin-ferroportin interaction, of acting directly on the regulation mechanism of hepcidin and therefore of preventing via this route blocking of the iron transport pathway from tissue macrophages, liver cells and mucosa cells into the serum via the transport protein ferroportin. With such hepcidin antagonists or ferroportin expression inhibitors, substances are therefore available which are suitable for the preparation of pharmaceutical compositions or medicaments in the treatment of anaemias, in particular anaemias with chronic inflammatory diseases. These substances can be employed for treatment of such disorders and the resulting diseases, since these have a direct influence on the increase in the release of recycled haem iron by macrophages and effect an increase in the iron absorption of iron released from food in the intestinal tract. Such substances, inhibitors of hepcidin expression or hepcidin antagonists, can therefore be used for treatment of iron metabolism disorders, such as iron deficiency diseases, anaemias and anaemia-related diseases. In particular, this also includes those anaemias which are caused by acute or chronic inflammatory diseases, such as, for example, osteoarticular diseases, such as rheumatoid polyarthritis, or diseases which are associated with inflammatory syndromes. Such substances can therefore be of particular benefit in particular in the indications of cancer, in particular colorectal cancer, multiple myeloma, ovarian and endometrial cancer and prostate cancer, CKD 3-5 (chronic kidney disease stage 3-5) CHF (chronic heart failure), RA (rheumatoid arthritis), SLE (systemic lupus erythematosus) and IBD (inflammatory bowel disease).

### PRIOR ART

Hepcidin antagonists or compounds which have an inhibiting or assisting action on the biochemical regulation pathways in iron metabolism are known in principle from the prior art.

Thus, for example, WO 2008/036933 describes double-stranded dsRNA which has an inhibiting action on the expression of human HAMP genes in cells and therefore already suppresses the formation of hepcidin, which is coded by the HAMP gene, at a very early stage in the iron metabolism signal pathway. As a result, less hepcidin is formed, so that hepcidin is not available for the inhibition of ferroportin, so that the transport of iron from the cell into the blood by ferroportin can take place unimpeded.

Further compounds which aim directly at reduction of hepcidin expression are known from US 2005/020487, which describes compounds which have an HIF-α stabilizing action and therefore lead to a reduction in hepcidin expression.

The subject matter of US 2007/004618 is siRNA, which has a directly inhibiting action on hepcidin mRNA expression.

All these compounds or methods are therefore those which start in the iron metabolism pathway before formation of the hepcidin and already regulate its general formation downwards. In addition, however, also such substances and compounds are also known and described in the prior art which bind in the body to hepcidin which has already formed and therefore inhibit its binding action on the membrane transport protein ferroportin, so that an inactivation of ferroportin by hepcidin is no longer possible. Such compounds are therefore so-called hepcidin antagonists, those based on hepcidin antibodies being known in particular from this group. Such documents are furthermore known in the prior art which describe various mechanisms for action on hepcidin expression, for example by antisense RNA or DNA molecules, ribozymes and anti-hepcidin antibodies. Such mechanisms are described, for example, in EP 1 392 345.

WO09/058,797 furthermore discloses anti-hepcidin antibodies and the use thereof for specific binding to human hepcidin-25, and therefore the use thereof for therapeutic treatment of low iron contents, in particular of anaemias.

Further compounds which act as hepcidin antagonists and are formed from the group of hepcidin antibodies are known from EP 1 578 254, WO08/097,461, US2006/01 9339, WO09/044,284 or WO09/027,752.

In addition, antibodies which bind to ferroportin-1 and therefore activate ferroportin in order to assist in the iron transport from the cell into the serum by this means are also known. Such ferroportin-1 antibodies are known, for example, from US2007/218055.

All these compounds described which can act as hepcidin antagonists or can display an inhibiting action in hepcidin expression are higher molecular weight compounds, in particular those which are chiefly obtainable by genetic engineering processes.

In addition, low molecular weight compounds which play a role in iron metabolism and which can have either an inhibiting or also an assisting action are also known.

WO08/109,840 thus describes certain tricyclic compounds which can be employed in particular for treatment of disorders in iron metabolism, such as, for example, ferroportin disorders, these compounds being able to act by regulation of DMT-1 in the form of inhibition or activation. In this context, the compounds of this WO08/109,840 are described in particular as DMT-1 inhibitors, whereby they can preferably be employed on diseases with increased iron accumulation or iron storage diseases, such as haemochromatosis.

WO08/121,861 also discloses low molecular weight compounds which have a regulating action on the DMT-1 mechanism. Certain pyrazole and pyrrole compounds are dealt with here, treatment of iron overloading disorders, for example on the basis of ferroportin disorders, also being described here in particular.

The subject matter of US2008/234384 is furthermore certain diaryl and diheteroaryl compounds for treatment of disorders in iron metabolism, such as, for example, ferroportin disorders, which likewise by their action as DMT-1 inhibitors can be employed in particular for treatment of disorders on the basis of increased iron accumulation. In this document, however, possible DMT-1 regulatory mechanisms which can be employed for use on iron deficiency symptoms are also mentioned quite generally.

The same applies to WO08/151,288, which describes certain aromatic and heteroaromatic compounds with an action on DMT-1 regulation and therefore for treatment of disorders in iron metabolism.

The low molecular weight compounds described in the prior art which have an action on iron metabolism are therefore based on DMT-1 regulatory mechanisms and are disclosed in particular for use as agents for treatment of iron accumulation disorders or iron overloading syndromes, such as haemochromatosis.

“Hepcidin—Central-regulator of iron-metabolism” (Atanasiu Valeriu et al., European Journal of Haematology, 78 (1), 2007) gives an overview of hepcidin and its function. However, no indications of low molecular weight antagonists, in particular those with an ethanediamine structure, emerge from this.

Chemical compounds on the structural basis of ethanediamines thus have not yet hitherto been described in connection with treatment of disorders in iron metabolism. Furthermore, no low molecular weight chemical structures which display their action as hepcidin antagonists and as a result are suitable for treatment of disorders in iron metabolism have yet been described hitherto.

The present invention also provides novel ethanediamine compounds of the general structural formula (I) as well as (Ia) according to the present invention.

EP 1468990 A1 and EP 1295608 A1 disclose piperazine derivatives and the use thereof as MC4 receptor antagonists and therefore the use thereof in the treatment of anxiety disorders, neuroses and depression. However, these disclose generically exclusively those piperazine derivatives which contain an alkyl substituent in position R⁶, corresponding to the formula (Ia) of the present invention, and a second heterocyclic ring, for example a second piperazine ring, corresponding to one of the preferred meanings for the substituents R¹ and R² of the compounds of the present invention. Compounds disclosed herein which fall under the general formula (I) of the present invention thus relate exclusively to bis-piperazines which are substituted by optionally substituted alkyl in the position of R⁶, corresponding to formula (Ia) of the present invention. An action of such specific bis-piperazine derivatives in the treatment of disorders in iron metabolism does not emerge from these documents.

“Structure-activity relationships of novel piperazines as antagonists for the melanocortin-4 receptor” (Dai Nozawa et al., Bioorganic & Medicinal Chemistry; 15, 2007) also discloses such specific bis-piperazine derivatives which contain an arylalkyl substituent in position R⁶, corresponding to the formula (Ia) of the present invention, and the use thereof in the treatment of diseases of the central nervous system (CNS), such as, inter alia, anxiety disorders and depression. From here also no indication of an action of such bis-piperazine compounds in the treatment of disorders in iron metabolism emerges.

“Amides of Piperidine, Morpholine and piperazine Substituted 1-Phenylethylamines: Inhibitors of AcylCoA: cholesterol Acyltransferase (ACAT) Activity in vitro and in vivo” (S. Dugar et al., Bioorganic and Medicinal Chemistry; vol. 3, no. 9 1995) furthermore discloses selected low molecular weight piperidine-, piperazine- or morpholine-substituted phenylethylamides, also including in particular some selected compounds corresponding to compounds of the general formula (I) of the present invention wherein the substituents R¹ and R² have the meaning of a common 6-membered ring which can optionally contain further hetero atoms and wherein the substituents R⁴ and R⁵ are different and denote hydrogen and acyl, and the use thereof as acetylCoA inhibitors, e.g. in the treatment of coronary arterial diseases. In this context, however, only those compounds wherein acyl in the position of R⁴ or R⁵ is chosen from the group of long-chain alkanoyls (at least C₁₀-alkanoyl), in particular oleoyl, and from diphenylacetyl. From here also no indication of an action of such specific compounds in the treatment of disorders in iron metabolism emerges.

US 2004/0044033 A1 discloses specific benzoyl-piperidine compounds and the use thereof for treatment of diseases of the CNS, such as e.g. depression and anxiety states. Compounds which fall under the general formula (I) of the present invention in this context relate exclusively to those wherein the substituent X, corresponding to formula (I) of the present invention, denotes CH and R⁶ denotes benzoyl. Furthermore, only such compounds, corresponding to formula (I) of the present invention are concretely disclosed, wherein the substituents R⁴ and R⁵, corresponding to formula (I) of the present invention, moreover together form an aromatic 5-membered ring which contains at least one further nitrogen-hetero atom. An action of such specific benzoylpiperidine derivatives in the treatment of disorders in iron metabolism does not emerge from these documents.

WO 02/16308 A1 discloses, inter alia, di-alkylphenyl-substituted ethanediamines and the use thereof as blockers of the voltage-dependent sodium channel, in particular for treatment of diseases based on dysfunction caused by hyperexcitation. Compounds which fall under the general formula (I) of the present invention in this context relate exclusively to those wherein the substituent R³, corresponding to formula (I) of the present invention, denotes 2,6-dimethylphenyl. Furthermore, only such compounds, corresponding to the formula (I) of the present invention, wherein the substituents R¹ and R², corresponding to formula (I) of the present invention, moreover together form an aliphatic 5- or 6-membered ring without further hetero atoms, and the concrete compound [3-(2,6-difluorophenyl)-propyl]-[1-(2,6-dimethyl-phenyl)-2-cyclohexylamine-ethyl]amine, wherein the substituents R¹ and R², corresponding to formula (I) of the present invention, are different and denote hydrogen and cyclohexanyl and wherein R⁴ and R⁵, corresponding to formula (I) of the present invention, are different and denote hydrogen and 2,6-difluorophenyl-propyl, are concretely disclosed. An action of such specific di-methylphenyl-ethanediamines in the treatment of disorders in iron metabolism does not emerge from these documents.

U.S. Pat. No. 5,486,518 describes 4-indolylpiperazinyl derivatives and the use thereof as an anxiolytic or antidepressant. Compounds disclosed in this specification which fall under the general formula (I) of the present invention in this context relate exclusively to those wherein the substituents R⁴ and R⁵, corresponding to formula (I) of the present invention, are different and denote hydrogen and optionally substituted acyl, and wherein the substituents R¹ and R², corresponding to formula (I) of the present invention, together form a piperazine ring which contains heterocyclyl-substituted aryl, chosen from indolyl, in the position of the substituent R⁶. An action of such specific piperazine derivatives in the treatment of disorders in iron metabolism does not emerge from these documents.

### OBJECT

The object of the present invention was to provide in particular such compounds which can be employed for use for iron deficiency disorders or anaemias, in particular ACD and AI and which act in iron metabolism in particular as hepcidin antagonists and therefore display an antagonistic and via this a regulating action in the hepcidin-ferroportin interaction in iron metabolism. It was furthermore in particular an object of the present invention to provide in this context such compounds which are chosen from the group of low molecular weight compounds and which generally can be prepared by simpler synthesis routes than the antagonistic or hepcidin-inhibiting compounds obtainable by genetic engineering processes, such as RNA, DNA or antibodies.

### DESCRIPTION OF THE INVENTION

The inventors have found that certain compounds from the group of ethanediamines have an action as hepcidin antagonists.

The invention provides compounds of the general structural formula (I)

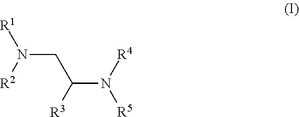

wherein

R¹ and R² are identical or different and are each chosen from the group consisting of:
hydrogen,
optionally substituted acyl,
optionally substituted alkyl,
optionally substituted aryl, and
optionally substituted heterocyclyl; or

R¹ and R² together with the nitrogen atom to which they are bonded form a saturated or unsaturated, optionally substituted 5- to 8-membered ring which can optionally contain further hetero atoms;

R³ is chosen from the group consisting of:
optionally substituted aryl, and
optionally substituted heterocyclyl;

R⁴ and R⁵ are identical or different and are each chosen from the group consisting of:
hydrogen,
optionally substituted alkyl-, aryl- or heterocyclylsulfonyl,
optionally substituted acyl,
optionally substituted alkyl,
optionally substituted alkenyl,
optionally substituted alkynyl,
optionally substituted aryl, and
optionally substituted heterocyclyl; or

R⁴ and R⁵ together with the nitrogen atom to which they are bonded form a saturated or unsaturated, optionally substituted 5- to 8-membered ring which can optionally contain further hetero atoms;

or pharmaceutically acceptable salts thereof.

In the context of the entire invention, the abovementioned substituent groups are defined as follows:

Optionally substituted alkyl preferably includes:

straight-chain or branched alkyl having preferably 1 to 8, more preferably 1 to 6, particularly preferably 1 to 4 carbon atoms. In one embodiment of the invention, optionally substituted straight-chain or branched alkyl can also include such alkyl groups in which preferably 1 to 3 carbon atom(s) are replaced by corresponding hetero-analogous groups which contain nitrogen, oxygen or sulfur. This means in particular that, for example, one or more methylene groups in the alkyl radicals mentioned can be replaced by NH, O or S.

Optionally substituted alkyl furthermore includes cycloalkyl having preferably 3 to 8, more preferably 5 or 6, particularly preferably 6 carbon atoms.

Substituents of the optionally substituted alkyl defined above preferably include 1 to 3 identical or different substituents which are chosen, for example, from the group which consists of: optionally substituted cycloalkyl, as defined below, hydroxyl, halogen, cyano, alkoxy, as defined below, optionally substituted aryloxy, as defined below, optionally substituted heterocyclyloxy, as defined below, carboxyl, optionally substituted acyl, as defined below, optionally substituted aryl, as defined below, optionally substituted heterocyclyl, as defined below, optionally substituted amino, as defined below, mercapto, optionally substituted alkyl-, aryl- or heterocyclylsulfonyl (R—SO₂—), as defined below.

Examples of alkyl radicals having 1 to 8 carbon atoms include: a methyl group, an ethyl group, an n-propyl group, an i-propyl group, an n-butyl group, an i-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4-methylpentyl group, a 1-ethylbutyl group, a 2-ethylbutyl group, a 3-ethylbutyl group, a 1,1-dimethylbutyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propylpentyl group, a 2-propylpentyl group etc. Those having 1 to 6 carbon atoms, in particular methyl, ethyl, n-propyl and i-propyl and butyl, are preferred. C₁ to C₄ alkyl, such as, in particular, methyl and ethyl, propyl, i-propyl and butyl, are most preferred.

Examples of alkyl groups which arise by replacement with one or more hetero-analogous groups, such as —O—, —S— or —NH—, are preferably those in which one or more methylene groups are replaced by —O— to form an ether group, such as methoxymethyl, ethoxymethyl, 2-methoxyethyl, 3-methoxypropyl, 2-ethoxyethyl etc., 2-methoxyethyl, 3-methoxypropyl and 2-ethoxyethyl being particularly preferred.

According to the invention, polyether groups, such as poly(ethylenoxy) groups, are also included in the definition of alkyl.

Cycloalkyl radicals having 3 to 8 carbon atoms preferably include: a cyclopropyl group, a cyclobutyl group, a cyclopentyl group, a cyclohexyl group, a cycloheptyl group and a cyclooctyl group. A cyclopropyl group, a cyclobutyl group, a cyclopentyl group and a cyclohexyl group are preferred. A cyclopentyl group and a cyclohexyl group are particularly preferred.

In the context of the present invention, halogen includes fluorine, chlorine, bromine and iodine, preferably fluorine or chlorine.

Examples of a linear or branched alkyl radical having 1 to 8 carbon atoms and substituted by halogen include:

a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a 1,2-difluoroethyl group, a 1,2-dichloroethyl group, a 1,2-dibromoethyl group, a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptyl group, a 7-fluoroheptyl group, a 7-chloroheptyl group, a 7-bromoheptyl group, a perfluoroheptyl group, etc. A trifluoromethyl group is preferred.

Examples of a cycloalkyl radical having 3 to 8 carbon atoms and substituted by halogen include: a 2-fluorocyclopentyl group, a 2-chlorocyclopentyl group, a 2-bromocyclopentyl group, a 3-fluorocyclopentyl group, a 3-chlorocyclopentyl group, a 3-bromocyclopentyl group, a 2-fluorocyclohexyl group, a 2-chlorocyclohexyl group, a 2-bromocyclohexyl group, a 3-fluorocyclohexyl group, a 3-chlorocyclohexyl group, a 3-bromocyclohexyl group, a 4-fluorocyclohexyl group, a 4-chlorocyclohexyl group, a 4-bromocyclohexyl group, a di-fluorocyclopentyl group, a di-chlorocyclopentyl group, a di-bromocyclopentyl group, a di-fluorocyclohexyl group, a di-chlorocyclohexyl group, a di-bromocyclohexyl group, a tri-fluorocyclohexyl group, a tri-chlorocyclohexyl group, a tri-bromocyclohexyl group etc.

Examples of an alkyl radical substituted by hydroxyl include the abovementioned alkyl radicals which contain 1 to 3 hydroxyl radicals, such as, for example, hydroxymethyl, 2-hydroxyethyl, 3-hydroxypropyl etc.

Examples of an alkyl radical substituted by alkoxy include the abovementioned alkyl radicals which contain 1 to 3 alkoxy radicals, as defined below, such as, for example, methoxymethyl, ethoxymethyl, 2-methoxyethyl, 2-ethoxyethyl, 2-methoxypropyl, 3-methoxypropyl etc., 2-methoxyethylene etc. 2-Methoxyethyl, 2-ethoxyethyl and 3-methoxypropyl are preferred.

Examples of an alkyl radical substituted by aryloxy include the abovementioned alkyl radicals which contain 1 to 3 aryloxy radicals, as defined below, such as, for example, phenoxymethyl, 2-phenoxyethyl and 2- or 3-phenoxypropyl etc. 2-Phenoxyethyl is preferred.

Examples of an alkyl radical substituted by heterocyclyloxy include the abovementioned alkyl radicals which contain 1 to 3 heterocyclyloxy radicals, as defined below, such as, for example, pyridin-2-yloxymethyl,-ethyl or -propyl, pyridin-3-yloxymethyl, -ethyl or -propyl, thiophen-2-yloxymethyl, -ethyl or -propyl, thiophen-3-yloxymethyl, -ethyl or propyl, furan-2-yloxymethyl, -ethyl or -propyl, furan-3-yloxymethyl, -ethyl or -propyl.

Examples of an alkyl radical substituted by acyl include the abovementioned alkyl radicals which contain 1 to 3 acyl radicals, as defined below.

Examples of an alkyl group substituted by cycloalkyl include the abovementioned alkyl radicals which contain 1 to 3, preferably one

(optionally substituted) cycloalkyl group, such as, for example: cyclohexylmethyl, 2-cyclohexylethyl, 2- or 3-cyclohexylpropyl etc.

Examples of an alkyl group substituted by aryl include the abovementioned alkyl radicals which contain 1 to 3, preferably one

(optionally substituted) aryl group, as defined below, such as, for example, phenylmethyl, 2-phenylethyl, 2- or 3-phenylpropyl etc., phenylmethyl being preferred. Alkyl groups, as defined above, which are substituted by substituted aryl, as defined below, in particular by alkoxy-substituted aryl, are furthermore particularly preferred, such as particularly preferably para-methoxyphenylmethyl:

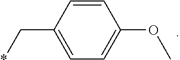

Examples of an alkyl group substituted by heterocyclyl include the abovementioned alkyl radicals which contain 1 to 3, preferably one (optionally substituted) heterocyclyl group, as defined below, such as, for example, 2-pyridin-2-yl-ethyl, 2-pyridin-3-yl-ethyl, pyridin-2-yl-methyl, pyridin-3-yl-methyl, 2-furan-2-yl-ethyl, 2-furan-3-yl-ethyl, furan-2-yl-methyl, furan-3-yl-methyl, 2-thiophen-2-yl-ethyl, 2-thiophen-3-yl-ethyl, thiophen-2-yl-methyl, thiophen-3-yl-methyl, imidazol-1-yl-methyl, imidazol-2-yl-methyl, 2-imidazol-1-yl-ethyl, 2-imidazol-2-yl-ethyl, 2-morpholinylethyl, such as 2-morpholin-4-yl-ethyl, morpholinylmethyl, such as morpholin-4-yl-methyl, 2-tetrahydrofuranylethyl, such as 2-tetrahydrofuran-2-yl-ethyl, tetrahydrofuranylmethyl, such as tetrahydrofuran-2-yl-methyl etc. Pyridin-2-yl-methyl:

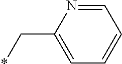

and 2-morpholin-4-yl-ethyl:

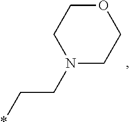

and thiophen-2-yl-methyl:

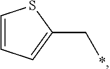

2-imidazol-1-yl-ethyl:

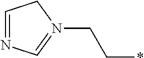

and tetrahydrofuran-2-yl-methyl:

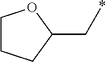

(*Bonding position to the base skeleton).
are particularly preferred.

Examples of an alkyl radical substituted by amino include the abovementioned alkyl radicals which contain 1 to 3, preferably one (optionally substituted) amino group, as defined below, such as, for example, methylaminomethyl, methylaminoethyl, methylaminopropyl, 2-methylaminomethyl (di-methylaminomethyl), 2-ethylaminomethyl (di-ethylaminomethyl), 3-ethylaminomethyl, 2-methylaminoethyl (di-methylaminoethyl), 2-ethylaminoethyl (di-ethylaminoethyl), 3-ethylaminoethyl etc. 2-Ethylaminoethyl (di-ethylaminoethyl) is preferred. (N-Methyl)(N-pyrazin-2-yl)aminoethyl:

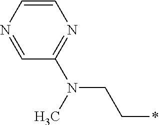

is furthermore particularly preferred.

Optionally substituted alkoxy includes an optionally substituted alkyl-O group, wherein reference may be made to the above definition with respect to the definition of the alkyl group. Preferred alkoxy groups are linear or branched alkoxy groups having up to 6 carbon atoms, such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group, and cycloalkyloxy groups, such as a cyclopentyloxy group or a cyclohexyloxy group. A methoxy group, an ethoxy group, an n-propyloxy group, an i-propyloxy group, an n-butyloxy group, an i-butyloxy group, a sec-butyloxy group, a t-butyloxy group are preferred. The methoxy group, the ethoxy group and the i-propyloxy group are particularly preferred.

Optionally substituted aryloxy includes an optionally substituted aryl-O group, wherein reference may be made to the following definition of optionally substituted aryl with respect to the definition of the aryl group. Preferred aryloxy groups include 5- and 6-membered aryl groups, among which phenoxy, which can be optionally substituted, is preferred.

Optionally substituted heterocyclyloxy includes an optionally substituted heterocyclyl-O group, wherein reference may be made to the following definition of heterocyclyl with respect to the definition of the heterocyclyl group. Preferred heterocyclyloxy groups include 5- and 6-membered heterocyclyloxy groups, among which pyridin-2-yloxy, pyridin-3-yloxy, thiophen-2-yloxy, thiophen-3-yloxy, furan-2-yloxy, furan-3-yloxy are preferred.

Optionally substituted alkenyl in the entire context of the invention preferably includes:

straight-chain or branched-chain alkenyl having 2 to 8 carbon atoms and cycloalkenyl having 3 to 8 carbon atoms, which can optionally be substituted by preferably 1 to 3 identical or different substituents, such as hydroxyl, halogen or alkoxy. Examples include: vinyl, 1-methylvinyl, allyl, 1-butenyl, isopropenyl, cyclopropenyl, cyclobutenyl, cyclopentenyl, cyclohexenyl. Vinyl or allyl are preferred.

Optionally substituted alkynyl in the entire context of the invention preferably includes:

straight-chain or branched-chain alkynyl having 2 to 8 carbon atoms and cycloalkynyl having 5 to 8 carbon atoms, which can optionally be substituted by preferably 1 to 3 identical or different substituents. With respect to the definition of the optionally substituted alkynyl, reference is made to the above definition of the optionally substituted alkyl having more than one carbon atom, wherein the optionally substituted alkynes include at least one C≡C triple bond. Examples include: ethynyl, propynyl, butynyl, pentynyl and variants thereof optionally substituted as defined above. Ethynyl and optionally substituted ethynyl are preferred.

Optionally substituted aryl in the entire context of the invention preferably includes:

aromatic hydrocarbon radicals having 6 to 14 carbon atoms (the carbon atoms of the possible substituents not being included), which can be mono- or bicyclic and which can be substituted by preferably 1 to 3 identical or different substituents chosen from hydroxyl, halogen, as defined above, cyano, optionally substituted amino, as defined below, mercapto, optionally substituted alkyl, as defined above, optionally substituted acyl, as defined below, and optionally substituted alkoxy, as defined above, optionally substituted aryloxy, as defined above, optionally substituted heterocyclyloxy, as defined above, optionally substituted aryl, as defined here, optionally substituted heterocyclyl, as defined below. Aromatic hydrocarbon radicals having 6 to 14 carbon atoms include, for example: phenyl, naphthyl, phenanthrenyl and anthracenyl, which can optionally be substituted once or several times by identical or different radicals. Phenyl and optionally substituted phenyl, such as, in particular, halogen-, cyano-, alkyl- and alkoxy-substituted phenyl, are preferred.

Examples of an aryl group substituted by alkyl preferably include: aryl, as described above, which is substituted by straight-chain or branched alkyl having 1 to 8, preferably 1 to 4 carbon atoms, as described above. Preferred alkylaryl is toluoyl and trifluoromethylbenzene (benzotrifluoride).

Examples of an aryl group substituted by halogen preferably include: aryl, as described above, which is substituted by halogen, as described above.

Examples of an aryl radical having 3 to 8, preferably 6 carbon atoms in the aromatic ring system and substituted by halogen include: a 2-fluorophenyl group, a 2-chlorophenyl group, a 2-bromophenyl group, a 3-fluorophenyl group, a 3-chlorophenyl group, a 3-bromophenyl group, a 4-fluorophenyl group, a 4-chlorophenyl group, a 4-bromophenyl group, a 2,4-di-fluorophenyl group, a 2,4-di-chlorophenyl group, a 2,4-di-bromophenyl group, a 3,5-di-fluorophenyl group, a 3,5-di-chlorophenyl group, a 3,5-di-bromophenyl group etc., a 2,4,6-tri-fluorophenyl group, a 2,4,6-tri-chlorophenyl group, a 2,4,6-tri-bromophenyl group etc. 2-Fluorophenyl, 2-chlorophenyl, 3-fluorophenyl, 3-chlorophenyl, 4-fluorophenyl and 4-chlorophenyl are preferred. 2-Fluorophenyl, 3-fluorophenyl and 4-fluorophenyl are particularly preferred, especially 4-fluorophenyl.

Examples of an aryl group substituted by cyano preferably include: aryl, as described above, which is substituted by 1 to 3 cyano radicals, such as, preferably, benzonitrile.

Examples of an aryl group substituted by hydroxyl preferably include: aryl, as described above, which is substituted by 1 to 3 hydroxyl radicals, such as, for example, 2-hydroxyphenyl, 3-hydroxyphenyl, 4-hydroxyphenyl, 2,4-di-hydroxyphenyl, 2,5-di-hydroxyphenyl, 2,6-di-hydroxyphenyl, 3,5-di-hydroxyphenyl, 3,6-di-hydroxyphenyl, 2,4,6-tri-hydroxyphenyl etc. 2-Hydroxyphenyl, 3-hydroxyphenyl and 2,4-di-hydroxyphenyl are preferred.

Examples of an aryl group substituted by alkoxy preferably include: aryl, as described above, which is substituted by 1 to 3 alkoxy radicals, as described above, such as, preferably, 2-methoxyphenyl, 3-methoxyphenyl, 4-methoxyphenyl, 2-ethoxyphenyl, 3-ethoxyphenyl, 4-ethoxyphenyl, 2-propyloxyphenyl, 3-propyloxyphenyl, 4-propyloxyphenyl, 2-i-propyloxyphenyl, 3-i-propyloxyphenyl, 4-i-propyloxyphenyl, 2,4-di-methoxyphenyl etc., 2-methoxyphenyl, 3-methoxyphenyl, 4-methoxyphenyl, 4-ethoxyphenyl and 4-i-propyloxyphenyl being particularly preferred.

Optionally substituted heterocyclyl in the entire context of the invention preferably includes: aliphatic, saturated or unsaturated heterocyclic 5- to 8-membered cyclic radicals which contain 1 to 3, preferably 1 to 2 hetero atoms chosen from N, O or S, and which can optionally be substituted, preferably by 1 to 3 substituents, wherein reference may be made to the definition of the possible substituents of alkyl with respect to possible substituents. 5- or 6-membered and 7-membered saturated or unsaturated, optionally substituted heterocyclic radicals are preferred, such as tetrahydrofuran-2-yl, tetrahydrofuran-3-yl, tetrahydro-thiophen-2-yl, tetrahydro-thiophen-3-yl, pyrrolidin-1-yl, pyrrolidin-2-yl, pyrrolidin-3-yl, morpholin-1-yl, morpholin-2-yl, morpholin-3-yl, morpholin-4-yl, piperidin-1-yl, piperidin-2-yl, piperidin-3-yl, piperidin-4-yl, piperazin-1-yl, piperazin-2-yl, tetrahydropyran-2-yl, tetrahydropyran-3-yl, tetrahydropyran-4-yl, azepan-2-yl, azepan-3-yl, azepan-4-yl, diazepan-1-yl, diazepan-2-yl, diazepan-3-yl, diazepan-5-yl, etc., which can optionally be fused with aromatic rings, etc. Morpholinyl, such as morpholin-4-yl, tetrahydrofuranyl, such as tetrahydrofuran-2-yl, pyrrolidine, such as pyrrolidin-1-yl:

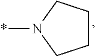

piperidinyl, such as piperidin-1-yl:

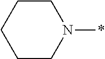

or piperazin-1-yl, such as

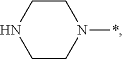

and diazepan, such as diazepan-1-yl:

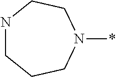

(* Bonding position to the base skeleton),
which can optionally be substituted on the 4-nitrogen atom, wherein with respect to possible substituents reference may be made to those of optionally substituted amino, are particularly preferred.

Optionally substituted heterocyclyl in the entire context of the invention moreover includes heteroaromatic hydrocarbon radicals having 4 to 9 ring carbon atoms, which additionally preferably contain 1 to 3 identical or different hetero atoms from the series S, O, N in the ring, and which therefore preferably form 5- to 12-membered heteroaromatic radicals, which can preferably be monocyclic, but also bicyclic. Preferred aromatic heterocyclic radicals include: pyridinyl, such as pyridin-2-yl, pyridin-3-yl and pyridin-4-yl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl. 5- or 6-membered aromatic heterocyclyls, such as e.g. pyridinyl, pyrimidyl, pyridazinyl, pyrazinyl, imidazolyl, furyl and thienyl, are preferred, and

pyrazinyl:

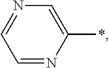

and pyridin-2-yl:

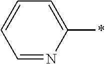

and pyridin-4-yl:

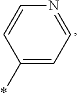

### Chien-2-yl:

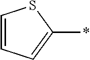

and imidazol-1-yl:

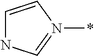

(* Bonding position to the base skeleton),
are particularly preferred.

The heterocyclyl radicals according to the invention can be substituted by preferably 1 to 3 identical or different substituents chosen, for example, from hydroxyl, halogen, as defined above, cyano, amino, as defined below, mercapto, alkyl, as defined above, acyl, as defined below, and alkoxy, as defined above, aryloxy, as defined above, heterocyclyloxy, as defined above, aryl, as defined above, heterocyclyl, as defined here.

Heterocyclyl preferably includes: tetrahydrofuranyl, pyrrolidinyl, morpholinyl, piperidinyl or tetrahydropyranyl, piperazinyl, diazepanyl, pyridinyl, pyridyl N-oxide, pyrimidyl, pyridazinyl, pyrazinyl, thienyl, furanyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, quinoxazolinyl. 5- or 6-membered heterocyclyls, such as e.g. morpholinyl or piperidinyl, such as, in particular, piperidin-1-yl, and pyrrolidine, such as pyrrolidin-1-yl, or piperazine, such as piperazin-1-yl, and 7-membered heterocyclyls, such as e.g. diazepan, such as diazepan-1-yl, and aromatic heterocyclyls, such as e.g. pyridyl, pyridyl N-oxide, pyrimidyl, pyridazinyl, pyrazinyl, imidazolyl, furanyl and thienyl, are preferred. Particularly preferred heterocyclyl includes: piperidine, such as piperidin-1-yl, piperidin-2-yl, piperidin-3-yl, piperidin-4-yl, piperidin-1-yl being very particularly preferred, and piperazine, piperazine-1-yl being very particularly preferred, pyrrolidine, pyrrolidin-1-yl being very particularly preferred, tetrahydrofuranyl, tetrahydrofuran-2-yl being very particularly preferred, diazepanyl, diazepan-1-yl being very particularly preferred, pyrazinyl, pyrazin-2-yl being very particularly preferred, pyridinyl, pyridin-2-yl and pyridin-4-yl being very particularly preferred, thienyl, thien-2-yl being very particularly preferred, imidazolyl, very particularly preferably imidazol-1-yl, and morpholinyl, such as, preferably, morpholin-4-yl.

Examples of a heterocyclyl group substituted by alkyl preferably include: heterocyclyl, as described above, which is substituted by optionally substituted straight-chain or branched alkyl having 1 to 8, preferably 1 to 4 carbon atoms, as described above. Preferred alkylheterocyclyl are methylpyrazinyl, ethylpyrazinyl, methylpiperidinyl and ethylpiperidinyl, methylpiperazinyl, ethylpiperazinyl, propylpiperazinyl, iso-propylpiperazinyl, butylpiperazinyl, cyclopentylpiperazinyl, cyclohexylpiperazinyl.

Examples of a heterocyclyl group substituted by alkoxyalkyl preferably include: heterocyclyl, as described above, which is substituted by alkoxy-substituted alkyl, as described above. Preferred alkoxyalkylheterocyclyl are methoxymethylpiperidinyl, methoxyethylpiperidinyl, methoxymethylpiperazinyl, methoxyethylpiperazinyl, methoxypropylpiperazinyl, ethoxymethylpiperazinyl, ethoxyethylpiperazinyl, methoxymethyldiazepanyl, methoxyethyldiazepanyl etc.

Examples of a heterocyclyl group substituted by heterocyclylalkyl preferably include: heterocyclyl, as described above, which is substituted by heterocyclyl-substituted alkyl, as described above. Preferred heterocyclylalkyl-substituted heterocyclyl are tetrahydrofuran-2-yl-methylpiperazinyl, tetrahydrofuran-2-yl-ethylpiperazinyl, imidazol-1-yl-methylpiperazinyl or imidazol-1-yl-ethylpiperazinyl.

Examples of a heterocyclyl group substituted by aminoalkyl preferably include: heterocyclyl, as described above, which is substituted by amino-substituted alkyl, as described above. Preferred aminoalkyl-substituted heterocyclyl are methyl-, ethyl-, di-methyl- or di-ethylaminomethylheterocyclyl or methyl-, ethyl-, di-methyl- or di-ethylaminoethylheterocyclyl, in particular di-ethylaminoethylpiperazinyl.

Very particularly preferred alkylheterocyclyl are methylpiperidinyl, methoxyethylpiperidinyl, methylpiperazinyl, iso-propylpiperazinyl, butylpiperazinyl, cyclopentylpiperazinyl, methoxyethylpiperazinyl, methoxypropylpiperazinyl, ethoxyethylpiperazinyl, methoxyethyldiazepanyl, tetrahydrofuran-2-yl-methylpiperazinyl, imidazol-1-yl-ethylpiperazinyl and di-ethylaminoethylpiperazinyl.

Examples of a heterocyclyl group substituted by hydroxyl preferably include: heterocyclyl, as described above, which is substituted by 1 to hydroxyl radicals, such as, for example, 3-hydroxypyridyl, 4-hydroxypyridyl 3-hydroxyfuryl, 2-hydroxypyrimidyl 5-hydroxypyrimidyl, 3-hydroxypyrrolyl, 3,5-di-hydroxypyridyl, 2,5-di-hydroxypyrimidyl etc.

Examples of a heterocyclyl group substituted by alkoxy preferably include:

heterocyclyl, as described above which is substituted by 1 to 3 alkoxy radicals, as described above, such as, preferably, 3-alkoxypyridyl, 4-alkoxypyridyl 3-alkoxyfuryl, 2-alkoxypyrimidyl 5-alkoxypyrimidyl, 3-alkoxypyrrolyl, 3-, 4- or 6-alkoxypyrazinyl, 3,5-di-alkoxypyridin-2-yl, 2,5-di-alkoxypyrimidyl, 2-, 3- or 4-alkoxypiperidinyl etc.

Examples of a heterocyclyl group substituted by acyl preferably include:

heterocyclyl, as described above, which is substituted by 1 to 3 acyl radicals, as described below, such as, preferably, tetrahydrofuran-2-oyl-piperazinyl or tetrahydrofuran-2-oyl-piperidinyl, tetrahydrofuran-2-oyl-piperazinyl being preferred.

Examples of a heterocyclyl group substituted by heterocyclyl preferably include:

heterocyclyl, as described above, which is substituted by 1 to 3 heterocyclyl radicals, as described above, such as, preferably, pyridin-2-yl-piperidinyl, pyridin-3-yl-piperidinyl, pyridin-2-yl-piperazinyl, pyridin-3-yl-piperazinyl, pyrazin-2-yl-piperidinyl, pyrazin-3-yl-piperidinyl, pyrazin-2-yl-piperazinyl, pyrazin-3-yl-piperazinyl etc. Pyridin-2-yl-piperazinyl and pyrazin-2-yl-piperazinyl are particularly preferred.

Optionally substituted acyl here and in the following includes: optionally substituted aliphatic acyl (alkanoyl=alkyl-CO—, wherein reference may be made to the above definition of optionally substituted alkyl with respect to the alkyl group), optionally substituted aromatic acyl (aroyl=aryl-CO—, wherein reference may be made to the above definition of optionally substituted aryl with respect to the aryl group) or heterocyclic acyl (heterocycloyl=heterocyclyl-CO—, wherein reference may be made to the above definition of optionally substituted heterocyclyl with respect to the heterocyclic group). Aliphatic acyl (alkyl-CO—) and heterocyclic acyl (heterocyclyl-CO—) are preferred.

In this context, optionally substituted aliphatic acyl (alkanoyl) preferably includes: C₁ to C₆ alkanoyl, such as formyl, acetyl, propionyl, iso-propionyl (i-propionyl), butyryl, Isobutyryl, valeryl, isovaleryl, pivaloyl, hexanoyl, cyclohexanoyl etc. Formyl, acetyl, iso-propionyl and cyclohexanoyl are particularly preferred.

Examples of substituted aliphatic acyl include, for example: optionally aryl- or heterocyclyl-substituted C₂ to C₆ alkanoyl, wherein reference may be made to the above definitions with respect to the definitions of aryl, heterocyclyl and C₂ to C₆ alkanoyl, such as phenylacetyl, thiophen-2-yl-acetyl, thiophen-3-yl-acetyl, furan-2-yl-acetyl, furan-3-yl-acetyl, 2- or 3-phenylpropionyl, 2- or 3-thiophen-2-yl-propionyl, 2- or 3-thiophen-3-yl-propionyl, 2- or 3-furan-2-yl-propionyl, 2- or 3-furan-3-yl-propionyl.

Optionally substituted aromatic acyl (aroyl) includes in particular: C₆ to C₁₀ aroyl, such as benzoyl, toluoyl, xyloyl, alkoxybenzoyl, such as methoxybenzoyl, ethoxybenzoyl etc. Methoxybenzoyl, such as 2-methoxybenzoyl:

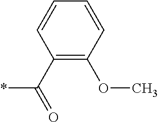

is preferred.

Optionally substituted heterocyclic acyl (heterocycloyl) includes in particular: C₆ to C₁₀ heterocycloyl, such as furanoyl, pyridinoyl, such as pyridin-2-oyl, pyrrolidinoyl, piperidinoyl, tetrahydrofuranoyl, such as tetrahydrofuran-2-oyl etc. Pyridin-2-oyl or pyridine-2-carbonyl:

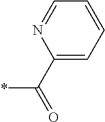

and tetrahydrofuran-2-oyl or tetrahydrofuran-2-carbonyl:

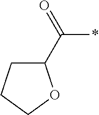

(*Bonding position to the base skeleton)
are preferred.

Optionally substituted amino in the entire context of the invention preferably includes: amino, mono- or dialkylamino, mono- or diarylamino, (N-alkyl)(N-aryl)amino, mono- or diheterocyclylamino, (N-alkyl)(N-heterocyclyl)amino, (N-aryl)(N-heterocyclyl)amino, mono- or diacylamino etc., wherein reference may be made to the corresponding above definition for optionally substituted alkyl, optionally substituted aryl, optionally substituted heterocyclyl and optionally substituted acyl with respect to alkyl, aryl, heterocyclyl and acyl.

Mono- or dialkylamino in this context includes in particular: straight-chain or branched mono- or dialkylamino having 1 to 8, preferably 1 to 6, more preferably 1 to 4 saturated or unsaturated carbon atoms, optionally substituted as described above, in each alkyl group, in particular methylamino, dimethylamino, ethylamino, diethylamino, cyclopentylamino or cyclohexylamino, wherein the alkyl groups can be substituted by preferably one substituent.

Mono- or diarylamino in this context includes in particular: mono- or diarylamino with 3- to 8-, preferably 5- to 6-membered aryl radicals which are optionally substituted as described above, in particular phenylamino or diphenylamino, wherein the aryl groups can be substituted by preferably one or two substituents.

(N-Alkyl)(N-aryl)amino describes in particular a substituted amino which is substituted in each case on the nitrogen atom by an alkyl radical and by an aryl radical, such as, in particular, (N-methyl)(N-phenyl)amino.

Mono- or diheterocyclylamino includes in particular: mono- or diheterocyclylamino with 3- to 8-, preferably 5- to 6-membered heterocyclyl radicals which are optionally substituted as described above, in particular pyridylamino or dipyridylamino.

(N-Alkyl)(N-heterocyclyl)amino describes in particular a substituted amino which is substituted in each case on the nitrogen atom by an alkyl radical and by a heterocyclyl radical, such as, in particular, (N-methyl)(N-pyrazin-2-yl)amino.

(N-Aryl)(N-heterocyclyl)amino describes in particular a substituted amino which is substituted in each case on the nitrogen atom by an aryl radical and by a heterocyclyl radical.

Mono- or diacylamino includes in particular a substituted amino which is substituted by one or two (optionally substituted) acyl radicals, as defined above, such as, in particular, acetylamino, iso-propionylamino, cyclohexanoylamino, benzoylamino etc.

Optionally substituted aminocarbonyl in the context of the entire invention represents optionally substituted amino-CO, wherein reference may be made to the above definition with respect to the definition of optionally substituted amino. Optionally substituted aminocarbonyl preferably represents optionally substituted carbamoyl (H₂NCO—), such as H₂NCO—, mono- or dialkylaminocarbonyl (H(alkyl)N—CO— or (alkyl)₂N—CO—), mono- or diarylaminocarbonyl (H(aryl)N—CO— or (aryl)₂N—CO—) or mono- or diheterocyclylaminocarbonyl (H(heterocyclyl)N—CO— or (heterocyclyl)₂N—CO—), wherein reference may be made to the above explanations for optionally substituted alkyl, aryl or heterocyclyl with respect to the definition of alkyl, aryl or heterocyclyl. Methylaminocarbonyl:

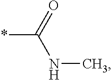

cyclohexylaminocarbonyl:

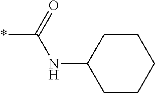

and phenylaminocarbonyl:

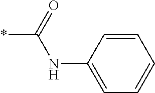

(*Bonding position to the base skeleton)
are preferred.

Optionally substituted aminosulfonyl in the context of the entire invention furthermore represents optionally substituted amino-SO₂—, wherein reference may be made to the above definition with respect to the definition of optionally substituted amino. Optionally substituted sulfamoyl (H₂N—SO₂—), such as sulfamoyl (H₂N—SO₂—) or mono- or dialkylaminosulfonyl (alkyl)₂N—SO₂, are preferred, wherein reference may be made to the above explanations for optionally substituted alkyl with respect to the definition of alkyl.

Optionally substituted alkyl-, aryl- or heterocyclylsulfonyl (R—SO₂—, wherein R is optionally substituted alkyl, aryl or heterocyclyl as defined above) furthermore preferably represents methylsulfonyl, ethylsulfonyl, phenylsulfonyl, tolylsulfonyl or benzylsulfonyl. Phenylsulfonyl is particularly preferred.

Optionally substituted alkoxycarbonyl (RO(O═)C—) includes the optionally substituted alkoxy mentioned above with respect to the definition of alkoxy, and includes, for example, methoxycarbonyl, ethoxycarbonyl etc. Ethoxycarbonyl is preferred.

Optionally substituted acyloxy (R—C(O═)—O—) includes the optionally substituted acyl mentioned above with respect to the definition of acyl.

In the general formula (Ia), the style of writing for the substituent(s) R⁷:

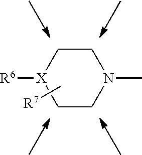

means that R⁷ denotes the four substituent positions (2, 3, 5 and 6) of the heterocyclic substituent identified with the arrows. In this context, R⁷ can be hydrogen, which means that the heterocyclic ring is not substituted at the positions mentioned, or R⁷ in the context of the definitions given in claim 3 can include one, two, three or four identical or different substitutions on the positions mentioned.

### Preferred Embodiments

In a preferred embodiment, the compound of the formula (I) has the following substituent definitions:

R¹ and R² are identical or different and are each chosen from the group consisting of:
hydrogen,
optionally substituted alkyl, optionally substituted aryl, and

optionally substituted heterocyclyl; or

R¹, and R² together form, together with the nitrogen atom to which they are bonded, a saturated or unsaturated, optionally substituted 5- to 6-membered ring which can optionally contain further hetero atoms;

R³ is chosen from the group consisting of:
optionally substituted aryl, and
optionally substituted heterocyclyl;

R⁴ and R⁵ are identical or different and are each chosen from the group consisting of:
hydrogen,
optionally substituted alkyl,
optionally substituted aryl, and
optionally substituted heterocyclyl; or

R⁴ and R⁵ together with the nitrogen atom to which they are bonded form a saturated or unsaturated, optionally substituted 5- to 6-membered ring which can optionally contain further hetero atoms.

In a further more preferred embodiment, the substituents R¹ and R² of the compound of the formula (I) together with the nitrogen atom to which they are bonded form a saturated, optionally substituted 6-membered ring, and thus form a preferred compound which corresponds to the formula (Ia):

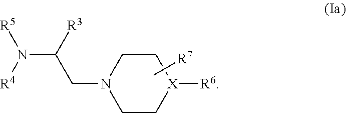

In this, the substituents have the following definitions:
X is chosen from: O, N or CH;
R⁶ is chosen from the group consisting of:
   hydrogen,
   optionally substituted alkyl,
   optionally substituted alkenyl,
   optionally substituted alkynyl,
   optionally substituted acyl,
   optionally substituted alkoxycarbonyl,
   optionally substituted amino,
   optionally substituted aminocarbonyl,
   optionally substituted alkyl-, aryl- or heterocyclylsulfonyl,
   optionally substituted aryl, and

optionally substituted heterocyclyl;
R⁷ is chosen from the group consisting of:
   hydrogen,
   hydroxyl,
   halogen,
   cyano,
   nitro,
   carboxyl,
   sulfonic acid radical (—SO₃H),
   optionally substituted amino,
   optionally substituted aminocarbonyl,
   optionally substituted aminosulfonyl,
   optionally substituted acyl,
   optionally substituted acyloxy,
   optionally substituted alkoxy,
   optionally substituted alkoxycarbonyl,
   optionally substituted alkyl,
   optionally substituted alkenyl,
   optionally substituted alkynyl,
   optionally substituted aryl, and
   optionally substituted heterocyclyl.

In a more preferred embodiment, the substituents in this have the following definitions:

X is chosen from: N or CH;

R⁶ is chosen from the group consisting of:
hydrogen,
optionally substituted alkyl,
optionally substituted alkenyl,
optionally substituted alkynyl,
optionally substituted acyl,
optionally substituted alkoxycarbonyl,
optionally substituted aryl, and
optionally substituted heterocyclyl;

R⁷ is chosen from the group consisting of:
hydrogen,
hydroxyl,
halogen,
cyano,
nitro,
carboxyl,
sulfonic acid radical (—SO₃H),
optionally substituted amino,
optionally substituted aminocarbonyl,
optionally substituted aminosulfonyl,
optionally substituted acyl,
optionally substituted acyloxy,
optionally substituted alkoxy,
optionally substituted alkoxycarbonyl,
optionally substituted alkyl,
optionally substituted alkenyl,
optionally substituted alkynyl,
optionally substituted aryl, and
optionally substituted heterocyclyl;

and R³, R⁴ and R⁵ have one of the meanings defined above.

In a further more preferred embodiment, the compound of the formula (Ia) has the following substituent definitions:

X is chosen from: N or CH;
R⁶ is chosen from the group consisting of:
   hydrogen,
   optionally substituted alkyl,
   optionally substituted acyl,
   optionally substituted alkoxycarbonyl,
   optionally substituted amino,
   optionally substituted aminocarbonyl,
   optionally substituted alkyl-, aryl- or heterocyclylsulfonyl,
   optionally substituted aryl, and
   optionally substituted heterocyclyl;
R⁷ is chosen from the group consisting of:
   hydrogen,
   halogen,
   optionally substituted amino,
   optionally substituted acyl,
   optionally substituted alkoxy,
   optionally substituted alkyl,
   optionally substituted aryl, and
   optionally substituted heterocyclyl;
R³ is chosen from the group consisting of:
   optionally substituted aryl, and
   optionally substituted heterocyclyl;
R⁴ and R⁵ are identical or different and are each chosen from the group consisting of:
   hydrogen,
   optionally substituted alkyl,
   optionally substituted aryl, and
   optionally substituted heterocyclyl; or
R⁴ and R⁵ together with the nitrogen atom to which they are bonded form a saturated or unsaturated, optionally substituted 5- to 7-membered ring which can optionally contain further hetero atoms;
or pharmaceutically acceptable salts thereof.

In a further more preferred embodiment, the substituents have the following definitions:

X is chosen from: N or CH;

R⁶ is chosen from the group consisting of:
hydrogen,
optionally substituted alkyl,
optionally substituted acyl,
optionally substituted aryl, and
optionally substituted heterocyclyl;

R⁷ is chosen from the group consisting of:
hydrogen,
halogen,
optionally substituted amino,
optionally substituted acyl,
optionally substituted alkoxy,
optionally substituted alkyl,
optionally substituted aryl, and
optionally substituted heterocyclyl;

R³ is chosen from the group consisting of:
optionally substituted aryl, and
optionally substituted heterocyclyl;

R⁴ and R⁵ are identical or different and are each chosen from the group consisting of:
hydrogen,
optionally substituted alkyl,
optionally substituted aryl, and
optionally substituted heterocyclyl; or

R⁴ and R⁵ together with the nitrogen atom to which they are bonded form a saturated or unsaturated, optionally substituted 5- to 6-membered ring which can optionally contain further hetero atoms.

In a further more preferred embodiment, the compound of the formula (Ia) has the following substituent definitions:
X has the meaning N;
R⁶ is chosen from the group consisting of:
   optionally substituted acyl,
   optionally substituted alkyl-, aryl- or heterocyclylsulfonyl, optionally substituted aryl, and
   optionally substituted heterocyclyl;
R⁷ is hydrogen;
R³ is chosen from the group consisting of:
   optionally substituted aryl, and
   optionally substituted heterocyclyl;
R⁴ and R⁵ are identical or different and are each chosen from the group consisting of:
   hydrogen or
   optionally substituted alkyl, or
R⁴ and R⁵ together with the nitrogen atom to which they are bonded form a saturated or unsaturated, optionally substituted 6-membered ring which can optionally contain further hetero atoms.
or pharmaceutically acceptable salts thereof.

In a further more preferred embodiment, the substituents have the following definitions:

X has the meaning N;
R⁶ is chosen from the group consisting of:
optionally substituted acyl,
optionally substituted aryl, and
optionally substituted heterocyclyl;

R⁷ is hydrogen;

R³ is chosen from the group consisting of:
optionally substituted aryl, and
optionally substituted heterocyclyl;

R⁴ and R⁵ are identical or different and are each chosen from the group consisting of:
hydrogen or
optionally substituted alkyl.

In a further more preferred embodiment, the compound of the formula (Ia) has the following substituent definitions:
X has the meaning CH; and
R⁶ is chosen from the group consisting of:
   hydrogen,
   optionally substituted alkyl,
   optionally substituted alkoxycarbonyl,
   optionally substituted amino,
   optionally substituted aminocarbonyl,
   optionally substituted aryl, and
   optionally substituted heterocyclyl;
R⁷ is hydrogen;
R³ is chosen from the group consisting of:
   optionally substituted aryl, and
   optionally substituted heterocyclyl;
R⁴ and R⁵ are identical or different and are each chosen from the group consisting of:
   hydrogen,
   optionally substituted alkyl; or
R⁴ and R⁵ together with the nitrogen atom to which they are bonded form a saturated or unsaturated, optionally substituted 6- or 7-membered ring which can optionally contain further hetero atoms;
or pharmaceutically acceptable salts thereof.

In a further more preferred embodiment, the substituents have the following definitions:

X has the meaning CH; and

R⁶ is chosen from the group consisting of:
optionally substituted aryl, and
optionally substituted heterocyclyl;

R⁷ is hydrogen;

R³ is chosen from the group consisting of:
optionally substituted aryl, and
optionally substituted heterocyclyl;

R⁴ and R⁵ are identical or different and are each chosen from the group consisting of:
hydrogen,
optionally substituted alkyl; or

R⁴ and R⁵ together with the nitrogen atom to which they are bonded form a saturated or unsaturated, optionally substituted 6-membered ring which can optionally contain further hetero atoms.

In preferred embodiments of the general formula (I) or (Ia), the individual substituents each have the following definitions:
1. R¹ and R² together with the nitrogen atom to which they are bonded form an optionally substituted, saturated or unsaturated 6-membered ring, which can optionally contain one to 3 further hetero atoms, such as N, O or S, and which optionally contains a substituent R⁶ in the para-position to the commonly bonded nitrogen atom, wherein R⁶ has one of the meanings defined above and wherein the substituents R³, R⁴, R⁵ and R⁷ have the meaning of one of the embodiments described above. Preferably, such a 6-membered ring which is formed from R¹ and R² with the common nitrogen atom to which they are bonded contains no or one further hetero atom, which is optionally preferably a nitrogen atom or an oxygen atom, particularly preferably a nitrogen atom, and which is particularly preferably positioned in the para-position to the commonly bonded nitrogen atom, as shown in formula (Ia). It is furthermore preferable for a substituent R⁶ optionally bonded in the para-position to be chosen from the group which includes hydrogen, optionally substituted alkyl, optionally substituted acyl, optionally substituted alkoxycarbonyl, optionally substituted amino, optionally substituted aminocarbonyl, optionally substituted alkyl-, aryl- or heterocyclylsulfonyl, optionally substituted aryl and optionally substituted heterocyclyl, including cycloaliphatic heterocyclic groups and heteroaromatic groups.
   If such a 6-membered ring which is formed from R¹ and R² with the common nitrogen atom to which they are bonded contains no further hetero atom in the para-position to the commonly bonded nitrogen atom, as shown in formula (Ia), it is furthermore preferable for a substituent R⁶ optionally bonded in the para-position to be chosen from the group which includes hydrogen, optionally substituted alkyl, optionally substituted alkoxycarbonyl, optionally substituted amino, optionally substituted aminocarbonyl, optionally substituted aryl and optionally substituted heterocyclyl, including cycloaliphatic heterocyclic groups and heteroaromatic groups. In this context, R⁶ is preferably chosen from the group which includes optionally substituted aryl and optionally substituted heterocyclyl, including cycloaliphatic heterocyclic groups and heteroaromatic groups.
   If such a 6-membered ring which is formed from R¹ and R² with the common nitrogen atom to which they are bonded contains a further hetero atom, which is optionally preferably a nitrogen atom, which is particularly preferably positioned in the para-position to the commonly bonded nitrogen atom, as shown in formula (Ia), it is furthermore preferable for a substituent R⁶ optionally bonded in the para-position to be chosen from the group which includes optionally substituted acyl, optionally substituted alkyl-, aryl- or heterocyclylsulfonyl, optionally substituted aryl and optionally substituted heterocyclyl, including cycloaliphatic heterocyclic groups and heteroaromatic groups. Preferably, R⁶ is chosen from the group which includes optionally substituted acyl, optionally substituted aryl and optionally substituted heterocyclyl, including cycloaliphatic heterocyclic groups and heteroaromatic groups.
2. R³ is optionally substituted aryl or optionally substituted heterocyclyl and the substituents R¹, R², R⁴, R⁵ and optionally R⁶ and R⁷ have the meaning of one of the embodiments described above.
3. R⁴ and R⁵ are identical and denote hydrogen, or one of the radicals R⁴ or R⁵ is hydrogen, and the other radical of the radicals R⁴ or R⁵ is optionally substituted alkyl.
   Preferably, one of the radicals R⁴ or R⁵ is hydrogen and the other radical of the radicals R⁴ or R⁵ is optionally substituted alkyl and the substituents R¹, R², R³ and optionally R⁶ and R⁷ have the meaning of one of the embodiments described above.
4. R⁴ and R⁵ together with the nitrogen atom to which they are bonded form a saturated or unsaturated, optionally substituted 6- or 7-membered, preferably a 6-membered ring, which can optionally contain further hetero atoms and the substituents R¹, R², R³ and optionally R⁶ and R⁷ have the meaning of one of the embodiments described above.
5. R¹ and R², R⁴, R⁵ and optionally R⁶ and R⁷ have the meaning of one of the embodiments described above.

In preferred embodiments of the general formula (I) or (Ia), the individual substituents each have the following definitions:

R¹ and R² denote hydrogen, optionally substituted alkyl, such as, in particular, methyl and aminoalkyl, such as, preferably, (N-methyl)(N-pyrazin-2-yl)aminoethyl, or R¹ and R² preferably form, together with the nitrogen atom to which they are bonded, a saturated 6- or 7-membered, preferably a 6-membered ring, which can optionally contain further hetero atoms and which optionally contains a substituent R⁶ in the para-position to the commonly bonded nitrogen atom, and therefore forms compounds according to the general formula (Ia), wherein

X has the meaning O, N or CH, preferably N or CH;

R⁶ is preferably chosen from the group consisting of:
hydrogen,
optionally substituted alkyl, in particular cycloalkyl, such as, preferably, cyclohexyl,
optionally substituted acyl, in particular alkanoyl, such as, preferably, acetyl, and/or aroyl, such as, preferably, 2-methoxybenzoyl, and/or heterocycloyl, such as, preferably, tetrahydrofuran-2-oyl and pyridin-2-oyl,
optionally substituted alkoxycarbonyl, such as, preferably, ethoxycarbonyl,
optionally substituted amino, in particular alkylamino, such as, preferably, cyclopentylamino, and/or dialkylamino, such as, preferably, dimethylamino and diethylamino, and/or arylamino, such as, preferably, phenylamino, and/or acylamino, such as alkanoylamino, such as, preferably, acetylamino and isopropionylamino and cyclohexanoylamino, and aroylamino, such as, preferably, benzoylamino,
optionally substituted aminocarbonyl, such as, preferably, methylaminocarbonyl, cyclohexylaminocarbonyl, phenylaminocarbonyl,
optionally substituted alkyl-, aryl- or heterocyclylsulfonyl, in particular arylsulfonyl, such as, preferably, phenylsulfonyl,
optionally substituted aryl, such as, preferably, phenyl, and
optionally substituted heterocyclyl, in particular optionally substituted saturated heterocyclyl, such as, preferably, piperidin-1-yl, piperazin-1-yl, pyrrolidin-1-yl, morpholinyl or optionally substituted aromatic heterocyclyl, such as, preferably, pyrazinyl and pyridinyl; and

R⁷ denotes hydrogen,

R³ denotes optionally substituted aryl, such as, in particular, phenyl or halogen-substituted aryl, such as, in particular, 4-fluorophenyl,

R⁴ and R⁵ are identical or different and are each chosen from the group consisting of:
hydrogen,
optionally substituted alkyl, such as, in particular
   aryl-substituted alkyl, such as straight-chain C₁-, C₂- or C₃-alkyl, which can be substituted by an optionally substituted aryl group, such as an alkoxy-substituted aryl group, such as particularly preferably phenylmethyl and para-methoxyphenylmethyl, or
   heterocyclyl-substituted alkyl, such as straight-chain C₁-, C₂- or C₃-alkyl, which is substituted by an optionally substituted heterocyclyl group, such as particularly preferably 2-morpholin-4-yl-ethyl, thiophen-2-yl-methyl or pyridin-2-yl-methyl,
   alkoxy-substituted alkyl, such as straight-chain C₁-, C₂- or C₃-alkyl, which is substituted by an optionally substituted alkoxy group, such as particularly preferably 2-methoxyethyl, or

R⁴ and R⁵ together with the nitrogen atom to which they are bonded form a saturated, optionally substituted 6- or 7-membered, preferably 6-membered saturated ring, which preferably contains a further hetero atom, such as, in particular, a 6- or 7-membered, preferably 6-membered ring substituted by an optionally substituted alkyl, acyl or heterocyclyl group, such as, in particular
a 6-membered ring substituted by a straight-chain or branched C₁, C₂, C₃ or C₄ alkyl or a cycloalkyl-substituted 6-membered ring, such as a 6-membered ring substituted by straight-chain butyl, isopropyl or cyclopentyl,
a 6-membered ring substituted by an alkoxy-substituted alkyl groups, such as a 6-membered ring substituted by 2-methoxyethyl, 3-methoxypropyl or 2-ethoxyethyl, or a 2-methoxyethyl-substituted 7-membered ring,
a 6-membered ring substituted by an amino-substituted alkyl group, such as a 6-membered ring substituted by dialkylaminoalkyl, in particular a diethylaminoethyl-substituted 6-membered ring,
a 6-membered ring substituted by a heterocyclyl-substituted alkyl group, such as, in particular, a tetrahydrofuran-2-ylmethyl- or an imidazol-1-yl-ethyl-substituted 6-membered ring,
a 6-membered ring substituted by an acyl group, such as a 6-membered ring substituted by a heterocycloyl group, in particular a tetrahydrofuran-2-oyl-substituted 6-membered ring,
a 6-membered ring substituted by a heterocyclyl group, such as a 6-membered ring substituted by an aromatic heterocycloyl group, in particular a pyridin-2-yl- and pyrazin-2-yl-substituted 6-membered ring,
and R⁴ and R⁵ particularly preferably form, together with the nitrogen atom to which they are bonded, the following radicals:
   a 4-(2-methoxyethyl)-piperidinyl radical:

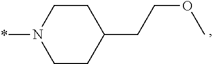

a 4-isopropyl-piperazinyl radical:

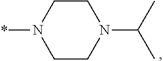

a 4-(4-butyl)-piperazinyl radical:

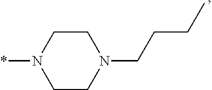

a cyclopentyl-piperazinyl radical:

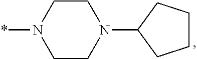

a 4-(2-methoxyethyl)-piperazinyl radical:

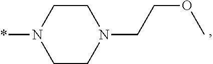

a 4-(3-methoxypropyl)-piperazinyl radical:

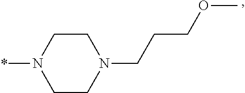

a 4-(2-ethoxyethyl)-piperazinyl radical:

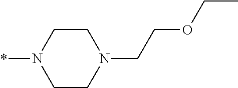

a diethylaminoethyl-piperazinyl radical:

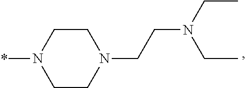

a tetrahydrofuran-2-yl-methyl-piperazinyl radical:

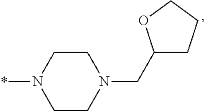

an imidazol-1-yl-ethyl-piperazinyl radical:

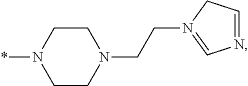

a tetrahydrofuran-2-oyl-piperazinyl radical:

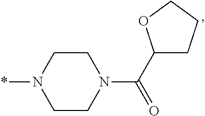

a pyridin-2-yl-piperazinyl radical:

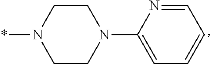

a pyrazin-2-yl-piperazinyl radical:

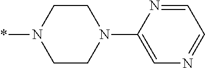

and
a 4-(2-methoxyethyl)-diazepanyl radical:

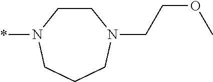

(*Bonding position to the base skeleton).

Particularly preferred compounds of the general formula (I) are shown in the following table:

| | | (I) | | | | | | | |
|---|---|---|---|---|---|---|---|---|---|
| Ex- | | 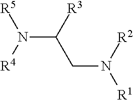 | | | | | | | |
| ample | Compound | R¹ | R² | X | R⁶ | R⁷ | R³ | R⁴ | R⁵ |
| 1 | 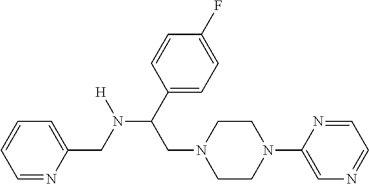 | 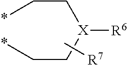 | | N | 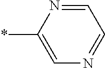 | H | 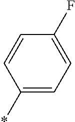 | 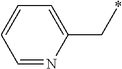 | H |
| 2 | 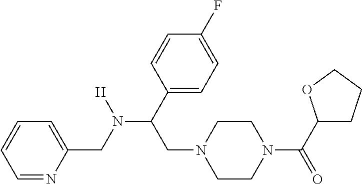 | 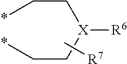 | | N | 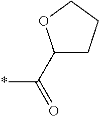 | H | 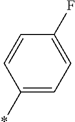 | 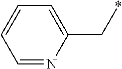 | H |
| 3 | 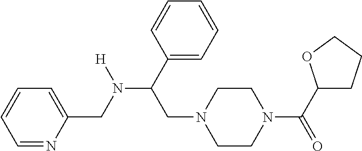 | 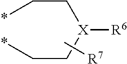 | | N | 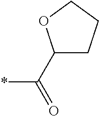 | H | 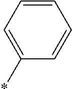 | 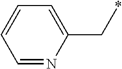 | H |
| 4 | 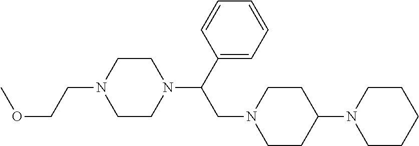 | 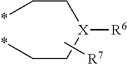 | | CH | 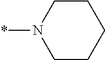 | H | 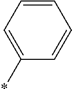 | 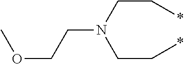 | |
| 5 | 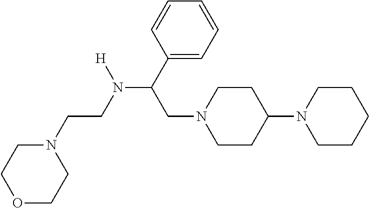 | 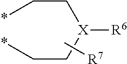 | | CH | 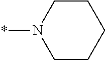 | H | 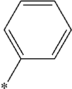 | 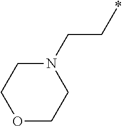 | H |
| 6 | 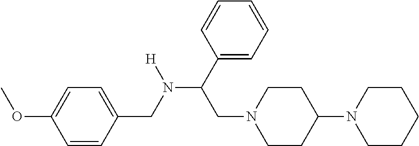 | 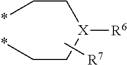 | | CH | 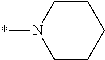 | H | 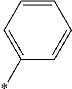 | 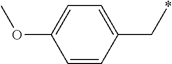 | H |
| 7 | 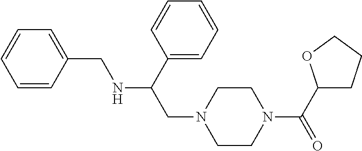 | 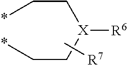 | | N | 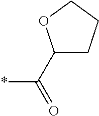 | H | 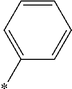 | 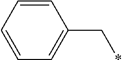 | H |
| 8 | 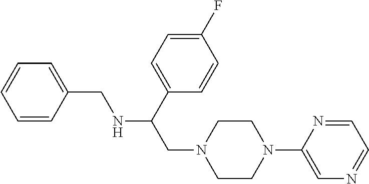 | 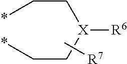 | | N | 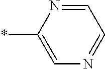 | H | 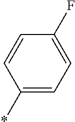 | 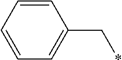 | H |
| 9 | 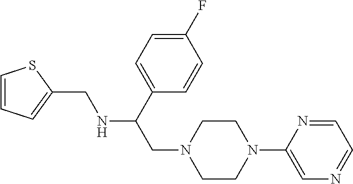 | 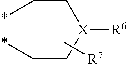 | | N | 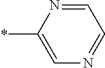 | H | 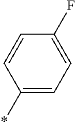 | 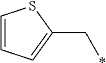 | H |
| 10 | 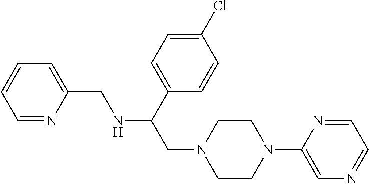 | 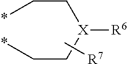 | | N | 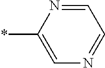 | H | 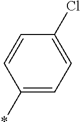 | 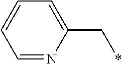 | H |
| 11 | 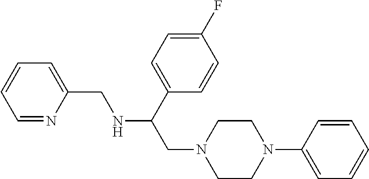 | 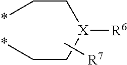 | | N | 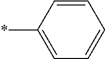 | H | 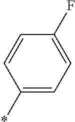 | 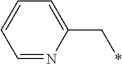 | H |
| 12 | 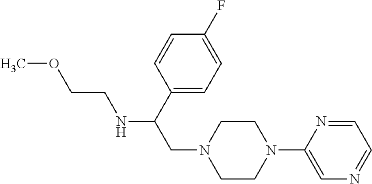 | 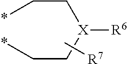 | | N | 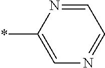 | H | 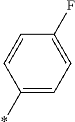 | 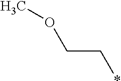 | H |
| 13 | 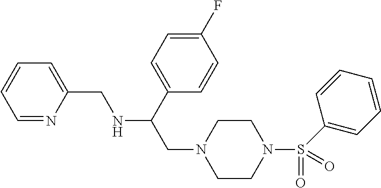 | 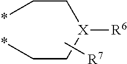 | | N | 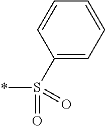 | H | 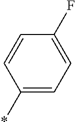 | 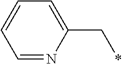 | H |
| 14 | 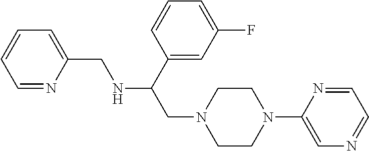 | 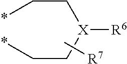 | | N | 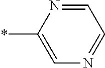 | H | 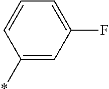 | 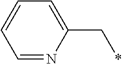 | H |
| 15 | 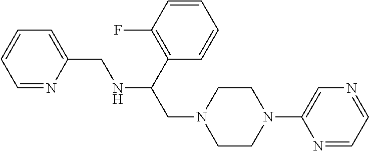 | 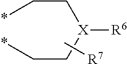 | | N | 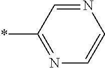 | H | 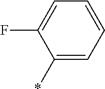 | 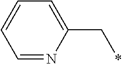 | H |
| 16 | 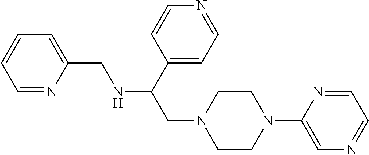 | 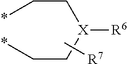 | | N | 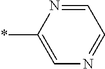 | H | 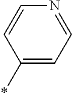 | 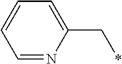 | H |
| 17 | 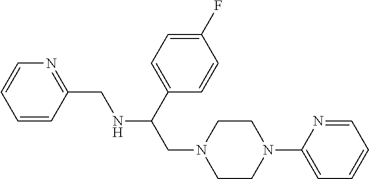 | 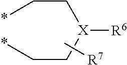 | | N | 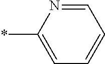 | H | 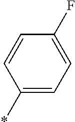 | 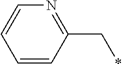 | H |
| 18 | 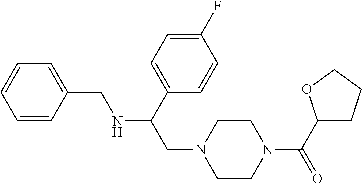 | 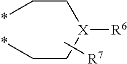 | | N | 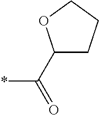 | H | 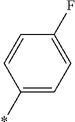 | 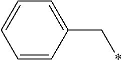 | H |
| 19 | 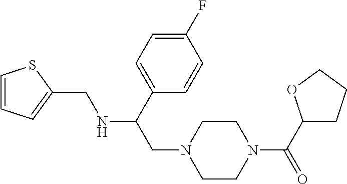 | 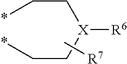 | | N | 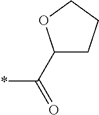 | H | 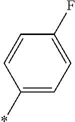 | 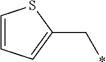 | H |
| 20 | 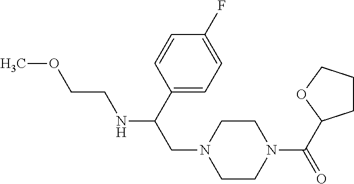 | 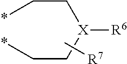 | | N | 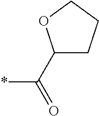 | H | 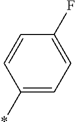 | 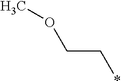 | H |
| 21 | 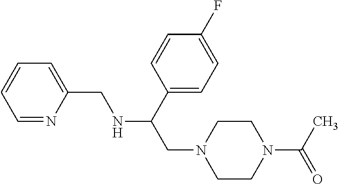 | 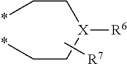 | | N | 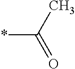 | H | 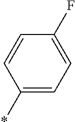 | 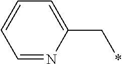 | H |
| 22 | 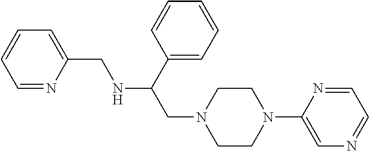 | 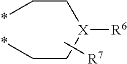 | | N | 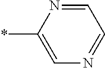 | H | 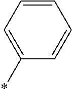 | 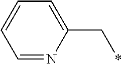 | H |
| 23 | 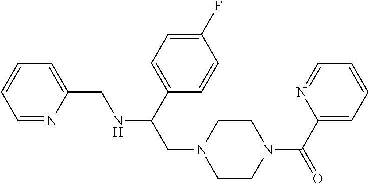 | 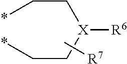 | | N | 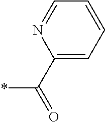 | H | 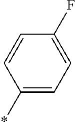 | 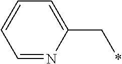 | H |
| 24 | 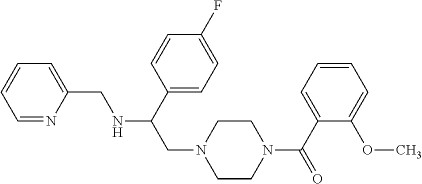 | 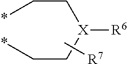 | | N | 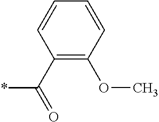 | H | 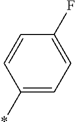 | 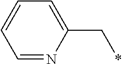 | H |
| 25 | 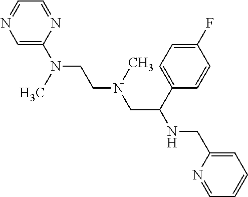 | CH₃ | 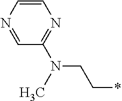 | — | — | — | 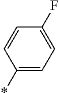 | 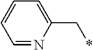 | H |

| 26 | 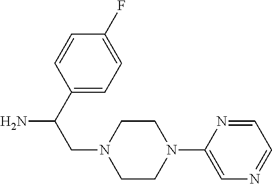 | 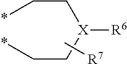 | N | 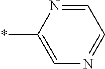 | H | 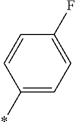 | H | H |
|---|---|---|---|---|---|---|---|---|
| 27 | 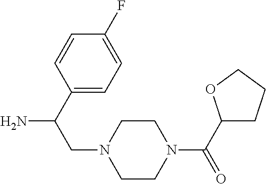 | 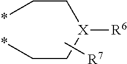 | N | 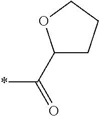 | H | 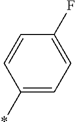 | H | H |
| 28 | 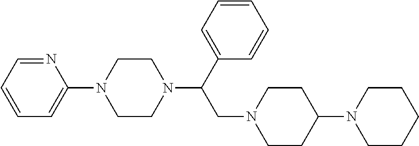 | 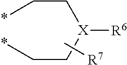 | CH | 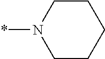 | H | 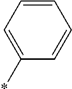 | 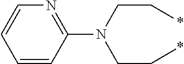 | |
| 29 | 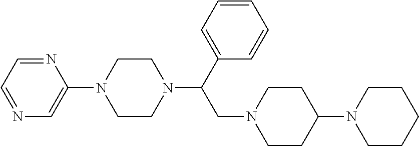 | 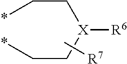 | CH | 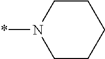 | H | 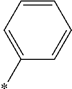 | 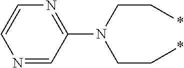 | |
| 30 | 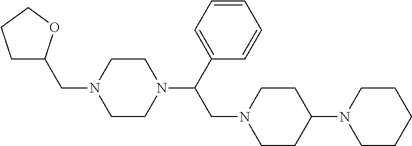 | 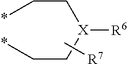 | CH | 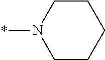 | H | 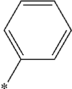 | 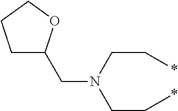 | |
| 31 | 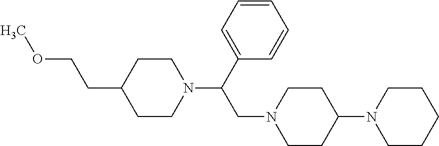 | 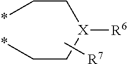 | CH | 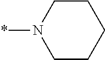 | H | 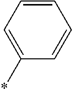 | 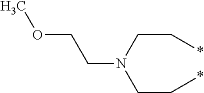 | |
| 32 | 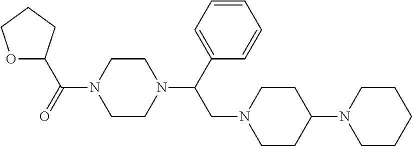 | 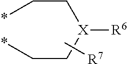 | CH | 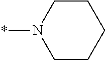 | H | 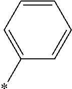 | 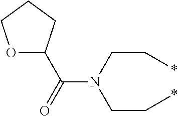 | |
| 33 | 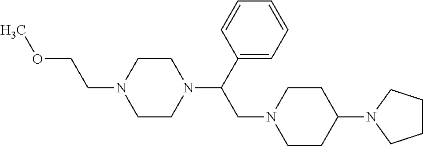 | 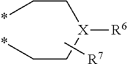 | CH | 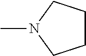 | H | 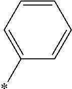 | 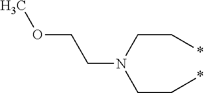 | |
| 34 | 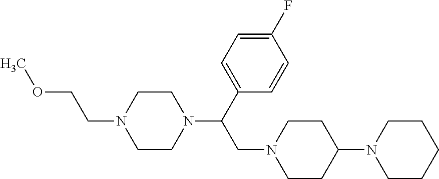 | 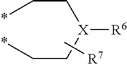 | CH | 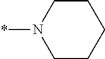 | H | 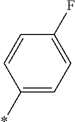 | 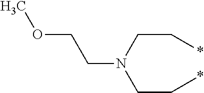 | |
| 35 | 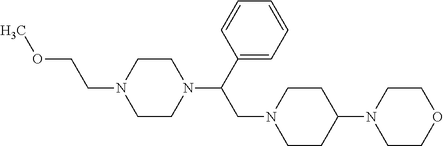 | 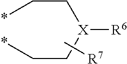 | CH | 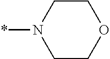 | H | 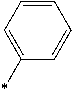 | 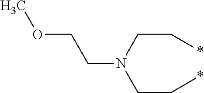 | |
| 36 | 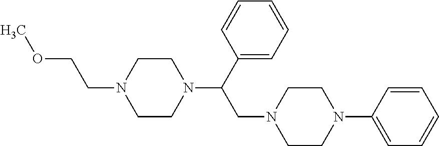 | 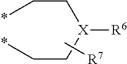 | N | 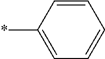 | H | 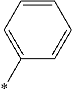 | 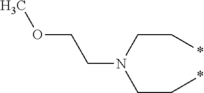 | |
| 37 | 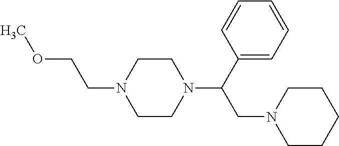 | 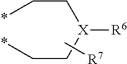 | CH | H | H | 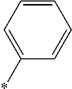 | 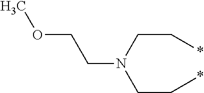 | |
| 38 | 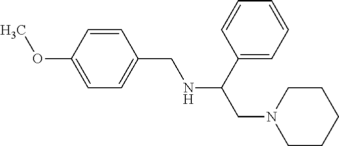 | 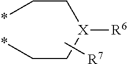 | CH | H | H | 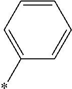 | 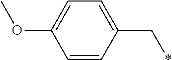 | H |
| 39 | 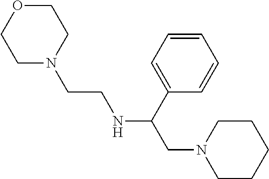 | 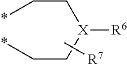 | CH | H | H | 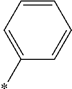 | 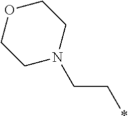 | H |

| | | | | | | | |
|---|---|---|---|---|---|---|---|
| 40 | 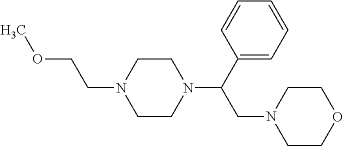 | 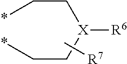 | O | — | H | 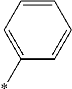 | 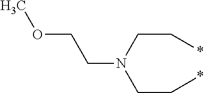 |
| 41 | 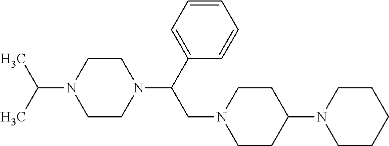 | 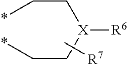 | CH | 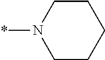 | H | 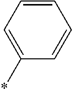 | 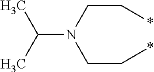 |
| 42 | 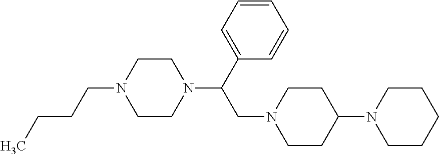 | 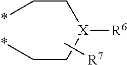 | CH | 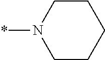 | H | 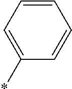 | 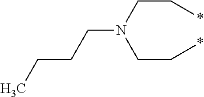 |
| 43 | 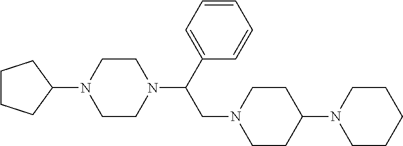 | 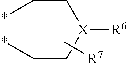 | CH | 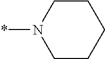 | H | 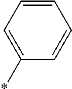 | 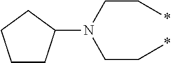 |
| 44 | 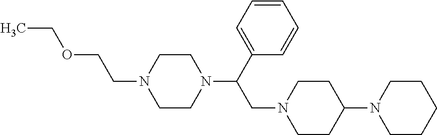 | 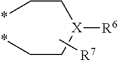 | CH | 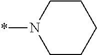 | H | 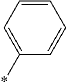 | 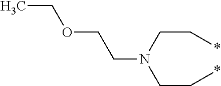 |
| 45 | 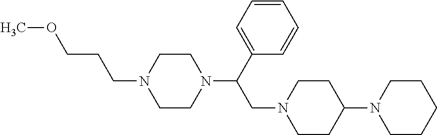 | 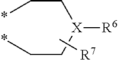 | CH | 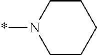 | H | 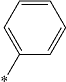 | 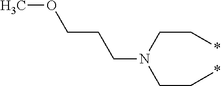 |
| 46 | 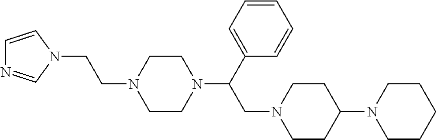 | 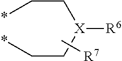 | CH | 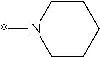 | H | 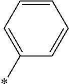 | 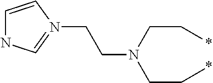 |
| 47 | 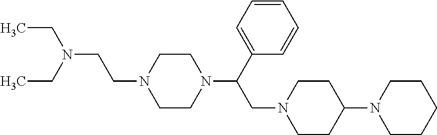 | 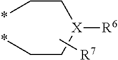 | CH | 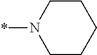 | H | 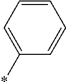 | 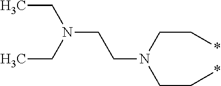 |
| 48 | 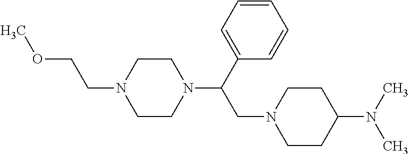 | 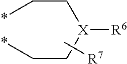 | CH | 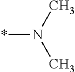 | H | 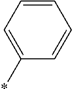 | 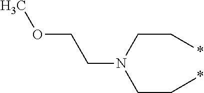 |
| 49 | 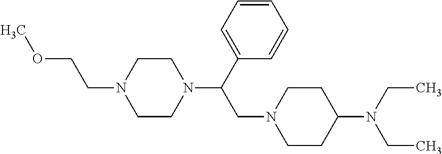 | 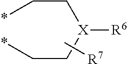 | CH | 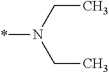 | H | 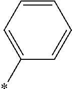 | 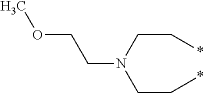 |
| 50 | 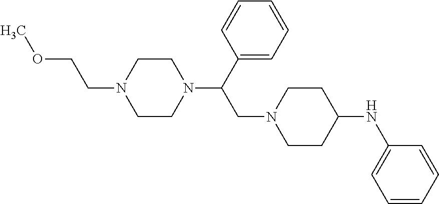 | 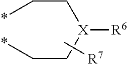 | CH | 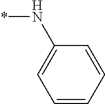 | H | 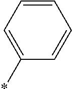 | 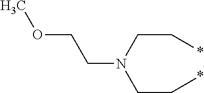 |
| 51 | 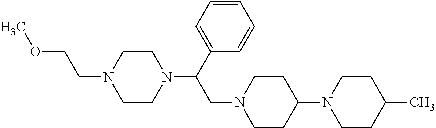 | 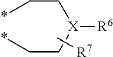 | CH | 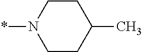 | H | 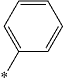 | 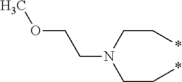 |
| 52 | 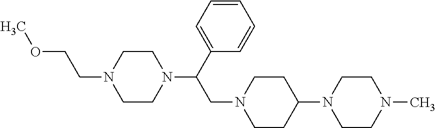 | 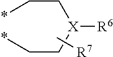 | CH | 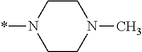 | H | 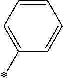 | 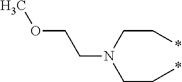 |
| 53 | 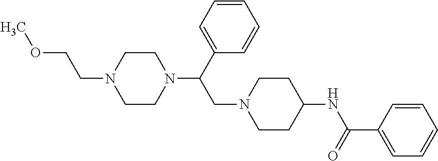 | 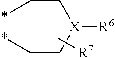 | CH | 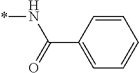 | H | 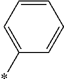 | 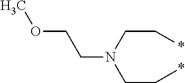 |
| 54 | 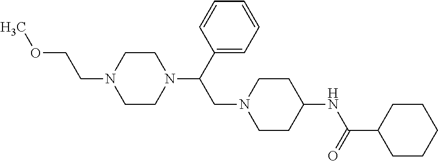 | 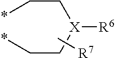 | CH | 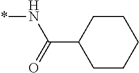 | H | 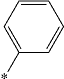 | 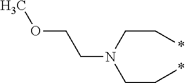 |
| 55 | 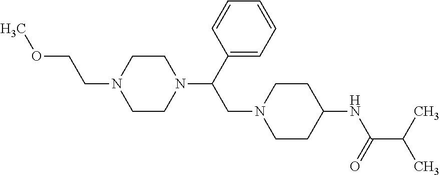 | 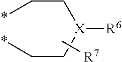 | CH | 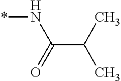 | H | 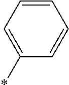 | 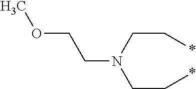 |
| 56 | 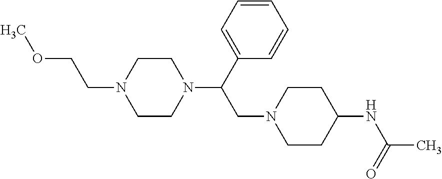 | 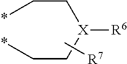 | CH | 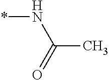 | H | 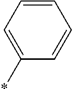 | 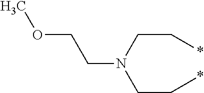 |
| 57 | 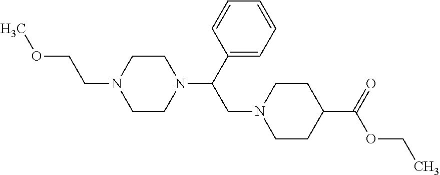 | 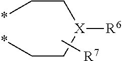 | CH | 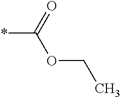 | H | 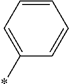 | 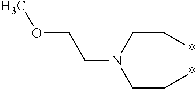 |
| 58 | 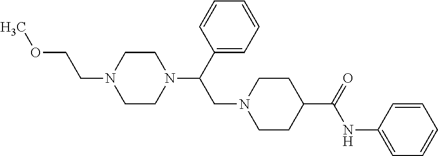 | 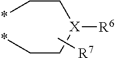 | CH | 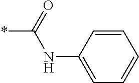 | H | 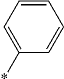 | 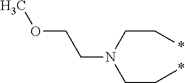 |
| 59 | 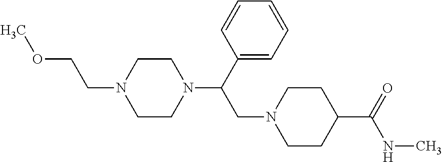 | 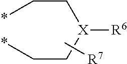 | CH | 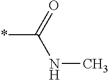 | H | 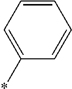 | 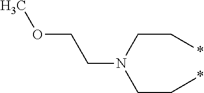 |
| 60 | 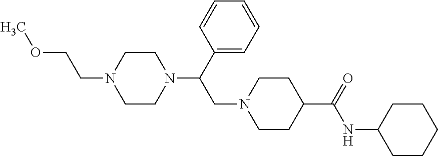 | 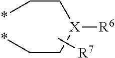 | CH | 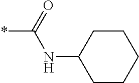 | H | 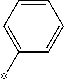 | 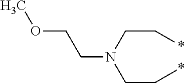 |
| 61 | 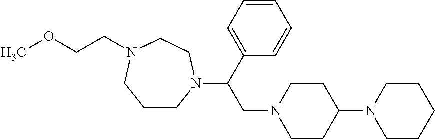 | 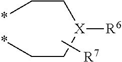 | CH | 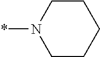 | H | 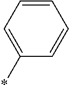 | 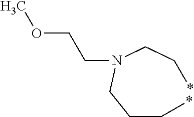 |
| 62 | 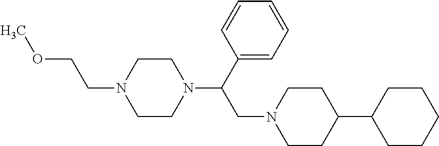 | 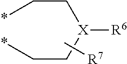 | CH | 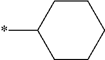 | H | 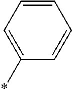 | 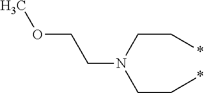 |
| 63 | 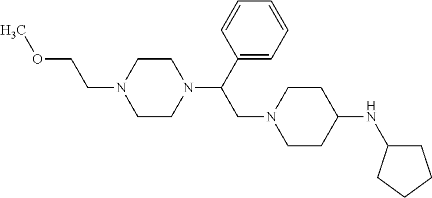 | 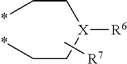 | CH | 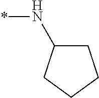 | H | 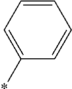 | 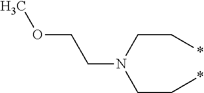 |
| 64 | 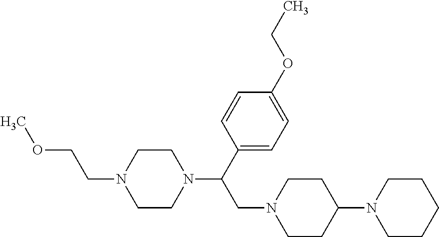 | 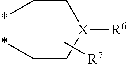 | CH | 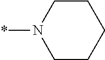 | H | 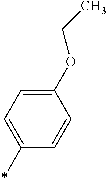 | 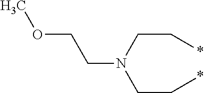 |
| 65 | 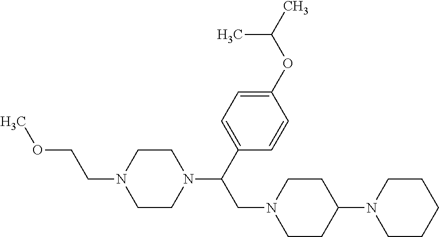 | 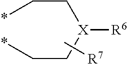 | CH | 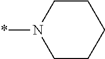 | H | 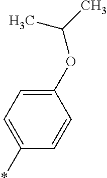 | 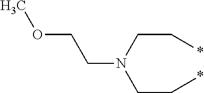 |
| 66 | 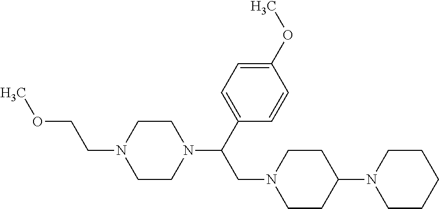 | 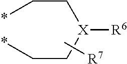 | CH | 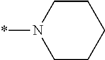 | H | 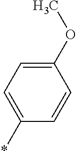 | 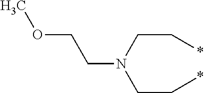 |
| 67 | 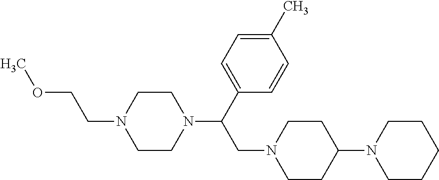 | 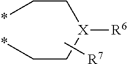 | CH | 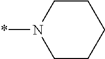 | H | 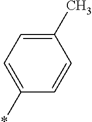 | 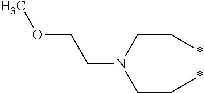 |
| 68 | 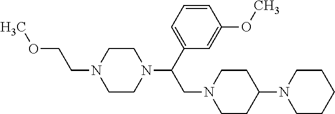 | 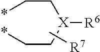 | CH | 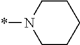 | H | 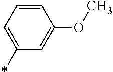 | 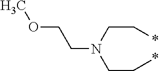 |
| 69 | 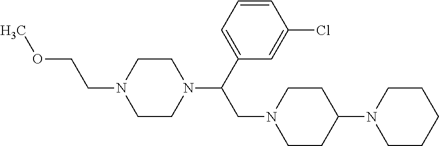 | 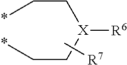 | CH | 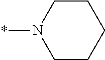 | H | 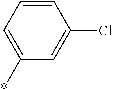 | 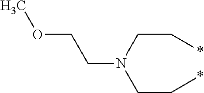 |
| 70 | 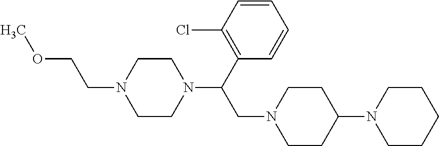 | 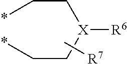 | CH | 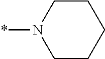 | H | 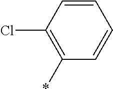 | 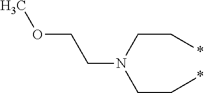 |
| 71 | 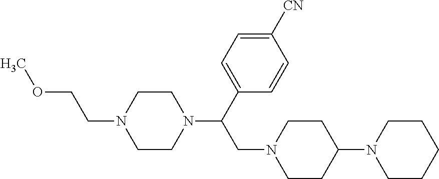 | 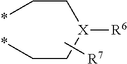 | CH | 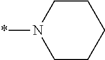 | H | 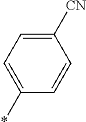 | 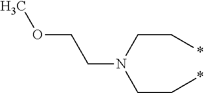 |
| 72 | 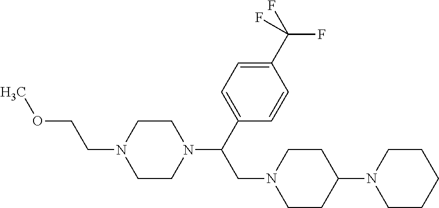 | 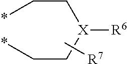 | CH | 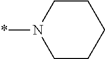 | H | 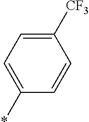 | 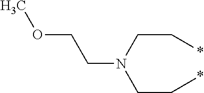 |
| 73 | 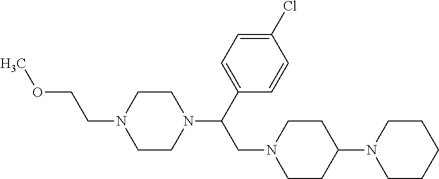 | 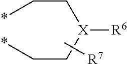 | CH | 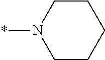 | H | 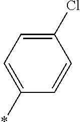 | 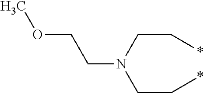 |

(* Bonding position to the base skeleton)

and pharmaceutically acceptable salts thereof.

In particular, the present invention also relates to novel compounds of the general formula (I) with the meaning of the substituents as described above, one or more of the following compounds being excluded: a)

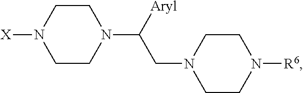

wherein R⁶ has the meaning of optionally substituted alkyl, as defined above, and wherein X represents hydrogen or optionally a further substituent. In particular
b) compounds of the general formula

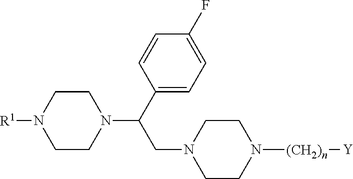

are excluded,
with the meaning of:
A=N (nitrogen),
n=1-8,
R¹=iso-propyl and
—(CH₂)ₙ—Y has one of the following meanings:

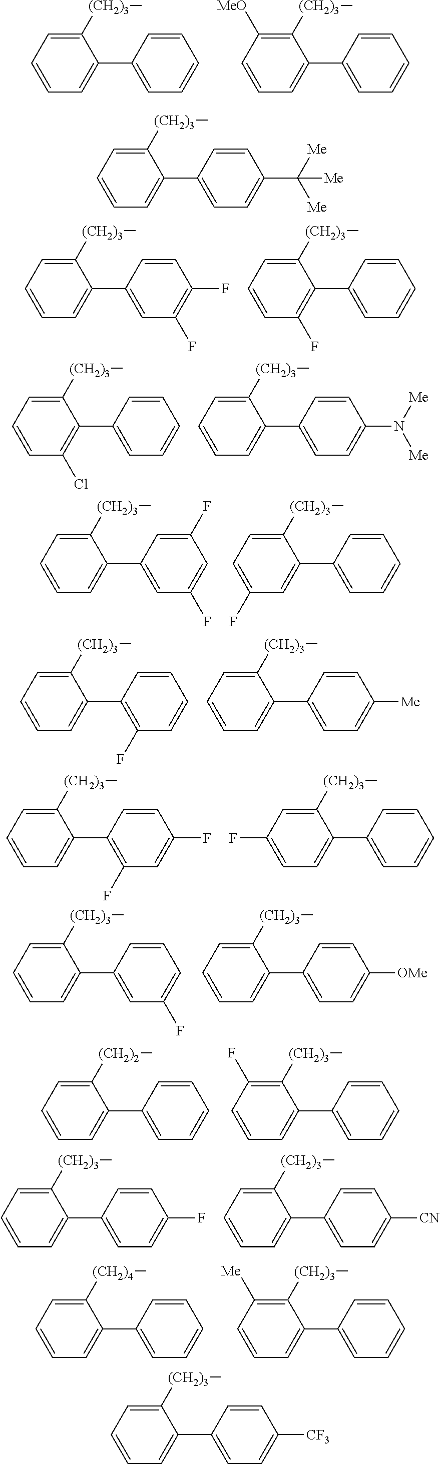

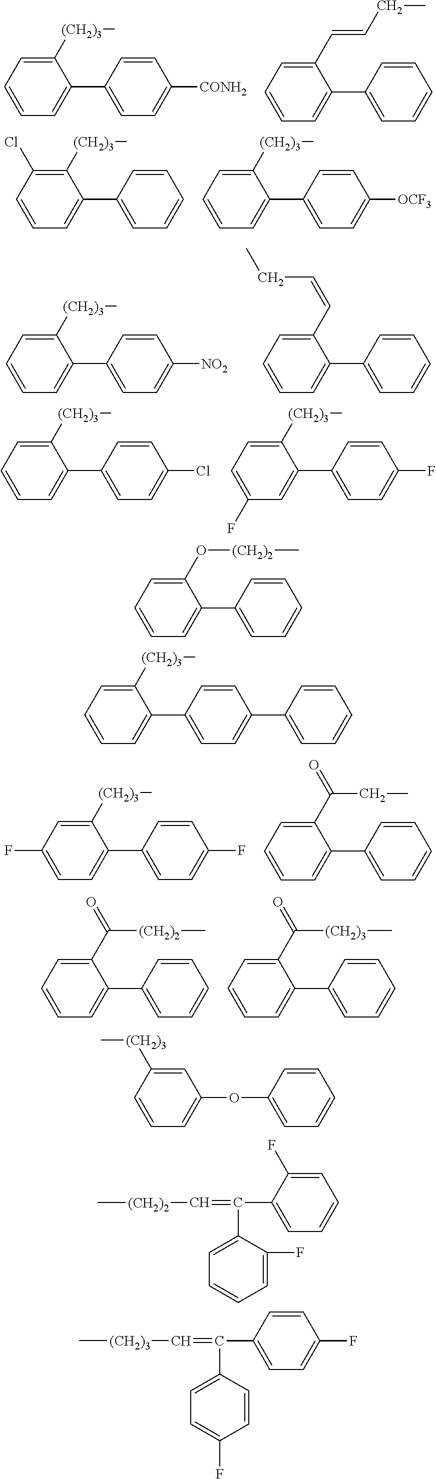

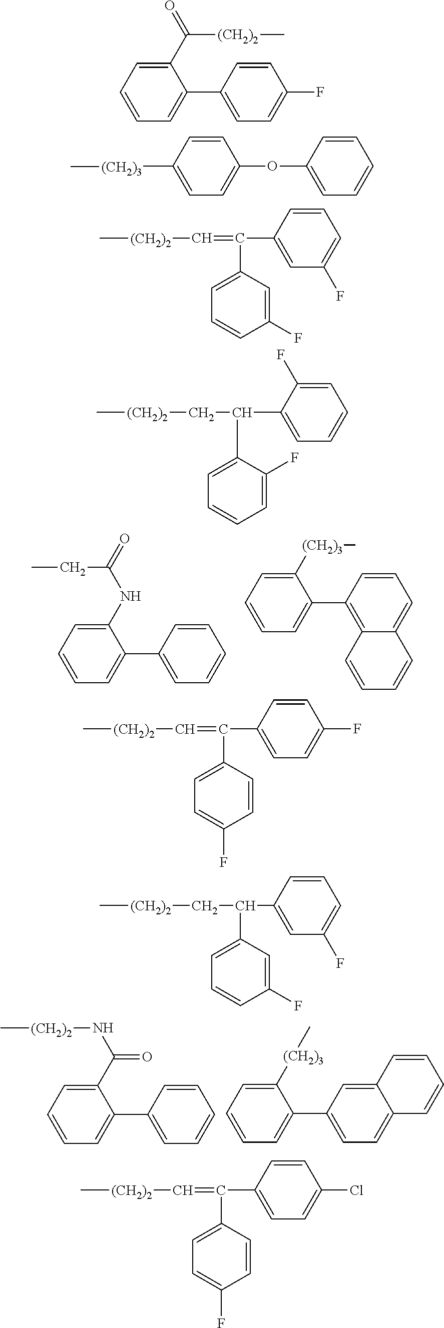

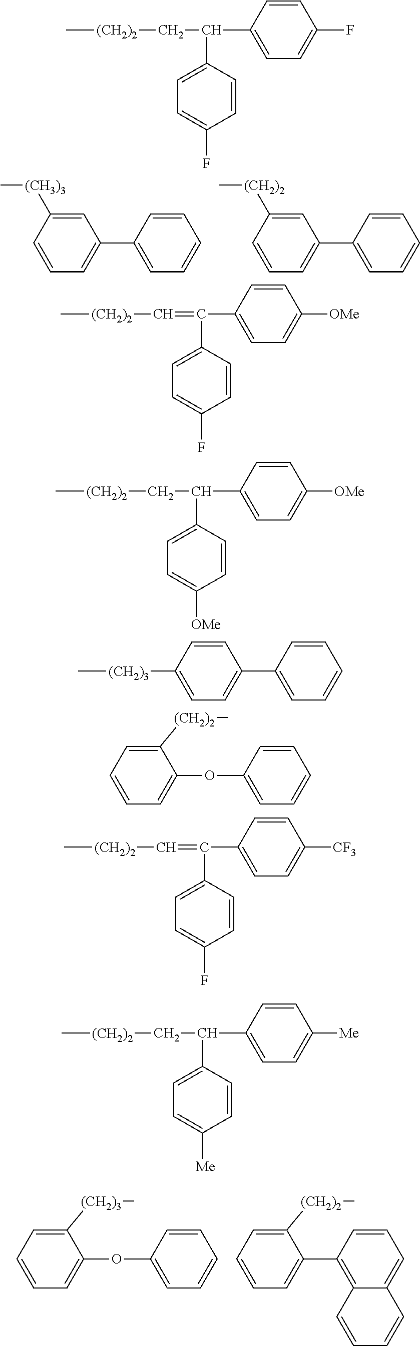

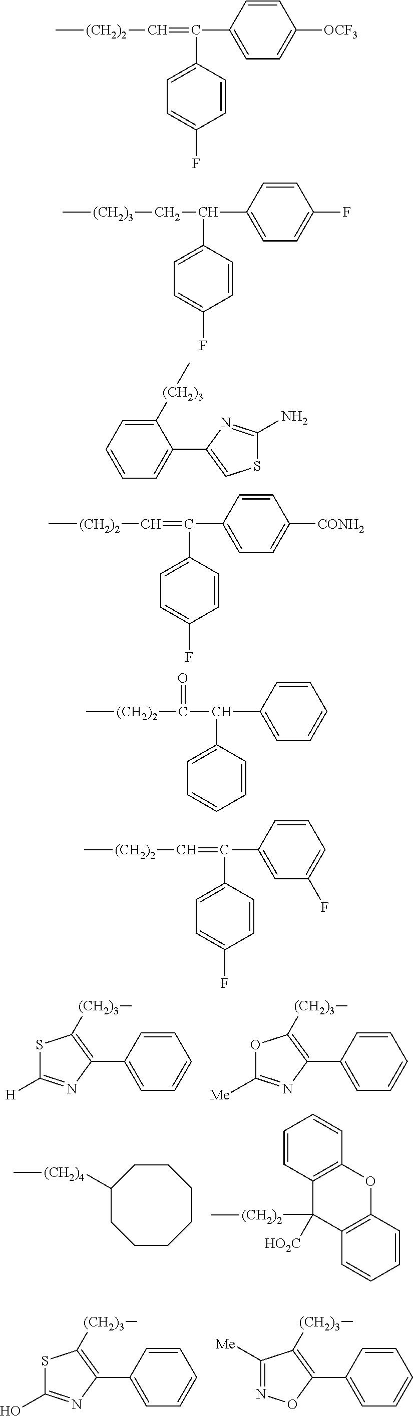

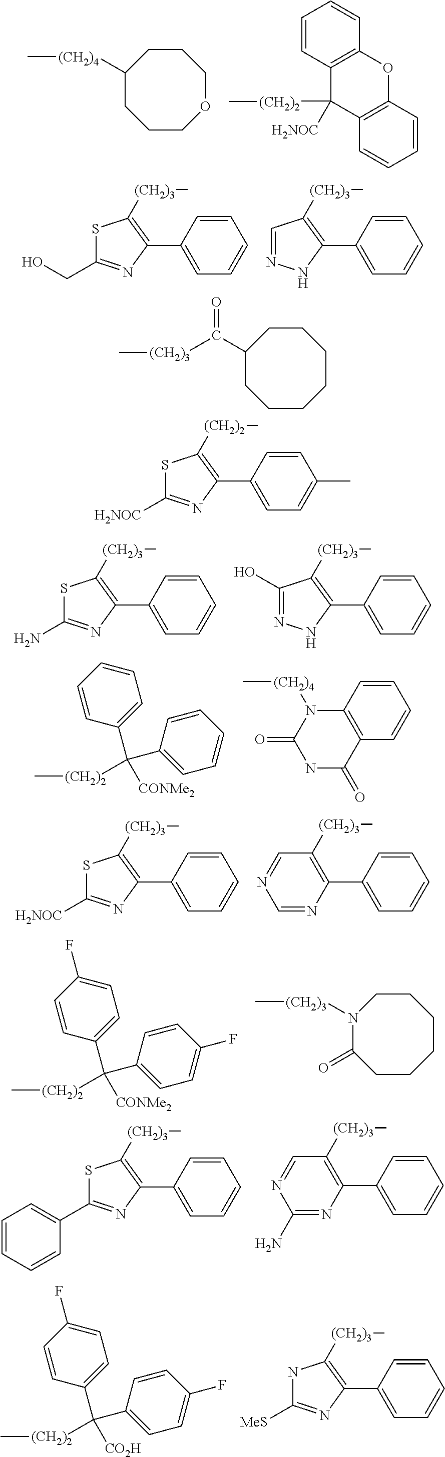

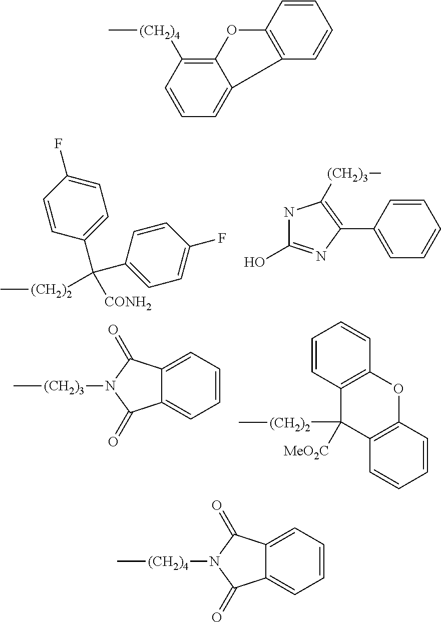

and
c) compounds of the general formula

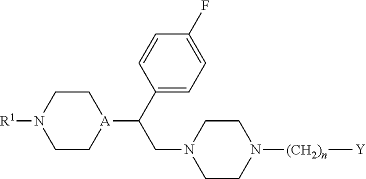

with the meaning of:
A=N (nitrogen),
n=1-8,
R¹=methyl and
wherein —(CH₂)ₙ—Y has the following meaning:

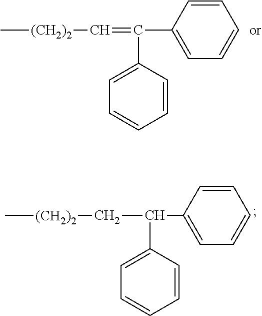

and
d) compounds of the general formula

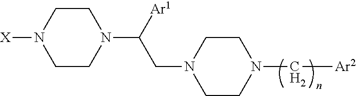

wherein the substituents X, Ar¹, Ar², and n have the following meaning:
(Ph=phenyl, Naph=naphthyl, Me=methyl, Et=ethyl, Pr=propyl, Hex=hexyl, Bn=benzyl, iPr=isopropyl, cPr=cyclopropyl, cHex=cyclohexyl, tBu=tert-butyl)

| Ar¹ | Ar² | X | n |
|---|---|---|---|
| 4-F—Ph | 1-Naph | Me | 1 |
| 4-F—Ph | 1-Naph | Me | 2 |
| 4-F—Ph | 1-Naph | Me | 3 |
| 4-F—Ph | 1-Naph | Me | 4 |
| 4-F—Ph | 1-Naph | Me | 5 |
| 4-F—Ph | 1-Naph | Me | 6 |
| 4-F—Ph | 2-Naph | Me | 1 |
| 4-F—Ph | 2-Naph | Me | 2 |
| 4-F—Ph | 2-Naph | Me | 3 |
| 4-F—Ph | 2-Naph | Me | 4 |
| 4-F—Ph | 1-Naph | H | 4 |
| 4-F—Ph | 1-Naph | Et | 4 |
| 4-F—Ph | 1-Naph | Pr | 4 |
| 4-F—Ph | 1-Naph | iPr | 4 |
| 4-F—Ph | 1-Naph | cPr | 4 |
| 4-F—Ph | 1-Naph | cHex | 4 |
| 4-F—Ph | 1-Naph | Ph | 4 |
| 4-F—Ph | 1-Naph | Amidyl | 4 |
| 4-F—Ph | 1-Naph | Pyrimidin-2-yl | 4 |
| 1-Naph | 1-Naph | Me | 4 |
| 1-Naph | 2-Naph | Me | 4 |
| 2-Naph | 1-Naph | Me | 4 |
| 2-Naph | 2-Naph | Me | 4 |
| Ph | 1-Naph | Me | 4 |
| 3-F—Ph | l-Naph | Me | 4 |
| 4-Cl—Ph | 1-Naph | Me | 4 |
| 4-Me—Ph | 1-Naph | Me | 4 |
| 2-MeO—Ph | 1-Naph | Me | 4 |
| 3-MeO—Ph | 1-Naph | Me | 4 |
| 4-MeO—Ph | 1-Naph | Me | 4 |
| 2-Br—Ph | 1-Naph | Me | 4 |
| 3-Br—Ph | 1-Naph | Me | 4 |
| 4-Br—Ph | 1-Naph | Me | 4 |
| 4-Biphenyl | 1-Naph | Me | 4 |
| 4-CF₃—Ph | 1-Naph | Me | 4 |
| 4-N0₂—Ph | 1-Naph | Me | 4 |
| 4-NH₂—Ph | 1-Naph | Me | 4 |
| 4-BnO—Ph | 1-Naph | Me | 4 |
| 4-F—Ph | 4-Quinolyl | Me | 4 |
| 4-F—Ph | 4-Me₂N—I-Naph | Me | 4 |
| 4-F—Ph | Benzo[b]furan-3-yl | Me | 4 |
| 4-F—Ph | Indol-3-yl | Me | 4 |
| 4-F—Ph | 5-Cl-Benzothiophen-3-yl | Me | 4 |
| 4-F—Ph | 6-F-1,2-Benzisoxazol-3-yl | Me | 4 |
| 4-F—Ph | 4-Methoxy-6H- | iPr | 3 |
| | dibenzo[b,d]pyran-1-yl | | |
| 4-F—Ph | 4-Methoxy-6H- | iPr | 4 |
| | dibenzo[b,d]pyran-1-yl | | |
| 4-F—Ph | 4-Me₂N-1-Naph | iPr | 4 |
| 4-F—Ph | 4-MeO-1-Naph | Me | 4 |
| 4-F—Ph | 2-MeO-1-Naph | Me | 4 |
| 4-F—Ph | 4-Me-1-Naph | Me | 4 |
| 4-F—Ph | 4-F-1-Naph | Me | 4 |
| 4-F—Ph | 2-OH-1-Naph | iPr | 4 |
| 4-F—Ph | 2-iPrO-1-Naph | iPr | 4 |
| 4-F—Ph | 2-EtO-1-Naph | iPr | 4 |
| 4-F—Ph | 2-MeO-1-Naph | Cyclopentyl | 4 |
| 4-F—Ph | 2-MeO-1-Naph | 1-Ethylpropyl | 4 |
| 4-F—Ph | 2-MeO-1-Naph | Allyl | 4 |
| 4-F—Ph | 2-MeO-1-Naph | iPr | 4 |
| 4-F—Ph | 2-MeO-1-Naph | tBu | 4 |
| 4-F—Ph | 2-MeO-1-Naph | Me | 4 |
| 4-F—Ph | 2-MeO-l-Naph | Me | 4 |
| 4-F—Ph | 2-MeO-1-Naph | H | 4 |
| 4-F—Ph | 2-MeO-1-Naph | H | 4 |
| 4-F—Ph | 2-MeO-1-Naph | H | 4 |
| 4-F—Ph | 2-iPrO-1-Naph | iPr | 4 |
| 4-F—Ph | 2-iPrO-1-Naph | iPr | 4 |
| 4-F—Ph | 4-Methoxy-6H-di- | iPr | 1 |
| | benzo[b,d]pyran-1-yl | | |
| 4-F—Ph | 4-Methoxy-6H-di- | iPr | 2 |
| | benzo[b,d]pyran-1-yl | | |
| 4-F—Ph | 2-Br-1-Naph | iPr | 4 |
| 3-CN—Ph | 2-MeO-1-Naph | iPr | 4 |
| 4-CONH₂—Ph | 2-MeO-1-Naph | iPr | 4 |
| 3-CONH₂—Ph | 2-MeO-1-Naph | iPr | 4 |
| 4-F—Ph | 2-MeO-1-Naph | 1-Cyanoethyl | 4 |
| 4-F—Ph | 2-Methoxycarbonyl- | iPr | 4 |
| | methoxy-l-Naph | | |
| 4-F—Ph | 2-Carbamoyl-methoxy-1- | iPr | 4 |
| | Naph | | |
| 4-F—Ph | 2-Ph—Ph | iPr | 2 |
| 4-F—Ph | 2-Ph—Ph | iPr | 3 |
| 4-F—Ph | 2-Ph—Ph | iPr | 4 |
| 4-F—Ph | 3-Ph—Ph | iPr | 3 |
| 4-F—Ph | 4-Ph—Ph | iPr | 3 |
| 4-F—Ph | 2-Ph-3-F—Ph | iPr | 3 |
| 4-F—Ph | 2-Ph-4-F—Ph | iPr | 3 |
| 4-F—Ph | 2-Ph-5-F—Ph | iPr | 3 |
| 4-F—Ph | 2-Ph-6-F—Ph | iPr | 3 |
| 4-F—Ph | 2-Ph-6-Cl—Ph | iPr | 3 |
| 4-F—Ph | 2-Ph-6-Me—Ph | iPr | 3 |
| 4-F—Ph | 2-Ph-6-MeO—Ph | iPr | 3 |
| 4-F—Ph | 2-(2-F—Ph)—Ph | iPr | 3 |
| 4-F—Ph | 2-(3-F—Ph)—Ph | iPr | 3 |
| 4-F—Ph | 2-(4-F—Ph)—Ph | iPr | 3 |
| 4-F—Ph | 2-(4-Cl—Ph)—Ph | iPr | 3 |
| 4-F—Ph | 2-(4-Me—Ph)—Ph | iPr | 3 |
| 4-F—Ph | 2-(4-MeO—Ph)—Ph | iPr | 3 |
| 4-F—Ph | 2-(4-(tBu—Ph)—Ph | iPr | 3 |
| 4-F—Ph | 2-(4-Ph—Ph)—Ph | iPr | 3 |
| 4-F—Ph | 2-(4-CF₃—Ph)—Ph | iPr | 3 |
| 4-F—Ph | 2-(4-CF₃O—Ph)—Ph | iPr | 3 |
| 4-F—Ph | 2-(4-Me₂N—Ph)—Ph | iPr | 3 |

and
e) compounds of the formula

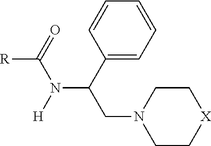

with the meaning for R=oleyl, diphenylacetyl and alkyl having at least 10 C atoms in the carbon chain (C_{(≧10)}-alkyl) and wherein X has the meaning N, C or CH and O;
and
f) compounds which correspond to the general formula (Ia)

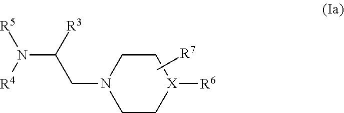

of the present invention and wherein
X has the meaning CH, R⁶ is chosen from optionally substituted benzoyl, R⁷ denotes hydrogen, R³ denotes optionally substituted aryl or alkyl and wherein R⁴ and R⁵ together with the nitrogen atom to which they are bonded form an aromatic 5-membered heterocyclyl ring which contains at least one further hetero atom chosen from nitrogen;
and in particular
g) the following compounds:

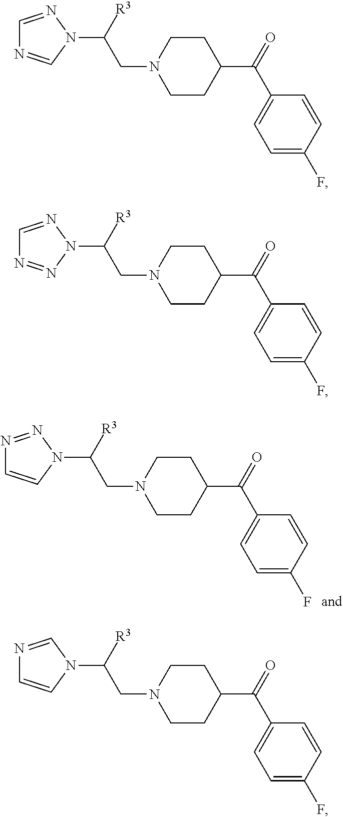

wherein in each case R³ denotes optionally substituted aryl or alkyl;
and
h) compounds which correspond to the following formulae:

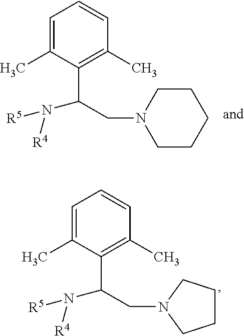

wherein the substituent R³ according to compound (I) of the present invention thus corresponds to 2,6-dimethylphenyl, and wherein furthermore in each case R⁴ and R⁵ are identical or different and have the meaning H, alkyl and acyl, as defined in the context of the present invention;
and
i) the compound

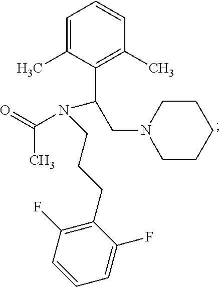

and
j) compounds of the formula:

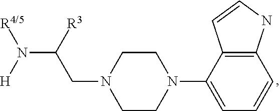

wherein one of the substituents R⁴ and R⁵ denotes hydrogen and the other denotes optionally substituted acyl, as defined in the context of the present invention, and wherein R³ has the meaning of optionally substituted phenyl or benzyl, reference being made to the above definition of substituted phenyl and benzyl (or aryl-substituted alkyl) with respect to possible substituents.

In principle, in the context of the present invention it is possible to combine the individual preferred, more preferred or particularly preferred meanings for the substituents R¹ to R⁷ with one another. That is to say that the present invention includes compounds of the general formula (I) in which, for example, the substituent R⁶ and/or the substituents R¹ and R² have a preferred or more preferred meaning and the substituents R⁴ and R⁵ have the general meaning or the substituent R⁶ and/or the substituents R¹ and R² have a general meaning and the substituents R⁴ and R⁵ have a preferred or more preferred meaning etc.

Depending on their structure, if asymmetric carbon atoms are present the compounds according to the invention can exist in stereoisomeric forms (enantiomers, diastereomers). The invention therefore includes the use of the enantiomers or diastereomers and their particular mixtures. The enantiomerically pure forms can optionally be obtained by conventional processes of optical resolution, such as by fractional crystallization of diastereomers therefrom by reaction with optically active compounds. If the compounds according to the invention can occur in tautomeric forms, the present invention includes the use of all the tautomeric forms.

An asymmetric carbon atom can be present, for example, at the marked position:

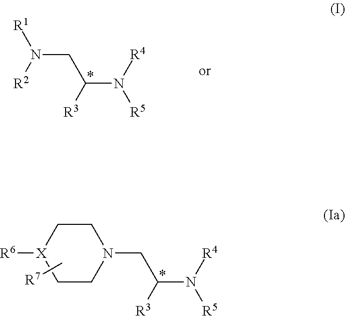

The compounds provided according to the invention can be present as mixtures of various possible isomeric forms, in particular of stereoisomers, such as e.g. E and Z, syn and anti, and optical isomers. Both the E and the Z isomers and the optical isomers, and any desired mixtures of these isomers are claimed.

The compounds according to the invention of the general structural formula (I) can in principle be obtained by the processes explained in the following.

Reaction of compounds of the formula (II):

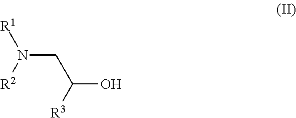

wherein R¹, R² and R³ are as defined above, with compounds of the formula

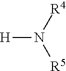

wherein R⁴ and R⁵ are as defined above,
to give compounds of the formula (I)

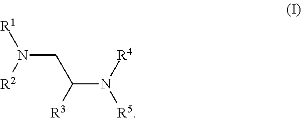

In particular, compounds of the general formula (I) according to the invention wherein R¹ and R² together with the nitrogen atom to which they are bonded form a saturated, optionally substituted 6-membered ring and thus form a compound according to the general formula (Ia) can be obtained by the processes explained in the following, the compound (IIa) being obtained as an intermediate product analogously to the above reaction equation:

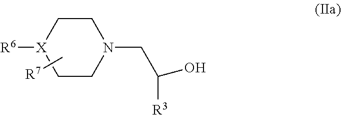

### Synthesis Route 1a):

The starting point for the synthesis of compounds of the general formula (Ia) wherein X represents N and wherein R⁷ is hydrogen and wherein R⁶ has one of the meanings as defined above is the commercially obtainable piperazine of the general formula (IIIa). This can be alkylated or arylated by standard methods known to the person skilled in the art to give the compound of the general formula (IIa) wherein X represents N and wherein R⁷ is hydrogen [see e.g.: M. B. Smith, J. March, March's advanced organic chemistry, 5th. ed, Wiley, NY, 2001. 499-501: for a review of alkylation of amino groups].

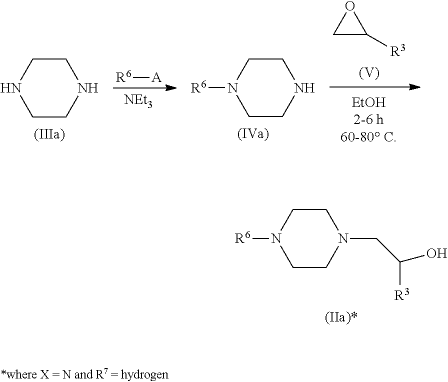

### Synthesis Route 1b):

Compounds of the general formula (Ia) wherein X represents N and wherein R⁷ is hydrogen and wherein R⁶ denotes an acyl group, as defined above, where R⁸=alkyl, aryl or heterocyclyl, alkoxy or amino, as defined above, can be prepared as follows:

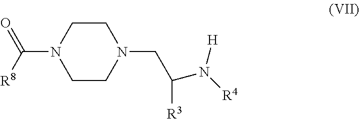

The starting point for the synthesis of such compounds (VII) is likewise the commercially obtainable piperazine of the general formula (IIIa), which can be reacted analogously to the synthesis route described above to give the compound of the general formula (VI). In this reaction, piperazine (IIa) is reacted with compounds of the formula R⁸—(C═O)-A under basic reaction conditions. Compounds of the formula R⁸—(C═O)-A are preferably those wherein A represents a conventional leaving group, such as, in particular, halogen, preferably chlorine, and are thus chosen from the group of acyl halides, preferably from the group of acyl chlorides. The reaction of piperazine (111a) with such compounds R⁸—(C═O)-A, in particular with acyl halides, to give compounds of the general formula (VI) is carried out by standard methods known to the person skilled in the art [see e.g.: M. B. Smith, J. March, March's advanced organic chemistry, 5th. ed, Wiley, NY, 2001. 506-512: for a review of acylation of amino groups; S. Paul, THL, 43, 2002, 4261-4266; S. Chaurasia, Journal of the Indian Chemical Society, 69, 1992,45-46]. The acylpiperazine (VI) obtainable in this way can then be reacted with epoxides of the general formula (V) to give the compounds of the general formula (VIIa) [see e.g.: A. Franke, Liebigs Annalen der Chemie, 4, 1982, 794-804; K. G. Estep, Journal of Medicinal Chemistry, 38, 1995, 2582-2595; L. Korzycka, Journal of Pharmacy and Pharmacology, 54, 2002, 445-450].

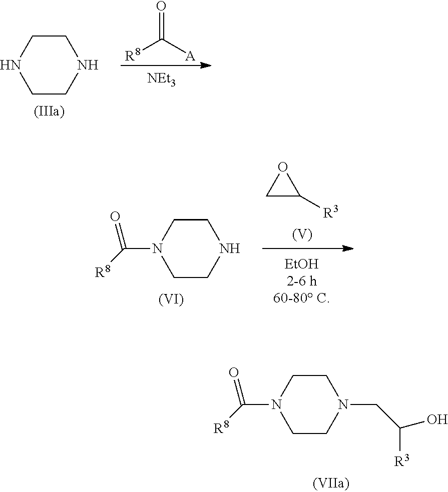

Two alternative synthesis routes 2) and 3) are then available for converting the compounds of the general formulae (IIa) where X═N and R⁷═H and (VIIa) into the target compounds of the general formula (Ia) where X═N and R⁷═H or (VII), both of which fall under the general formula (I) according to the invention.

### Synthesis Route 2):

In this, the OH group of compound (IIa) wherein X═N and R⁷═H or of the compound (VIIa) is converted into a better leaving group under basic conditions by standard methods known to the person skilled in the art, for which mesyl and tosyl groups are particularly suitable [see e.g.: B. Cope; JACS, 74,1952,611-614; Campbell et al, JOC, 14, 1949, 346-349], in order then to be subsequently converted by a nucleophilic substitution reaction into the corresponding amino compounds of the general formula (Ia) where X═N and R⁷═H or (VII) [see e.g.: C. Verbruggen, Bioorganic & Medicinal—Chemistry, 6, 1996, 253-258].

Reaction of the compound (IIa) to give the compound (Ia) wherein in each case X═N and R⁷═H:

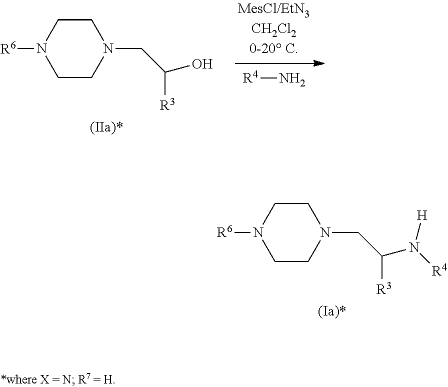

This reaction mechanism can similarly also be applied to compounds of the formula (VIIa) and conversion thereof into compounds of the formula (VII), wherein R⁸ has the meaning defined above:

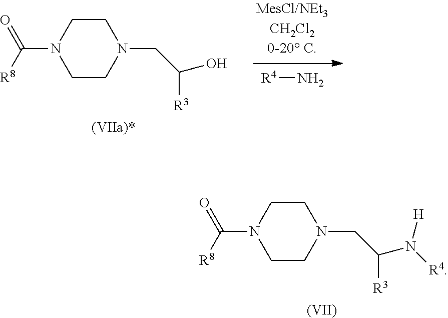

In the synthesis routes 2) shown, in each case R⁴ has one of the meanings defined above and R⁵ denotes hydrogen. In principle, the same reaction mechanism also applies to compounds (Ia) where X═N and R⁷═H and (VII) wherein in each case R⁴ denotes hydrogen and R⁵ has one of the meanings defined above and which are obtainable in a corresponding manner by reaction with compounds R⁵—NH₂.

Alternatively, the reaction of the compounds (IIa) wherein X═N and R⁷═H and of the compound (VIIa) can also proceed according to synthesis route 3) as follows:

### Synthesis Route 3):

In synthesis route 3), the OH group of the compound of the formula (IIa) where X═N and R⁷═H or of the compound (VIIa) is oxidized to the keto group by standard methods known to the person skilled in the art [see e.g.: C. Carite, THL, 31, 1990, 7011-7014] and the ketones are then converted by a reductive amination by standard methods known to the person skilled in the art [see e.g.: M. Adrover, Bioorganic & Medicinal —Chemistry, 16, 2008, 5557-5569; M. B. Smith, J. March, March's advanced organic chemistry, 5th. ed, Wiley, NY, 2001, 1187-1189: for a review of reductive amination] into the corresponding target compound (Ia) where X═N and R⁷═H or (VII).

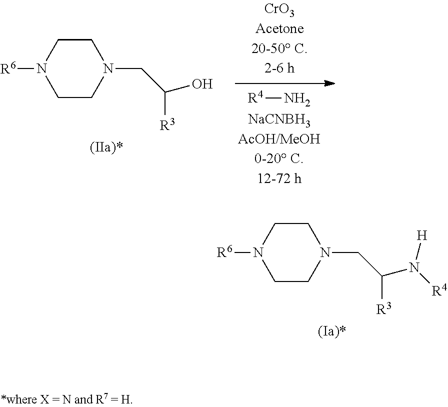

This reaction mechanism can similarly also be applied to compounds of the formula (VIIa) and conversion thereof into compounds of the formula (VII), wherein R⁸ has the meaning defined above:

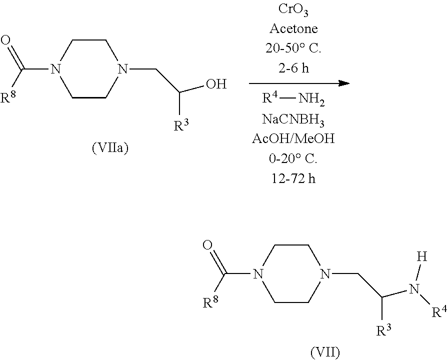

In the synthesis routes 3) shown, in each case R⁴ has one of the meanings defined above and R⁵ denotes hydrogen. In principle, the same reaction mechanism also applies to compounds (Ia) where X═N and R⁷═H and (VII) wherein in each case R⁴ denotes hydrogen and R⁵ has one of the meanings defined above and which are obtainable in a corresponding manner by reaction with compounds R⁵—NH₂.

In particular, the compounds according to the invention according to Examples 1, 2 and 3 are also obtainable by these synthesis routes described. In this context, compounds according to Example 1 are obtainable in principle according to synthesis route 1a) and optionally also 1b) and by subsequent reaction according to synthesis route 2) or 3), whereas the compounds according to Examples 2 and 3 are obtainable in particular via synthesis route 1b) and subsequent reaction according to synthesis route 2) or 3).

A further process route according to the invention is moreover available which is suitable for the preparation of the compounds of the general formula (I) according to the invention wherein R¹ and R² together with the nitrogen atom to which they are bonded form a saturated, substituted 6-membered ring and thus form compounds according to the general formula (Ia) wherein X represents CH and wherein R⁷ is hydrogen and wherein R⁶ has one of the meanings as defined above.

### Synthesis Route 4):

The starting point for the synthesis of such compounds according to the invention are commercially obtainable piperidines of the general formula (IIIb), such as 4-chloropiperidine, which can be converted into compounds of the general formula (IIa) where X═CH and R⁷═H and further into compounds of the formula (Ia) where X═CH and R⁷═H.

### Synthesis Route 4a):

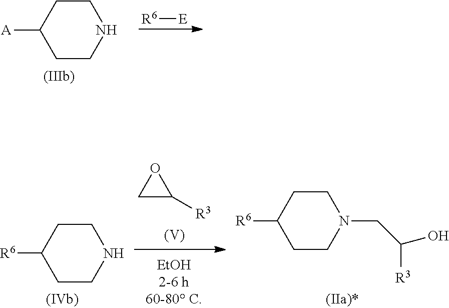

wherein A is a leaving group, such as halogen, in particular chlorine, and E here and in the following is a suitable group or a suitable element which makes R⁶ a nucleophile, such as, for example, H (in particular if R⁶ is an amino group), metals (in particular if R⁶ is a hydrocarbon radical), such as, in particular, alkali metals, such as lithium, sodium and potassium, alkaline earth metals, such as calcium or magnesium, —MgBr (Grignard compounds), which render possible nucleophilic substitution of A by R⁶, and the substituents R³ and R⁶ have one of the meanings defined above.

It is furthermore of course also possible to employ as the starting point commercially obtainable piperidines which already contain the desired substituents R⁶.

### Synthesis Route 4b):

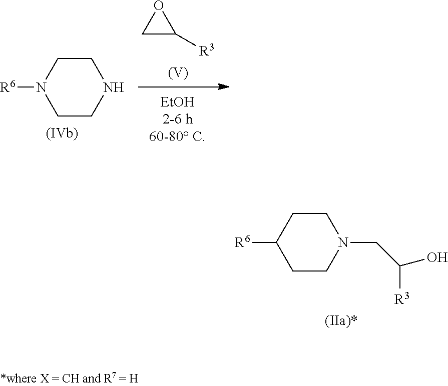

In this, R³ and R⁶ have one of the meanings defined above.

The conversion of the compounds obtainable in this way, of the general formula (IIa) where X═CH and R⁷═H, into the target compounds of the general formula (Ia) where X═CH and R⁷═H is carried out analogously according to the synthesis route 2) or 3) described above, which is referred to herewith.

In particular, the compounds according to the invention according to Examples 4, 5 and 6 are also obtainable by these synthesis routes described.

It is to be noted that epoxides of the general formula (V) which are not commercially accessible can be synthesized as follows:

Alkenes of the general formula

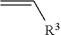

are converted by standard oxidation methods known to the person skilled in the art [see e.g.: S. Sheffer-Dee-Noor, TH, 50, 1994, 7009-7018; G. Miao, JOC, 60, 1995, 8424-8427] into the corresponding epoxides of the general formula (V).

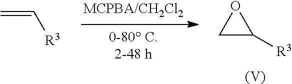

In particular, processes according to the synthesis routes A) and B) described in detail in the following, wherein the meaning of the substituents R¹ to R⁷ corresponds to the above definitions and wherein the abbreviations used have the meaning as defined in the following preparation examples, are preferred.

### Preferred Processes According to Synthesis Routes a)

A) Synthesis Route I

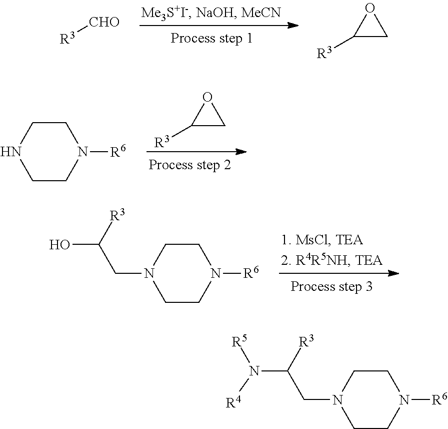

A) Synthesis Route II

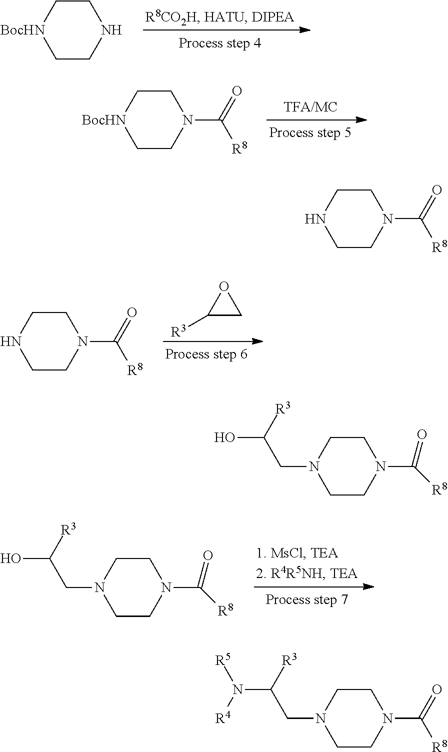

with the meaning for R⁸ of optionally substituted alkyl, aryl, heterocyclyl, alkoxy and amino, in each case as defined above.

A) Synthesis Route III

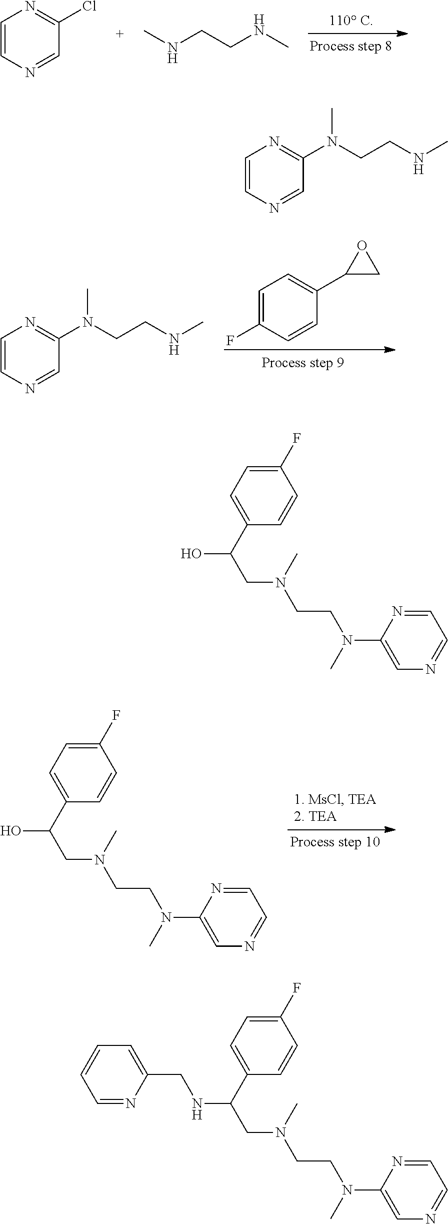

A) Synthesis Route IV

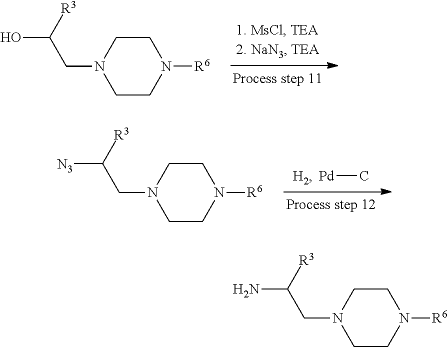

### Preferred Processes According to Synthesis Routes B)

B) Synthesis Route I

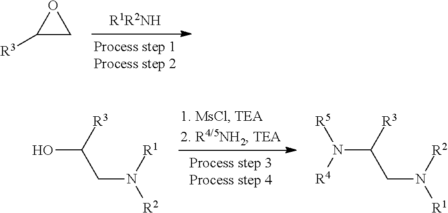

B) Synthesis Route II

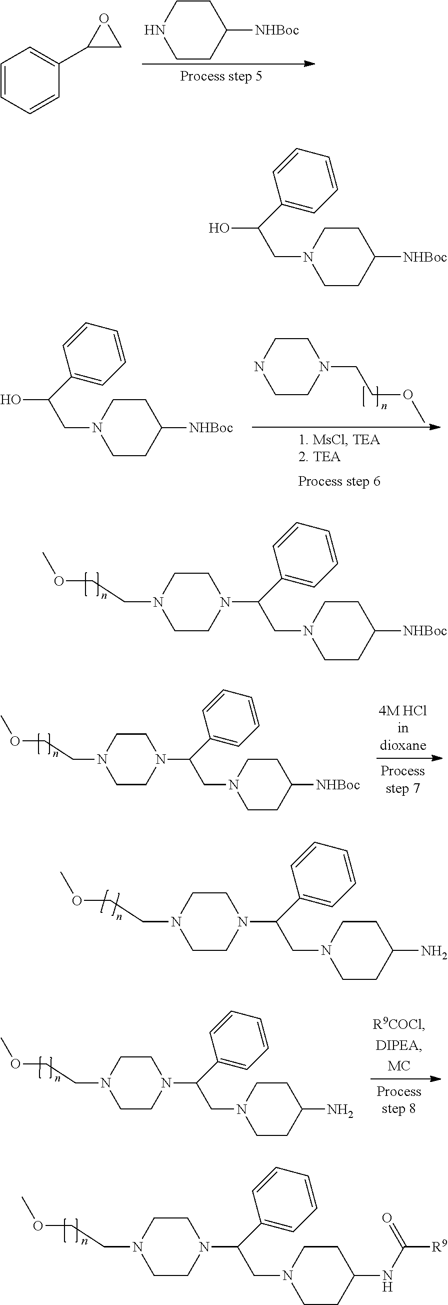

with the meaning for R⁹ of optionally substituted alkyl and aryl, in each case as defined above, and of n=0-3.

B) Synthesis Route III

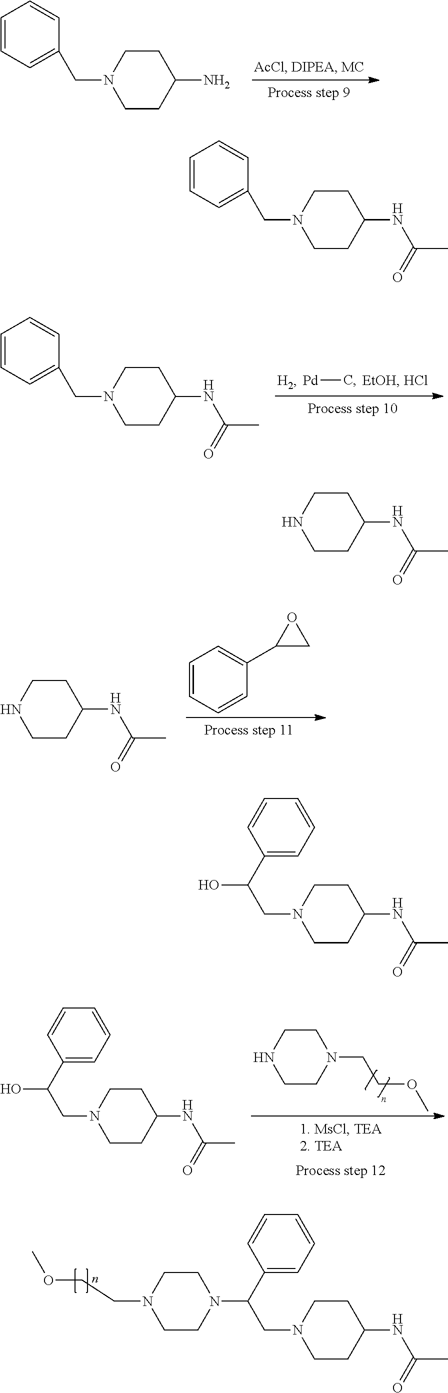

with the meaning for n=0-3.

B) Synthesis route IV

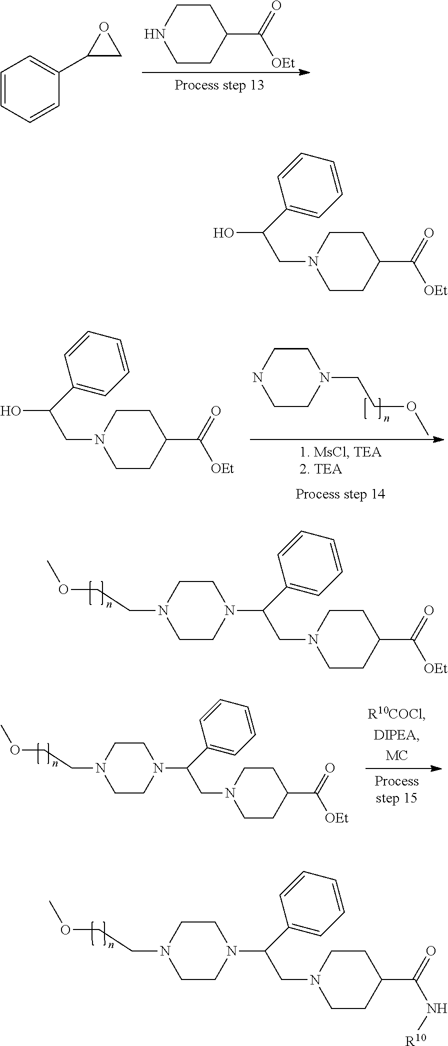

with the meaning for R¹⁰ of optionally substituted alkyl and aryl, in each case as defined above, and of n=0-3.

B) Synthesis Route V

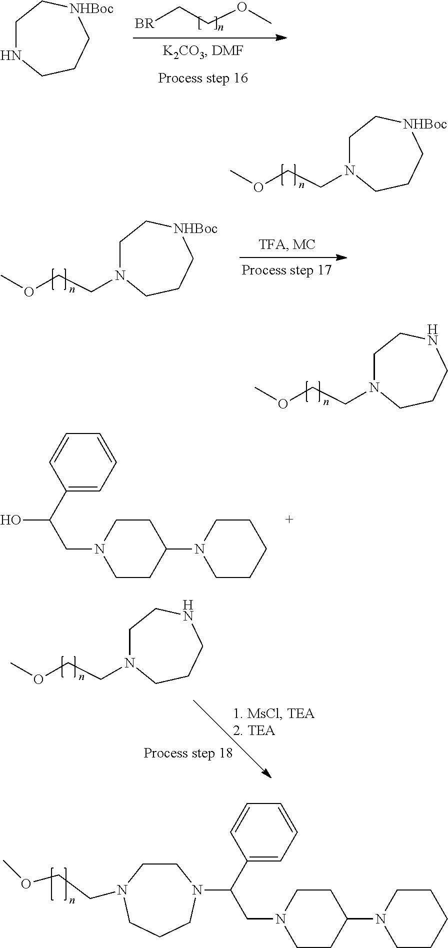

with the meaning for n=0-3.

B) Synthesis Route VI

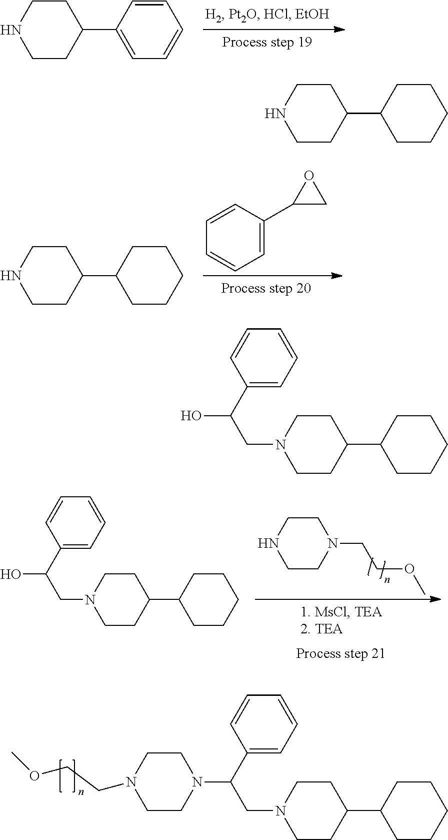

with the meaning for n=0-3.

B) Synthesis Route VII

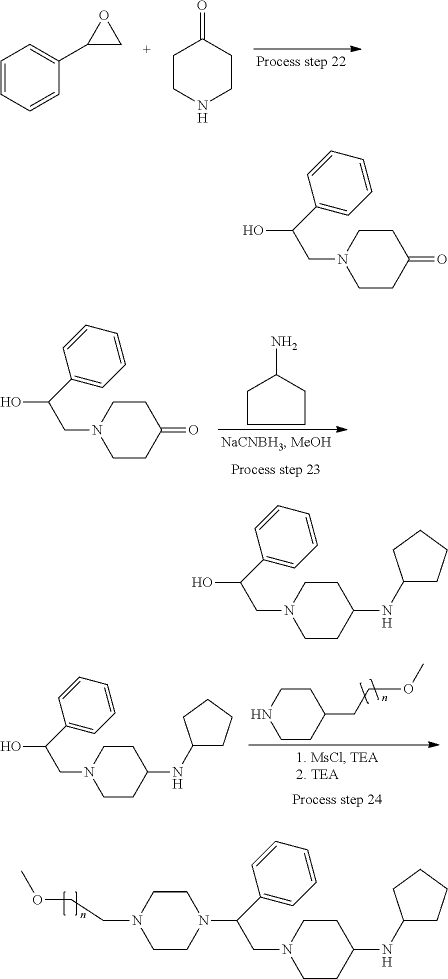

with the meaning for n=0-3.

B) Synthesis Route VIII

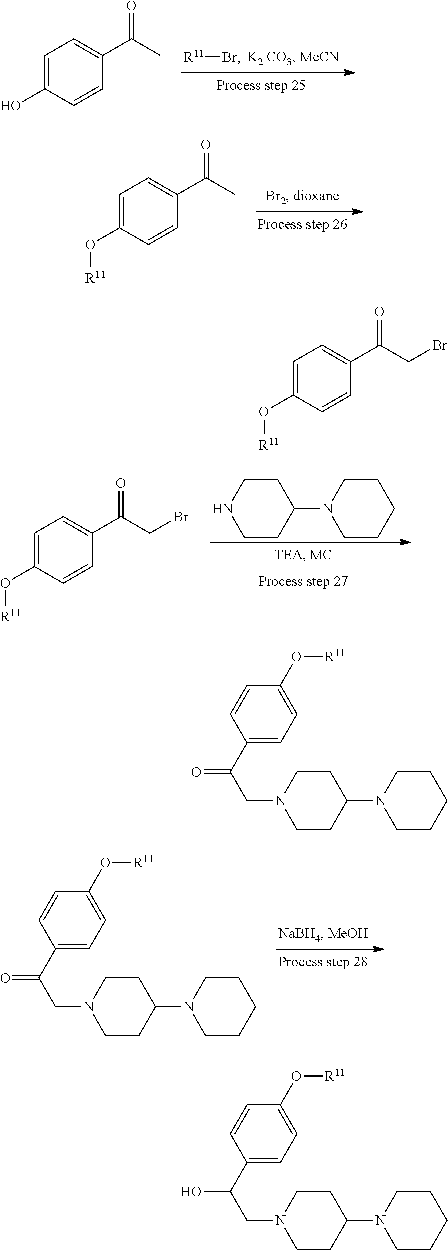

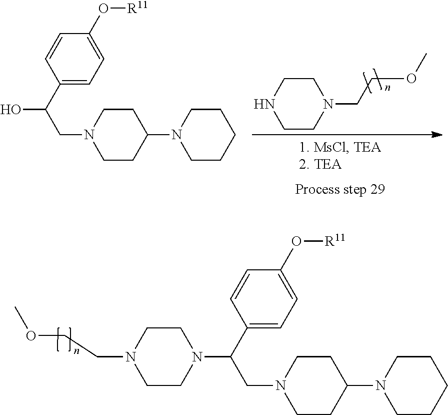

with the meaning for R¹¹ of halogen, cyano and optionally substituted alkyl and alkoxy, in each case as defined above, and of n=0-3.

B) Synthesis Route IX

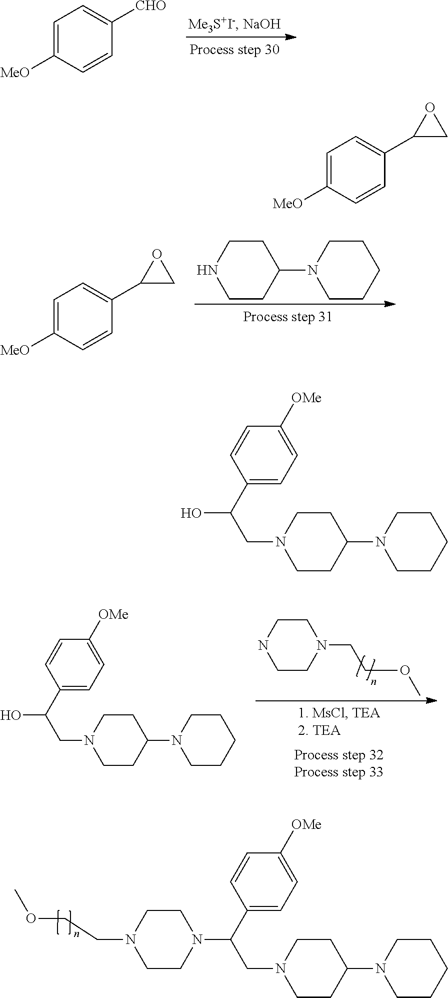

with the meaning for n=0-3.

B) Synthesis Route X

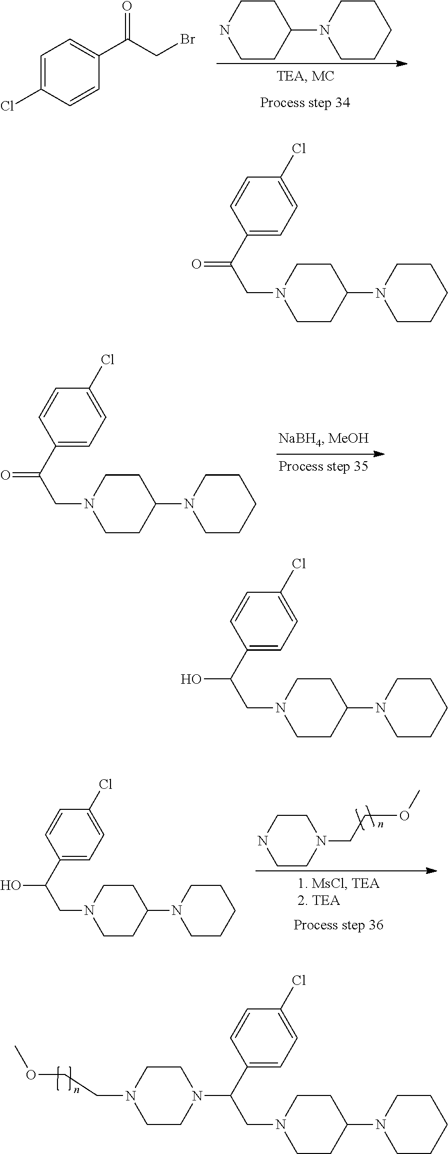

with the meaning for n=0-3.

The reaction paths shown here are reaction types which are known per se and which can be carried out in a manner known per se. By reaction with a pharmaceutical acceptable base or acid, corresponding salts are obtained.

The reaction of the various reaction partners can be carried out in various solvents, and in this respect is not subject to a particular limitation. Corresponding examples of suitable solvents are thus water, methanol, ethanol, acetone, dichloroethane, methylene chloride, dimethoxyethane, diglyme, acetonitrile, butyronitrile, THF, dioxane, ethyl acetate, butyl acetate, dimethylacetamide, toluene, chlorobenzene etc. Methanol, ethanol, acetone and methylene chloride are preferred, and in particular the solvents used in the preferred processes described above according to synthesis routes A) and B).

It is moreover possible to carry out the reaction in an essentially homogeneous mixture of water and solvents if the organic solvent is miscible with water.

The reaction according to the invention of the reaction partners is carried out, for example, at room temperature. However, temperatures above room temperature, for example up to 80 or 90° C., and temperatures below room temperature, for example down to −20° C. or less, can also be used.

The pH at which the reaction according to the invention of the reaction partners is carried out is suitably adjusted.

The pH adjustment, in particular in the reaction of the starting compounds from the group of piperazines or of piperidines in synthesis route 1a), 1b) and 4) and in the basic reaction with mesyl and tosyl groups and subsequent amination with R⁴—NH₂ or R⁵—NH₂ in synthesis route 2), is preferably carried out by addition of a base. Both organic and inorganic bases can be used as bases. Preferably, inorganic bases, such as, for example, LiOH, NaOH, KOH, Ca(OH)₂, Ba(OH)₂, Li₂CO₃, K₂CO₃, Na₂CO₃, NaHCO₃, or organic bases, such as amines (such as, for example, preferably triethylamine, diethylisopropylamine), Bu₄NOH, piperidine, morpholine, alkylpyridines, are used. Particularly preferably, organic bases, very particularly preferably triethylamine (NEt₃), are used.

The pH adjustment can optionally also be carried out by means of acids, such as, in particular, in the reductive amination of the ketones in synthesis route 3). Both organic and inorganic acids can be used as acids. Preferably, inorganic acids, such as, for example, HCl, HBr, HF, H₂SO₄, H₃PO₄, or organic acids, such as CF₃COOH, acetic acid (CH₃COOH, AcOH), p-toluenesulfonic acid, and salts thereof are used. Organic acids, such as acetic acid (CH₃COOH, AcOH), are particularly preferably used.

The pH adjustment is particularly preferably carried out by means of the pH-adjusting agents used in the preferred processes described above according to synthesis routes A) and B).

A person skilled in the art is in a position here to choose the most suitable solvent and the optimum reaction conditions, in particular with respect to temperature, pH, catalyst and solvent, for the corresponding synthesis route or for the corresponding reaction step.

The present invention thus also provides novel intermediate products which are accessible with the preparation processes according to the invention, such as, in particular, the intermediate products 1 to 71 described concretely in the examples which are obtainable from the process steps 1 to 35 described.

The inventors have found, surprisingly, that the compounds provided by the present invention and represented by the general structural formula (I) and in particular (Ia) show an action as a hepcidin antagonist and are therefore suitable for use as medicaments for treatment of hepcidin-mediated diseases and the symptoms accompanied by these or associated with these. In particular, the compounds according to the invention are suitable in use for treatment of disorders in iron metabolism, in particular for treatment of iron deficiency diseases and/or anaemias, in particular ACD and AI.

The medicaments containing the compounds of the general structural formula (I) are suitable in this context for use in human and veterinary medicine.

The present invention thus also provides the compounds of the general structural formula (I) according to the invention with the above substituent meanings for use as medicaments.

In particular, such compounds of the general structural formula (I) according to the invention with the above substituent meanings are preferably suitable for use as medicaments, one or more compounds from the group of compounds a) to j) which are excluded in the preferred embodiment described above being excluded.

The compounds according to the invention are therefore also suitable for the preparation of a medicament for treatment of patients suffering from symptoms of an iron deficiency anaemia, such as, for example: tiredness, lack of drive, lack of concentration, low cognitive efficiency, difficulties in finding the correct words, forgetfulness, unnatural pallor, irritability, accelerated heart rate (tachycardia), sore or swollen tongue, enlarged spleen, pregnancy cravings (pica), headaches, loss of appetite, increased susceptibility to infections, depressive moods or suffering from ACD or AL.

The compounds according to the invention are therefore also suitable for the preparation of a medicament for treatment of patients suffering from symptoms of an iron deficiency anaemia.

Administration can take place over a period of several months until the iron status improves, reflected, for example, by the haemoglobin value, the transferrin saturation and the ferritin value of the patient, or until the desired improvement is achieved in an impairment of the state of health caused by iron deficiency anaemia or by ACD or AI.

The preparation according to the invention can be taken by children, adolescents and adults.

The compounds of the present invention can furthermore also be used in combination with further active compounds or medicaments known in the treatment of disorders in iron metabolism and/or with active compounds or medicaments which are administered concomitantly with agents for treatment of diseases which are associated with disorders in iron metabolism, in particular with iron deficiency and/or anaemias. Examples of such agents for treatment of disorders in iron metabolism and further diseases associated with iron deficiency and/or anaemias which can be used in combination can include, for example, iron-containing compounds, such as e.g. iron salts, iron-carbohydrate complex compounds, such as iron-maltose or iron-dextrin complex compounds, vitamin D and/or derivatives thereof.

The compounds used in combination with the compounds according to the invention can be administered in this context either orally or parenterally, or the administration of the compounds according to the invention and of the compounds used in combination can take place by combination of the administration possibilities mentioned.

The compounds according to the invention and the combinations of the compounds according to the invention with further active compounds or medicaments can be employed in the treatment of disorders in iron metabolism, such as, in particular, iron deficiency diseases and/or anaemias, in particular anaemias with cancer, anaemia induced by chemotherapy, anaemia induced by inflammation (AI), anaemias with congestive cardiac insufficiency (CHF; congestive heart failure), anaemia with chronic renal insufficiency stage 3-5 (CKD 3-5; chronic kidney diseases stage 3-5), anaemia induced by chronic inflammation (ACD), anaemia with rheumatic arthritis (RA; rheumatoid arthritis), anaemia with systemic lupus erythematosus (SLE) and anaemia with inflammatory intestinal diseases (IBD; inflammatory bowel disease) or used for the preparation of medicaments for treatment of these diseases.

The compounds according to the invention and the above-mentioned combinations of the compounds according to the invention with further active compounds or medicaments can be used in particular for the preparation of medicaments for treatment of iron deficiency anaemia, such as iron deficiency anaemias in pregnant women, latent iron deficiency anaemia in children and adolescents, iron deficiency anaemia as a result of gastrointestinal abnormalities, iron deficiency anaemia as a result of blood losses, such as by gastrointestinal haemorrhages (e.g. as a result of ulcers, carcinomas, haemorrhoids, inflammatory disorders, intake of acetylsalicylic acid), menstruation, injuries, iron deficiency anaemia as a result of psilosis (sprue), iron deficiency anaemia as a result of reduced uptake of iron from the diet, in particular in selectively eating children and adolescents, weak immune system caused by iron deficiency anaemia, impaired cerebral performance caused by iron deficiency anaemia, restless leg syndrome.

The use according to the invention leads to an improvement in the iron, haemoglobin, ferritin and transferrin values which, especially in adolescents and children, but also in adults, are accompanied by an improvement in the short term memory test (STM), in the long term memory test (LTM), in the Raven's progressive matrices test, in the Wechsler adult intelligence scale (WAIS) and/or in the emotional coefficient (Baron EQ-i, YV test; youth version), or to an improvement in neutrophile levels, antibody levels and/or lymphocyte function.

The present invention furthermore relates to pharmaceutical compositions comprising one or more compounds of the formula (I) according to the invention and optionally one or more further pharmaceutically active compounds and optionally one or more pharmacologically acceptable carriers and/or auxiliary substances and/or solvents.

In this context, the pharmaceutical carriers, auxiliary substances or solvents are conventional substances. The pharmaceutical compositions mentioned are suitable, for example, for intravenous, intraperitoneal, intramuscular, intravaginal, intrabuccal, percutaneous, subcutaneous, mucocutaneous, oral, rectal, transdermal, topical, intradermal, intragastral or intracutaneous administration and are present, for example, in the form of pills, tablets, tablets resistant to gastric juice, film-coated tablets, layered tablets, sustained release formulations for oral, subcutaneous or cutaneous administration (in particular as patches), depot formulation, sugar-coated tablets, small suppositories, gels, ointments, syrup, granules, suppositories, emulsions, dispersions, microcapsules, microformulations, nanoformulations, liposomal formulations, capsules, capsules resistant to gastric juice, powders, powders for inhalation, microcrystalline formulations, sprays for inhalation, dusting powders, drops, nasal drops, nasal sprays, aerosols, ampoules, solutions, juices, suspensions, infusion solutions or injection solutions etc.

Preferably, the compounds according to the invention and pharmaceutical compositions comprising such compounds are administered orally and/or parenterally, in particular intravenously.

For this, the compounds according to the invention are preferably present in pharmaceutical compositions in the form of pills, tablets, tablets resistant to gastric juice, film-coated tablets, layered tablets, sustained release formulations for oral administration, depot formulations, sugar-coated tablets, granules, emulsions, dispersions, microcapsules, microformulations, nanoformulations, liposomal formulations, capsules, capsules resistant to gastric juice, powders, microcrystalline formulations, dusting powders, drops, ampoules, solutions, suspensions, infusion solutions or injection solutions.

The compounds according to the invention can be administered in a pharmaceutical composition which can comprise various organic or inorganic carrier materials and/or auxiliary materials such as are conventionally used for pharmaceutical purposes, in particular for solid medicament formulations. such as, for example, excipients (such as sucrose, starch, mannitol, sorbitol, lactose, glucose, cellulose, talc, calcium phosphate, calcium carbonates), binders (such as cellulose, methylcellulose, hydroxypropylcellulose, polypropylpyrrolidone, gelatine, gum arabic, polyethylene glycol, sucrose, starch), disintegrating agents (such as starch, hydrolysed starch, carboxymethylcellulose, calcium salt of carboxymethylcellulose, hydroxypropyl-starch, sodium glycol starch, sodium bicarbonate, calcium phosphate, calcium citrate), lubricants and slip agents (such as magnesium stearate, talc, sodium lauryl sulfate), a flavouring agent (such as citric acid, menthol, glycine, orange powder), preservatives (such as sodium benzoate, sodium bisulfite, methylparaben, propylparaben), stabilizers (such as citric acid, sodium citrate, acetic acid, and multicarboxylic acids from the Titriplex series, such as e.g. diethylenetriaminepentaacetic acid (DTPA)), suspending agents (such as methylcellulose, polyvinylpyrrolidone, aluminium stearate), dispersing agents, diluents (such as water, organic solvents), beeswax, cacao butter, polyethylene glycol, white petrolatum etc.

Liquid medicament formulations, such as solutions, suspensions and gels, conventionally contain a liquid carrier, such as water and/or pharmaceutically acceptable organic solvents. Such liquid formulations can furthermore also contain pH-adjusting agents, emulsifiers or dispersing agents, buffering agents, preservatives, wetting agents, gelling agents (for example methylcellulose), colouring agents and/or aroma substances. The compositions can be isotonic, that is to say these can have the same osmotic pressure as blood. The isotonicity of the composition can be adjusted using sodium chloride or other pharmaceutically acceptable agents, such as, for example, dextrose, maltose, boric acid, sodium tartrate, propylene glycol or other inorganic or organic soluble substances. The viscosity of the liquid compositions can be adjusted using a pharmaceutically acceptable thickening agent, such as methylcellulose. Other suitable thickening agents include, for example, xanthan, carboxymethylcellulose, hydroxypropylcellulose, carbomer and the like. The preferred concentration of the thickening agent will depend on the agent chosen. Pharmaceutically acceptable preservatives can be used to increase the life of the liquid composition. Benzyl alcohol may be suitable, although a large number of preservatives, including, for example, paraben, thimerosal, chlorobutanol or benzalkonium chloride, can likewise be used.

The active compound can be administered, for example, with a unit dose of from 0.001 mg/kg to 500 mg/kg of body weight, for example up to 1 to 4 times a day. However, the dosage can be increased or reduced, depending on the age, weight, condition of the patient, severity of the disease or nature of the administration.

A preferred embodiment relates to the use of the compounds according to the invention and of the compositions according to the invention comprising the compounds according to the invention and of the combination preparations according to the invention comprising the compounds and compositions according to the invention for the preparation of a medicament for oral or parenteral administration.

Particular embodiments of the invention relate to:
1. Compounds of the general formula (I)

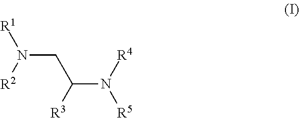

wherein
R¹ and R² are identical or different and are each chosen from the group consisting of:
   hydrogen,
   optionally substituted acyl,
   optionally substituted alkyl,
   optionally substituted aryl, and
   optionally substituted heterocyclyl; or
R¹ and R² together with the nitrogen atom to which they are bonded form a saturated or unsaturated, optionally substituted 5- to 8-membered ring which can optionally contain further hetero atoms;
R³ is chosen from the group consisting of:
   optionally substituted aryl, and
   optionally substituted heterocyclyl;
R⁴ and R⁵ are identical or different and are each chosen from the group consisting of:
   hydrogen,
   optionally substituted alkyl-, aryl- or heterocyclylsulfonyl,
   optionally substituted acyl,
   optionally substituted alkyl,
   optionally substituted alkenyl,
   optionally substituted alkynyl,
   optionally substituted aryl, and
   optionally substituted heterocyclyl; or
R⁴ and R⁵ together with the nitrogen atom to which they are bonded form a saturated or unsaturated, optionally substituted 5-to 8-membered ring which can optionally contain further hetero atoms;
or pharmaceutically acceptable salts thereof.
2. Compounds according to embodiment 1, wherein
   R¹ and R² are identical or different and are each chosen from the group consisting of:
      hydrogen,
      optionally substituted alkyl,
      optionally substituted aryl, and
      optionally substituted heterocyclyl; or
   R¹ and R² together with the nitrogen atom to which they are bonded form a saturated or unsaturated, optionally substituted 5- to 6-membered ring which can optionally contain further hetero atoms;
   R³ is chosen from the group consisting of:
      optionally substituted aryl, and
      optionally substituted heterocyclyl;
   R⁴ and R⁵ are identical or different and are each chosen from the group consisting of:
      hydrogen,
      optionally substituted alkyl,
      optionally substituted aryl, and
      optionally substituted heterocyclyl; or
   R⁴ and R⁵ together with the nitrogen atom to which they are bonded form a saturated or unsaturated, optionally substituted 5-to 6-membered ring which can optionally contain further hetero atoms;
   or pharmaceutically acceptable salts thereof.
3. Compounds according to embodiment 1 or 2, wherein

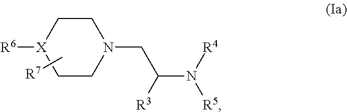

wherein
X is chosen from: N or CH;
R⁶ is chosen from the group consisting of:
   hydrogen,
   optionally substituted alkyl,
   optionally substituted alkenyl,
   optionally substituted alkynyl,
   optionally substituted acyl,
   optionally substituted alkoxycarbonyl,
   optionally substituted aryl, and
   optionally substituted heterocyclyl;
R⁷ is chosen from the group consisting of:
   hydrogen,
   hydroxyl,
   halogen,
   cyano,
   nitro,
   carboxyl,
   sulfonic acid radical (−SO₃H),
   optionally substituted amino,
   optionally substituted aminocarbonyl,
   optionally substituted aminosulfonyl,
   optionally substituted acyl,
   optionally substituted acyloxy,
   optionally substituted alkoxy,
   optionally substituted alkoxycarbonyl,
   optionally substituted alkyl,
   optionally substituted alkenyl,
   optionally substituted alkynyl,
   optionally substituted aryl, and
   optionally substituted heterocyclyl;
or pharmaceutically acceptable salts thereof.
4. Compounds according to embodiment 3, wherein
   X is chosen from: N or CH;
   R⁶ is chosen from the group consisting of:
      hydrogen,
      optionally substituted alkyl,
      optionally substituted acyl,
      optionally substituted aryl, and
      optionally substituted heterocyclyl;
   R⁷ is chosen from the group consisting of:
      hydrogen,
      halogen,
      optionally substituted amino,
      optionally substituted acyl,
      optionally substituted alkoxy,
      optionally substituted alkyl,
      optionally substituted aryl, and
      optionally substituted heterocyclyl;
   R³ is chosen from the group consisting of:
      optionally substituted aryl, and
      optionally substituted heterocyclyl;
   R⁴ and R⁵ are identical or different and are each chosen from the group consisting of:
      hydrogen,
      optionally substituted alkyl,
      optionally substituted aryl, and
      optionally substituted heterocyclyl; or
   R⁴ and R⁵ together with the nitrogen atom to which they are bonded form a saturated or unsaturated, optionally substituted 5- to 6-membered ring which can optionally contain further hetero atoms;
   or pharmaceutically acceptable salts thereof.
5. Compounds according to embodiment 3 or 4, wherein
   X has the meaning N;
   R⁶ is chosen from the group consisting of:
      optionally substituted acyl,
      optionally substituted aryl, and
      optionally substituted heterocyclyl;
   R⁷ is hydrogen;
   R³ is chosen from the group consisting of:
      optionally substituted aryl, and
      optionally substituted heterocyclyl;
   R⁴ and R⁵ are identical or different and are each chosen from the group consisting of:
      hydrogen or
      optionally substituted alkyl,
   or pharmaceutically acceptable salts thereof.
6. Compounds according to embodiment 3 or 4, wherein
   X has the meaning CH; and
   R⁶ is chosen from the group consisting of:
      optionally substituted aryl, and
      optionally substituted heterocyclyl;
   R⁷ is hydrogen;
   R³ is chosen from the group consisting of:
      optionally substituted aryl, and
      optionally substituted heterocyclyl;
   R⁴ and R⁵ are identical or different and are each chosen from the group consisting of:
      hydrogen,
      optionally substituted alkyl; or
   R⁴ and R⁵ together with the nitrogen atom to which they are bonded form a saturated or unsaturated, optionally substituted 6-membered ring which can optionally contain further hetero atoms;
   or pharmaceutically acceptable salts thereof.
7. Compounds according to one or more of embodiments 1 to 6, wherein R¹ and R² together with the nitrogen atom to which they are bonded form an optionally substituted, saturated or unsaturated 6-membered ring which can optionally contain one to 3 further hetero atoms.
8. Compounds according to one or more of embodiments 1 to 7, wherein R³ is optionally substituted aryl or optionally substituted heterocyclyl.
9. Compounds according to one or more of embodiments 1 to 8, wherein one of the radicals R⁴ or R⁵ is hydrogen, and the other radical of the radicals R⁴ or R⁵ is optionally substituted alkyl, or R⁴ and R⁵ together with the nitrogen atom to which they are bonded form an optionally substituted, saturated or unsaturated 6-membered ring which can optionally contain one to 3 further hetero atoms.
10. Compounds according to one or more of the embodiments, chosen from:

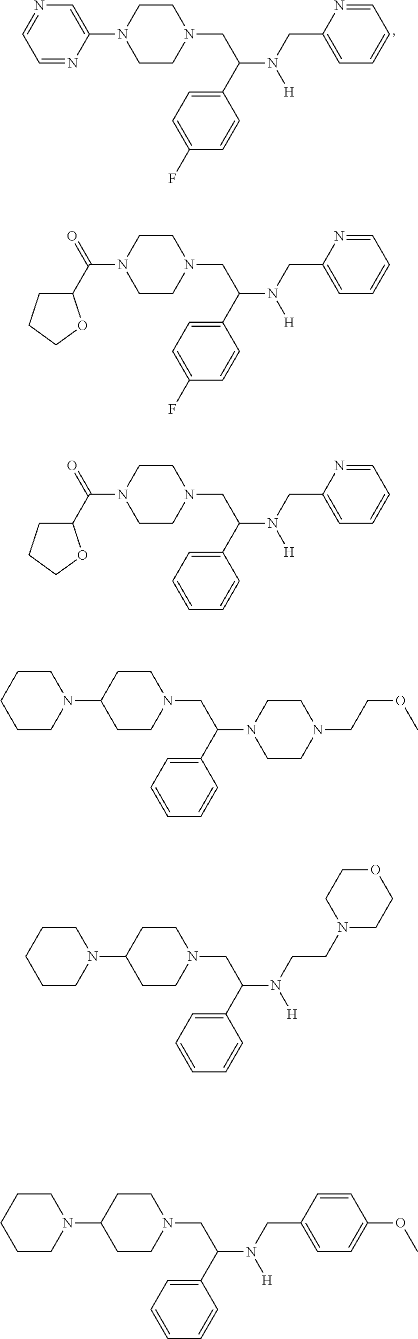

or pharmaceutically acceptable salts thereof.
11. Process for the preparation of the compounds of the general formula (I) according to one of embodiments 1 to 10, wherein compounds of the formula (II):

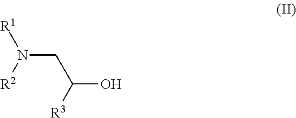

wherein R¹, R² and R³ are as defined above, are reacted with compounds of the formula

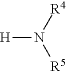

to give compounds of the formula (I).
12. Compounds according to one or more of embodiments 1 to 10 for use as medicaments.
13. Compounds according to one or more of embodiments 1 to 10 for use in the treatment of disorders in iron metabolism, in particular for use for iron deficiency diseases and/or anaemias, in particular anaemias with cancer, anaemia induced by chemotherapy, anaemia induced by inflammation (AI), anaemias with congestive cardiac insufficiency (CHF; congestive heart failure), anaemia with chronic renal insufficiency stage 3-5 (CKD 3-5; chronic kidney diseases stage 3-5), anaemia induced by chronic inflammation (ACD), anaemia with rheumatic arthritis (RA; rheumatoid arthritis), anaemia with systemic lupus erythematosus (SLE) and anaemia with inflammatory intestinal diseases (IBD; inflammatory bowel disease).
14. Composition comprising one or more of the compounds according to one or more of embodiments 1 to 10 and one or more pharmaceutical carriers and/or auxiliary substances and/or solvents
15. Combination preparation comprising one or more of the compounds according to one or more of embodiments 1 to 10 and at least one further pharmaceutically active compound, which is, in particular, a compound for treatment of disorders in iron metabolism and the accompanying symptoms, preferably an iron-containing compound.
16. Use of the compounds according to one or more of embodiments 1 to 10, of the composition according to embodiment 14 and of the combination preparation according to embodiment 15 for the preparation of a medicament for treatment of hepcidin-mediated diseases and the accompanying symptoms, in particular for treatment of disorders in iron metabolism, in particular iron deficiency diseases and/or anaemias, in particular ACD and AI, and the accompanying symptoms.
17. Use of the compounds according to one or more of embodiments 1 to 10, of the composition according to embodiment 14 and of the combination preparation according to embodiment 15 for the preparation of a medicament for oral or parenteral administration.

The invention is illustrated in more detail by the following examples. The examples are given merely by way of example and the person skilled in the art is in a position to extend the specific examples to further compounds claimed.

### EXAMPLES

### Pharmacological Action Studies

The following materials were used:

| Reagents | Batch no. | Comments |
|---|---|---|
| MDCK-FPN-HaloTag clone 7 | | |
| Hepcidin 100 μM stock | Lot# 571007 | Peptides International |
| solution in water | | |
| HaloTag ®TMR ligand | Lot# 257780 | Promega, cat# G8251 |
| Opera confocal plate imager | | PerkinElmer |
| Perkin Elmer 384 Cell carrier | | cat# 6007430 |
| plates | | |
| Paraformaldehyde | Lot# 080416 | Electron Microscopy |
| | | Sciences cat# 15710-S |
| Draq5 | | Biostatus, cat no: DR51000 |

The hepcidin-antagonistic action of the ethanediamine compounds of the present invention was determined by means of the “ferroportin internalization assay” described in the following.

### Principle of the “Ferroportin Internalization Assay”

Organic compounds of low molecular weight which counteract the biological actions of hepcidin on its receptor, the iron exporter ferroportin (Fpn), were identified on the basis of their ability to inhibit hepcidin-induced internalization of Fpn in living cells. For this purpose, a stable cell line (Madin-Darby canine kidney, MDCK) was produced which constitutively expresses human ferroportin fused recombinantly at its C terminus with a fluorescent reporter protein (HaloTag®, Promega Corp.). The internalization of Fpn was monitored by labelling these cells with fluorescent ligands (HaloTag®-TMR, tetramethylrhodamine) which join covalently on to the HaloTag reporter gene fused with the Fpn. Imaging by confocal fluorescence microscopy showed a cell surface location of Fpn in the absence of hepcidin and the absence of Fpn surface staining in the presence of hepcidin. Optimized image analysis algorithms were used to ascertain the cell surface and to quantify the corresponding membrane fluorescence associated with the Fpn-HaloTag fusion protein. This assay allows a quantitative image-based analysis in order to quickly evaluate compounds which can block hepcidin-induced internalization of Fpn. This assay is a direct in vitro pendant of the in vivo action mechanism proposed for medicament candidates and is therefore suitable as an initial assay with a high throughput for identifying compounds which counteract the action of hepcidin on its receptor ferroportin.

### Detailed Assay Procedure

7,500 cells per well (MDCK-FPN-HaloTag) were transinoculated in 50 μl of DMEM medium (Dulbeccos Modified Eagle Medium with 10% foetal bovine serum (FBS), which contained 1% penicillin, 1% streptomycin and 450 μg/ml of G-418) in microtitre plates with 384 wells (384 Cell carrier plates, Perkin Elmer, cat. no. 6007430), followed by incubation overnight at 37° C./5% CO₂.
The volume of the medium was reduced to 10 μl, and 10 μl of 5 μM HaloTag-TMR ligands (Promega, cat. no. G 8251) were added in DMEM medium in order to stain the Fpn-HaloTag fusion protein.
15 min incubation at 37° C./5% CO₂
The HaloTag-TMR ligand was removed and the cells were washed with fresh DMEM medium and the volume was reduced to 20 μl of DMEM medium.
3 μl per well of a solution of the test compound (dissolved DMSO) were added (10 μl final volume).
7 μl of 43 μM hepcidin (Peptides International, cat. no. PLP-4392-s, 100 μM stock solution diluted in water in DMEM medium) were added per well up to a final hepcidin concentration of 100 nM.
The cells were incubated overnight at 37° C./5% CO₂.
The cells were fixed by adding paraformaldehyde (PFA, Electron Microscopy Sciences, cat. no. 15710-S) directly to the cells up to a final concentration of 4%, followed by incubation at room temperature for 15-20 minutes.
The PFA solution was removed and the cells were washed with PBS (phosphate-buffered saline solution), in each case 30 μl remaining in the plate.
20 μl of Draq5 (Biostatus, cat. no. DR 51000) were added up to a final concentration of 2.5 μM in order to stain the cell nuclei, and the plates were sealed with a foil plate seal.
The plates were analysed with the Opera Plate Imager (Opera Confocal Plate Imager, Perkin Elmer) with 7 images per well; 440 ms exposure time per image, 1 μM focal point height.

### Analysis of the Data

Optimized algorithms were used for the image analysis to ascertain and quantify the fluorescence associated with the cell surface as a measure of the cell surface location of Fpn-HaloTag.
The final display corresponded to the percentage content of cells which showed membrane fluorescence: wells treated with 100 nM hepcidin gave the lowest values (negative control display=0% inhibition of the Fpn internalization) and wells which were not treated with hepcidin resulted in the maximum percentage content of cells with membrane fluorescence (positive control display 100% inhibition of the Fpn internalization).
On each plate, the median value of the 6 positive and 6 negative control values was used to calculate the percentage inhibition of the compounds tested according to the following formula:

$$I = 100 \times \frac{{R}_{neg} - {R}_{compound}}{{R}_{neg} - {R}_{pos}}$$

where: Rₚₒₛ positive control display value (median)
   R_{neg} negative control display value (median)
   R_{compound} display value of the compound investigated
   I percentage inhibition by the particular compound
In dose/effect studies, dilution series (11 concentrations, 1:2 dilution steps) of the compounds were tested (concentration range from 0.04 to 40 μM), and standardized signal values of replicated tests (average of 6 titrations on independent plates) were used to fit the curves by a robust standard dose/effect model with four parameters (lower asymptote, upper asymptote, IC50, gradient).

The following results were obtained:

| | | | I [%] |
|---|---|---|---|
| | | | (median |
| | | | inhibition [%] |
| | | | at 10 μm |
| | | Ferroportin | substance |
| Example | Compound | IC50 [μM] | conc.) |
| 1 | 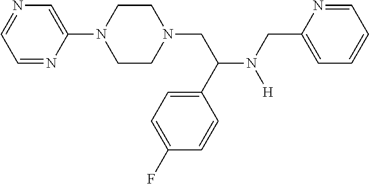 | <50 | >50 |
| 2 | 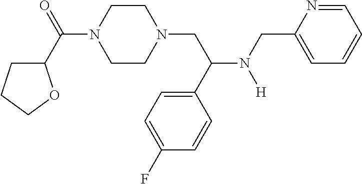 | — >100 | <50 |
| 3 | 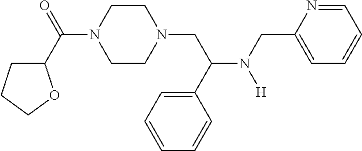 | — <50 | <50 |
| 4 | 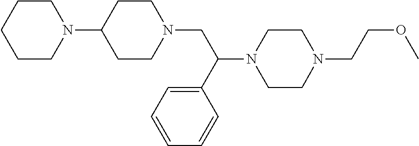 | <50 | >50 |
| 5 | 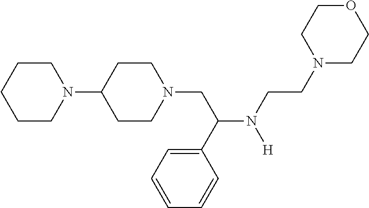 | <100 | <50 |
| 6 | 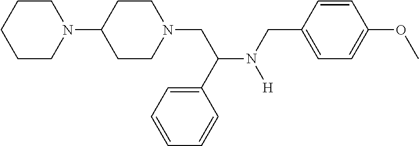 | <100 | <50 |
| 7 | 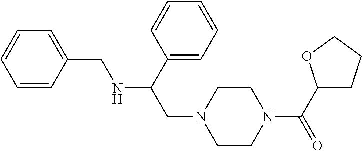 | >100 | |
| 8 | 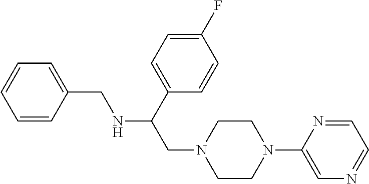 | >50 | |
| 9 | 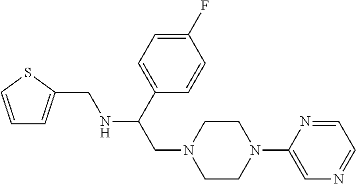 | >50 | |
| 10 | 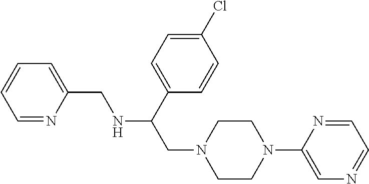 | >100 | |
| 11 | 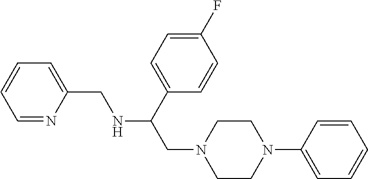 | >100 | |
| 12 | 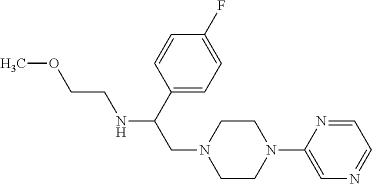 | >100 | |
| 13 | 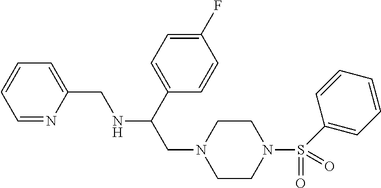 | >100 | |
| 14 | 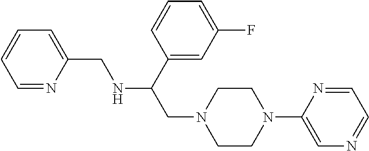 | >100 | |
| 15 | 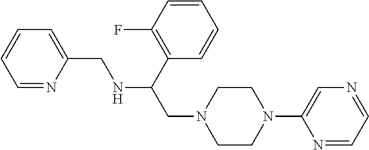 | >100 | |
| 16 | 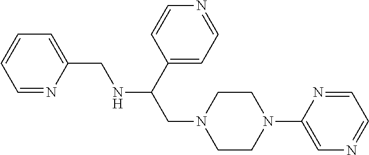 | >100 | |
| 17 | 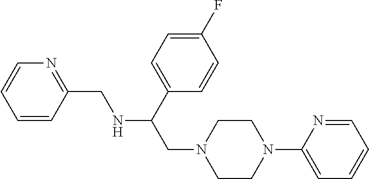 | >100 | |
| 18 | 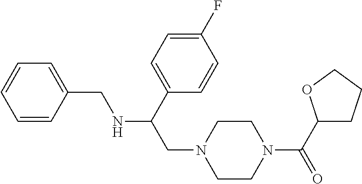 | >100 | |
| 19 | 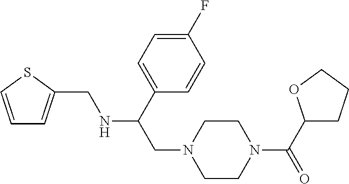 | >100 | |
| 20 | 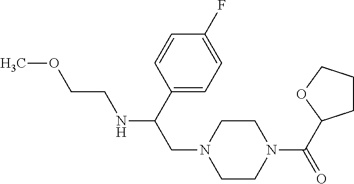 | >100 | |
| 21 | 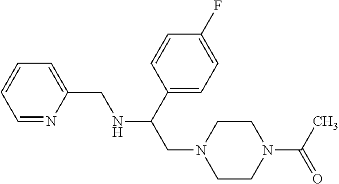 | >100 | |
| 22 | 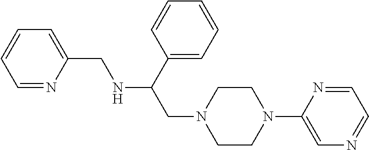 | >100 | |
| 23 | 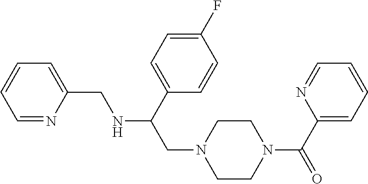 | >100 | |
| 24 | 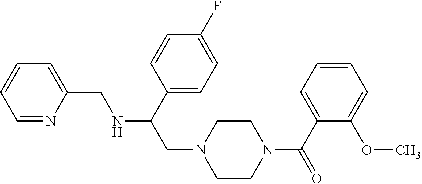 | <50 | |
| 25 | 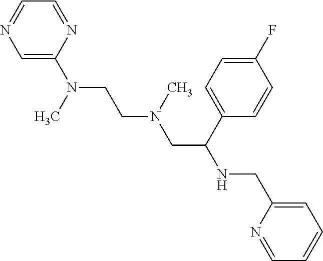 | <50 | |
| 26 | 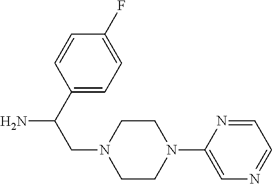 | >100 | |
| 27 | 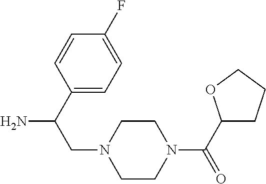 | >100 | |
| 28 | 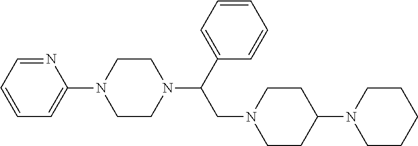 | >100 | |
| 29 | 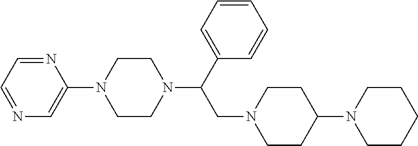 | >100 | |
| 30 | 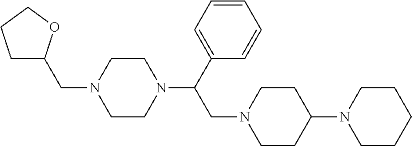 | <50 | |
| 31 | 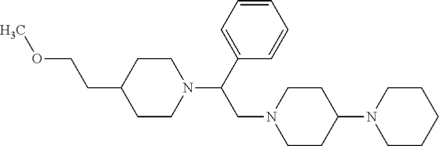 | >100 | |
| 32 | 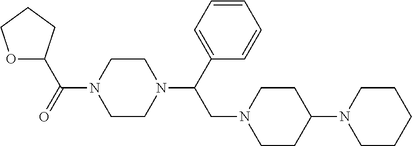 | >100 | |
| 33 | 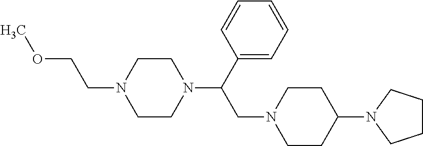 | <50 | |
| 34 | 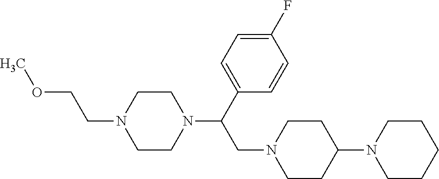 | <50 | |
| 35 | 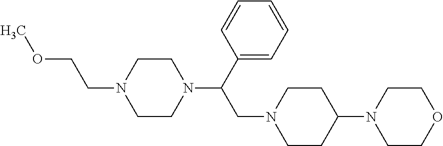 | <50 | |
| 36 | 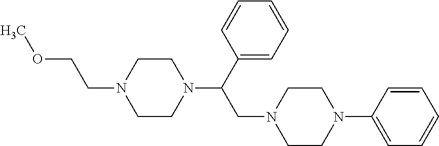 | >100 | |
| 37 | 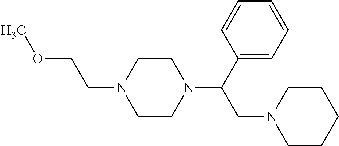 | <100 | |
| 38 | 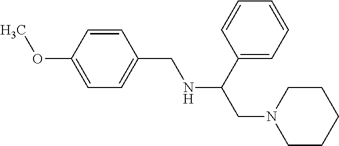 | >100 | |
| 39 | 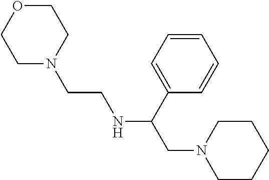 | <100 | |
| 40 | 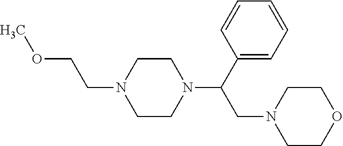 | >100 | |
| 41 | 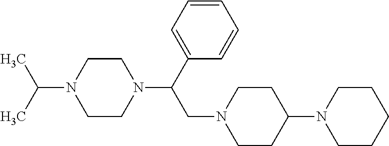 | >100 | |
| 42 | 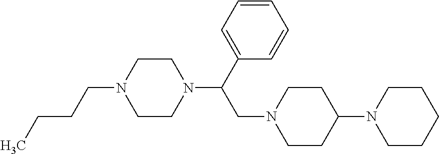 | >100 | |
| 43 | 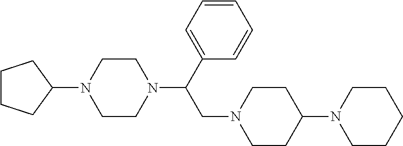 | >100 | |
| 44 | 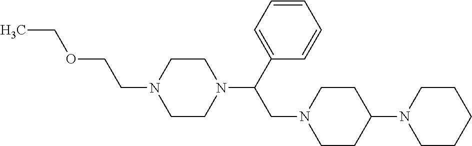 | <50 | |
| 45 | 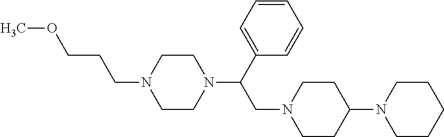 | <50 | |
| 46 | 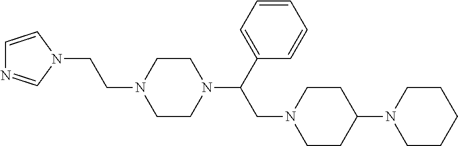 | >100 | |
| 47 | 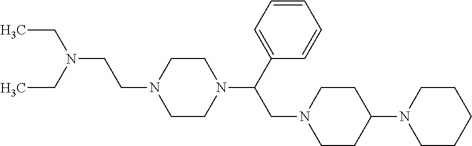 | >100 | |
| 48 | 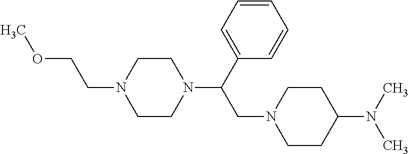 | <50 | |
| 49 | 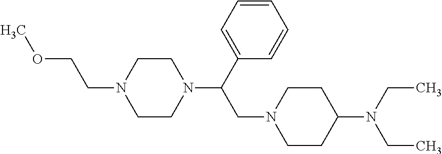 | <50 | |
| 50 | 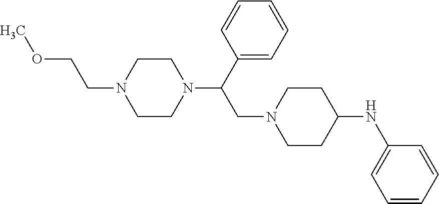 | >100 | |
| 51 | 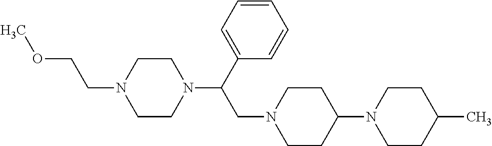 | <100 | |
| 52 | 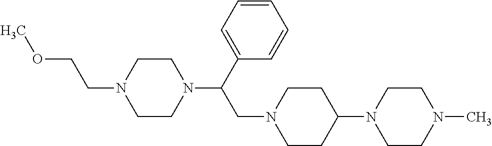 | >100 | |
| 53 | 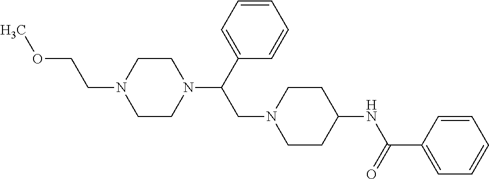 | >100 | |
| 54 | 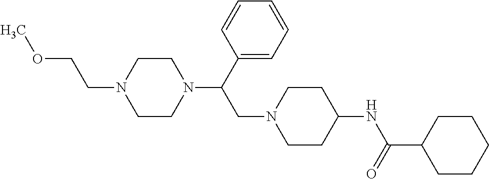 | <50 | |
| 55 | 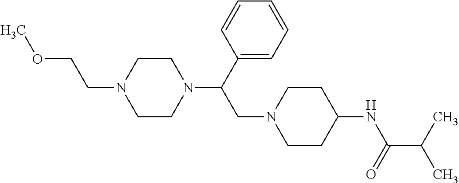 | <100 | |
| 56 | 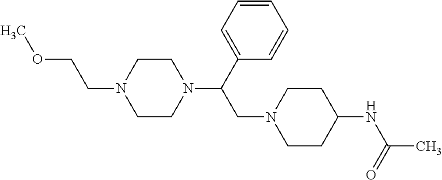 | <100 | |
| 57 | 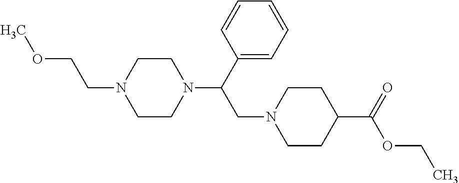 | >100 | |
| 58 | 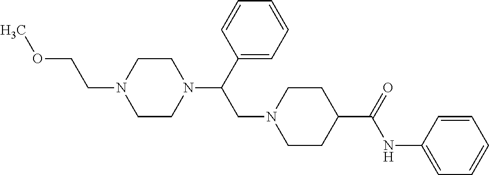 | <100 | |
| 59 | 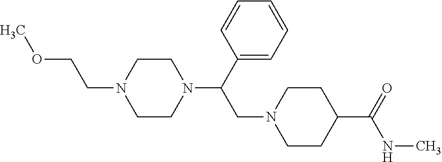 | <100 | |
| 60 | 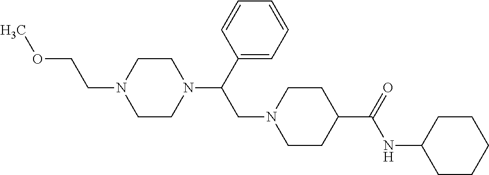 | >100 | |
| 61 | 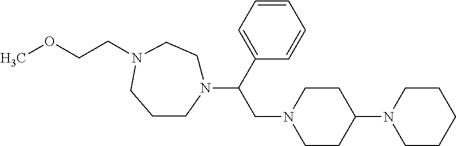 | <50 | |
| 62 | 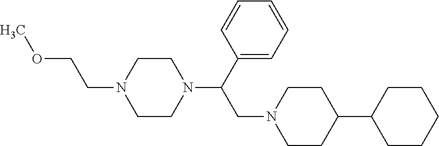 | >100 | |
| 63 | 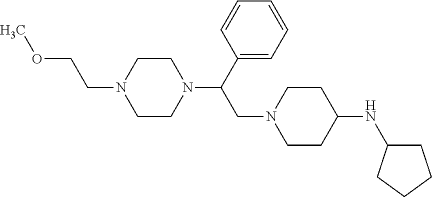 | <50 | |
| 64 | 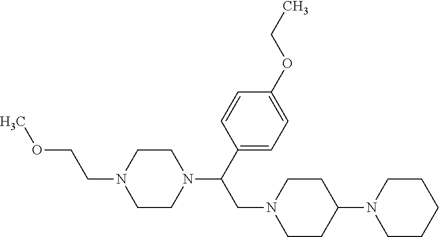 | >100 | |
| 65 | 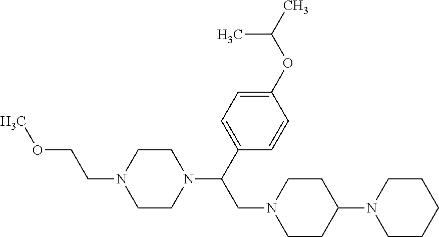 | >100 | |
| 66 | 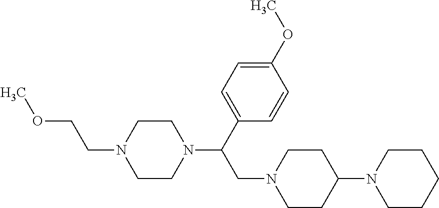 | <50 | |
| 67 | 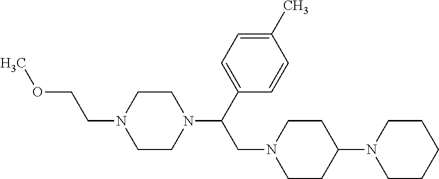 | <50 | |
| 68 | 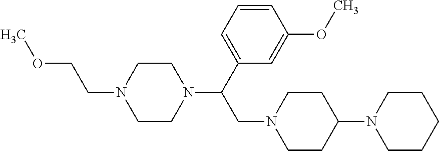 | <100 | |
| 69 | 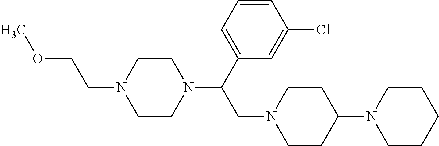 | >100 | |
| 70 | 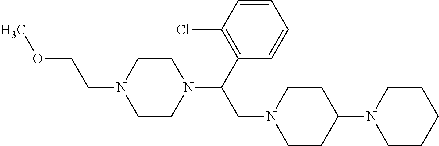 | >100 | |
| 71 | 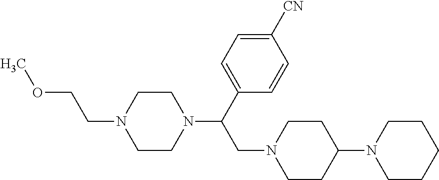 | <50 | |
| 72 | 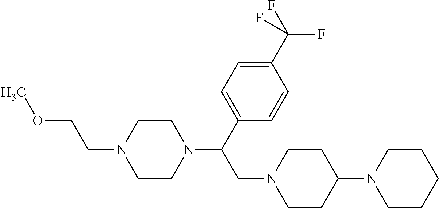 | <50 | |
| 73 | 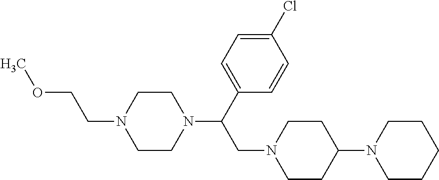 | >100 | |

### Preparation Examples

### I. Purification by Means of Preparative HPLC and Column Chromatography

The following preparation examples were carried out according to the preparation process according to the invention optionally with subsequent purification by means of preparative HPLC and/or by means of column chromatography under the following conditions:

### I.I Preparative HPLC (Basic Conditions)

Method: Gilson semi-prep HPLC with 119 UV detector and 5.11 Unipoint control software
Stationary phase/column: XBridge Prep C18 OBD (5 μm 19×100 mm), room temperature
Mobile phase: A: water+0.2% ammonium hydroxide
   B: acetonitrile+0.2% ammonium hydroxide
Flow rate: 20 ml/min
Injection volume: 1,000 μl
Detection: UV
Eluent:

| Time (minutes). | Solvent |
|---|---|
| 0.0 to 2.0 | 5% B + 95% A |
| 2.0 to 2.5 | constant gradient to 10% B + 90% A |
| 2.5 to 14.5 | constant gradient to 100% B |
| 14.5 to 16.5 | 100% B |
| 16.5 to 16.7 | constant gradient to 5% B + 95% A |
| 16.7 to 17.2 | 5% B + 95% A |

### I.II Preparative HPLC (Acid Conditions)

Method: Gilson 215 autosampler and fraction collector
Stationary phase/column: Waters SunFire Prep C18 OBD (5 μm 19×100 mm), room temperature
Mobile phase: A: 0.1% TFA/water
   B: 0.1% TFA/acetonitrile
Flow rate: 26 ml/min
Injection volume: 1,000 μl
Detection: Waters Micromass Platform LCZ single quadrupole mass spectrometer
Waters 600 solvent delivery module
Waters 515 ancillary pumps
Waters 2487 UV-detector
Eluent:

| Time (minutes) | Solvent |
|---|---|
| 0.0 to 1.0 | 90% A + 10% B |
| 1.0 to 7.5 | constant gradient from 90% A + 10% B to 100% B |
| 7.5 to 9.0 | 100% B |
| 9.0 to 9.1 | constant gradient from 100% B to 90% A + 10% B |
| 9.1 to 10.0 | 90% A + 10% B |

### I.III Column Chromatography:

The “flash” silica gel chromatography was carried out by means of silica gel 230 to 400 mesh or on prepacked silica columns.

### II. Analytical HPLC-MS

The detection and determination of the purity of the compounds were in each case carried out by means of HPLC MS (high performance liquid chromatography with mass spectrometry (MS)) or by means of HPLC with UV detection (PDA; photo diode array).
Method: MS19_{—}7MIN_HIRES_POS/high resolution method
MS detection: TIC (total ion count)
HPLC-MS system: Shimadzu LCMS (Liquid Chromatography with mass spectrometry (MS)) 2010EV system
Mass range: 100-1,000 m/z
Scan speed: 2,000 amu/sec

In detail, the following methods were used in particular.

### II.I Method A

Stationary phase/column: Waters Atlantis dC18 (2.1×100 mm, 3 μm column); 40° C.
Flow rate: 0.6 ml/min
Mobile phase: A: 0.1% formic acid/water
   B: 0.1% formic acid/acetonitrile
Flow rate: 0.6 ml/min
Injection volume: 3 μl
Detection: UV, wavelength 215 nm
Eluent:

Gradient

| Time (min) | Organic content (%) |
|---|---|
| 0.00 | 5 |
| 5.00 | 100 |
| 5.40 | 100 |
| 5.42 | 5 |

| Time (minutes) | Solvent |
|---|---|
| 0 to 5 | constant gradient from 95% A + 5% B to 100% B |
| 5.0 to 5.4 | 100% B |
| 5.4 to 5.42 | constant gradient from 100% B to 95% A + 5% B |
| 5.42 to 7.0 | 95% A + 5% B |

### II.II Method B

Stationary phase/column: Waters Atlantis dC18 (2.1×50 mm, 3 μm)
Mobile phase: A: 0.1% formic acid/water
   B: 0.1% formic acid/acetonitrile
Flow rate: 1 ml/min
Injection volume: 3 μl
Detection: UV, wavelength 215 nm
Eluent

| Time (minutes) | Solvent |
|---|---|
| 0.0 to 2.5 | constant gradient from 95% A + 5% B to 100% B |
| 2.5 to 2.7 | 100% B |
| 2.71 to 3.0 | 95% A + 5% B |

### II.III Method C

Stationary phase/column: Waters Atlantis dC18 (2.1×30 mm, 3 μm column);
Flow rate: 1 ml/min
Mobile phase: A: 0.1% formic acid/water
   B: 0.1% formic acid/acetonitrile
Injection volume: 3 μl
Detection: UV, wavelength 215 nm
Eluent

| Time (minutes) | Solvent |
|---|---|
| 0.0 to 1.5 | constant gradient from 95% A + 5% B to 100% B |
| 1.5 to 1.6 | 100% B |
| 1.60 to 1.61 | constant gradient from 100% B to 95% A + 5% B |
| 1.61 to 2.00 | 95% A + 5% B |

MS detection: Waters LCT or LCT Premier or ZQ or ZMD
UV detection: Waters 2996 photodiode array or Waters 2787 UV or Waters 2788 UV

### II.IV Method D

Stationary phase/column: Waters Atlantis dC18 (50 mm×3 mm; 3 μm); 35° C.
Mobile phase: A: 0.1% formic acid/water
   B: 0.1% formic acid/acetonitrile
Flow rate: 0.8 ml/min
Injection volume: 5 μl
Detection wavelength: diode array Spectrum I max (with scan in the range of from 210 to 350 nm)
Sampling rate: 5
Eluent

| Time (minutes) | Solvent |
|---|---|
| 0.0 | 95% A + 5% B |
| 0.2 | 95% A + 5% B |
| 0.2 to 3.2 | 95% A + 5% B |
| 5.0 | constant gradient from 95% A + 5% B to 5% A + 95% B |
| 5.0 | 5% A + 95% B |
| 5.0 to 5.2 | constant gradient from 5% A + 95% B to 95% A + 5% B |
| 5.5 | 95% A + 5% B |

MS detection: Waters LCT or LCT Premier or ZQ or ZMD
UV detection: Waters 2996 photodiode array or Waters 2787 UV or Waters 2788 UV

### II.V Method E

Stationary phase/column: Phenomenex Gemini C18 (100 mm×2.0 mm; 3 μm); 60° C.
Mobile phase: A: 2 mM ammonium bicarbonate, buffered to pH 10
   B: Acetonitrile
Flow rate: 0.5 ml/min
Injection volume: 3 μl
Detection: UV, wavelength 215 nm
Eluent

| Time (minutes) | Solvent |
|---|---|
| 0.0 | 95% A + 5% B |
| 0.2 to 5.5 | constant gradient from 95% A + 5% B to 5% A + 95% B |
| 5.50 to 5.90 | 100% B |
| 5.90 to 5.92 | constant gradient from 100% B to 95% A + 5% B |

### II.VI Method F

Stationary phase/column: ZORBAX Extend-C18 (50 mm×2.1 mm; 5 μm); 25° C.
Mobile phase: A: 2 mM ammonium bicarbonate, buffered to pH 10
   B: 5% 2 mM ammonium bicarbonate/acetonitrile
Flow rate: 4 ml/min
Injection volume: 15 μl
Detection: UV, wavelength 215 nm
Eluent

| Time (minutes) | Solvent |
|---|---|
| 0.0 | 99% A + 1% B |
| 0.2 to 1.8 | constant gradient from 95% A + 5% B to 100% B |
| 1.8 to 2.1 | 100% B |
| 2.11 to 2.30 | constant gradient from 100% B to 99% A + 1% B |
| 2.39 to 3.50 | 99% A + 1% B |

### III. Microwave Treatment

Microwave reactions were carried out by means of CEM Discover or Explorer focussed microwave apparatuses.

### IV. Designation of the Compounds

Some of the compounds described in the following were isolated as the TFA or HCl salt, which is not reflected by the chemical names stated. In the context of the present invention, the chemical names stated designate the corresponding compound in neutral form and the TFA salt or other salts thereof, in particular pharmaceutically acceptable salts, where applicable.

### V. Abbreviations

aq. aqueous
DCE 1,2-dichloroethane
MC methylene chloride
DIPEA N,N-diisopropylethylamine
DMF N,N-dimethylformamide
DMSO dimethylsulfoxide
eq equivalents
Et₂O diethyl ether
EtOAc ethyl acetate
EtOH ethanol
h hour(s)
HATU 1-[bis(dimethylamino)methylene]-1H-1,2,3-triazole[4,5-b]-pyridinium 3-oxide
HPLC high performance liquid chromatography
MeCN acetonitrile
MeOH methanol min minute(s)
MsCl methanesulfonyl chloride
MW molecular weight
Pd—C 10% palladium on charcoal
TEA triethylamine
TFA trifluoroacetic acid
THF tetrahydrofuran

### VI. Preparation Examples According to A) Synthesis Route 1

### VI.I Intermediate Products According to A) Synthesis Route 1

### Intermediate Product 1: 4-Oxiranyl-pyridine

### (Process Step 1)

Trimethylsulfonium chloride (2.17 g, 10 mmol) and potassium hydroxide (3.57 g, 60 mmol) were suspended in MeCN.

Water (48 μl, 2.65 ml) was added and the mixture was stirred at room temperature for 10 min. 4-Pyridinecarboxaldehyde (1.9 ml, 10 mmol) was added and the resulting reaction mixture was heated at 60° C. for 1.5 hours. After cooling, the mixture was filtered and the filtrate was concentrated in vacuo to give the crude yield, which was purified by means of column chromatography with MC/MeOH (99:1-98:2) as the eluent to give intermediate product 1 (607 mg, 47%). It was not possible to detect the compound by means of HPLCMS and the structure was therefore confirmed by means of ¹H NMR (nuclear magnetic resonance).

### Intermediate Product 2: (4-[2-(4-Fluoro-phenyl)-2-hydroxy-ethyl]-piperazin-1-yl}-(tetrahydro-furan-2-yl)-methanone

### (Process Step 2)

2-(4-Fluoro-phenyl)-oxirane (2.00 g, 14.48 mmol) and 1-(tetrahydro-2-furoyl)-piperazine (2.67 g, 14.48 mmol) were heated in a closed tube at 90° C. for three hours to give intermediate product 2 (4.67 g, 100%).

MW: 322.38

HPLCMS (method B): [m/z]: 323

### Intermediate Product 3: 1-(4-Fluoro-phenyl)-2-(2,3,5,6-tetrahydro-[1,2′]bipyrazinyl-4-yl)-ethanol

The preparation was carried out analogously to intermediate product 2 according to process step 2 using:

2,2-(4-fluorophenyl)-oxirane (2.00 g, 14.48 mmol) and 1-(2-pyrazinyl)-piperazine (2.38 g, 14.48 mmol) to give intermediate product 3 (4.38 g, 100%).

MW: 302.35

HPLCMS (method B): [m/z]: 303

### Intermediate Product 4: [4-(2-Hydroxy-2-phenyl-ethyl)-piperazin-1-yl]-(tetrahydro-furan-2-yl)-methanone

The preparation was carried out analogously to intermediate product 2 according to process step 2 using:

styrene oxide (2.00 g, 16.65 mmol) and 1-(tetrahydro-2-furoyl)-piperazine (3.06 g, 16.65 mmol) to give intermediate product 4 (5.07 g, 100%).

MW: 304.39

HPLCMS (method B): [m/z]: 305

### Intermediate Product 5: 1-(4-Chloro-phenyl)-2-(2,3,5,6-tetrahydro-[1,2′]bipyrazinyl-4-yl)-ethanol

The preparation was carried out analogously to intermediate product 2 according to process step 2 using:

2-(4-chloro-phenyl)-oxirane (779.42 μl, 6.47 mmol) and 1-(2-pyrazinyl)piperazine (1.06 g, 6.47 mmol) to give intermediate product 5 (2.00 g, 84%)

MW: 318.8

HPLCMS (method B): [m/z]: 319

### Intermediate Product 6: 1-(4-Fluoro-phenyl)-2-(4-phenyl-piperazin-1-yl)-ethanol

The preparation was carried out analogously to intermediate product 2 according to process step 2 using:

2-(4-fluorophenyl)-oxirane (1.00 g, 7.24 mmol) and 1-phenylpiperazine (1.106 ml, 7.24 mmol) to give intermediate product 6 (2.17 g, 100%).

MW: 300.37

HPLCMS (method B): [m/z]: 301

### Intermediate Product 7: 2-(4-Benzenesulfonyl-piperazin-1-yl)-1-(4-fluoro-phenyl)-ethanol

The preparation was carried out analogously to intermediate product 2 according to process step 2 using:

2-(4-fluorophenyl)-oxirane (1.00 g, 7.24 mmol) and 1-phenylsulfonylpiperazine (1.54 ml, 7.24 mmol) to give intermediate product 7 (2.5 g, 95%)

MW: 364.43

HPLCMS (method B): [m/z]: 365

### Intermediate Product 8: 1-(3-Fluoro-phenyl)-2-(2,3,5,6-tetrahydro-[1,2′]bipyrazinyl-4-yl)-ethanol

The preparation was carried out analogously to intermediate product 2 according to process step 2 using:

2-(3-fluorophenyl)-oxirane (560 mg, 4.05 mmol) and 1-(2-pyrazinyl)piperazine (666 mg, 4.05 mmol) to give intermediate product 8 (1.2 g 98%)

MW: 302.35

HPLCMS (method B): [m/z]: 303

### Intermediate Product 9: 1-(2-Fluoro-phenyl)-2-(2,3,5,6-tetrahydro-[1,2′]bipyrazinyl-4-yl)-ethanol

The preparation was carried out analogously to intermediate product 2 according to process step 2 using:

2-(1-fluorophenyl)-oxirane (440 mg, 3.19 mmol) and 1-(2-pyrazinyl)piperazine (523 mg, 3.19 mmol) to give intermediate product 9 (950 mg 98%)

MW: 302.35

HPLCMS (method B): [m/z]: 303

### Intermediate Product 10:1-Pyridin-4-yl-2-(2,3,5,6-tetrahydro-[1,2′]bipyrazinyl-4-yl)-ethanol

The preparation was carried out analogously to intermediate product 2 according to process step 2 using:

4-oxiranyl-pyridine (intermediate product 1) (607 mg, 5.0 mmol) and 1-(2-pyrazinyl)piperazine (823 mg, 5.0 mmol).

Purification was carried out by means of column chromatography with MC/MeOH (99:1-95:5) as the eluent to give intermediate product 10 (478 mg, 33%). It was not possible to detect the compound by means of HPLCMS and the structure was therefore confirmed by means of ¹H NMR.

### Intermediate Product 11: 1-(4-Fluoro-phenyl)-2-(4-pyridin-2-yl-piperazin-1-yl)-ethanol

The preparation was carried out analogously to intermediate product 2 according to process step 2 using:

2-(4-fluorophenyl)-oxirane (350 mg, 2.52 mmol) and 1-(2-pyridyl)piperazine (390 μl, 2.54 mmol) to give intermediate product 11 (765 mg, 100%).

MW: 301.37

HPLCMS (method B): [m/z]: 302

### Intermediate Product 12: 1-{4-[2-(4-Fluoro-phenyl)-2-hydroxy-ethyl]-piperazin-1-yl}-ethanone

The preparation was carried out analogously to intermediate product 2 according to process step 2 using:

2-(4-fluorophenyl)-oxirane (259 mg, 1.87 mmol) and 1-acetyl-piperazine (240 mg, 1.87 mmol) to give intermediate product 12 (498 mg, 100%).

MW: 266.32

HPLCMS (method B): [m/z]: 267

### Intermediate Product 13: 1-Phenyl-2-(2,3,5,6-tetrahydro-[1,2′]bipyrazinyl-4-yl)-ethanol

The preparation was carried out analogously to intermediate product 2 according to process step 2 using:

styrene oxide (1.00 g, 8.32 mmol) and 1-(2-pyrazinyl)piperazine (1.37 g, 8.32 mmol) to give intermediate product 13 (2.3 g, 87%).

MW: 284.36

HPLCMS (method B): [m/z]: 285

### VI.II Example Compounds According to A) Synthesis Route I

### Example Compound 2: (4-{2-(4-Fluoro-phenyl)-2-[(pyridin-2-ylmethyl)-amino]-ethyl}-piperazin-1-yl)-(tetrahydro-furan-2-yl)-methanone

### (Process Step 3)

{4-[2-(4-Fluoro-phenyl)-2-hydroxy-ethyl]-piperazin-1-yl}-(tetrahydro-furan-2-yl)-methanone (intermediate product 2) (1.40 g, 4.34 mmol) was dissolved in a stock solution of 11% TEA in THF (25 ml). Methanesulfonyl chloride (504 μl, 6.52 mmol) was added and the reaction was stirred at room temperature for one hour; the reaction was monitored by means of LCMS in order to confirm the reaction of the starting material. TEA (1.21 ml, 8.68 mmol) was added, followed by 2-aminomethylpyridine (542 μl, 5.21 mmol) in THF (2 ml). The reaction mixture was stirred at room temperature for 30 minutes. Water (2 ml) was added to the reaction mixture and stirring was continued for a further 18 hours or until the reaction of all the starting materials and reaction intermediate products was confirmed by means of LCMS. The reaction mixture was concentrated in vacuo. The crude yield was dissolved in MC and the solution was washed with water and brine. The resulting organic phase was dried (Na₂SO₄) and concentrated in vacuo. The crude material was purified by means of preparative HPLC (basic conditions) to give Example Compound 2 (135 mg, 8%).

EOAI3028737 VIT-1012

OP-18184-E04 (manufacturer: EVOTEC)

MW: 412.51 or 412.50

HPLCMS (method A): [m/z]: 413

UV spectrum: δ max [ηm]: 260

FIG. 2 shows the result.

### Example Compound 1: [1-(4-Fluoro-phenyl)-2-(2,3,5,6-tetrahydro-[1,2′]bipyrazinyl-4-yl)-ethyl]-pyridin-2-ylmethyl-amine

The preparation was carried out analogously to Example Compound 2 according to process step 3 using:

1-(4-fluoro-phenyl)-2-(2,3,5,6-tetrahydro-[1,2′]bipyrazinyl-4-yl)-ethanol (intermediate product 3) (1.00 g, 3.31 mmol), methanesulfonyl chloride (384 μl, 4.96 mmol), TEA (11% TEA in THF, 25 ml) and 2-aminomethylpyridine (413 μl, 3.97 mmol).

Purification was carried out by means of column chromatography with MC/MeOH (99:1-93:7) as the eluent to give Example Compound 1 (231 mg, 18%).

Salt formation: The yield was dissolved in MeOH (3 ml) and the solution was cooled to 0° C. HCl (3 eq)) was added and the reaction mixture was stirred at 0° C. for 20 minutes. The mixture was concentrated in vacuo to give Example Compound 1 in the form of the HCl salt.

EOAI3094765 VIT-1026

OP-19909-004 (manufacturer: EVOTEC)

MW: 392.50 or 392.47

HPLCMS (method A): [m/z]: 393

UV spectrum: δ max [ηm]: 193, 245, 327

FIG. 1 shows the result.

### Example Compound 3: (4-{2-Phenyl-2-[(pyridin-2-ylmethyl)-amino]-ethyl}-piperazin-1-yl)-(tetrahydro-furan-2-yl)-methanone

The preparation was carried out analogously to Example Compound 2 according to process step 3 using:

[4-(2-hydroxy-2-phenyl-ethyl)-piperazin-1-yl]-(tetrahydro-furan-2-yl)-methanone (intermediate product 4) (1.00 g, 3.29 mmol), methanesulfonyl chloride (381 μl, 4.93 mmol), TEA (11% TEA in THF, 25 ml) and 2-aminomethylpyridine (410 μl, 3.94 mmol).

Purification was carried out by means of column chromatography with MC/MeOH (99:1-95:5) as the eluent to give Example Compound 3 (379 mg, 29%).

Salt formation: The yield was dissolved in MeOH (3 ml) and the solution was cooled to 0° C. HCl (3 eq)) was added and the reaction mixture was stirred at 0° C. for 20 minutes. The mixture was concentrated in vacuo to give Example Compound 3 in the form of the HCl salt.

EOAI3094816 VIT-1027

OP-19909-D03 (manufacturer: EVOTEC)

MW: 394.52 or 394.51

HPLCMS (method A): [m/z]: 395

UV spectrum: δ max [ηm]: 193,259

FIG. 3 shows the result.

### Example Compound 7: [4-(2-Benzylamino-2-phenyl-ethyl)-piperazin-1-yl]-(tetrahydro-furan-2-yl)-methanone

The preparation was carried out analogously to Example Compound 2 according to process step 3 using:

[4-(2-hydroxy-2-phenyl-ethyl)-piperazin-1-yl]-(tetrahydro-furan-2-yl)-methanone (intermediate product 4) (500 mg, 1.64 mmol), methanesulfonyl chloride (190 μl, 2.46 mmol), TEA (11% TEA in THF, 15 ml), followed by benzylamine (215 μl, 1.97 mmol) and TEA (456 μl 3.29 mmol).

Purification was carried out by means of column chromatography with MC/MeOH (99:1-97:3) as the eluent to give Example Compound 7 (100 mg, 14%).

Salt formation: The yield was dissolved in MeOH (3 ml) and the solution was cooled to 0° C. HCl (3 eq)) was added and the reaction mixture was stirred at 0° C. for 20 minutes. The mixture was concentrated in vacuo to give Example Compound 7 in the form of the HCl salt.

EOAI3330478 VIT-1092

MW: 393.53

HPLCMS (method A): [m/z]: 394

FIG. 7 shows the result.

### Example Compound 8: Benzyl-[1-(4-fluoro-phenyl)-2-(2,3,5,6-tetrahydro-[1,2′]bipyrazinyl-4-yl)-ethyl]-amine

The preparation was carried out analogously to Example Compound 2 according to process step 3 using:

1-(4-fluoro-phenyl)-2-(2,3,5,6-tetrahydro-[1,2′]bipyrazinyl-4-yl)-ethanol (intermediate product 3) (500 mg, 1.65 mmol), methanesulfonyl chloride (192 μl, 2.48 mmol) and TEA (11% TEA in THF, 15 ml), followed by benzylamine (216.76 μl, 1.98 mmol) and TEA (461 μl, 3.31 mmol)

Purification was carried out by means of trituration from MeOH to give Example Compound 8 (61 mg, 9%).

EOAI3330479 VIT-1091

MW: 391.50

HPLCMS (method A): [m/z]: 392

FIG. 8 shows the result.

### Example Compound 9: [1-(4-Fluoro-phenyl)-2-(2,3,5,6-tetrahydro-[1,2′]bipyrazinyl-4-yl)-ethyl]-thiophen-2-ylmethyl-amine

The preparation was carried out analogously to Example Compound 2 according to process step 3 using:

1-(4-fluoro-phenyl)-2-(2,3,5,6-tetrahydro-[1,2′]bipyrazinyl-4-yl)-ethanol (intermediate product 3) (500 mg, 1.65 mmol), methanesulfonyl chloride (192 μl, 2.48 mmol) and TEA (11% TEA in THF, 15 ml), followed by 2-thiophenemethylamine (204 μl, 1.10 mmol) and TEA (461 μl, 3.31 mmol).

Purification was carried out by means of column chromatography with MC/MeOH (99:1-97:3) as the eluent to give Example Compound 9 (81 mg, 12%).

EOAI3330480 VIT-1090

MW: 397.52

HPLCMS (method A): [m/z]: 398

FIG. 9 shows the result.

### Example Compound 10: [1-(4-Chloro-phenyl)-2-(2,3,5,6-tetrahydro-[1,2′]bipyrazinyl-4-yl)-ethyl]-pyridin-2-ylmethyl-amine

The preparation was carried out analogously to Example Compound 2 according to process step 3 using:

1-(4-chloro-phenyl)-2-(2,3,5,6-tetrahydro-[1,2′]bipyrazinyl-4-yl)-ethanol (intermediate product 5) (500 mg, 1.57 mmol), methanesulfonyl chloride (182 μl, 2.55 mmol) and TEA (11% TEA in THF, 15 ml), followed by 2-aminomethylpyridine (196 μl, 1.88 mmol) and TEA (437 μl, 3.14 mmol).

Purification was carried out by means of column chromatography with MC/MeOH (99:1-94:6) as the eluent to give Example Compound 10 (77 mg, 12%).

EOAI3330566 VIT-1107

MW: 408.94

HPLCMS (method A): [m/z]: 409

FIG. 10 shows the result.

### Example Compound 11: [1-(4-Fluoro-phenyl)-2-(4-phenyl-piperazin-1-yl)-ethyl]-pyridin-2-ylmethyl-amine

The preparation was carried out analogously to Example Compound 2 according to process step 3 using:

1-(4-fluoro-phenyl)-2-(4-phenyl-piperazin-1-yl)-ethanol (intermediate product 6) (500 mg, 1.66 mmol), methanesulfonyl chloride (193 μl, 2.50 mmol) and TEA (232 μl, 1.67 mmol) and 2-aminomethylpyridine (208 μl, 1.99 mmol).

Purification was carried out by means of trituration from MeOH to give Example Compound 11 (275 mg, 42%).

EOAI3330736 VIT-1122

MW: 390.51

HPLCMS (method A): [m/z]: 391

FIG. 11 shows the result.

### Example Compound 12: [1-(4-Fluoro-phenyl)-2-(2,3,5,6-tetrahydro-[1,2′]pyrazinyl-4-yl)-ethyl]-(2-methoxy-ethyl)-amine

The preparation was carried out analogously to Example Compound 2 according to process step 3 using:

1-(4-fluoro-phenyl)-2-(2,3,5,6-tetrahydro-[1,2]bipyrazinyl-4-yl)-ethanol (intermediate product 3) (500 mg, 1.65 mmol), methanesulfonyl chloride (192 μl, 2.48 mmol), TEA (461 μl, 3.31 mmol) and 2-methoxyethylamine (172.52 μl, 1.98 mmol).

Purification was carried out by means of column chromatography with MC/MeOH (99:1-97:3) as the eluent to give Example Compound 12 (63 mg, 11%).

EOAI3094725 VIT-1124

MW: 359.45

HPLCMS (method A): [m/z]: 360

FIG. 12 shows the result.

### Example Compound 13: [2-(4-Benzenesulfonyl-piperazin-1-yl)-1-(4-fluoro-phenyl)-ethyl]-pyridin-2-ylmethyl-amine

The preparation was carried out analogously to Example Compound 2 according to process step 3 using:

2-(4-benzenesulfonyl-piperazin-1-yl)-1-(4-fluoro-phenyl)-ethanol (intermediate product 7) (500 mg, 1.372 mmol), methanesulfonyl chloride (159 μl, 2.06 mmol), TEA (382 μl, 2.74 mmol) and 2-aminomethylpyridine (171 μl, 1.64 mmol).

Purification was carried out by means of column chromatography with MC/MeOH (99:1-97:3) as the eluent to give Example Compound 13 (115 mg, 18%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes. The mixture was concentrated in vacuo to give Example Compound 13 in the form of the HCl salt.

EOAI3331307 VIT-1152

MW: 454.57

HPLCMS (method A): [m/z]: 455

FIG. 13 shows the result.

### Example Compound 14: [1-(3-Fluoro-phenyl)-2-(2,3,5,6-tetrahydro-[1,2′]bipyrazinyl-4-yl)-ethyl]-pyridin-2-ylmethyl-amine

The preparation was carried out analogously to Example Compound 2 according to process step 3 using:

1-(3-fluoro-phenyl)-2-(2,3,5,6-tetrahydro-[1,2′]bipyrazinyl-4-yl)-ethanol (intermediate product 8) (500 mg, 1.65 mmol), methanesulfonyl chloride (192 μl, 2.48 mmol), TEA (461 μl, 3.31 mmol) and 2-aminomethylpyridine (206 μl, 1.98 mmol).

Purification was carried out by means of column chromatography with MC/MeOH (99:1-97:3) as the eluent to give Example Compound 14 (51 mg, 8%).

EOAI3331308 VIT-1153

MW: 392.48

HPLCMS (method A): [m/z]: 393

FIG. 14 shows the result.

### Example Compound 15: [1-(2-Fluoro-phenyl)-2-(2,3,5,6-tetrahydro-[1,2′]bipyrazinyl-4-yl)-ethyl]-pyridin-2-ylmethyl-amine

The preparation was carried out analogously to Example Compound 2 according to process step 3 using:

1-(2-fluoro-phenyl)-2-(2,3,5,6-tetrahydro-[1,2′]dipyrazinyl-4-yl)-ethanol (intermediate product 9) (500 mg, 1.65 mmol), methanesulfonyl chloride (192 μl, 2.48 mmol), TEA (461 μl, 3.31 mmol) and 2-aminomethylpyridine (206.35 μl, 1.98 mmol).

After purification by means of preparative HPLC (acid conditions), Example Compound 15 was obtained in the form of the TFA salt.

Formation of the free base: The yield was dissolved in MeOH (3 ml), carbonate resin (5 eq) was added and the mixture was stirred at room temperature for 1 hour. The solution was filtered and the resin was washed with MeOH. The filtrate was concentrated in vacuo to give Example Compound 15 (110 mg, 17%).

Salt formation: The yield was dissolved in a minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 15 in the form of the HCl salt.

EOA3331581 VIT-1167

MW: 392.48

HPLCMS (method A): [m/z]: 393

FIG. 15 shows the result.

### Example Compound 16: Pyridin-2-ylmethyl-[1-pyridin-4-yl-2-(2,3,5,6-tetrahydro-[1,2′]bipyrazinyl-4-yl)-ethyl]-amine

The preparation was carried out analogously to Example Compound 2 according to process step 3 using:

1-pyridin-4-yl-2-(2,3,5,6-tetrahydro-[1,2′]bipyrazinyl-4-yl)-ethanol (intermediate product 10) (477 mg, 1.67 mmol), methanesulfonyl chloride (190 μl, 2.51 mmol), TEA (470 μl, 3.3 mmol) and 2-aminomethylpyridine (200 μl, 2.00 mmol).

Purification was carried out by means of preparative HPLC (basic conditions) to give Example Compound 16 (20 mg, 3%).

EOAI3332898 VIT-1173

MW: 375.48

HPLCMS (method A): [m/z]: 376

FIG. 16 shows the result.

### Example Compound 17: [1-(4-Fluoro-phenyl)-2-(4-pyridin-2-yl-piperazin-1-yl)-ethyl]-pyridin-2-ylmethyl-amine

The preparation was carried out analogously to Example Compound 2 according to process step 3 using:

1-(4-fluoro-phenyl)-2-(4-pyridin-2-yl-piperazin-1-yl)-ethanol (intermediate product 11) (765 mg, 2.54 mmol), methanesulfonyl chloride (0.29 ml, 3.8 mmol) and TEA (11% TEA in THF, 2 ml) followed by 2-aminomethylpyridine (310 μl, 3.05 mmol) and TEA (0.71 ml, 5.1 mmol).

Purification was carried out by means of column chromatography with MC/MeOH (99:1-98:2) as the eluent to give Example Compound 17 (400 mg, 40%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes.

The mixture was concentrated in vacuo to give Example Compound 17 in the form of the HCl salt.

EOAI3028733 VIT-1106

MW: 391.50

HPLCMS (method A): [m/z]: 392

FIG. 17 shows the result.

### Example Compound 18: {4-[2-Benzylamino-2-(4-fluoro-phenyl)-ethyl]-piperazin-1-yl}-(tetrahydro-furan-2-yl)-methanone

The preparation was carried out analogously to Example Compound 2 according to process step 3 using:

{4-[2-(4-fluoro-phenyl)-2-hydroxy-ethyl]-piperazin-1-yl}tetrahydro-furan-2-yl)-methanone (intermediate product 2) (523 mg, 1.62 mmol), methanesulfonyl chloride (0.19 ml, 2.43 mmol) and TEA (11% TEA in THF, 25 ml) followed by benzylamine (210 μl, 1.94 mmol) and TEA (0.45 ml, 3.24 mmol).

Purification was carried out by means of column chromatography with MC/MeOH (99:1-98:2) as the eluent to give Example Compound 18 (310 mg, 47%).

Salt formation: The yield was dissolved in MeOH (3 ml) and the solution was cooled to 0° C. HCl (3 eq)) was added and the reaction mixture was stirred at 0° C. for 20 minutes. The mixture was concentrated in vacuo to give Example Compound 18 in the form of the HCl salt.

EOAI3330573 VIT-1108

MW: 411.52

HPLCMS (method A): [m/z]: 412

FIG. 18 shows the result.

### Example Compound 19: (4-{2-(4-Fluoro-phenyl)-2-[(thiophen-2-ylmethyl)-amino]-ethyl}-piperazin-1-yl)-(tetrahydro-furan-2-yl)-methanone

The preparation was carried out analogously to Example Compound 2 according to process step 3 using:

{4-[2-(4-fluoro-phenyl)-2-hydroxy-ethyl]-piperazin-1-yl}-(tetrahydro-furan-2-yl)-methanone (intermediate product 2) (703 mg, 2.18 mmol), methanesulfonyl chloride (0.25 ml, 3.27 mmol) and TEA (11% TEA in THF, 1.9 ml) followed by 2-thiophenemethylamine (270 μl, 2.62 mmol) and TEA (0.6 ml, 4.36 mmol).

Purification was carried out by means of column chromatography with MC/MeOH (99:1-98:2) as the eluent to give Example Compound 19 (254 mg, 28%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes. The mixture was concentrated in vacuo to give Example Compound 19 in the form of the HCl salt.

EOAI3330742 VIT-1121

MW: 417.55

HPLCMS (method A): [m/z]: 418

FIG. 19 shows the result.

### Example Compound 20: {4-[2-(4-Fluoro-phenyl)-2-(2-methoxy-ethylamino)-ethyl]-piperazin-1-yl}-(tetrahydro-furan-2-yl)-methanone

The preparation was carried out analogously to Example Compound 2 according to process step 3 using:

{4-[2-(4-fluoro-phenyl)-2-hydroxy-ethyl]-piperazin-1-yl}-(tetrahydro-furan-2-yl)-methanone (intermediate product 2) (542 mg, 2.18 mmol), methanesulfonyl chloride (0.2 ml, 2.52 mmol) and TEA (11% TEA in THF, 1.5 ml) followed by 2-methoxyethylamine (175 μl, 2.00 mmol) and TEA (0.47 ml, 3.36 mmol).

Purification was carried out by means of column chromatography with MC/MeOH (99:1-98:2) as the eluent to give Example Compound 20 (235 mg, 37%).

Salt formation: The yield was dissolved in a minimum amount of Et₂O/MC and the solution was cooled to 0° C. 2 M HCl in Et₂O (2 eq) was added dropwise and the reaction mixture was stirred for 30 minutes. The mixture was concentrated in vacuo to give Example Compound 20 in the form of the HCl salt.

EOAI3094726 VIT-1125

MW: 379.48

HPLCMS (method A): [m/z]: 380

FIG. 20 shows the result.

### Example Compound 21: 1-(4-{2-(4-Fluoro-phenyl)-2-1[(pyridin-2-ylmethyl)-amino]-ethyl}-piperazin-1-yl)-ethanone

The preparation was carried out analogously to Example Compound 2 according to process step 3 using:

1-{4-[2-(4-fluoro-phenyl)-2-hydroxy-ethyl]-piperazin-1-yl}-ethanone (intermediate product 12) (458 mg, 1.87 mmol), methanesulfonyl chloride (0.22 ml, 2.8 mmol) and TEA (11% TEA in THF, 1.5 ml) followed by 2-aminomethylpyridine (230 μl, 2.24 mmol) and TEA (0.52 ml, 3.74 mmol).

Purification was carried out by means of column chromatography with MC/MeOH (99:1-90:10) as the eluent to give Example Compound 21 (327 mg, 49%).

Salt formation: The yield was dissolved in a minimum amount of Et₂O/MC and the solution was cooled to 0° C. 2 M HCl in Et₂O (2 eq) was added dropwise and the reaction mixture was stirred for 30 minutes. The mixture was concentrated in vacuo to give Example Compound 21 in the form of the HCl salt.

EOAI3028732 VIT-1127

MW: 356.45

HPLCMS (method A): [m/z]: 357

FIG. 21 shows the result.

### Example Compound 22: [1-Phenyl-2-(2,3,5,6-tetrahydro-[1,2′]bipyrazinyl-4-yl)-ethyl]-pyridin-2-ylmethyl-amine

The preparation was carried out analogously to Example Compound 2 according to process step 3 using:

1-phenyl-2-(2,3,5,6-tetrahydro-[1,2′]bipyrazinyl-4-yl)-ethanol (intermediate product 13) (500 mg, 1.76 mmol), methanesulfonyl chloride (204 μl, 2.64 mmol), TEA (11% TEA in THF, 15 ml) and 2-aminomethylpyridine (219 μl, 2.11 mmol).

Purification was carried out by means of column chromatography with MC/MeOH (99:1-90:10) as the eluent to give Example Compound 22 (150 mg, 23%).

Salt formation: The yield was dissolved in MeOH (3 ml) and the solution was cooled to 0° C. HCl (3 eq)) was added and the reaction mixture was stirred at 0° C. for 20 minutes. The mixture was concentrated in vacuo to give Example Compound 22 in the form of the HCl salt.

EOAI3028909 VIT-1123

MW: 374.49

HPLCMS (method A): [m/z]: 375

FIG. 22 shows the result.

### VII.I Intermediate Products According to A) Synthesis Route II

### Intermediate Product 14: 4-(Pyridine-2-carbonyl)-piperazine-1-carboxylic acid tert-butyl ester

### (Process Step 4)

Picolinc acid (1.38 g, 11.2 mmol), HATU (4.46 g, 11.7 mmol) and DIPEA (5.56 ml, 33.5 mmol) were dissolved in DMF (30 ml) and the solution was stirred at room temperature for 1 minute. The mixture was cooled to 0° C. and tert-butyl 1-piperazinecarboxylate (2.19 g, 11.7 mmol) was added. The resulting mixture was left to stand until it had warmed to room temperature and was stirred for 2 hours. Water (30 ml) was added and the mixture was extracted with EtOAc (×2). The combined organic phases were washed with saturated aqueous NaHCO₃ and brine, dried (Na₂SO₄) and concentrated in vacuo. The crude yield was purified by means of column chromatography with heptane/EtOAc (2:1-3:2-1:1) as the eluent, followed by recrystallization from EtOAc/heptane to give intermediate product 14 (2.19 g, 67%).

MW: 291.35

HPLCMS (method B): [m/z]: 292

### Intermediate Product 15: 4-(2-Methoxy-benzoyl)-piperazine-1-carboxylic acid tert-butyl ester

The preparation was carried out analogously to intermediate product 14 according to process step 4 using:

2-methoxybenzoic acid (870 mg, 5.72 mmol), HATU (2.28 g, 6.0 mmol), DIPEA (2.8 ml, 17.15 mmol) and tert-butyl 1-piperazinecarboxylate (1.12 g, 6.0 mmol). Purification was carried out by means of column chromatography with heptane/EtOAc (2:1-1:1) as the eluent to give intermediate product 15 (1.58 g, 86%).

MW: 320.39

HPLCMS (method B): [m/z]: 321

### Intermediate Product 16: piperazin-1-yl-pyridin-2-yl-methanone

### (Process Step 5)

### 4-(Pyridine-2-carbonyl)-piperazine-1-carboxylic acid tert-butyl ester (intermediate product 14) (928 mg, 3.19 mmol) was dissolved in MC (24 ml). TFA (20% TFA in MC, 6 ml) was added and the reaction mixture was stirred at room temperature for 2 hours. The mixture was diluted with MC (50 ml) and washed with 1 M sodium hydroxide (×2). The resulting aqueous phase was extracted with MC (×4) and the combined organic phases were dried (MgSO₄) and concentrated in vacuo to give intermediate product 16 (236 mg, 39%). It was not possible to detect the compound by means of HPLCMS and the structure was therefore confirmed by means of ¹H NMR.

### Intermediate Product 17: (2-Methoxy-phenyl)-piperazin-1-yl-methanone

The preparation was carried out analogously to intermediate product 16 according to process step 5 using 4-(2-methoxy-benzoyl)-piperazine-1-carboxylic acid tert-butyl ester (intermediate product 15) (1.58 g, 4.95 mmol) and TFA (20% in MC, 7 ml) to give intermediate product 17 (1.09 g, 100%).

MW: 220.27

HPLCMS (method B): [m/z]: 221

### Intermediate Product 18: {4-[2-(4-Fluoro-phenyl)-2-hydroxy-ethyl]-piperazin-1-yl}-pyridin-2-yl-methanone

### (Process Step 6)

### 2-(4-Fluorophenyl)-oxirane (132 mg, 0.96 mmol) and piperazin-1-yl-pyridin-2-yl-methanone (intermediate product 16) (183 mg, 0.96 mmol) were heated in a closed tube at 90° C. for 3 hours to give intermediate product 18 (117 mg, 37%).

MW: 329.38

HPLCMS (method B): [m/z]: 330

### Intermediate Product 19: {4-[2-(4-Fluoro-phenyl)-2-hydroxy-ethyl]-piperazin-1-yl}-(2-methoxy-phenyl)-methanone

The preparation was carried out analogously to intermediate product according to process step 6 using 2-(4-fluorophenyl)-oxirane (248 mg, 1.79 mmol) and (2-methoxy-phenyl)-piperazin-1-yl-methanone (intermediate product 17) (395 mg, 1.79 mmol) to give intermediate product 19 (571 mg, 89%).

MW: 358.42

HPLCMS (method B): [m/z]: 359

### VI.II Example Compounds According to A) Synthesis Route II

### Example Compound 23: (4-{2-(4-Fluoro-phenyl)-2-[(pyridin-2-ylmethyl)-amino]-ethyl}-piperazin-1-yl)-pyridin-2-yl-methanone

### (Process Step 7)

{4-[2-(4-Fluoro-phenyl)-2-hydroxy-ethyl]-piperazin-1-yl}-pyridin-2-yl-methanone (intermediate product 18) (382 mg, 1.16 mmol) was dissolved in a stock solution of 11% TEA in THF (1 ml). Methanesulfonyl chloride (0.13 ml, 1.74 mmol) was added and the reaction mixture was stirred at room temperature for 1 hour; the reaction was monitored by means of LCMS in order to confirm the conversion of the starting materials. TEA (0.32 ml, 2.32 mmol) was added, followed by 2-aminomethylpyridine (140 μl, 1.39 mmol) in THF (2 ml). The reaction mixture was stirred at room temperature for 30 minutes. Water (2 ml) was added to the reaction mixture and stirring was continued for a further 18 hours or until the reaction of all the starting materials and reaction intermediate products was confirmed by means of LCMS. The reaction mixture was concentrated in vacuo. The crude yield was dissolved in EtOAc and the solution was washed with water and brine.

The resulting organic phase was dried (MgSO₄) and concentrated in vacuo. The crude yield was purified by means of column chromatography with MC/MeOH (99:1-98:2) as the eluent to give Example Compound 23 (276 mg, 57%).

Salt formation: The yield was dissolved in a minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 23 in the form of the HCl salt.

EOAI3330998 VIT-1126

MW: 419.51

HPLCMS (method A): [m/z]: 420

FIG. 23 shows the result.

### Example Compound 24: (4-{2-(4-Fluoro-phenyl)-2-[(pyridin-2-ylmethyl)-amino]-ethyl}-piperazin-1-yl)-(2-methoxy-phenyl)-methanone

The preparation was carried out analogously to Example Compound 23 according to process step 7 using {4-[2-(4-fluoro-phenyl)-2-hydroxy-ethyl]-piperazin-1-yl}-(2-methoxy-phenyl)-methanone (intermediate product 19) (616 mg, 1.79 mmol), methanesulfonyl chloride (0.21 ml, 2.69 mmol) and TEA (11% TEA in THF, 1.7 ml), followed by 2-aminomethylpyridine (220 μl, 2.15 mmol) and TEA (0.5 ml, 3.58 mmol).

Purification was carried out by means of column chromatography with MC/MeOH (99:1-96:4) as the eluent to give Example Compound 24 (384 mg, 48%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes.

The mixture was concentrated in vacuo to give Example Compound 24 in the form of the HCl salt.

EOAI3331313 VIT-1155

MW: 448.55

HPLCMS (method A): [m/z]: 449

FIG. 24 shows the result.

### VII.I Intermediate Products According to A) Synthesis Route III

### Intermediate Product 20: N,N′-Dimethyl-N-pyrazin-2-yl-ethane-1,2-diamine

### (Process Step 8)

A mixture of chloropyrazine (257 mg, 2.24 mmol) and N,N′-dimethyl-ethylenediamine (1.93 ml, 17.9 mmol) was heated in a microwave oven for 15 minutes (110° C.). After cooling, the mixture was diluted with EtOAc and washed with 2 M sodium hydroxide (×3). The aqueous phase was washed with iso-propanol/chloroform (1:1, x4) and the combined organic phases were dried (MgSO₄) and concentrated in vacuo. The crude yield was purified by means of column chromatography with MC/2 M NH₃ in EtOH (100-95:5) as the eluent to give intermediate product 20 (373 mg, 100%). It was not possible to detect the compound by means of HPLCMS and the structure was therefore confirmed by means of ¹H NMR.

### Intermediate Product 21: 1-(4-Fluoro-phenyl)-2-{methyl-[2-(methyl-pyrazin-2-yl-amino)-ethyl]amino}-ethanol

2-(4-Fluorophenyl)-oxirane (296 mg, 2.14 mmol) and N,N′-dimethyl-N-pyrazin-2-yl-ethane-1,2-diamine (intermediate product 20) (357 mg, 2.14 mmol) were heated in a closed tube at 90° C. for 3 hours to give intermediate product 21 (521 mg, 80%).

MW: 304.37

HPLCMS (method B): [m/z]: 305

### VII.II Example Compounds According to A) Synthesis Route III

### Example Compound 25: 1-(4-Fluoro-phenyl)-N*2*-methyl-N*2*-[2-(methyl-pyrazin-2-yl-amino)-ethyl]-N*1*-pyridin-2-ylmethyl-ethane-1,2-diamine

1-(4-Fluoro-phenyl)-2-{methyl-[2-(methyl-pyrazin-2-yl-amino)-ethyl]-amino}-ethanol (intermediate product 21) (650 mg, 2.14 mmol) (382 mg, 1.16 mmol) was dissolved in a stock solution of 11% TEA in THF (1.9 ml). Methanesulfonyl chloride (0.25 ml, 3.21 mmol) was added and the reaction mixture was stirred at room temperature for 1 hour. The reaction was monitored by means of LCMS to confirm the reaction of the starting material. TEA (0.31 ml, 4.28 mmol) was added, followed by 2-aminomethylpyridine (260 μl, 2.57 mmol) in THF (2 ml). The reaction mixture was stirred at room temperature for 30 minutes. Water (2 ml) was added to the reaction mixture and stirring was continued for a further 18 hours or until the reaction of all the starting materials and reaction intermediate products was confirmed by means of LCMS. The reaction mixture was concentrated in vacuo. The crude yield was dissolved in EtOAc and the solution was washed with water and brine. The resulting organic phase was dried (MgSO₄) and concentrated in vacuo. The crude yield was purified by means of column chromatography with MC/MeOH (99:1-90:10) as the eluent to give Example Compound 25 (290 mg, 34%).

Salt formation: The yield was dissolved in a minimum amount of Et₂O/MC and the solution was cooled to 0° C. 2 M HCl in Et₂O (6 eq) was added dropwise and the reaction mixture was stirred for 30 minutes. The mixture was concentrated in vacuo to give Example Compound 25 in the form of the HCl salt.

EOAI3331314 VIT-1156

MW: 394.50

HPLCMS (method A): [m/z]: 395

FIG. 25 shows the result.

### IX.I Intermediate Products According to A) Synthesis Route IV

### Intermediate Product 22: 4-[2-Azido-2-(4-fluoro-phenyl)-ethyl]-3,4,5,6-tetrahydro-2H-[1,2′]bipyrazinyl

### (Process Step 11)

1-(4-Fluoro-phenyl)-2-(2,3,5,6-tetrahydro-[1,2]bipyrazinyl-4-yl)-ethanol (intermediate product 3) (500 mg, 1.65 mmol) was dissolved in a stock solution of 11% TEA in THF (1.9 ml). Methanesulfonyl chloride (192 μl, 2.48 mmol) was added and the reaction mixture was stirred at room temperature for 1 hour. The reaction was monitored by means of LCMS to confirm the reaction of the starting material. TEA (461 μl, 3.31 mmol) was added, followed by sodium azide (131 mg, 1.98 mmol) in THF (2 ml). The reaction mixture was stirred at room temperature for 30 minutes. Water (2 ml) was added to the reaction mixture and stirring was continued for a further 18 hours or until the reaction of all the starting materials and reaction intermediate products was confirmed by means of LCMS. The reaction mixture was concentrated in vacuo. The crude yield was dissolved in EtOAc and the solution was washed with water and brine. The resulting organic phase was dried (MgSO₄) and concentrated in vacuo. The crude yield was purified by means of column chromatography with MC/MeOH (99:1-97:3) as the eluent to give intermediate product 22 (167 mg, 37%).

MW: 327.37

HPLCMS (method B): [m/z]: 328

### Intermediate Product 23: {4-[2-Azido-2-(4-fluoro-phenyl)-ethyl]-piperazin-1-yl}-(tetrahydro-furan-2-yl)-methanone

The preparation was carried out analogously to intermediate product 22 according to process step 11 using:

{4-[2-(4-fluoro-phenyl)-2-hydroxy-ethyl]-piperazin-1-yl}(tetrahydro-furan-2-yl)-methanone (intermediate product 2) (650 mg, 2.0 mmol), methanesulfonyl chloride (0.23 ml, 3 mmol) and TEA (11% TEA in THF, 1.9 ml), followed by sodium azide (156 mg, 2.40 mmol) and TEA (0.56 ml, 4 mmol).

Purification was carried out by means of column chromatography with MC/MeOH (99:1-98:2) as the eluent to give intermediate product 23 (481 mg, 69%).

MW: 347.40

HPLCMS (method B): [m/z]: 348

### IX.II Example Compounds According to A) Synthesis Route IV

### Example Compound 26: 1-(4-Fluoro-phenyl)-2-(2,3,5,6-tetrahydro-[1,2′]-bipyrazinyl-4-yl)-ethylamine (process step 12)

Pd—C (10%, 3 mg) was added to a solution of 4-[2-azido-2-(4-fluoro-phenyl)-ethyl]-3,4,5,6-tetrahydro-2H-[1,2]bipyrazinyl (intermediate product 22) (80 mg, 0.24 mmol) in EtOH (10 ml) and the mixture was stirred under a hydrogen atmosphere for 7 hours. The mixture was diluted with MeOH (40 ml) and filtered through Celite (kieselguhr) and the filtrate was concentrated in vacuo. The crude yield was purified by means of column chromatography with MC/MeOH (99:1-94:6) as the eluent to give Example Compound 26 (25 mg, 34%).

EOAI3331311 VIT-1154

MW: 301.37

HPLCMS (method A): [m/z]: 302

FIG. 26 shows the result.

### Example Compound 27: {4-[2-Amino-2-(4-fluoro-phenyl)-ethyl]-piperazin-1-yl}-(tetrahydro-furan-2-yl)-methanone

The preparation was carried out analogously to Example Compound 26 according to process step 12 using:

{4-[2-azido-2-(4-fluoro-phenyl)-ethyl]-piperazin-1-yl}-(tetrahydro-furan-2-yl)-methanone (intermediate products 23) (123 mg, 0.35 mmol) and Pd—C (10%, 15 mg).

Purification was carried out by means of column chromatography with MC/MeOH (99:1-95:5) as the eluent to give Example Compound 27 (65 mg, 58%).

Salt formation: The yield was dissolved with the minimum amount of Et₂O/MC and the solution was cooled to 0° C. 2 M HCl in Et₂O (3 eq) was added dropwise and the reaction mixture was stirred for 30 minutes. The mixture was concentrated in vacuo to give Example Compound 27 in the form of the HCl salt.

EOAI3331583 VIT-1165

MW: 321.40

HPLCMS (method A): [m/z]: 322

FIG. 27 shows the result.

### X.I Intermediate Products According to B) Synthesis Route I

### Intermediate Product 24: 2-[1,4′]Bipiperidinyl-1′-yl-1-phenyl-ethanol

### (Process Step 1)

Styrene oxide (2.00 g, 16.65 mmol) and 4-(piperidinyl-1-yl)piperidine (2.80 g, 16.65 mmol) were heated in a closed tube at 90° C. for 3 hours to give intermediate product 24 (4.80 g, 100%). It was not possible to detect the compound by means of HPLCMS and the structure was therefore confirmed by means of ¹H NMR.

### Alternative Preparation Process for Intermediate product 24: 2-[1,4′]Bipiperidinyl-1′-yl-1-phenyl-ethanol

### (Process Step 2)

Styrene oxide (0.95 ml, 8.3 mmol) and 4-(piperidin-1-yl)piperidine (1.4 g, 8.3 mmol) were mixed and the mixture was heated thoroughly in a closed tube at 90° C. for 1.5 hours. The reaction mixture was diluted with MC (60 ml). The organic phase was washed with water and brine, dried (Na₂SO₄) and concentrated in vacuo. The crude yield was purified by means of trituration from hexane to give intermediate product 24 (1.75 g, 73%).

MW: 288.44

HPLCMS (method F): [m/z]: 289

### Intermediate Product 25: 1-Phenyl-2-(4-pyrrolidin-1-yl-piperidin-1-yl)-ethanol

The preparation was carried out analogously to intermediate product 24 according to process step 1 using:

styrene oxide (1.00 g, 8.32 mmol) and 4-(1-pyrrolidinyl)-piperidine (1.28 g, 8.32 mmol) to give intermediate product 25 (2.20 g, 87%)

MW: 274.4

HPLCMS (method B): [m/z]: 275

### Intermediate Product 26: 2-[1,4′]-Bipiperidinyl-1′-yl-1-(4-fluoro-phenyl)-ethanol

The preparation was carried out analogously to intermediate product 24 according to process step 1 using:

2-(4-fluorophenyl)-oxirane (500 mg, 3.62 mmol) and 4-(piperidin-1-yl)piperidine (609 mg, 3.62 mmol) to give intermediate product 26 (1.1 g, 89%).

MW: 306.42

HPLCMS (method B): [m/z]: 307

### Intermediate Product 27: 2-(4-Morpholin-4-yl-piperidin-1-yl)-1-phenyl-ethanol

The preparation was carried out analogously to intermediate product 24 and according to process step 1 using:

styrene oxide (1.00 g, 8.32 mmol) and 4-morpholinepiperidine (1.41 g, 8.32 mmol) to give intermediate product 27 (2.40 g, 99%)

MW: 290.41

HPLCMS (method C): [m/z]: 291

### Intermediate Product 28: 1-Phenyl-2-(4-phenyl-piperazin-1-yl)-ethanol

The preparation was carried out analogously to intermediate product 24 and according to process step 1 using:

styrene oxide (1.00 g, 8.32 mmol) and 1-phenylpiperazine (1.27 ml, 8.32 mmol) to give intermediate product 28 (2.34 g, 99%)

MW: 282.32

HPLCMS (method C): [m/z]: 283

### Intermediate Product 29: 1-Phenyl-2-piperidin-1-yl-ethanol

The preparation was carried out analogously to intermediate product 24 according to process step 1 using:

styrene oxide (1.0 g, 8.8 mmol) and piperidine (0.75 g, 8.8 mmol), which were reacted with one another for 3 hours to give intermediate product 29 (1,800 mg, 99%).

MW: 205.30

HPLCMS (method B): [m/z]: 206

### Intermediate Product 30: 2-Morpholin-4-yl-1-phenyl-ethanol

The preparation was carried out analogously to intermediate product 24 according to process step 1 using:

styrene oxide (316 mg, 2.6 mmol) and morpholine (230 mg, 2.6 mmol), which were reacted with one another for 3 hours to give intermediate product 30 (545 mg, 99%).

MW: 207.27

HPLCMS (method B): [m/z]: 208

### Intermediate Product 31: 2-(4-Dimethylamino-piperidin-1-yl)-1-phenyl-ethanol

The preparation was carried out analogously to intermediate product 24 according to process step 2 using:

styrene oxide (0.20 g, 1.67 mmol) and dimethyl-piperidin-4-yl-amine (0.21 g, 1.67 mmol) to give intermediate product 31 (0.22 g, 50%) after repeated washing with hexane.

MW: 248.37

HPLCMS (method F): [m/z]: 248

### Intermediate Product 32: 2-(4-Diethylamino-piperidin-1-yl)-1-phenyl-ethanol

The preparation was carried out analogously to intermediate product 24 according to process step 2 using:

styrene oxide (0.20 ml, 1.67 mmol) and diethyl-piperidin-4-yl-amine (0.26 g, 1.67 mmol) to give intermediate product 32 (0.30 g, 62%) after repeated washing with hexane.

MW: 276.43

HPLCMS (method F): [m/z]: 277

### Intermediate Product 33: 1-Phenyl-2-(4-phenylamino-piperidin-1-yl)-ethanol

The preparation was carried out analogously to intermediate product 24 according to process step 2 using:

styrene oxide (0.20 ml, 1.6 mmol) and phenyl-piperidin-4-yl-amine (0.29 g, 1.6 mmol).

Purification was carried out by means of column chromatography with MC/MeOH (100:0-90:10) as the eluent to give intermediate product 33 (0.40 g, 85%).

MW: 296.42

HPLCMS (method F): [m/z]: 297

### Intermediate Product 34: 2-(4-Methyl-[1,4′]bipiperidinyl-1′-yl)-1-phenyl-ethanol

The preparation was carried out analogously to intermediate product 24 according to process step 2 using:

styrene oxide (0.20 ml, 1.67 mmol) and 1-methyl-4-(piperidin-4-yl)piperazine (0.30 g, 1.67 mmol) to give intermediate product 34 (0.25 g, 50%) after repeated washing with hexane.

MW: 302.46

HPLCMS (method F): [m/z]: 303

### Intermediate Product 35: 2-[4-(4-Methyl-piperazin-1-yl)-piperidin-1-yl]-1-phenyl-ethanol

The preparation was carried out analogously to intermediate product 24 according to process step 2 using:

styrene oxide (0.3 ml, 1.67 mmol) and N-methyl-4-piperidinyl-piperazine (0.6 g, 1.67 mmol) to give intermediate product 35 (0.6 g, 80%) after repeated washing with hexane.

MW: 303.45

HPLCMS (method F): [m/z]: 304

### X.II. Example Compounds According to B) Synthesis Route I

### Example Compound 5: (2-[1,4′]-Bipiperidinyl-1′-yl-1-phenyl-ethyl)-(2-morpholin-4-yl-ethyl)-amine

### (Process Step 3)

### 2-[1,4′]Bipiperidinyl-1′-yl-1-phenyl-ethanol (intermediate product 24) (1.00 g, 3.47 mmol) was dissolved in a stock solution of 11% TEA in THF (25 ml). Methanesulfonyl chloride (403 μl, 5.20 mmol) was added and the reaction mixture was stirred at room temperature for 1 hour. The reaction was monitored by means of LCMS to confirm the conversion of the starting materials. TEA (966 μl, 6.94 mmol) was added, followed by a solution of 4-(2-aminoethyl)morpholine (546 μl, 4.16 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude yield was purified by means of preparative HPLC (basic conditions) to give Example Compound 5 (120 mg, 8%).

EOAI3029070 VIT-1042

MW: 400.61

HPLCMS (method A): [m/z]: 401

FIG. 5 shows the result.

### Example Compound 6: (2-[1,4′]Bipipedidinyl-1-yl-phenyl-ethyl)-(4-methoxy-benzyl)-amine

The preparation was carried out analogously to Example Compound 5 according to process step 3 using:

2-[1,4′]bipiperidinyl-1′-yl-1-phenyl-ethanol (intermediate product 24) (1.00 g, 3.47 mmol), methanesulfonyl chloride (403 μl, 5.20 mmol), TEA (966 μl, 6.94 mmol) and 4-methoxybenzylamine (544 μl, 4.16 mmol).

Purification was carried out by means of column chromatography with MC/MeOH (99:1-90:10) as the eluent to give Example Compound 6 (200 mg, 14%).

EOAI3029082 VIT-1043

MW: 407.60

HPLCMS (method A): [m/z]: 408

FIG. 6 shows the result.

### Example Compound 4: 1′-{2-[4-(2-Methoxy-ethyl)-piperazin-1-yl]-2-phenyl-ethyl}-[1,4′]bipiperldinyl

The preparation was carried out analogously to Example Compound 5 according to process step 3 using:

2-[1,4′]bipiperidinyl-1′-yl-1-phenyl-ethanol (intermediate product 24) (1.00 g, 3.47 mmol), methanesulfonyl chloride (403 μl, 5.20 mmol), TEA (966 μl, 6.94 mmol) and 1-(2-methoxyethyl)-piperazine (619 μl, 4.16 mmol).

Purification was carried out by means of preparative HPLC (basic conditions) to give Example Compound 4 (60 mg, 4%).

Salt formation: The yield was dissolved in MeOH (3 ml) and the solution was cooled to 0° C. HCl (5 eq)) was added and the reaction mixture was stirred at 0° C. for 20 minutes. The mixture was concentrated in vacuo to give Example Compound 4 in the form of the HCl salt.

EOAI3094837 VIT-1041

OP-19909-A03 (manufacturer: EVOTEC)

MW: 414.64 or 414.63

HPLCMS (method A): [m/z]: 415

UV spectrum: δ, max [ηm]:--

FIG. 4 shows the result.

### Example Compound 28: 1′[2-Phenyl-2-(4-pyridin-2-yl-piperazin-1-yl)-ethyl]-[1,4′]bipiperidinyl

The preparation was carried out analogously to Example Compound 5 according to process step 3 using:

2-[1,4′]bipiperidinyl-1′-yl-1-phenyl-ethanol (intermediate product 24) (500 mg, 1.73 mmol), methanesulfonyl chloride (202 μl, 2.60 mmol), TEA (482 μl, 3.48 mmol) and 1-(2-pyridyl)-piperazine (319 μl, 2.09 mmol).

Purification was carried out by means of preparative HPLC (basic conditions) to give Example Compound 28 (22.4 mg, 3%).

Salt formation: The yield was dissolved with a minimum amount of Et₂O/MC and the solution was cooled to 0° C. 2 M HCl in Et₂O (5 eq) was added dropwise and the reaction mixture was stirred for 30 minutes. The mixture was concentrated in vacuo to give Example Compound 28 in the form of the HCl salt.

EOAI3094813 VIT-1159

MW: 433.63

HPLCMS (method A): [m/z]: 434

FIG. 28 shows the result.

### Example Compound 29: 4-(2-[1,4′]Bipiperidinyl-1′-yl-1-phenyl-ethyl)-3,4,5,6-tetrahydro-2H-[1,2′]bipyrazinyl

The preparation was carried out analogously to Example Compound 5 according to process step 3 using:

2-[1,4′]bipiperidinyl-1′-yl-1-phenyl-ethanol (intermediate product 24) (1.00 g, 3.47 mmol), methanesulfonyl chloride (403 μl, 5.20 mmol), TEA (966 μl, 6.94 mmol) and 1-(2-pyridyl)-piperazine (637 μl, 4.16 mmol).

Purification was carried out by means of preparative HPLC (basic conditions) to give Example Compound 29 (40 mg, 2%).

Salt formation: The yield was dissolved with a minimum amount of Et₂O/MC and the solution was cooled to 0° C. 2 M HCl in Et₂O (5 eq) was added dropwise and the reaction mixture was stirred for 30 minutes. The mixture was concentrated in vacuo to give Example Compound 29 in the form of the HCl salt.

EOAI3331309 VIT-1157

MW: 434.63

HPLCMS (method A): [m/z]: 435

FIG. 29 shows the result.

### Example Compound 30: 1′-{2-Phenyl-2-[4-(tetrahydro-furan-2-ylmethyl)-piperazin-1-yl]-ethyl}-[1,4′]bipiperidinyl

The preparation was carried out analogously to Example Compound 5 according to process step 3 using:

2-[1,4′]bipiperidinyl-1′-yl-1-phenyl-ethanol (intermediate product 24) (500 mg, 1.7 mmol), methanesulfonyl chloride (201 μl, 2.60 mmol), TEA (483 μl, 3.47 mmol) and 1-tetrahydro-furoyl-piperazine (295 mg, 1.73 mmol).

Purification was carried out by means of preparative HPLC (basic conditions) to give Example Compound 30 (100 mg, 13%).

Salt formation: The yield was dissolved with a minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 30 in the form of the HCl salt.

EOAI3331310 VIT-1158

MW: 440.68

HPLCMS (method A): [m/z]: 441

FIG. 30 shows the result.

### Example Compound 31: 1′-{2-[4-(2-Methoxy-ethyl)-piperidin-1-yl]-2-phenyl-ethyl}-[1,4′]bipiperidinyl

The preparation was carried out analogously to Example Compound 5 according to process step 3 using:

2-[1,4′]bipiperidinyl-1′-yl-1-phenyl-ethanol (intermediate product 24) (576 mg, 2.0 mmol), methanesulfonyl chloride (229 μl, 3.0 mmol), TEA (1.0 ml, 7.2 mmol) and 4-(2-methoxy-ethyl)-piperidine hydrochloride (429 mg, 2.40 mmol).

Purification was carried out by means of column chromatography with MC/7 N NH₃ in MeOH (100:0-90:10) as the eluent, followed by dilution with water and extraction with EtOAc (×2). The combined organic phases were dried (MgSO₄) and concentrated in vacuo to give Example Compound 31 (800 mg).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes. The material was concentrated in vacuo and triturated with ether (×3) to give Example Compound 31 the form of the HCl salt (10 mg, 1%).

EOAI3334568 VIT-1295

MW: 413.64

HPLCMS (method A): [m/z]: 414

FIG. 30 shows the result.

### Example Compound 32: [4-(2-[1,4′]Bipiperidinyl-1′-yl-1-phenyl-ethyl)-piperazin-1-yl]-(tetrahydro-furan-2-yl)-methanone

The preparation was carried out analogously to Example Compound 5 according to process step 3 using:

2-[1,4′]bipiperidinyl-1′-yl-1-phenyl-ethanol (intermediate product 24) (500 mg, 1.73 mmol), methanesulfonyl chloride (201 μl, 2.60 mmol), TEA (483 μl, 3.47 mmol) and 1-(tetrahydro-2-furoyl)-piperazine (383 mg, 2.08 mmol).

Purification was carried out by means of preparative HPLC (basic conditions) to give Example Compound 32 (20 mg, 2%).

EOAI3029018 VIT-1172

MW: 454.66

HPLCMS (method A): [m/z]: 477 (M+Na)

FIG. 32 shows the result.

### Example Compound 33: 1-(2-Methoxy-ethyl)-4-[1-phenyl-2-(4-pyrrolidin-1-yl-piperidin-1-yl)-ethyl]-piperazine

The preparation was carried out analogously to Example Compound 5 according to process step 3:

1-phenyl-2-(4-pyrrolidin-1-yl-piperidin-1-yl)-ethanol (intermediate product 25) (500 mg, 1.82 mmol), methanesulfonyl chloride (211 μl, 2.73 mmol), TEA (505 μl, 3.64 mmol) and 1-(2-methoxyethyl)-piperazine (324 μl, 2.18 mmol)

Purification was carried out by means of column chromatography with MC/7 M NH₃ in MeOH (100-95:5) as the eluent to give Example Compound 33 (320 mg, 44%).

Salt formation: The yield was dissolved in a minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 33 in the form of the HCl salt.

EOAI3331585 VIT-1160

MW: 400.60

HPLCMS (method A): [m/z]: 401

FIG. 33 shows the result.

### Example Compound 34: 1′-{2-(4-Fluoro-phenyl)-2-[4-(2-methoxy-ethyl)-piperazin-1-yl]-ethyl}-[1,4′]bipiperidinyl

The preparation was carried out analogously to Example Compound 5 according to process step 3:

2-[1,4′]bipiperidinyl-1′-yl-1-(4-fluoro-phenyl)-ethanol (intermediate product 26) (500 mg, 1.63 mmol), methanesulfonyl chloride (182 μl, 2.45 mmol), TEA (452 μl, 3.26 mmol) and 1-(2-methoxyethyl)-piperazine (291 μl, 1.96 mmol).

Purification was carried out by means of preparative HPLC (basic conditions) to give Example Compound 34 (76.8 mg, 11%).

Salt formation: The yield was dissolved in the minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 34 in the form of the HCl salt.

EOAI3331586 VIT-1161

MW: 432.62

HPLCMS (method A): [m/z]: 433

FIG. 34 shows the result.

### Example Compound 35: 4-(1-{2-[4-(2-Methoxy-ethyl)-piperazin-1-yl]-2-phenyl-ethyl}-piperidin-4-yl)-morpholine

The preparation was carried out analogously to Example Compound 5 according to process step 3:

2-(4-morpholin-4-yl-piperidin-1-yl)-1-phenyl-ethanol (intermediate product 27) (500 mg, 1.72 mmol), methanesulfonyl chloride (200 μl, 2.59 mmol), TEA (477 μl, 3.44 mmol) and 1-(2-methoxyethyl)-piperazine (306 μl, 2.06 mmol).

Purification was carried out by means of column chromatography with MC/7 M NH₃ in MeOH (100-95:5) as the eluent to give Example Compound 35 (159 mg, 22%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes. The mixture was concentrated in vacuo to give Example Compound 35 in the form of the HCl salt.

EOAI3331587 VIT-1162

MW: 416.6

HPLCMS (method A): [m/z]: 417

FIG. 35 shows the result.

### Example Compound 36: 1-(2-Methoxy-ethyl)-4-(1-phenyl-2-piperazin-1-yl-ethyl)-4-phenylpiperazine

The preparation was carried out analogously to Example Compound 5 according to process step 3:

1-phenyl-2-(4-phenyl-piperazin-1-yl)-ethanol (intermediate product 28) (500 mg, 1.77 mmol), methanesulfonyl chloride (206 μl, 2.66 mmol), TEA (491 μl, 3.54 mmol) and 1-(2-methoxyethyl)-piperazine (316 μl, 2.12 mmol).

Purification was carried out by means of column chromatography with MC/7 M NH₃ in MeOH (100:0-95:5) as the eluent to give Example Compound 36 (187 mg, 26%).

Salt formation: The yield was dissolved in a minimum amount of Et₂O/MC and the solution was cooled to 0° C. 2 M HCl in Et₂O (4 eq) was added dropwise and the reaction mixture was stirred for 30 minutes. The mixture was concentrated in vacuo to give Example Compound 36 in the form of the HCl salt.

EOAI3028982 VIT-1163

MW: 408.58

HPLCMS (method A): [m/z]: 409

FIG. 36 shows the result.

### Example Compound 37: 1-(2-Methoxy-ethyl)-4-(1-phenyl-2-piperidin-1-yl-ethyl)-piperazine

The preparation was carried out analogously to Example Compound 5 according to process step 3:

1-phenyl-2-piperidin-1-yl-ethanol (intermediate product 29) (643 mg, 3.13 mmol), methanesulfonyl chloride (360 μl, 4.7 mmol), TEA (870 μl, 6.26 mmol) and 1-(2-methoxy-ethyl)-piperazine (540 mg, 3.76 mmol).

Purification was carried out by means of column chromatography with MC/2 M NH₃ in MeOH (100:0-90:10) as the eluent to give Example Compound 37 (389 mg, 37%).

Salt formation: A portion of the material (85 mg 0.26 mmol) was dissolved in a minimum amount of MC and the solution was cooled to 0° C. 2 M HCl in Et₂O (3 eq) was added dropwise and the reaction mixture was stirred for 30 minutes. The mixture was concentrated in vacuo to give Example Compound 37 in the form of the HCl salt.

EOAI3331582 VIT-1164

MW: 331.51

HPLCMS (method E): [m/z]: 332

FIG. 37 shows the result.

### Example Compound 38: (4-Methoxy-benzyl)-(1-phenyl-2-piperidin-1-yl-ethyl)-amine

The preparation was carried out analogously to Example Compound 5 according to process step 3 using:

1-phenyl-2-piperidin-1-yl-ethanol (intermediate product 29) (634 mg, 3.09 mmol), methanesulfonyl chloride (360 μl, 4.7 mmol), TEA (860 μl, 6.18 mmol) and 4-methoxy-benzylamine (509 mg, 3.71 mmol).

Purification was carried out by means of column chromatography with MC/MeOH (100:0-99:1-98:2) as the eluent to give Example Compound 38 (210 mg, 21%).

EOAI3331584 VIT-1166

MW: 324.47

HPLCMS (method E): [m/z]: 325

FIG. 38 shows the result.

### Example Compound 39: (2-Morpholin-4-yl-ethyl)-(1-phenyl-2-piperidin-1-yl-ethyl)-amine

The preparation was carried out analogously to Example Compound 5 according to process step 3 using:

1-phenyl-2-piperidin-1-yl-ethanol (intermediate product 29) (645 mg, 3.14 mmol), methanesulfonyl chloride (360 μl, 4.7 mmol), TEA (880 μl, 6.28 mmol) and 2-morpholin-4-yl-ethylamine (490 mg, 3.77 mmol).

Purification was carried out by means of preparative HPLC (basic conditions) on half of the crude material to give Example Compound 39 (303 mg, 31%).

EOAI3332899 VIT-1174

MW: 317.48

HPLCMS (method E): [m/z]: 318

FIG. 39 shows the result.

### Example Compound 40: 4-{2-[4-(2-Methoxy-ethyl)-piperazin-1-yl]-2-phenyl-ethyl}-morpholine

The preparation was carried out analogously to Example Compound 5 according to process step 3 using:

2-morpholin-4-yl-1-phenyl-ethanol (intermediate product 30) (545 mg, 2.6 mmol), methanesulfonyl chloride (300 μl, 3.95 mmol), TEA (730 μl, 5.26 mmol) and 1-(2-methoxy-ethyl)-piperazine (455 mg, 3.16 mmol).

Purification was carried out by means of preparative HPLC (basic conditions) to give Example Compound 40 (275 mg, 31%).

Salt formation: A portion of the material (153 mg, 0.46 mmol) was dissolved in a minimum amount of ET₂O and the solution was cooled to 0° C. 2 M HCl in Et₂O (3 eq) was added dropwise and the reaction mixture was stirred for 30 minutes. The mixture was concentrated in vacuo to give Example Compound 40 in the form of the HCl salt.

EOAI3028981 VIT-1175

MW: 333.47

HPLCMS (method E): [m/z]: 334

FIG. 40 shows the result.

### Example Compound 41: 1-{1-Phenyl-2-[4-(piperidin-1-yl)piperidin-1-yl]ethyl}-4-(propan-2-yl)piperazine

### (Process Step 4)

Methanesulfonyl chloride (0.04 ml, 0.52 mmol) was added to a solution of 2-[1,4′]bipiperidinyl-1′-yl-1-phenyl-ethanol (intermediate product 24) (0.1 g, 0.35 mmol) and TEA (0.1 ml, 0.69 mmol) in THF (10 ml) at 0° C. and the mixture was stirred at room temperature for 3 hours. TEA (0.1 ml, 0.69 mmol) was added, followed by 1-iso-propyl-piperazine (0.05 ml, 0.35 mmol) and stirring was continued for a further 1.5 hours. Water (10 ml) was added and the mixture was stirred for 18 hours. The reaction mixture was extracted with MC and the organic phases were washed with brine, dried (Na₂SO₄) and concentrated in vacuo. The crude yield (containing alcohol as a non-separable impurity) was dissolved in pyridine (3 ml) and acetic anhydride (56 μl, 0.6 mmol) was added at 0° C. and the reaction mixture was stirred at room temperature for 3 hours. The reaction mixture was concentrated in vacuo. Purification of the crude material was carried out by means of column chromatography with MC/MeOH/aq. NH₃ (100:0:0-95:5:1) as the eluent to give Example Compound 41 (34 mg, 16.4%).

EOAI3334777 VIT-1311

MW: 398.64

HPLCMS (method F): [m/z]: 399

FIG. 41 shows the result.

### Example Compound 42: 1-Butyl-4-{1-phenyl-2-[4-(piperidin-1-yl)piperidin-1-yl]ethyl}piperazine (EV0828-110-001)

The preparation was carried out analogously to Example Compound 41 according to process step 4 using:

2-[1,4′]bipiperidinyl-1′-yl-1-phenyl-ethanol (intermediate product 24) (0.2 g, 0.69 mmol), TEA (0.19 ml, 1.38 mmol) and methanesulfonyl chloride (0.08 ml, 1.04 mmol), followed by 1-butyl-piperazine (0.1 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 42 (0.073 g, 26%).

EOAI3334932 VIT-1317

MW: 412.64

HPLCMS (method F): [m/z]: 413

FIG. 42 shows the result.

### Example Compound 43: 1-Cyclopentyl-4-{1-phenyl-2-[4-(piperidin-1-yl)piperidin-1-yl]ethyl}piperazine

The preparation was carried out analogously to Example Compound 41 according to process step 4 using:

2-[1,4′]bipiperidinyl-1′-yl-1-phenyl-ethanol (intermediate product 24) (0.20 g, 0.69 mmol), TEA (0.19 ml, 1.38 mmol) and methanesulfonyl chloride (0.08 ml, 1.04 mmol) followed by 1-cyclopentyl-piperazine (0.11 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 43 (0.04 g, 14%).

EOAI334933 VIT-1318

MW: 424.68

HPLCMS (method F): [m/z]: 425

FIG. 43 shows the result.

### Example Compound 44: 1-(2-Ethoxyethyl)-4-{1-phenyl-2-[4-(piperidin-1-yl)piperidin-1-yl]ethyl}piperazine

The preparation was carried out analogously to Example Compound 41 according to process step 4 using:

2-[1,4′]bipiperidinyl-1′-yl-1-phenyl-ethanol (intermediate product 24) (0.2 g, 0.69 mmol), TEA (0.19 ml, 1.38 mmol) and methanesulfonyl chloride (0.08 ml, 1.04 mmol), followed by 1-(2-ethoxy-ethyl)-piperazine (0.11 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 44 (0.11 g, 37%).

EOAI3335063 VIT-1325

MW: 428.67

HPLCMS (method F): [m/z]: 429

FIG. 44 shows the result.

### Example Compound 45: 1-(3-Methoxypropyl)-4-{1-phenyl-2-[4-(piperidin-1-yl)piperidin-1-yl]ethyl}piperazine

The preparation was carried out analogously to Example Compound 41 according to process step 4 using:

2-[1,4′]bipiperidinyl-1′-yl-1-phenyl-ethanol (intermediate product 24) (0.20 g, 0.69 mmol), TEA (0.19 ml, 1.38 mmol) and methanesulfonyl chloride (0.08 ml, 1.04 mmol), followed by 1-(3-methoxy-propyl)-piperazine (0.11 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 45 (0.038 g, 12%).

EOAI3335064 VIT-1326

MW: 428.67

HPLCMS (method F): [m/z]: 429

FIG. 45 shows the result.

### Example Compound 46: 1-[2-(1H-Imidazol-1-yl)ethyl]-4-{1-phenyl-2-[4-(piperidin-1-yl)piperidin-1-yl]ethyl}piperazine

The preparation was carried out analogously to Example Compound 41 according to process step 4 using:

2-[1,4′]bipiperidinyl-1′-yl-1-phenyl-ethanol (intermediate product 24) (0.20 g, 0.69 mmol), TEA (0.19 ml, 1.38 mmol) and methanesulfonyl chloride (0.08 ml, 1.04 mmol), followed by 1-(2-imidazol-1-yl-ethyl)-piperazine (0.124 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 46 (0.173 g, 55%).

EOAI3335065 VIT-1327

MW: 450.68

HPLCMS (method F): [m/z]: 451

FIG. 46 shows the result.

### Example Compound 47: Diethyl[2-(4-{1-phenyl-2-[4-(piperidin-1-yl)piperidin-1-yl]ethyl}piperazin-1-yl)ethyl]amine

The preparation was carried out analogously to Example Compound 41 according to process step 4 using:

2-[1,4′]bipiperidinyl-1′-yl-1-phenyl-ethanol (intermediate product 24) (0.20 g, 0.69 mmol), TEA (0.19 ml, 1.38 mmol) and methanesulfonyl chloride (0.08 ml, 1.04 mmol), followed by diethyl-(2-piperazin-1-yl-ethyl)-amine (0.128 g, 0.69 mmol) and TEA (0.19 ml, 1.38 mmol).

Purification was carried out by means of column chromatography with MC/MeOH/aq. NH₃ (100:0:0-95:5:2) as the eluent to give Example Compound 47 (0.059 g, 19%).

EOAI3335066 VIT-1328

MW: 455.74

HPLCMS (method F): [m/z]: 456

FIG. 47 shows the result.

### Example Compound 48: (1-{2-[4-(2-Methoxy-ethyl)-piperazin-1-yl]-2-phenyl-ethyl}-piperidin-4-yl)-dimethyl-amine

The preparation was carried out analogously to intermediate product 41 according to process step 4 using:

2-(4-dimethylamino-piperidin-1-yl)-1-phenyl-ethanol (intermediate product 31) (0.25 g, 1.008 mmol), TEA (0.29 ml, 2.016 mmol) and methanesulfonyl chloride (0.12 ml, 1.51 mmol), followed by 1-(2-methoxy-ethyl)-piperazine (0.15 ml, 1.01 mmol) and TEA (0.29 ml, 2.016 mmol).

Purification was carried out by means of column chromatography with MC/MeOH/aq. NH₃ (100:0:0-95:5:1) as the eluent to give Example Compound 48 (0.034 g, 16%).

EOAI3335296 VIT-1354

MW: 374.57

HPLCMS (method F): [m/z]: 375

FIG. 48 shows the result.

### Example Compound 49: Diethyl-(1-{2-[4-(2-methoxy-ethyl)-piperazin-1-yl]-2-phenyl-ethyl}-piperidin-4-yl)-amine

The preparation was carried out analogously to Example Compound 41 according to process step 4 using:

2-(4-diethylamino-piperidin-1-yl)-1-phenyl-ethanol (intermediate product 32) (0.25 g, 0.69 mmol), TEA (0.252 ml, 1.81 mmol) and methanesulfonyl chloride (0.11 ml, 1.95 mmol), followed by 1-(2-methoxy-ethyl)-piperazine (0.13 g, 0.90 mmol) and TEA (0.252 ml, 1.81 mmol).

Purification was carried out by means of column chromatography with MC/MeOH/aq. NH₃ (100:0:0-95:5:1) as the eluent to give Example Compound 49 (0.15 g, 41%).

EOAI3335297 VIT-1355

MW: 402.63

HPLCMS (method F): [m/z]: 403

FIG. 49 shows the result.

### Example Compound 50: (1-{2-[4-(2-Methoxy-ethyl)-piperazin-1-yl]-2-phenyl-ethyl}-piperidin-4-yl)-phenyl-amine

The preparation was carried out analogously to Example Compound 41 according to process step 4 using:

1-phenyl-2-(4-phenylamino-piperidin-1-yl)-ethanol (intermediate product 33) (0.40 g, 1.36 mmol), TEA (0.38 ml, 2.72 mmol) and methanesulfonyl chloride (0.16 ml, 2.02 mmol), followed by 1-(2-methoxy-ethyl)-piperazine (0.196 g, 1.36 mmol) and TEA (0.38 ml, 2.72 mmol).

Purification was carried out by means of column chromatography with MC/MeOH (90:10) as the eluent to give Example Compound 50 (0.10 g, 20%).

EOAI3335381 VIT-1372

MW: 422.62

HPLCMS (method F): [m/z]: 423

FIG. 50 shows the result.

### Example Compound 51: 1′-{2-[4-(2-Methoxy-ethyl)-piperazin-1-yl]-2-phenyl-ethyl}-4-methyl-[1,4′]bipiperidinyl The preparation was carried out analogously to intermediate product 24 according to process step 2 using:

2-(4-methyl-[1,4′]bipiperidinyl-1′-yl)-1-phenyl-ethanol (intermediate product 34) (0.25 g, 0.83 mmol), TEA (0.23 ml, 1.65 mmol) and methanesulfonyl chloride (0.10 ml, 1.24 mmol), followed by 1-(2-methoxy-ethyl)-piperazine (0.20 g, 1.36 mmol) and TEA (0.23 ml, 1.65 mmol).

Purification was carried out by means of column chromatography with MC/MeOH (100:0-90:10) as the eluent to give Example Compound 51 (0.04 g, 15%).

EOAI3335070 VIT-1332

MW: 428.67

HPLCMS (method F): [m/z]: 429

FIG. 51 shows the result.

### Example Compound 52: 1-(1-{2-[4-(2-Methoxyethyl)piperazin-1-yl]-2-phenylethyl}piperidin-4-yl)-4-methylpiperazine

The preparation was carried out analogously to Example Compound 41 according to process step 4 using:

2-[4-(4-Methyl-piperazin-1-yl)-piperidin-1-yl]-1-phenyl-ethanol (intermediate product 35) (0.60 g, 1.98 mmol), TEA (0.55 ml, 3.96 mmol) and methanesulfonyl chloride (0.23 ml, 2.02 mmol), followed by 1-(2-methoxy-ethyl)-piperazine (0.29 g, 1.98 mmol) and TEA (0.55 ml, 3.96 mmol).

Purification was carried out by means of column chromatography with MC/MeOH/aq. NH₃ (100:0:0-95:5:1) as the eluent to give Example Compound 52 (0.06 g, 7%).

EOAI3335298 VIT-1356

MW: 429.65

HPLCMS (method F): [m/z]: 430

FIG. 52 shows the result.

### XI.I Intermediate Products According to B) Synthesis Route II

### Intermediate Product 36: [1-(2-Hydroxy-2-phenyl-ethyl)-piperidin-4-yl]-carbamic acid tert-butyl ester

Styrene oxide (600 mg, 5.0 mmol) and piperidin-4-yl-carbamic acid tert-butyl ester (1.0 g, 5.0 mmol) were heated in a closed tube at 90° C. for hours. The crude yield was purified by means of column chromatography to give intermediate product 36 (4.80 g, 100%).

MW: 320.44

HPLCMS (method B): [m/z]: 321

### Intermediate Product 37: (1-{2-[4-(2-Methoxy-ethyl)-piperazin-1-yl]-2-phenyl-ethyl}-piperidin-4-yl)-carbamic acid Pert-butyl ester

### (Process Step 6)

### [1-(2-Hydroxy-2-phenyl-ethyl)piperidin-4-yl]-carbamic acid tert-butyl ester (intermediate product 36) (680 mg, 2.13 mmol) was dissolved in a stock solution of 11% TEA in THF (25 ml). Methanesulfonyl chloride (244 μl, 3.19 mmol) was added and the reaction mixture was stirred at room temperature for 1 hour; the reaction was monitored by means of LCMS in order to confirm the conversion of the starting material. TEA (1.0 ml, 7.22 mmol) was added, followed by a solution of 1-(2-methoxy-ethyl)-piperazine (368 mg, 2.55 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude material was purified by means of column chromatography with MC/7 M NH₃ in MeOH (100:0-90:10) as the eluent to give intermediate product 37 (530 mg, 56%).

MW: 446.64

HPLCMS (method B): [m/z]: 447

### Intermediate Product 38: 1-{2-[4-(2-Methoxy-ethyl)-piperazin-1-yl]-2-phenyl-ethyl}-piperidin-4-ylamine

### (Process Step 7)

A solution of (1-{2-[4-(2-methoxy-ethyl)-piperazin-1-yl]-2-phenyl-ethyl}-piperidin-4-yl)-carbamic acid tert-butyl ester (intermediate product 37) (430 mg, 0.96 mmol) in EtOAc (15 ml) was cooled to 0° C. and treated with HCl (4 N in dioxane, 2 ml, 8 mmol). The resulting suspension was stirred at 0° C. for 2 hours and left to stand overnight to warm to room temperature. Further HCl (4 N in dioxane, 2.0 ml, 8 mmol) was added and the suspension was stirred at room temperature for 16 hours. The mixture was concentrated in vacuo. The crude yield was purified by means of trituration from Et₂O, filtered under N2 and dried in vacuo to give intermediate product 38 in the form of the HCL salt (320 mg, 67%).

MW: 346.52

HPLCMS (method A): [m/z]: 347

### XI.II Example Compounds According to B) Synthesis Route II

### Example Compound 53: N-(1-{2-[4-(2-Methoxy-ethyl)-piperazin-1-yl]-2-phenyl-ethyl}-piperidin-4-yl)-benzamide

### (Process Step 8)

A solution of 1-{2-[4-(2-methoxy-ethyl)-piperazin-1-yl]-2-phenyl-ethyl}-piperidin-4-ylamine HCl (intermediate product 38) (30 mg, 0.06 mmol) in MC (2 ml) was treated with benzoyl chloride (15 μl, 0.12 mmol), followed by DIPEA (106 μl, 0.61 mmol) and the solution was stirred at room temperature for 3 hours. The reaction mixture was diluted with saturated aqueous NaHCO₃ (2 ml) and extracted with DCM (×3). The combined organic phases were dried (MgSO₄) and concentrated in vacuo. The crude yield was purified by means of column chromatography with MC/2 M NH₃ in MeOH (95:5-90:10) as the eluent, followed by trituration from Et₂O to give Example Compound 53 (12 mg, 44%).

EOAI3335056 VIT-1333

MW: 450.62

HPLCMS (method A): [m/z]: 451

FIG. 53 shows the result.

### Example Compound 54: Cyclohexanecarboxylic acid (1-{2-[4-(2-methoxy-ethyl)-piperazin-1-yl]-2-phenyl-ethyl}-piperidin-4-yl)-amide

The preparation was carried out analogously to Example Compound 53 according to process step 8 using:

1-{2-[4-(2-methoxy-ethyl)-piperazin-1-yl]-2-phenyl-ethyl}-piperidin-4-ylamine HCl (intermediate product 38) (30 mg, 0.06 mmol), DIPEA (106 μl, 0.61 mmol) and cyclohexanecarbonyl chloride (15 μl, 0.12 mmol). Purification was carried out by means of column chromatography with MC/2 M NH₃ in MeOH (95:0-90:10) as the eluent to give Example Compound 54 (12 mg, 43%).

EOAI3335135 VIT-1342

MW: 456.66

HPLCMS (method A): [m/z]: 457

FIG. 54 shows the result.

### Example Compound 55: N-(1-{2-[4-(2-Methoxy-ethyl)-piperazin-1-yl]-2-phenyl-ethyl}-piperidin-4-yl)-isobutyramide

The preparation was carried out analogously to Example Compound 53 according to process step 8 using:

1-{2-[4-(2-methoxy-ethyl)-piperazin-1-yl]-2-phenyl-ethyl}-piperidin-4-ylamine HCl (intermediate product 38) (30 mg, 0.06 mmol), DIPEA (106 μl, 0.61 mmol) and isobutyryl chloride (13 μl, 0.12 mmol).

Purification was carried out by means of column chromatography with MC/2 M NH₃ in MeOH (98:2) as the eluent, followed by trituration from Et₂O to give Example Compound 55 (9 mg, 35%).

EOAI3335136 VIT-1343

MW: 416.6

HPLCMS (method A): [m/z]: 417

FIG. 55 shows the result.

### XII.I Intermediate Products According to B) Synthesis Route III

### Intermediate Product 39: N-(1-Benzyl-piperidin-4-yl)-acetamide

### (Process Step 9)

Acetyl chloride (0.9 ml, 12.6 mmol) was added dropwise to a solution of 1-benzyl-piperidin-4-ylamine (2.0 g, 10.5 mmol) and DIPEA (3.6 ml, 21.0 mmol) in MC (20 ml) and the mixture was stirred at room temperature for 18 hours. The mixture was washed with water (×2) and brine, dried (Na₂SO₄) and concentrated in vacuo to give intermediate product 39 (2.3 g, 95%). It was not possible to detect the compound by means of HPLCMS and the structure was therefore confirmed by means of ¹H NMR.

### Intermediate Product 40: N-Piperidin-4-yl-acetamide

### (Process Step 10)

Pd—C (350 mg) was added to a solution of N-(1-benzyl-piperidin-4-yl)-acetamide (intermediate product 39) (2.3 g, 10.0 mmol) in EtOH (35 ml) and HCl (1 M, 5 ml) and the mixture was stirred under a hydrogen atmosphere for 5 hours. The catalyst was removed by means of filtration through Celite (kieselguhr), followed by washing with EtOH, followed by water. The filtrate was concentrated in vacuo and the yield was purified by means of column chromatography with MC/7 M NH₃ in MeOH (100:0-80:20) as the eluent to give intermediate product 40 (0.92 g, 65%). It was not possible to detect the compound by means of HPLCMS and the structure was therefore confirmed by means of NMR.

### Intermediate Product 41: N-[1-(2-Hydroxy-2-phenyl-ethyl)-piperidin-4-yl]-acetamide

### (Process Step 11)

Styrene oxide (740 μl, 6.8 mmol) and N-piperidin-4-yl-acetamide (intermediate product 40) (920 mg, 6.8 mmol) were heated in a closed tube at 90° C. for 2 hours. The crude yield was purified by means of column chromatography with MC/2 M NH₃ in MeOH (100:0-80:20) as the eluent, followed by preparative HPLC (basic conditions) to give intermediate product 41 (135 mg, 8%).

MW: 262.35

HPLCMS (method C): [m/z]: 263

### XII.II Example Compounds According to B) Synthesis Route III

### Example Compound 56: N-(1-{2-[4-(2-Methoxy-ethyl)-piperazin-1-yl]-2-phenyl-ethyl}-piperidin-4-yl)-acetamide

### (Process Step 12)

### N-[1-(2-Hydroxy-2-phenyl-ethyl)-piperidin-4-yl]-acetamide (intermediate product 41) (135 mg, 0.60 mmol) was dissolved in a stock solution of 11% TEA in THF (25 ml). Methanesulfonyl chloride (70 μl, 0.90 mmol) was added and the reaction mixture was stirred at room temperature for 1 hour. The reaction was monitored by means of LCMS to confirm the reaction of the starting materials. TEA (1.5 ml, 10.8 mmol) was added, followed by a solution of 1-(2-methoxyethyl)-piperazine (107 μl, 0.72 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude yield was purified by means of column chromatography with MC/7 M NH₃ in MeOH (100:0-80:20) as the eluent, followed by preparative HPLC (basic conditions).

Salt formation: The yield was dissolved in MeOH (3 ml) and the solution was cooled to 0° C. 2 M HCl in Et₂O (3 eq) was added dropwise and the reaction mixture was stirred for 30 minutes. The mixture was concentrated in vacuo to give Example Compound 56 in the form of the HCl salt (39 mg, 16%).

EOAI3333573 VIT-1204

MW: 388.56

HPLCMS (method A): [m/z]: 389

FIG. 56 shows the result.

### XII.I Intermediate Products According to B) Synthesis Route IV

### Intermediate Product 42: 1-(2-Hydroxy-2-phenyl-ethyl)-piperidine-4-carboxylic acid ethyl ester

### (Process Step 13)

Styrene oxide (2.40 g, 20.0 mmol) and piperidine-4-carboxylic acid ethyl ester (3.14 g, 20.0 mmol) were heated in a closed tube at 90° C. for 2 hours. The crude yield was purified by means of trituration from Et₂O to give intermediate product 42 (2.0 mg, 40%).

MW: 277.37

HPLCMS (method B): [m/z]: 278

### XIII.II Example Compounds According to B) Synthesis Route IV

### Example Compound 57: 1-{2-[4-(2-Methoxy-ethyl)-piperazin-1-yl]-2-phenyl-ethyl}-piperidine-4-carboxylic acid ethyl ester

### (Process Step 14)

### 1-(2-Hydroxy-2-phenyl-ethyl)-piperidine-4-carboxylic acid ethyl ester (intermediate product 42) (1.1 g, 4.0 mmol) was dissolved in a stock solution of 11% TEA in THF (25 ml). Methanesulfonyl chloride (458 μl, 6.0 mmol) was added and the reaction mixture was stirred at room temperature for 1 hour; the reaction was monitored by means of LCMS in order to confirm the reaction of the starting materials. TEA (1.5 ml, 10.8 mmol) was added, followed by a solution of 1-(2-methoxyethyl)-piperazine (692 mg, 4.8 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The crude yield was dissolved in MC and the solution was washed with water and brine. The organic phase was dried (Na₂SO₄) and concentrated in vacuo. The crude yield was purified by means of column chromatography with MC/7 M NH₃ in MeOH (100:0-90:10) as the eluent to give Example Compound 57 (0.75 g, 47%).

EOAI3334111 VIT-1241

MW: 403.57

HPLCMS (method A): [m/z]: 404

FIG. 57 shows the result.

### Example Compound 58: 1-{2-[4-(2-Methoxy-ethyl)-piperazin-1-yl]-2-phenyl-ethyl}-piperidine-4-carboxylic acid phenylamide

### (Process Step 15)

A solution of 1-{2-[4-(2-methoxy-ethyl)-piperazin-1-yl]-2-phenyl-ethyl}-piperidine-4-carboxylic acid ethyl ester (Example Compound 57) (100 mg, 0.25 mmol) and aniline (92 μl, 1.0 mmol) in DCE (3 ml) was cooled to 0° C. under a nitrogen atmosphere and trimethylaluminium (2 M in hexane, 250 μl, 0.5 mmol) was added. The mixture was stirred at 0° C. for 10 minutes and then heated under reflux for 2 hours. The mixture was diluted with saturated aqueous NaHCO₃ and solid residues were removed by means of filtration through Celite (kieselguhr). The filtrate of the combined organic phases was dried (MgSO₄) and concentrated in vacuo. The crude yield was purified by means of column chromatography with MC/2 M NH₃ in MeOH (100:0-96:4) as the eluent to give Example Compound 58 (70 mg).

Salt formation: The yield was dissolved in MeOH (3 ml) and the solution was cooled to 0° C. 2 M HCl in Et₂O (3 eq) was added dropwise and the reaction mixture was stirred for 30 minutes. The mixture was concentrated in vacuo to give Example Compound 58 in the form of the HCl salt (20 mg, 14%).

EOAI3334332 VIT-1265

MW: 450.63

HPLCMS (method B): [m/z]: 451

FIG. 58 shows the result.

### Example Compound 59: 1-{2-[4-(2-Methoxy-ethyl)-piperazin-1-yl]-2-phenyl-ethyl}-piperidine-4-carboxylic acid methylamide

The preparation was carried out analogously to Example Compound 58 according to process step 15 using:

1-{2-[4-(2-methoxy-ethyl)-piperazin-1-yl]-2-phenyl-ethyl}-piperidine-4-carboxylic acid ethyl ester (Example Compound 57) (100 mg, 0.25 mmol) trimethylaluminium (2 M in hexane, 250 μl, 0.5 mmol) and methylamine (2 M in THF, 0.5 ml, 1.0 mmol).

Purification was carried out by means of column chromatography with MC/7 M NH₃ in MeOH (100:0-90:10) as the eluent to give Example Compound 59 (50 mg).

Salt formation: The yield was dissolved in MeOH (3 ml) and the solution was cooled to 0° C. 2 M HCl in Et₂O (3 eq) was added dropwise and the reaction mixture was stirred for 30 minutes. The mixture was concentrated in vacuo to give Example Compound 59 in the form of the HCl salt (14 mg, 11%).

EOAI3334333 VIT-1266

MW: 388.56

HPLCMS (method B): [m/z]: 389

FIG. 59 shows the result.

### Example Compound 60:1-{2-[4-(2-Methoxy-ethyl)-piperazin-1-yl]-2-phenyl-ethyl}-piperidine-4-carboxylic acid cyclohexylamide

The preparation was carried out analogously to Example Compound 58 according to process step 15 using:

1-{2-[4-(2-methoxy-ethyl)-piperazin-1-yl]-2-phenyl-ethyl}-piperidine-4-carboxylic acid ethyl ester (Example Compound 57) (100 mg, 0.25 mmol) trimethylaluminium (2 M in hexane, 250 μl, 0.5 mmol) and cyclohexylamine (116 μl, 1.0 mmol).

Purification was carried out by means of column chromatography with MC/7 M NH₃ in MeOH (100:0-97:3), followed by extraction by shaking with isocyanate resin, filtration and concentration in vacuo to give Example Compound 60.

Salt formation: The yield was dissolved in MeOH (3 ml) and the solution was cooled to 0° C. 2 M HCl in Et₂O (3 eq) was added dropwise and the reaction mixture was stirred for 30 minutes. The mixture was concentrated in vacuo to give Example Compound 60 in the form of the HCl salt (20 mg, 15%).

EOAI3334567 VIT-1294

MW: 456.66

HPLCMS (method B): [m/z]: 457

FIG. 60 shows the result.

### XIV.I Intermediate Products According to B) Synthesis Route V

### Intermediate Product 42: 4-(2-Methoxy-ethyl)-[1,4]diazepan-1-carboxylic acid tert-butyl ester

### (Process Step 16)

[1,4]Diazepan-1-carboxylic acid tert-butyl ester (3.0 g, 15.0 mmol) and potassium carbonate (1.88 g, 13.6 mmol) were dissolved in DMF (20 ml). 2-Bromoethyl methyl ether (1.27 ml, 13.6 mmol) was added and the mixture was heated at 60° C. for 16 hours. After cooling, the mixture was diluted with EtOAc and the resulting organic phase was washed with water (×3) and brine and dried (Na₂SO₄) and concentrated in vacuo to give intermediate product 42 (3.31 g, 86%). It was not possible to detect the compound by means of HPLCMS and the structure was therefore confirmed by means of ¹H NMR.

### Intermediate Product 43: 1-(2-Methoxy-ethyl)-[1,4]diazepan

### (Process Step 17)

### 4-(2-Methoxy-ethyl)-[1,4]diazepan-1-carboxylic acid tert-butyl ester (intermediate product 42) (1.00 g, 3.87 mmol) was dissolved in 20% TFA in MC (10 ml) and the mixture was stirred at room temperature for 30 minutes. The reaction mixture was concentrated in vacuo to give intermediate product 43, which could be employed without further purification. It was not possible to detect the compound by means of HPLCMS and the structure was therefore confirmed by means of ¹H NMR.

### XIV.II Example Compounds According to B) Synthesis Route V

### Example Compound 61: 1′-{2-[4-(2-Methoxy-ethyl)[1,4′]diazepan-1-yl]-2-phenyl-ethyl)}-[1,4′]bipiperidinyl

### (Process Step 18)

2-[1,4′]Bipiperidinyl-1′-yl-1-phenyl-ethanol (intermediate product 24) (426 mg, 1.48 mmol) was dissolved in a stock solution of 11% TEA in THF (25 ml). Methanesulfonyl chloride (172 μl, 2.22 mmol) was added and the reaction mixture was stirred at room temperature for 1 hour; the reaction was monitored by means of LCMS in order to confirm the reaction of the starting materials. TEA (616 μl, 4.44 mmol) was added, followed by a solution of 1-(2-methoxy-ethyl)-[1,4]diazepan (intermediate product 43) (281 mg, 1.78 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude yield was purified by means of column chromatography with MC/7 M NH₃ in MeOH (100:0-95:5) as the eluent to give Example Compound 61 (80 mg, 13%).

Salt formation: The yield was dissolved in MeOH (3 ml) and the solution was cooled to 0° C. HCl (4 eq)) was added and the reaction mixture was stirred at 0° C. for 20 minutes. The mixture was concentrated in vacuo to give Example Compound 61 in the form of the HCl salt.

EOAI3332900 VIT-1180

MW: 428.65

HPLCMS (method A): [m/z]: 429

FIG. 61 shows the result.

### XV.I Intermediate Products According to B) Synthesis Route VI

### Intermediate Product 44: 4-Cyclohexyl-piperidine

### (Process Step 19)

4-Phenylpyridine (1.00 g, 6.44 mmol) was dissolved in dry EtOH (20 ml) in a 50 ml hydrogen pressure container. Concentrated HCL (2 ml) and platinum oxide (20 mol %) were added and the reaction vessel was placed under a hydrogen pressure of 55 psi and the mixture was stirred for 48 hours. The mixture was diluted with MeOH (100 ml) and filtered through Celite (kieselguhr) and the filtrate was concentrated in vacuo. The resulting crude yield was dissolved in water (40 ml) and the solution was brought to pH 10 with 1 M sodium hydroxide solution. The aqueous phase was extracted with MC (×3) and the combined organic phases were dried (Na₂SO₄) and concentrated in vacuo to give intermediate product 44 (470 mg, 44%).

### Intermediate Product 45: 2-(4-Cyclohexyl-piperidin-1-yl)-1-phenyl-ethanol

### (Process Step 20)

Styrene oxide (220 mg, 1.83 mmol) and 4-cyclohexyl-piperidine (intermediate product 44) (306 mg, 1.83 mmol) were heated in a closed tube at 90° C. for 3 hours. The crude yield was purified by means of column chromatography with MC/MeOH (99:1-94:6) as the eluent to give intermediate product 45 (69 mg, 12%).

MW: 287.44

HPLCMS (method A): [m/z]: 288

### XVII Example Compounds According to B) Synthesis Route VI

### Example Compound 62: 1-[2-(4-Cyclohexyl-piperidin-1-yl)-1-phenyl-ethyl]-4-(2-methoxy-ethyl)-piperazine

### (Process Step 21)

2-(4-Cyclohexyl-piperidin-1-yl)-1-phenyl-ethanol (intermediate product 45) (69 mg, 0.24 mmol) was dissolved in a stock solution of 11% TEA in THF (25 ml). Methanesulfonyl chloride (28 μl, 0.36 mmol) was added and the reaction mixture was stirred at room temperature for 1 hour; the reaction was monitored by means of LCMS in order to confirm the reaction of the starting materials. TEA (67 μl, 0.48 mmol) was added, followed by a solution of 1-(2-methoxyethyl)-piperazine (42.8 μl, 0.29 mmol) in THF (4 ml). The reaction mixture was stirred at room temperature for 30 minutes. Water (4 ml) was added and the mixture was stirred at room temperature for 18 hours or until the reaction of all the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude yield was purified by means of preparative HPLC (basic conditions) to give Example Compound 62 (28 mg, 28%).

Salt formation: The yield was dissolved in a minimum amount of Et₂O/MC and the solution was cooled to 0° C. 2 M HCl in Et₂O (3 eq) was added dropwise and the reaction mixture was stirred for 30 minutes. The mixture was concentrated in vacuo to give Example Compound 62 in the form of the HCl salt.

EOAI3333815 VIT-1230

MW: 413.65

HPLCMS (method A): [m/z]: 414

FIG. 62 shows the result.

### XVI.I Intermediate Products According to B) Synthesis Route VII

### Intermediate Product 46: 1-(2-Hydroxy-2-phenyl-ethyl)-piperidin-4-one

### (Process Step 22)

Styrene oxide (0.27 g, 1.67 mmol) and piperidin-4-one (0.226 g, 1.67 mmol) were heated thoroughly in a closed tube at 90° C. for 2 hours. After cooling, the reaction mixture was diluted with MC (40 ml). The organic phase was washed with brine, dried (Na₂SO₄) and concentrated in vacuo. The crude yield was purified by means of column chromatography with MC/MeOH (95:5) as the eluent to give intermediate product 46 (0.10 g, 28%).

MW: 219.29

HPLCMS (method D): [m/z]: 220

### Intermediate Product 47: 2-(4-Cyclopentylamino-piperidin-1-yl)-1-phenyl-ethanol

### (Process Step 23)

Cyclopentylamine (0.05 ml, 0.46 mmol) was added to a solution of 1-(2-hydroxy-2-phenyl-ethyl)-piperidin-4-one (intermediate product 46) (0.10 g, 0.46 mmol) in MeOH (1 ml) and the reaction mixture was stirred for 30 minutes. NaBH₃CN (43 mg, 0.68 mmol) was added and stirring was continued for 18 hours. The reaction mixture was concentrated in vacuo, water (10 ml) was added and the mixture was extracted with MC. The organic phase was washed with brine, dried (Na₂SO₄) and concentrated in vacuo. The crude yield was purified by means of column chromatography with MC/MeOH (90:10) as the eluent to give intermediate product 47 (90 mg, 69%).

MW: 288.24

HPLCMS (method F): [m/z]: 288

### XVI.II Example Compounds According to B) Synthesis Route VII

### Example Compound 63: Cyclopentyl-(1-{2-[4-(2-methoxy-ethyl)-piperazin-1-yl]-2-phenyl-ethyl}-piperidin-4-yl)-amine

### (Process Step 24)

Methanesulfonyl chloride (0.07 ml, 0.94 mmol) was added to a solution of 2-(4-cyclopentylamino-piperidin-1-yl)-1-phenyl-ethanol (intermediate product 46) (0.18 g, 0.63 mmol) and TEA (0.17 ml, 1.25 mmol) in THF (3 ml) at 0° C. and the mixture was stirred at room temperature for 3 hours. TEA (0.17 ml, 1.25 mmol) and 1-(2-methoxy-ethyl)-piperazine (90 mg, 0.63 mmol) were added and stirring was continued for a further 2 hours. The reaction mixture was concentrated in vacuo, water (10 ml) was added and the mixture was extracted with MC. The organic phase was washed with brine, dried (Na₂SO₄) and concentrated in vacuo. The crude yield was purified by means of column chromatography with MC/MeOH/aq. NH₃ (95:5:1) as the eluent to give Example Compound 63 (70 mg, 28%).

EOAI3335382 VIT-1373: (EV0927-070-001)

MW: 414.64

HPLCMS (method F): [m/z]: 415

FIG. 63 shows the result.

### XVII.I Intermediate Products According to B) Synthesis Route VIII

### Intermediate Product 48: 1-(4-Ethoxy-phenyl)-ethanone

### (Process Step 25)

K₂CO₃ (2.02 g, 15.0 mmol) was added to a solution of 1-(4-hydroxy-phenyl)-ethanone (1.00 g, 7.34 mmol) in MeCN (10 ml) with constant stirring. Ethyl bromide (1.2 ml, 15.0 mmol) was added and the reaction mixture was heated at 80° C. for 20 hours. Water (10 ml) was added and the reaction mixture was extracted with EtOAc. The organic phase was washed with brine, dried (Na₂SO₄) and concentrated in vacuo. The crude yield was purified by means of column chromatography with EtOAc/hexane (0:100-2:98) as the eluent to give intermediate product 48 (1.10 g, 91%).

MW: 164.21

HPLCMS (method D): [m/z]: 165

### Intermediate Product 49: 1-(4-Isopropoxy-phenyl)-ethanone

The preparation was carried out analogously to intermediate product 48 according to process step 25 using: 1-(4-hydroxy-phenyl)-ethanone (1.00 g, 7.34 mmol), K₂CO₃ (2.02 g, 15.0 mmol) and iso-propyl bromide (1.40 ml, 15.0 mmol) to give intermediate product 49 (1.27 g, 97%).

MW: 178.23

HPLCMS (method D): [m/z]: 179

### Intermediate Product 50: 2-Bromo-1-(4-ethoxyphenyl)ethanone

### (Process Step 26)

A dioxane dibromide solution was prepared by addition of bromine

(94 μl, 1.83 mmol) to dioxane (5 ml) at 0° C. and with continued stirring for 30 minutes. The resulting solution was added to a solution of 1-(4-ethoxy-phenyl)-ethanone (intermediate product 48) (0.30 g, 1.83 mmol) in dioxane (10 ml) and the resulting mixture was stirred at room temperature for 18 hours. Water (10 ml) was added and the reaction mixture was extracted with EtOAc. The organic phase was washed with brine, dried (Na₂SO₄) and concentrated in vacuo to give intermediate product 50 (0.60 g), which could be employed in further process steps without further purification.

MW: 243.10

HPLCMS (method D): [m/z]: 244

### Intermediate Product 51: 2-Bromo-1-(4-isopropoxy-phenyl)-ethanone

The preparation was carried out analogously to intermediate product 50 according to process step 26 using:

1-(4-isopropoxy-phenyl)-ethanone (intermediate product 49) (0.50 g, 2.8 mmol), Br₂ (0.14 ml, 2.8 mmol) in dioxane (15 ml) to give intermediate product 51 (0.78 g), which could be employed in the further process steps without further purification.

MW: 257.13

HPLCMS (method D): [m/z]: 258

### Intermediate Product 52: 2-[1,4′]Bipiperidinyl-1′-yl-1-(4-ethoxy-phenyl)-ethanone

### (Process Step 27)

### 4-(Piperidin-1-yl)piperidine (0.34 g, 2.0 mmol) was added to a solution of 2-bromo-1-(4-ethoxyphenyl)ethanone (intermediate product 50) (0.60 g, 2.5 mmol) and TEA (0.70 ml, 5.0 mmol) in MC (15 ml) at 0° C. and the mixture was stirred at room temperature for 18 hours. Water (10 ml) was added and the reaction mixture was extracted with MC. The organic phase was washed with brine, dried (Na₂SO₄) and concentrated in vacuo. The crude yield was purified by means of column chromatography with MC/MeOH/aq. NH₃ (99:1:1) as the eluent to give intermediate product 52 (0.35 g, 43%).

MW: 330.47

HPLCMS (method D): [m/z]: 331

### Intermediate Product 53: 2-[1,4′]Bipiperidinyl-1′-yl-1-(4-isopropoxy-phenyl)-ethanone

The preparation was carried out analogously to intermediate product 52 according to process step 27 using:

2-bromo-1-(4-isopropoxy-phenyl)-ethanone (intermediate product 51) (0.50 g, 1.94 mmol), TEA (0.55 ml, 3.88 mmol) and 4-(piperidin-1-yl)piperidine (0.33 g, 1.94 mmol) in MC (15 ml).

Purification was carried out by means of column chromatography with MC/MeOH/aq. NH₃ (99:1:2) as the eluent to give intermediate product 53 (0.33 g, 40%).

MW: 344.5

HPLCMS (method D): [m/z]: 345

### Intermediate Product 54: 2-[1,4′]Bipiperidinyl-1′-yl-1-(4-ethoxy-phenyl)-ethanol

### (Process Step 28)

NaBH₄ (48 mg, 1.27 mmol) was added to a stirred solution of 2-[1,4′]bipiperidinyl-1′-yl-1-(4-ethoxy-phenyl)-ethanone (intermediate product 52) (0.35 g, 1.5 mmol) in MeOH (10 ml) at 0° C. and the mixture was stirred at room temperature for 1 hour. MeOH was removed in vacuo, water was added and the mixture was extracted with EtOAc. The organic phase was washed with brine, dried (Na₂SO₄) and concentrated in vacuo. The crude yield was purified by means of trituration from n-hexane (0.19 g, 55%). It was not possible to detect the compound by means of HPLCMS and the structure was therefore confirmed by means of ¹H NMR.

### Intermediate Product 55: 2-[1,4′]Bipiperidinyl-1′-yl-1-(4-isopropoxy-phenyl)-ethanol

The preparation was carried out analogously to intermediate product 54 according to process step 28 using:

2-[1,4′]bipiperidinyl-1′-yl-1-(4-isopropoxy-phenyl)-ethanone (intermediate product 53) (0.33 g, 0.94 mmol) and NaBH₄ (43 mg, 1.13 mmol) in MeOH (10 ml) to give intermediate product 55 (0.17 g, 51%). It was not possible to detect the compound by means of HPLCMS and the structure was therefore confirmed by means of ¹H NMR.

### XVIII.I Example Compounds According to B) Synthesis Route VIII

### Example Compound 64: 1′-{2-(4-Ethoxy-phenyl)-2-[4-(2-methoxy-ethyl)-piperazin-1-yl]-ethyl}-[1,4′]bipiperidinyl

### (Process Step 29)

Methanesulfonyl chloride (65 μl, 0.83 mmol) was added to a solution of 2-[1,4′]bipiperidinyl-1′-yl-1-(4-ethoxy-phenyl)-ethanol (intermediate product 54) (190 mg, 0.56 mmol) and TEA (160 μl, 1.1 mmol) in THF (10 ml) at 0° C. and the mixture was stirred at room temperature for 3 hours. TEA (160 μl, 1.1 mmol) was added, followed by 1-(2-methoxy-ethyl)-piperazine (80 mg, 0.56 mmol), and stirring was continued for a further 1.5 hours. Water (10 ml) was added and the mixture was stirred for 18 hours. The reaction mixture was extracted with MC and the organic phase was washed with brine, dried (Na₂SO₄) and concentrated in vacuo. The crude yield (containing alcohol as a non-separable impurity) was dissolved in pyridine (5 ml), acetic anhydride (90 μl, 0.96 mmol) was added at 0° C. and the reaction mixture was stirred at room temperature for 3 hours. The reaction mixture was concentrated in vacuo and the crude yield was purified by means of column chromatography with MC/MeOH/aq. NH₃ (100:0:0-95:5:2) as the eluent to give Example Compound 64 (63 mg, 25%).

EOAI3335954 VIT-1429

MW: 458.68

HPLCMS (method F): [m/z]: 459

FIG. 64 shows the result.

### Example Compound 65: 1′-{2-(4-Isopropoxy-phenyl)-2-[4-(2-methoxy-ethyl)-piperazin-1-yl]-ethyl}[1,4′]bipiperidinyl

The preparation was carried out analogously to Example Compound 64 according to process step 29 using:

2-[1,4′]bipiperidinyl-1′-yl-1-(4-ethoxy-phenyl)-ethanol (intermediate product 55) (170 mg, 0.48 mmol), TEA (133 μl, 0.96 mmol) and methanesulfonyl chloride (60 μl, 0.73 mmol) followed by 1-(2-methoxy-ethyl)-piperazine (72 μl, 0.48 mmol) and TEA (133 μl, 0.96 mmol). Purification was carried out by means of column chromatography with MC/MeOH/aq. NH₃ (100:0:0-95:5:2) as the eluent to give Example Compound 65 (53 mg, 23%).

EOAI3335955 VIT-1430

MW: 472.71

HPLCMS (method F): [m/z]: 473

FIG. 65 shows the result.

### XVIII.I Intermediate Products According to B) Synthesis Route IX

### Intermediate Product 56: 2-(4-Methoxy-phenyl)-oxirane (process step 30)

A solution of trimethylsulfonium iodide (4.0 g, 19.8 mmol) in DMSO (40 ml) was added to sodium hydride (60% in mineral oil, 1.32 g, 33 mmol) in a dry 2 neck RB flask under a nitrogen atmosphere. The solution was stirred at room temperature for 30 minutes, before it was cooled to 0° C. and a solution of 4-methoxybenzaldehyde in DMSO (10 ml) was added dropwise. The mixture was stirred at room temperature for 18 hours. The reaction mixture was diluted with water and EtOAc and the organic phase was washed with water and brine, dried (Na₂SO₄) and concentrated in vacuo to give intermediate product 56 (2.1 g, 84%).

MW: 150.18

HPLCMS (method C): [m/z]: 151

### Intermediate Product 57: 2-p-Tolyl-oxirane

The preparation was carried out analogously to intermediate product 56 according to process step 30 using:

NaH (0.40 g, 0.99 mmol), trimethylsulfonium iodide (2.00 g, 0.99 mmol) and 4-methylbenzaldehyde (1.0 g, 8.3 mmol). Purification was carried out by means of column chromatography with hexane/EtOAc (98:2) as the eluent to give intermediate product 57 (0.47 g, 40%). It was not possible to detect the compound by means of HPLCMS and the structure was therefore confirmed by means of ¹H NMR.

### Intermediate Product 58: 2-(3-Methoxy-phenyl)-oxirane

The preparation was carried out analogously to intermediate product 56 according to process step 30 using:

3-methoxybenzaldehyde (0.90 ml, 7.3 mmol), trimethylsulfonium iodide (1.80 g, 8.7 mmol) and NaH (0.35 g, 8.7 mmol). Purification was carried out by means of column chromatography with hexane/EtOAc (98:2) as the eluent to give intermediate product 58 (0.80 g, 72%). It was not possible to detect the compound by means of HPLCMS and the structure was therefore confirmed by means of ¹H NMR.

### Intermediate Product 59: 2-(3-Chloro-phenyl)-oxirane

The preparation was carried out analogously to intermediate product 56 according to process step 30 using:

3-chlorobenzaldehyde (1.00 g, 7.1 mmol), trimethylsulfonium iodide (1.74 g, 8.5 mmol) and NaH (0.34 g, 8.5 mmol). Purification was carried out by means of column chromatography with hexane/EtOAc (98:2) as the eluent to give intermediate product 59 (0.40 g, 37%). It was not possible to detect the compound by means of HPLCMS and the structure was therefore confirmed by means of ¹H NMR.

### Intermediate Product 60: 2-(2-Chloro-phenyl)-oxirane

The preparation was carried out analogously to intermediate product 56 according to process step 30 using:

2-chlorobenzaldehyde (1.00 g, 7.1 mmol), trimethylsulfonium iodide (1.74 g, 8.5 mmol) and NaH (0.34 g, 8.5 mmol). Purification was carried out by means of column chromatography with hexane/EtOAc (98:2) as the eluent to give intermediate product 60 (0.40 g, 37%). It was not possible to detect the compound by means of HPLCMS and the structure was therefore confirmed by means of ¹H NMR.

### Intermediate Product 61: 4-Oxiranyl-benzonitrile

The preparation was carried out analogously to intermediate product 56 according to process step 30 using:

4-formylbenzonitrile (1.00 g, 7.6 mmol), trimethylsulfonium iodide (1.90 g, 7.6 mmol) and NaH (0.36 g, 9.1 mmol). Purification was carried out by means of column chromatography with hexane/EtOAc (98:2) as the eluent to give intermediate product 61 (0.15 g, 14%). It was not possible to detect the compound by means of HPLCMS and the structure was therefore confirmed by means of ¹H NMR.

### Intermediate Product 62: 2-(4-Trifluoromethyl-phenyl)-oxirane

The preparation was carried out analogously to intermediate product 56 according to process step 30 using:

4-trifluoromethylbenzaldehyde (1.00 g, 5.7 mmol), trimethylsulfonium iodide (1.40 g, 6.8 mmol) and NaH (0.27 g, 6.8 mmol). Purification was carried out by means of column chromatography with hexane/EtOAc (98:2) as the eluent to give intermediate product 62 (0.25 g, 25%). It was not possible to detect the compound by means of HPLCMS and the structure was therefore confirmed by means of ¹H NMR.

### Intermediate Product 63: 2-[1,4′]Bipiperidinyl-1′-yl-1-(4-methoxy-phenyl)-ethanol

### (Process Step 31)

2-(4-Methoxy-phenyl)-oxirane (intermediate product 56) (750 mg, 5.4 mmol) and [1,4′]bipiperidinyl (900 mg, 5.4 mmol) were heated in a closed tube at 90° C. for 4 hours. The crude yield was purified by means of column chromatography with MC/MeON (100:0-90:10) as the eluent to give intermediate product 63 (700 mg, 41%).

MW: 318.46

HPLCMS (method C): [m/z]: 319

### Intermediate Product 64: 2-[1,4′]Bipiperidinyl-1′-yl-1-p-tolyl-ethanol

The preparation was carried out analogously to intermediate product 63 according to process step 31 using:

2-p-tolyl-oxirane (intermediate product 57) (0.47 ml, 3.5 mmol) and 4-(piperidin-1-yl)piperidine (0.59 g, 3.5 mmol) to give intermediate product 64 (0.42 g, 40%). It was not possible to detect the compound by means of HPLCMS and the structure was therefore confirmed by means of ¹H NMR.

### Intermediate Product 65: 2-[1,4′]Bipiperidinyl-1′-yl-1-(3-methoxy-phenyl)-ethanol

The preparation was carried out analogously to intermediate product 63 according to process step 31 using:

2-(3-methoxy-phenyl)-oxirane (intermediate product 58) (0.40 g, 2.6 mmol) and 4-(piperidin-1-yl)piperidine (0.44 g, 2.6 mmol) to give intermediate product 65 (0.80 g, 97%). It was not possible to detect the compound by means of HPLCMS and the structure was therefore confirmed by means of ¹H NMR.

### Intermediate Product 66: 2-[1,4′]Bipiperidinyl-1′-yl-1-(3-chloro-phenyl)-ethanol

The preparation was carried out analogously to intermediate product 63 according to process step 31 using:

2-(3-chloro-phenyl)-oxirane (intermediate product 59) (0.40 g, 2.5 mmol) and 4-(piperidin-1-yl)piperidine (0.42 g, 2.5 mmol) to give intermediate product 66 (0.40 g, 50%). It was not possible to detect the compound by means of HPLCMS and the structure was therefore confirmed by means of ¹H NMR.

### Intermediate Product 67: 2-[1,4′]Bipiperidinyl-1′-yl-1-(2-chloro-phenyl)-ethanol

The preparation was carried out analogously to intermediate product 63 according to process step 31 using:

2-(2-chloro-phenyl)-oxirane (intermediate product 60) (0.30 g, 1.9 mmol) and 4-(piperidin-1-yl)piperidine (0.32 g, 1.9 mmol) to give intermediate product 67 (0.40 g, 65%). It was not possible to detect the compound by means of HPLCMS and the structure was therefore confirmed by means of ¹H NMR.

### Intermediate Product 68: 4-(2-[1,4′]Bipiperidinyl-1′-yl-1-hydroxy-ethyl)-benzonitrile

The preparation was carried out analogously to intermediate product 63 according to process step 31 using:

4-oxiranyl-benzonitrile (intermediate product 61) (0.15 ml, 1.03 mmol) and 4-(piperidin-1-yl)piperidine (0.17 g, 1.03 mmol) to give intermediate product 68 (0.23 g, 71%). It was not possible to detect the compound by means of HPLCMS and the structure was therefore confirmed by means of ¹H NMR.

### Intermediate Product 69: 2-[1,4′]Bipiperidinyl-1′-yl-1-(4-trifluoromethyl-phenyl)-ethanol

The preparation was carried out analogously to intermediate product 63 according to process step 31 using:

2-(4-trifluoromethyl-phenyl)-oxirane (intermediate product 62) (0.25 ml, 1.3 mmol) and 4-(piperidin-1-yl)piperidine (0.22 g, 1.3 mmol) to give intermediate product 69 (0.28 g, 67%). It was not possible to detect the compound by means of HPLCMS and the structure was therefore confirmed by means of ¹H NMR.

### XVIII.II Example Compounds According to B) Synthesis Route IX

### Example Compound 66: 1′-[2-[4-(2-Methoxy-ethyl)-piperazin-1-yl]-2-(4-methoxy-phenyl)-ethyl]-[1,4′]bipiperidinyl

### (Process Step 32)

2-[1,4′]Bipiperidinyl-1′-yl-1-(4-methoxy-phenyl)-ethanol (intermediate product 63) (105 mg, 0.51 mmol) was dissolved in a stock solution of 11% TEA in THF (25 ml). Methanesulfonyl chloride (60 μl, 0.78 mmol) was added and the reaction mixture was stirred at room temperature for 1 hour; the reaction was monitored by means of LCMS in order to confirm the reaction of the starting materials. TEA (1.5 ml, 10.8 mmol) was added, followed by a solution of 1-(2-methoxyethyl)-piperazine (93 μl, 0.62 mmol) in THF (4 ml). The reaction mixture was stirred at room temperature for 30 minutes. Water (4 ml) was added and the mixture was stirred at room temperature for 18 hours or until the reaction of the starting materials and reaction intermediate products was confirmed by means of LCMS. The reaction mixture was concentrated in vacuo. The crude yield was dissolved in MC and the solution was washed with water and brine. The organic phase was dried (Na₂SO₄) and concentrated in vacuo. The crude yield was purified by means of preparative HPLC (basic conditions) to give Example Compound 66.

Salt formation: The yield was dissolved in MeOH (3 ml) and the solution was cooled to 0° C. 2 M HCl in Et₂O (3 eq) was added dropwise and the reaction mixture was stirred for 30 minutes. The mixture was concentrated in vacuo to give Example Compound 66 in the form of the HCl salt (75 mg, 38%).

EOAI3333470 VIT-1191

MW: 444.67

HPLCMS (method A): [m/z]: 445

FIG. 66 shows the result.

### Example Compound 67: 1-(2-Methoxyethyl)-4,1-(4-methylphenyl)-2-[4-(piperidin-1-yl)piperidin-1-yl]ethyl]piperazine

### (Process Step 33)

Methanesulfonyl chloride (0.11 ml, 0.99 mmol) was added to a solution of 2-[1,4′]bipiperidinyl-1′-yl-1-p-tolylethanol (intermediate product 64) (0.20 g, 0.66 mmol) and TEA (0.19 ml, 1.32 mmol) in THF (10 ml) at 0° C. and the mixture was stirred at room temperature for 3 hours. TEA (0.19 ml, 1.32 mmol) was added, followed by 1-(2-methoxy-ethyl)-piperazine (0.10 ml, 0.66 mmol), and stirring was continued for a further 1.5 hours. Water (10 ml) was added and the mixture was stirred for 18 hours. The reaction mixture was extracted with MC and the organic phase was washed with brine, dried (Na₂SO₄) and concentrated in vacuo. The crude yield (containing alcohol as a non-separable impurity) was dissolved in pyridine (9 ml), acetic anhydride (0.16 ml, 1.7 mmol) was added at 0° C. and the reaction mixture was stirred at room temperature for 3 hours. The reaction mixture was concentrated in vacuo and the crude yield was purified by means of column chromatography with MC/MeOH/aq. NH₃ (100:0:0-95:5:2) as the eluent to give Example Compound 67 (90 mg, 32%).

EOAI3335300 VIT-1358

MW: 428.67

HPLCMS (method F): [m/z]: 428

FIG. 67 shows the result.

### Example Compound 68: 1-(2-Methoxyethyl)-4-[1-(3-methoxyphenyl)-2,4-(piperidin-1-yl)piperidin-1-yl]ethyl]piperazine

The preparation was carried out analogously to Example Compound 67 according to process step 33 using:

2-[1,4′]bipiperidinyl-1′-yl-1-(3-methoxy-phenyl)-ethanol (intermediate product 65) (0.40 g, 2.0 mmol), TEA (0.36 ml, 2.6 mmol) and methanesulfonyl chloride (0.16 ml, 2.0 mmol), followed by 1-(2-methoxy-ethyl)-piperazine (0.23 ml, 1.5 mmol) and TEA (0.36 ml, 2.6 mmol). Purification was carried out by means of column chromatography with MC/MeOH/aq. NH₃ (100:0:0-95:5:2) as the eluent to give Example Compound 68 (90 mg, 16%).

EOAI3335068 VIT-1330

MW: 444.67

HPLCMS (method F): [m/z]: 445

FIG. 68 shows the result.

### Example Compound 69: 111-(3-Chlorophenyl)-2-[4-(piperidin-1-yl)piperidin-1-yl]ethyl]-4-(2-methoxyethyl)piperazine

The preparation was carried out analogously to Example Compound 67 according to process step 33 using:

2-[1,4′]bipiperidinyl-1′-yl-1-(3-chloro-phenyl)-ethanol (intermediate product 66) (0.20 g, 0.7 mmol), TEA (0.17 ml, 1.2 mmol) and methanesulfonyl chloride (0.07 ml, 0.9 mmol), followed by 1-(2-methoxy-ethyl)-piperazine (0.09 ml, 0.6 mmol) and TEA (0.17 ml, 1.2 mmol). Purification was carried out by means of column chromatography with MC/MeOH/aq. NH₃ (100:0:0-95:5:2) as the eluent to give Example Compound 69. (0.14 g, 49%).

EOAI3335299 VIT-1357

MW: 449.08

HPLCMS (method F): [m/z]: 449

FIG. 69 shows the result.

### Example Compound 70: 1-[1-(2-Chlorophenyl)-2-[4-(piperidin-1-yl)piperidin-1-yl]ethyl]-4-(2-methoxyethyl)piperazine

The preparation was carried out analogously to Example Compound 67 according to process step 33 using:

2-[1,4′]bipiperidinyl-1′-yl-1-(2-chloro-phenyl)-ethanol (intermediate product 67) (0.20 g, 0.7 mmol), TEA (0.17 ml, 1.2 mmol) and methanesulfonyl chloride (0.07 ml, 0.9 mmol), followed by 1-(2-methoxy-ethyl)-piperazine (0.09 ml, 0.6 mmol) and TEA (0.17 ml, 1.2 mmol). Purification was carried out by means of column chromatography with MC/MeOH/aq. NH₃ (100:0:0-95:5:2) as the eluent to give Example Compound 70 (0.19 g, 68%).

EOAI3335067 VIT-1329

MW: 449.08

HPLCMS (method F): [m/z]: 449

FIG. 70 shows the result.

### Example Compound 71: 4-{2-[1,4′]Bipiperidinyl-1′-yl-1-[4-(2-methoxy-ethyl)-piperazin-1-yl]-ethyl}-benzonitrile

The preparation was carried out analogously to Example Compound 67 according to process step 33 using:

4-(2-[1,4′]bipiperidinyl-1′-yl-1-hydroxy-ethyl)-benzonitrile (intermediate product 68) (0.22 g, 0.7 mmol), TEA (0.20 ml, 1.4 mmol) and methanesulfonyl chloride (0.12 ml, 1.0 mmol) followed by 1-(2-methoxy-ethyl)-piperazine (0.10 g, 0.66 mmol) and TEA (0.20 ml, 1.4 mmol). Purification was carried out by means of column chromatography with MC/MeOH/aq. NH₃ (100:0:0-95:5:2) as the eluent to give Example Compound 71 (0.16 g, 50%).

EOAI3335302 VIT-1360

MW: 439.65

HPLCMS (method F): [m/z]: 439

FIG. 71 shows the result.

### Example Compound 72: 1-(2-Methoxyethyl)-4-{2-[4-(piperidin-1-yl)piperidin-1-yl]-1-[4-(trifluoromethyl)phenyl]ethyl}piperazine

The preparation was carried out analogously to Example Compound 67 according to process step 33 using:

2-[1,4′]bipiperidinyl-1′-yl-1-(4-trifluoromethyl-phenyl)-ethanol (intermediate product 69) (0.20 g, 0.56 mmol), TEA (0.16 ml, 1.12 mmol) and methanesulfonyl chloride (0.09 ml, 0.84 mmol) followed by 1-(2-methoxy-ethyl)-piperazine (0.08 g, 0.56 mmol) and (0.16 ml, 1.12 mmol). Purification was carried out by means of column chromatography with MC/MeOH/aq. NH₃ (100:0:0-95:5:2) as the eluent to give Example Compound 72 (0.08 g, 30%).

EOAI3335301 VIT-1359

MW: 482.64

HPLCMS (method F): [m/z]: 483

FIG. 72 shows the result.

### XIX.I Intermediate Products According to B) Synthesis Route X

### Intermediate Product 70: 2-[1,4′]Bipiperidinyl-1′-yl-1-(4-chloro-phenyl)-ethanone

### (Process Step 34)

### 2-Bromo-4′-chloroacetophenone (0.5 g, 2.1 mmol) was added to a solution of 4-(piperidin-1-yl)piperidine (0.36 g, 2.1 mmol) and TEA (0.6 ml, 4.2 mmol) in MC (20 ml) at 0° C. and the solution was stirred at room temperature for 15 hours. Water (10 ml) was added and the reaction mixture was extracted with MC (50 ml). The organic phase was washed with brine, dried (Na₂SO₄) and concentrated in vacuo. The crude yield obtained (0.6 g, 94%) had a purity of >90%, determined by means of LCMS, and could be used for the further process steps without further purification.

MW: 320.87

HPLCMS (method D): [m/z]: 321

### Intermediate Product 71: 2-[1,4′]Bipiperidinyl-1′-yl-1-(4-chloro-phenyl)-ethanol

### (Process Step 35)

NaBH₄ (42 mg, 1.12 mmol) was added to a solution of 2-[1,4′]bipiperidinyl-1′-yl-1-(4-chloro-phenyl)-ethanone (intermediate product 70) (0.3 g, 0.94 mmol) in MeOH (10 ml) at 0° C. and the reaction mixture was stirred at room temperature for 2 hours. The reaction mixture was concentrated in vacuo, water (10 ml) was added and the reaction mixture was extracted with EtOAc. The organic phase was washed with brine, dried (Na₂SO₄) and concentrated in vacuo. The crude yield was purified by means of trituration from hexane to give intermediate product 71 (0.21 g, 70%). It was not possible to detect the compound by means of HPLCMS and the structure was therefore confirmed by means of ¹H NMR.

### XIX.II Example Compounds According to B) Synthesis Route X

### Example Compound 73: 1′-{2-(4-Chloro-phenyl)-2-[4-(2-methoxy-ethyl)-piperazin-1-yl]-ethyl}-[1,4′]bipiperidinyl

### (Process Step 36)

Methanesulfonyl chloride (76 μl, 0.8 mmol) was added to a solution of 2-[1,4′]Dipiperidinyl-1′-yl-1-(4-chloro-phenyl)-ethanol (intermediate product 71) (0.21 g, 0.65 mmol) and TEA (0.182 ml, 1.3 mmol) in THF (10 ml) at 0° C. and the mixture was stirred at room temperature for 3 hours. TEA (0.18 ml, 1.3 mmol) was added, followed by 1-(2-methoxy-ethyl)-piperazine (94 μl, 0.65 mmol), and stirring was continued for a further 1.5 hours. Water (10 ml) was added and the mixture was stirred for 18 hours. The reaction mixture was extracted with MC and the organic phase was washed with brine, dried (Na₂SO₄) and concentrated in vacuo. The crude yield (0.1 g, 0.35 mmol) (containing alcohol as a non-separable impurity) was dissolved in pyridine (3 ml) and acetic anhydride (0.056 ml, 0.6 mmol) was added at 0° C. and the reaction mixture was stirred at room temperature for 3 hours. The reaction mixture was concentrated in vacuo and the crude yield was purified by means of column chromatography with MC/MeOH aq. NH₃ (100:0:0-95:5:2) as the eluent to give Example Compound 73 (175 mg, 60%).

EOAI333557 VIT-1396

MW: 449.08

HPLCMS (method F): [m/z]: 449

FIG. 73 shows the result.

### DESCRIPTION OF THE FIGURES

FIG. 1: HPLC-MS of Example Compound 1

FIG. 2: HPLC-MS of Example Compound 2

FIG. 3: HPLC-MS of Example Compound 3

FIG. 4: HPLC-MS of Example Compound 4

FIG. 5: H PLC-MS of Example Compound 5

FIG. 6: HPLC-MS of Example Compound 6

FIG. 7: HPLC-MS of Example Compound 7

FIG. 8: H PLC-MS of Example Compound 8

FIG. 9: HPLC-MS of Example Compound 9

FIG. 10: HPLC-MS of Example Compound 10

FIG. 11: H PLC-MS of Example Compound 11

FIG. 12: HPLC-MS of Example Compound 12

FIG. 13: HPLC-MS of Example Compound 13

FIG. 14: HPLC-MS of Example Compound 14

FIG. 15: HPLC-MS of Example Compound 15

FIG. 16: HPLC-MS of Example Compound 16

FIG. 17: HPLC-MS of Example Compound 17

FIG. 18: HPLC-MS of Example Compound 18

FIG. 19: HPLC-MS of Example Compound 19

FIG. 20: H PLC-MS of Example Compound 20

FIG. 21: HPLC-MS of Example Compound 21

FIG. 22: HPLC-MS of Example Compound 22

FIG. 23: HPLC-MS of Example Compound 23

FIG. 24: HPLC-MS of Example Compound 24

FIG. 25: HPLC-MS of Example Compound 25

FIG. 26: HPLC-MS of Example Compound 26

FIG. 27: HPLC-MS of Example Compound 27

FIG. 28: HPLC-MS of Example Compound 28

FIG. 29: HPLC-MS of Example Compound 29

FIG. 30: HPLC-MS of Example Compound 30

FIG. 31: HPLC-MS of Example Compound 31

FIG. 32: HPLC-MS of Example Compound 32

FIG. 34: HPLC-MS of Example Compound 34

FIG. 35: HPLC-MS of Example Compound 35

FIG. 36: HPLC-MS of Example Compound 36

FIG. 37: HPLC-MS of Example Compound 37

FIG. 38: HPLC-MS of Example Compound 38

FIG. 39: HPLC-MS of Example Compound 39

FIG. 40: HPLC-MS of Example Compound 40

FIG. 41: HPLC-MS of Example Compound 41

FIG. 42: HPLC-MS of Example Compound 42

FIG. 43: HPLC-MS of Example Compound 43

FIG. 44: HPLC-MS of Example Compound 44

FIG. 45: HPLC-MS of Example Compound 45

FIG. 46: HPLC-MS of Example Compound 46

FIG. 47: HPLC-MS of Example Compound 47

FIG. 48: HPLC-MS of Example Compound 48

FIG. 49: HPLC-MS of Example Compound 49

FIG. 50: HPLC-MS of Example Compound 50

FIG. 51: HPLC-MS of Example Compound 51

FIG. 52: HPLC-MS of Example Compound 52

FIG. 53: HPLC-MS of Example Compound 53

FIG. 54: HPLC-MS of Example Compound 54

FIG. 55: HPLC-MS of Example Compound 55

FIG. 56: HPLC-MS of Example Compound 56

FIG. 57: HPLC-MS of Example Compound 57

FIG. 58: HPLC-MS of Example Compound 58

FIG. 59: HPLC-MS of Example Compound 59

FIG. 60: HPLC-MS of Example Compound 60

FIG. 61: HPLC-MS of Example Compound 61

FIG. 62: HPLC-MS of Example Compound 62

FIG. 63: HPLC-MS of Example Compound 63

FIG. 64: HPLC-MS of Example Compound 64

FIG. 65: HPLC-MS of Example Compound 65

FIG. 66: HPLC-MS of Example Compound 66

FIG. 67: HPLC-MS of Example Compound 67

FIG. 69: HPLC-MS of Example Compound 69

FIG. 70: HPLC-MS of Example Compound 70

FIG. 71: HPLC-MS of Example Compound 71

FIG. 72: HPLC-MS of Example Compound 72

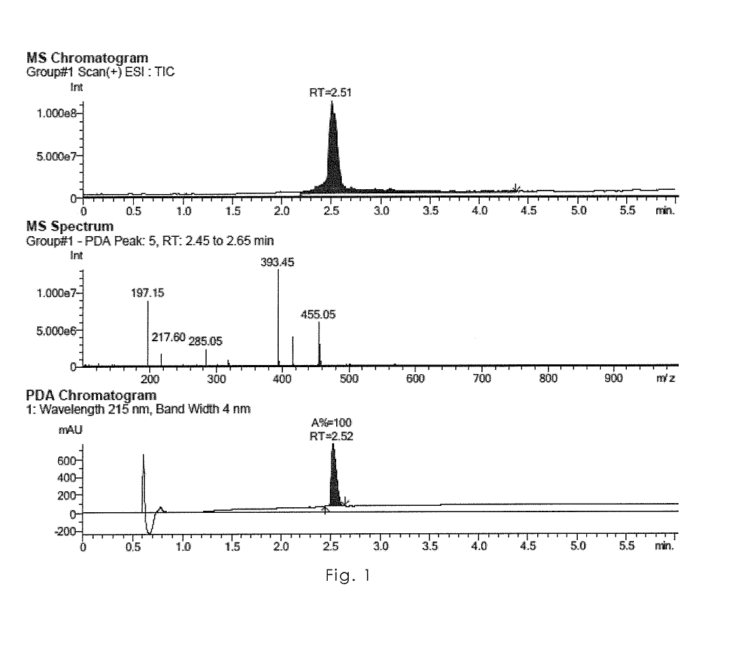

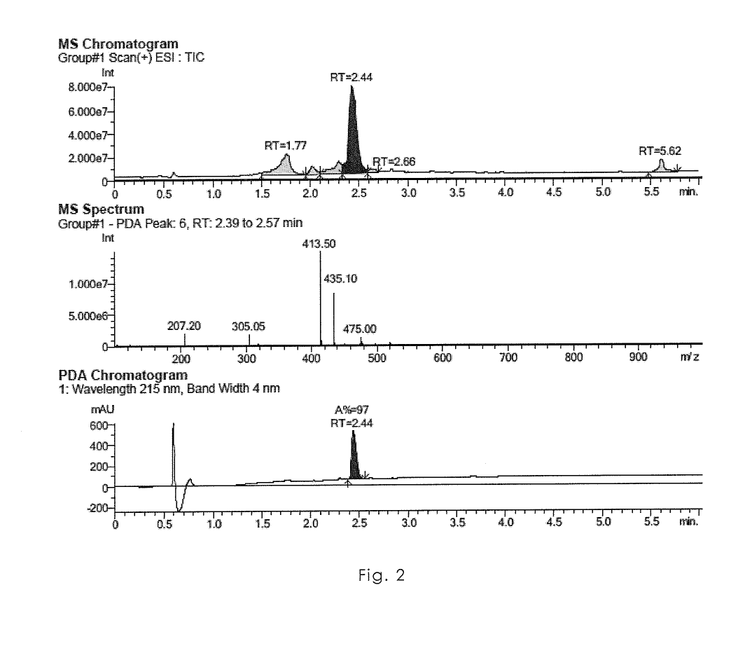

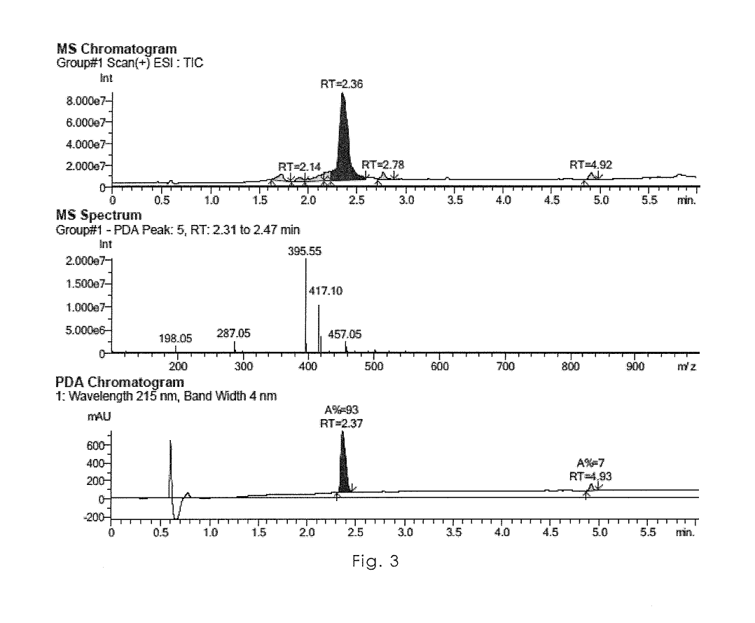

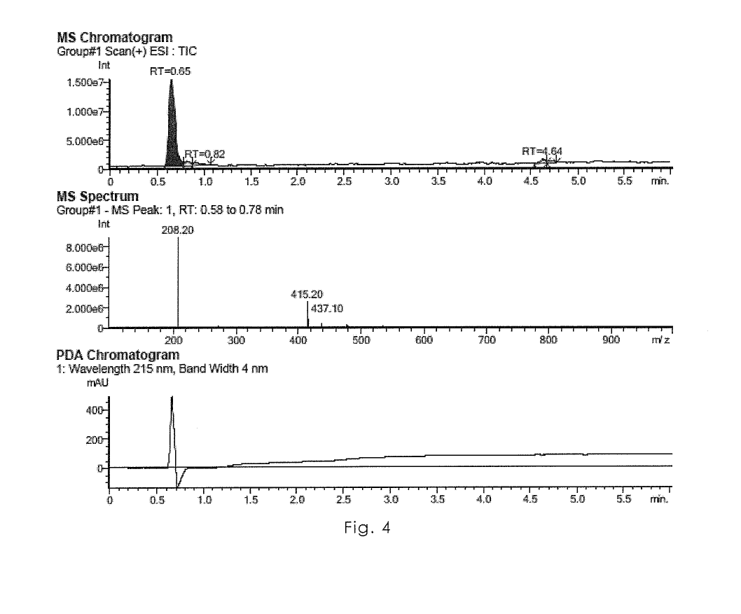

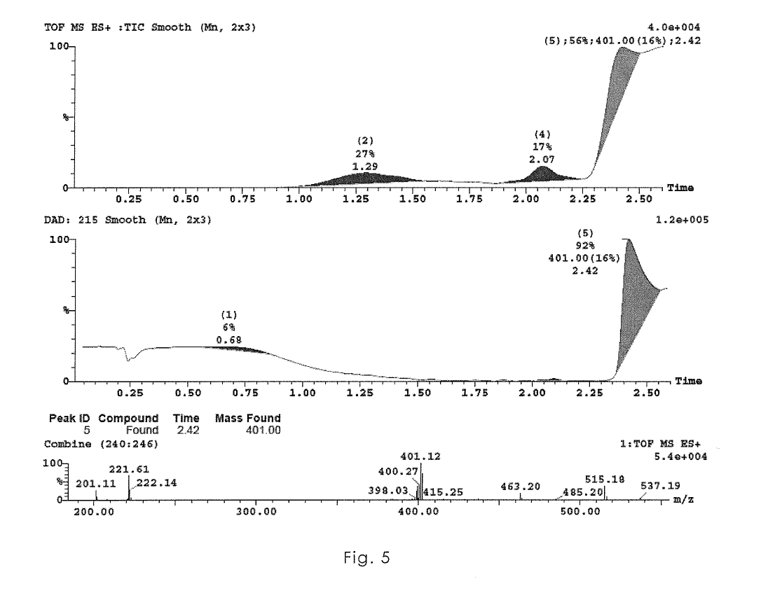

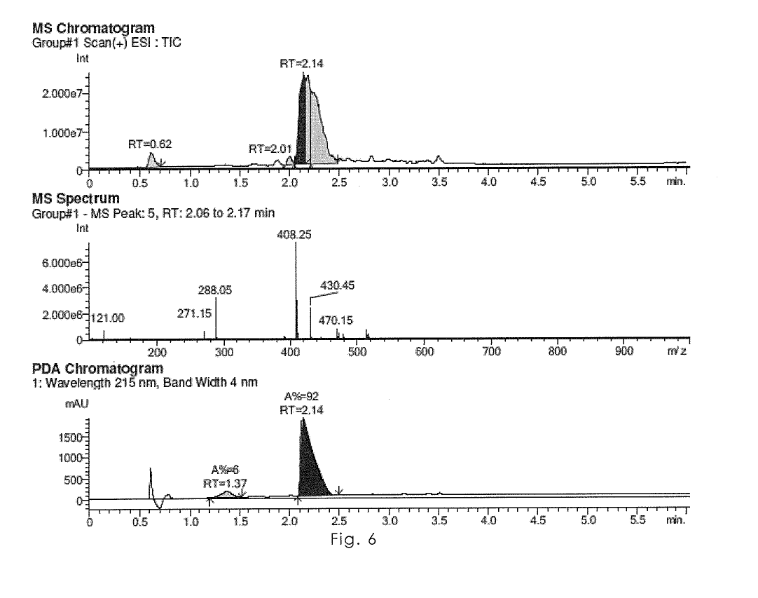

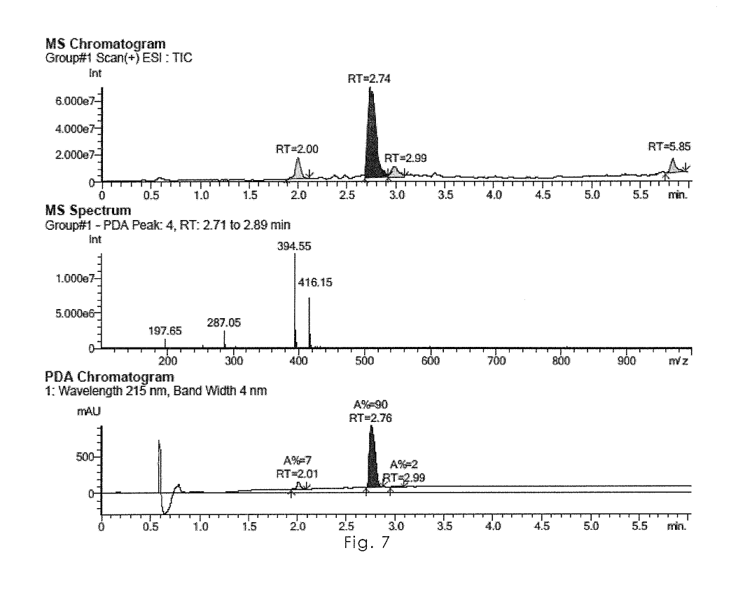

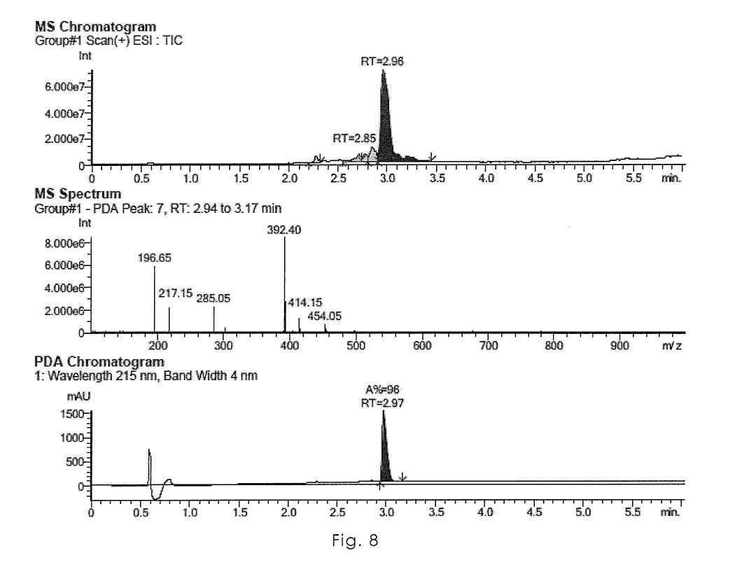

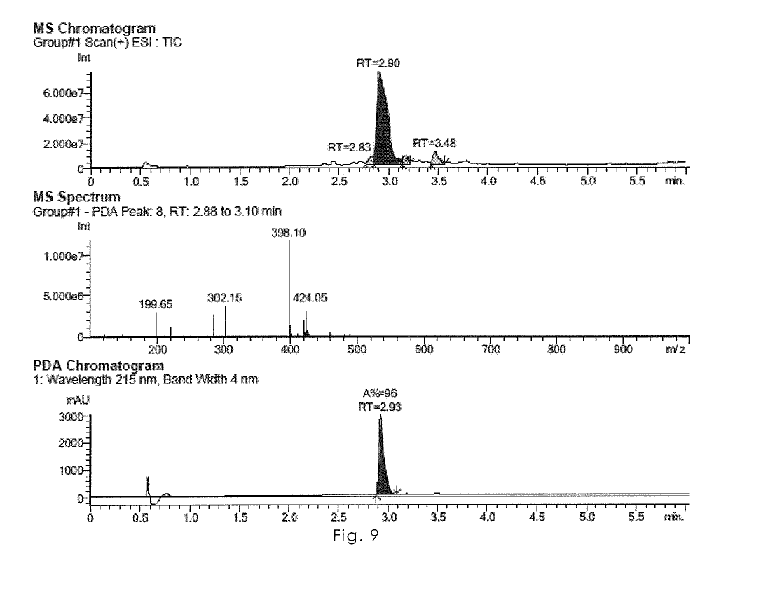

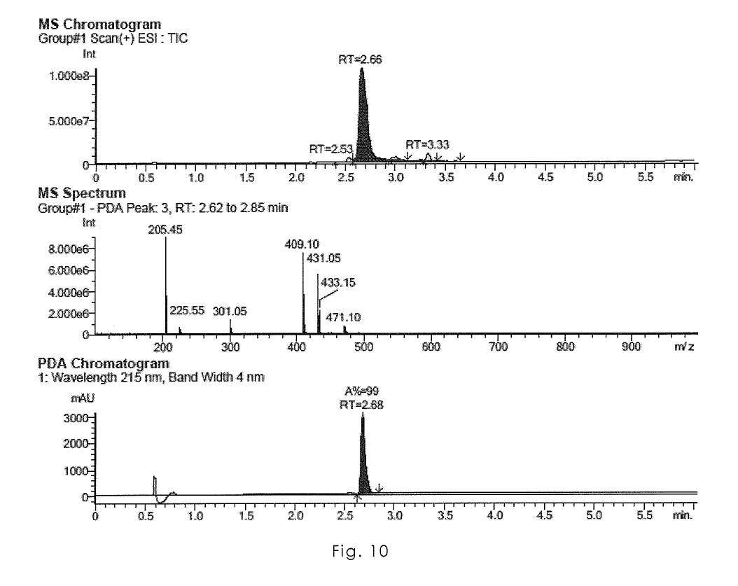

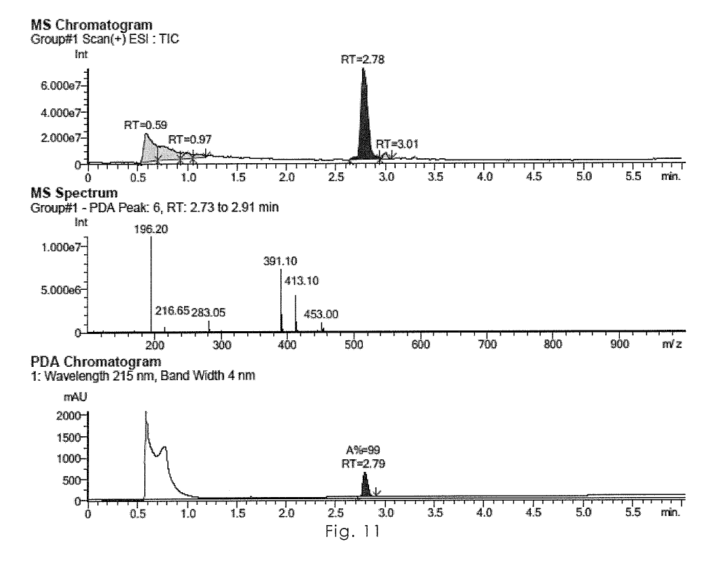

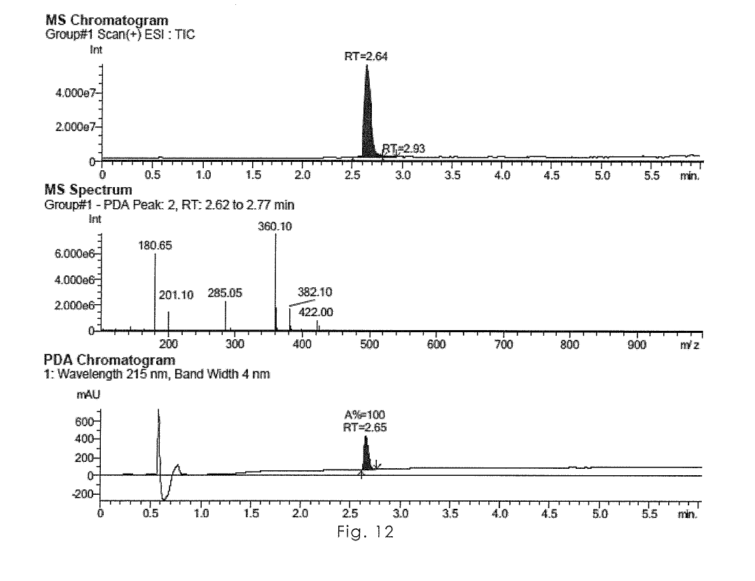

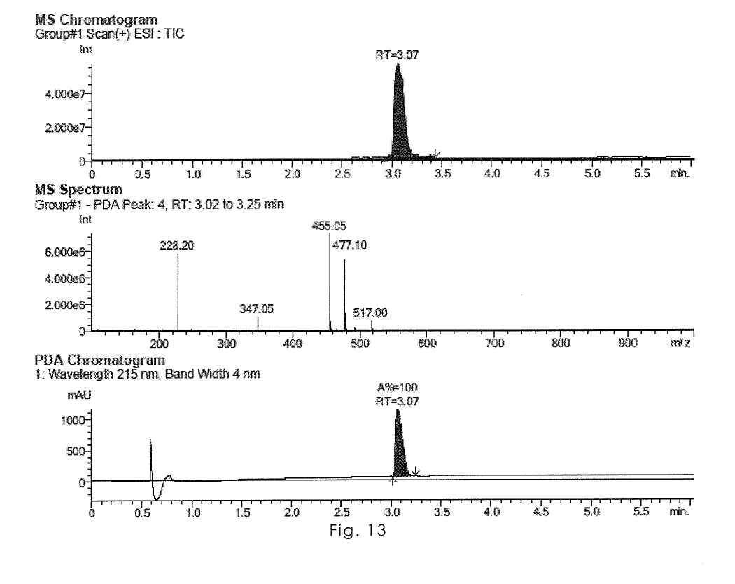

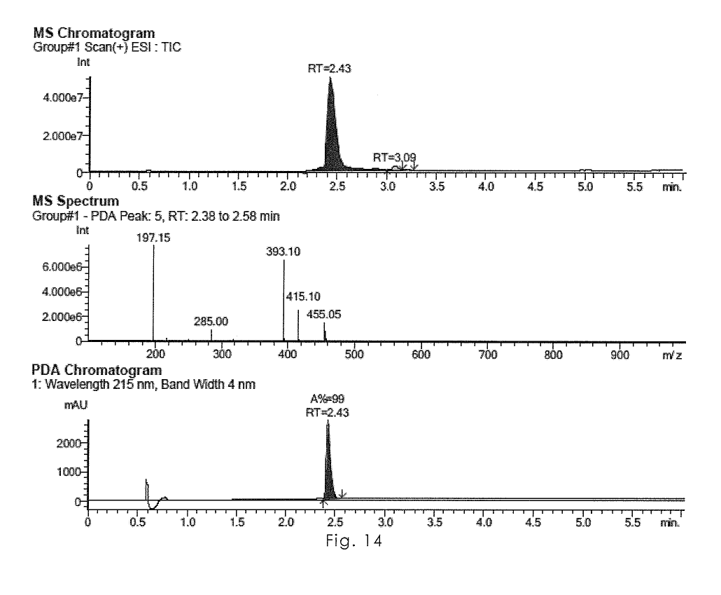

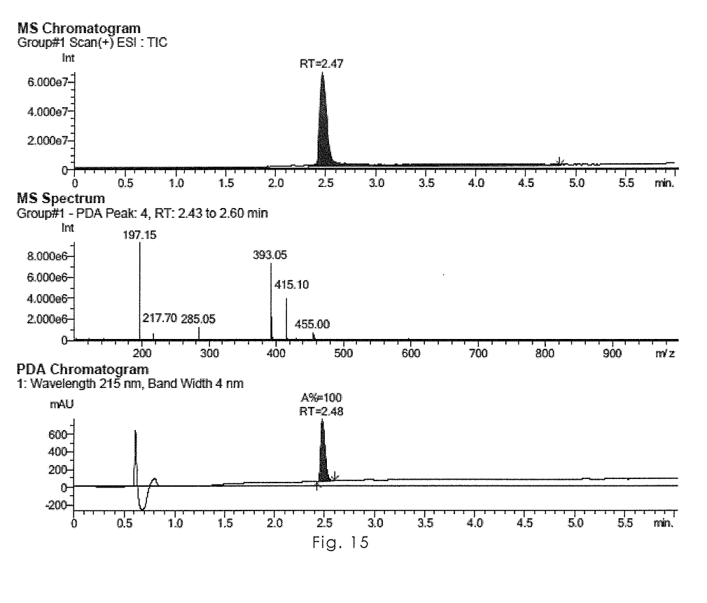

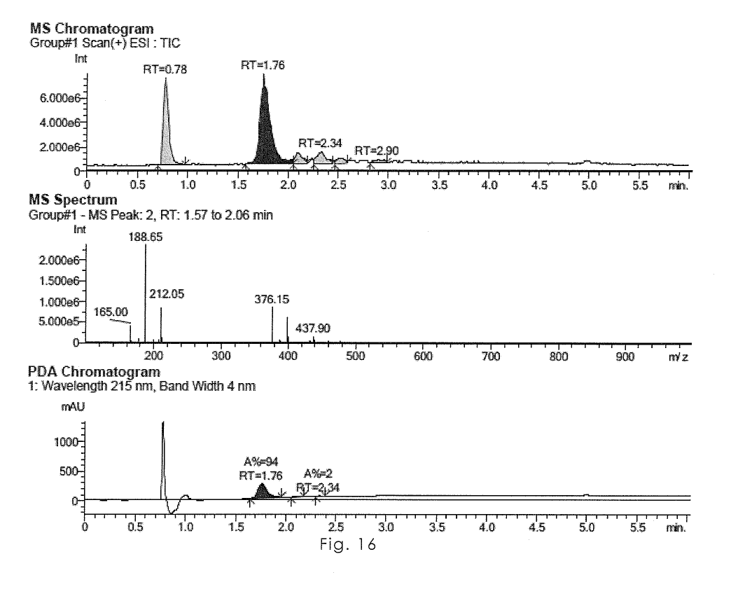

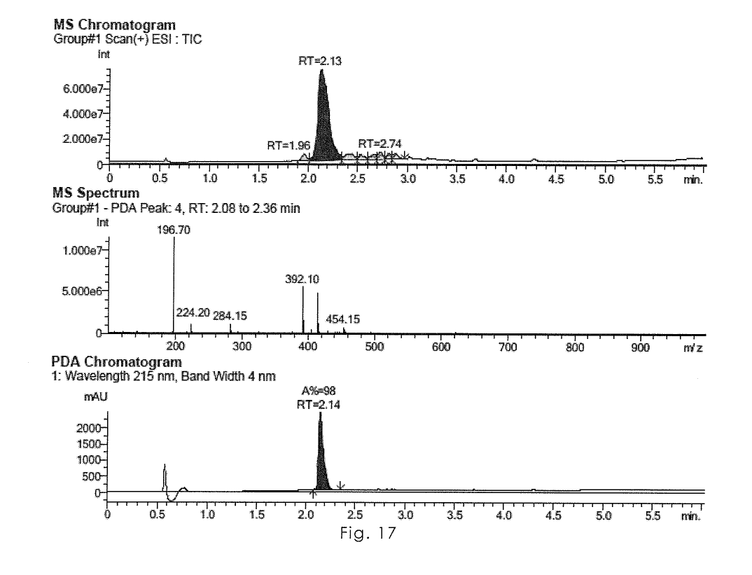

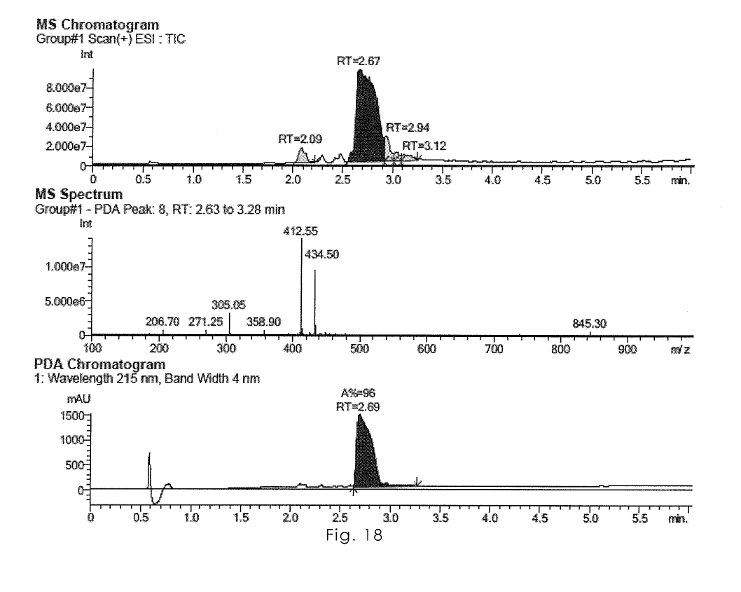

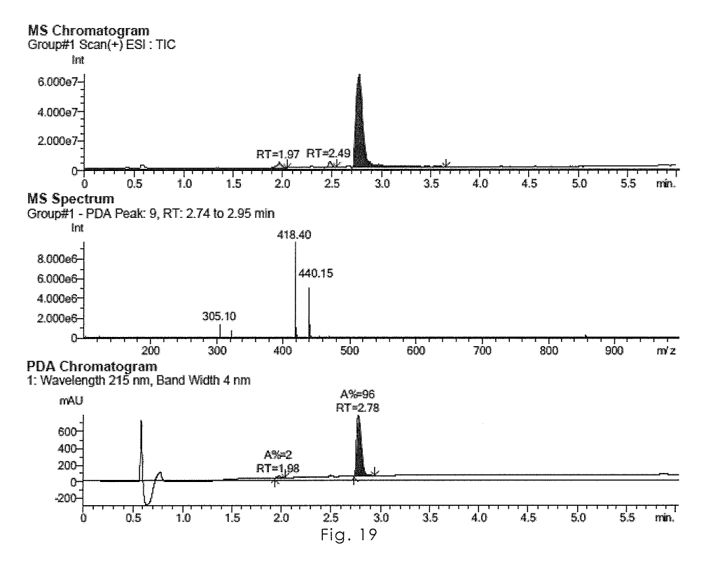

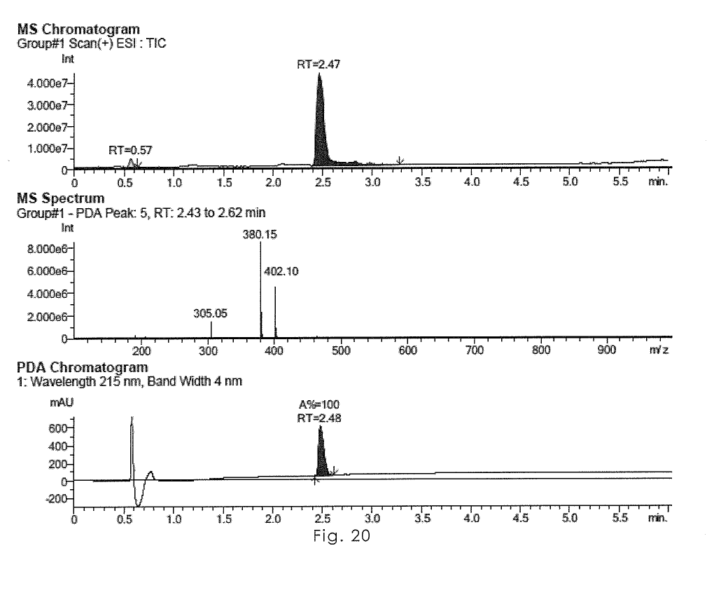

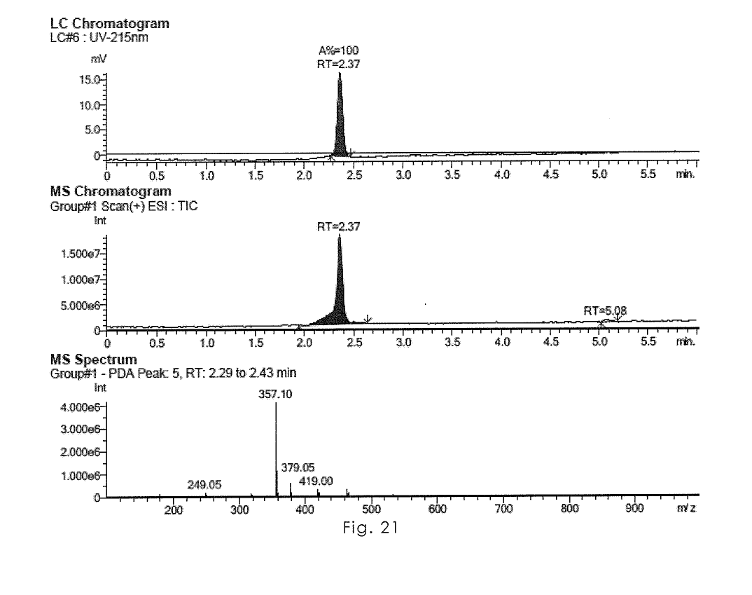

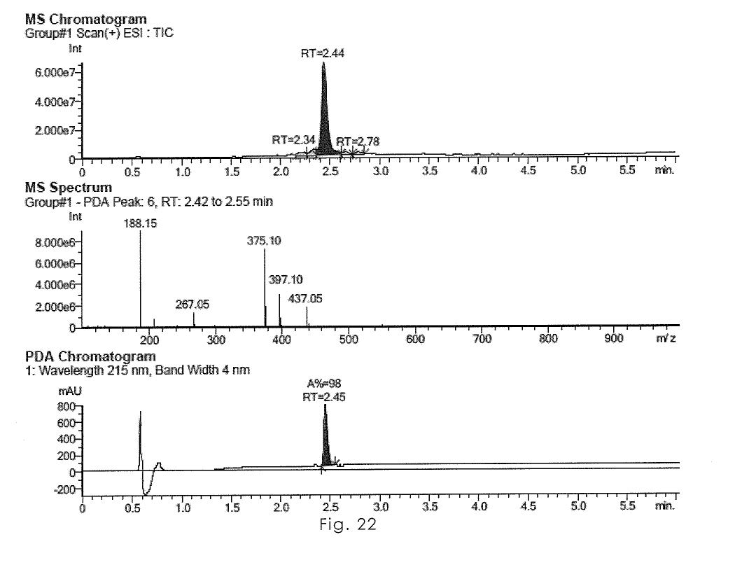

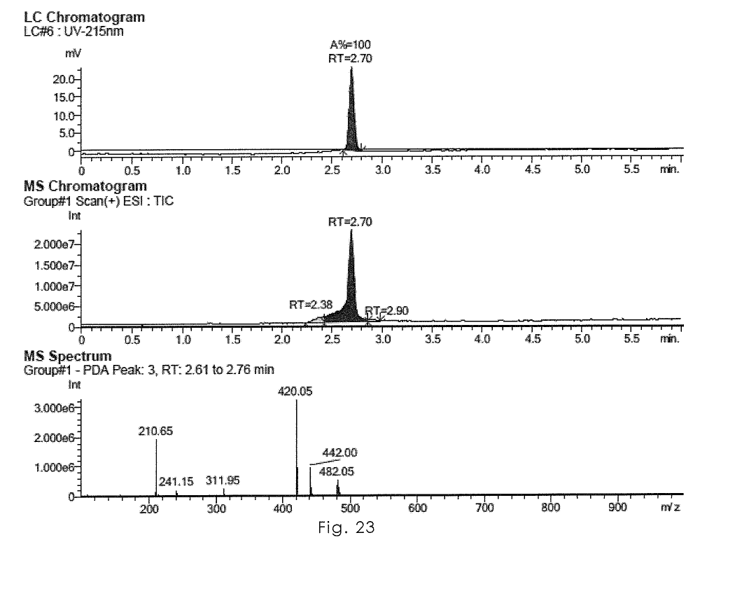

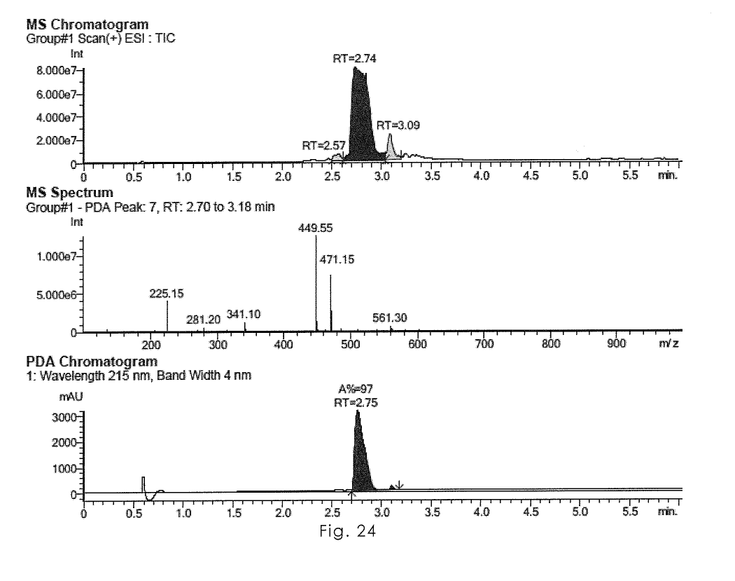

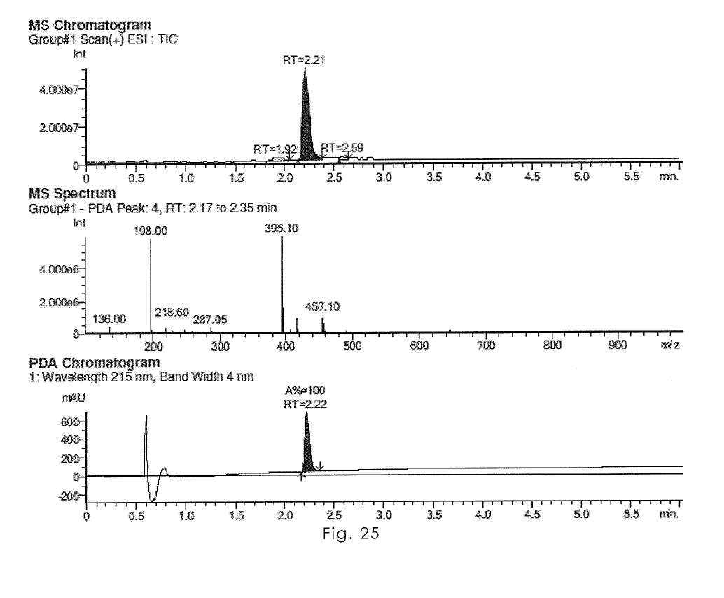

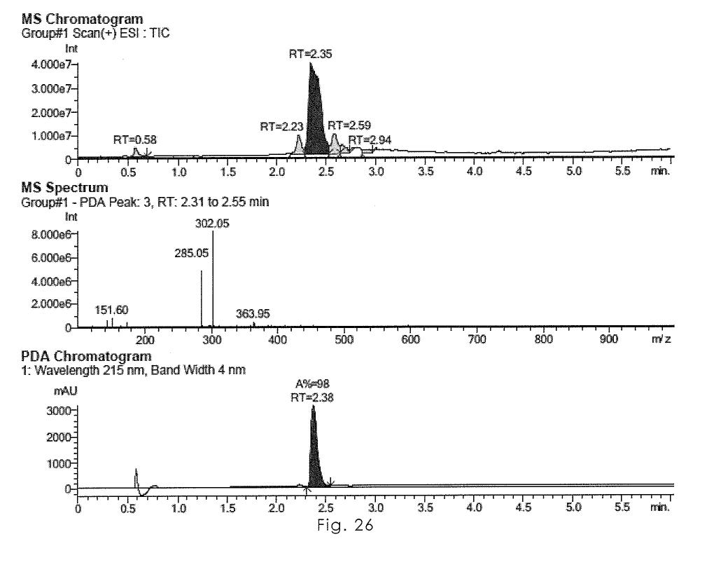

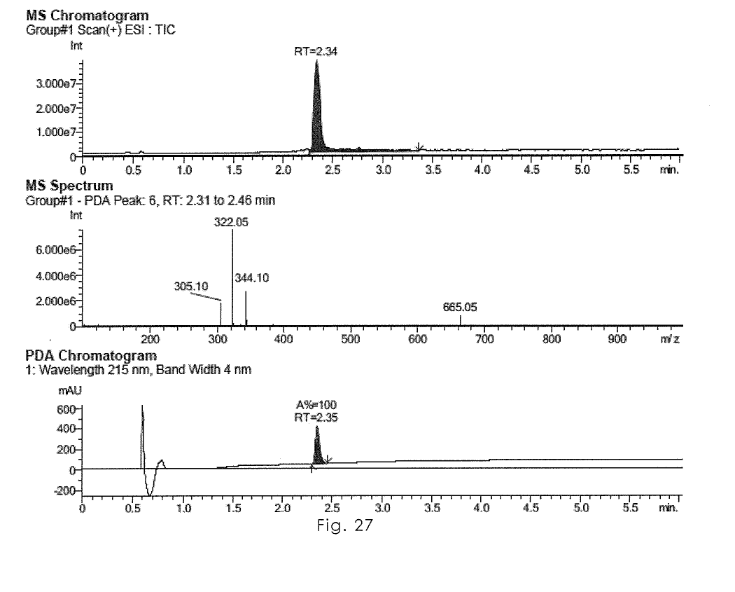

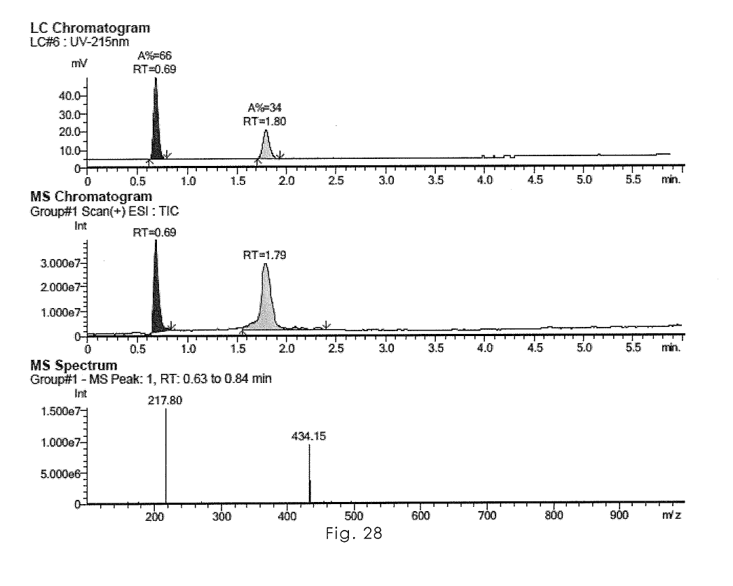

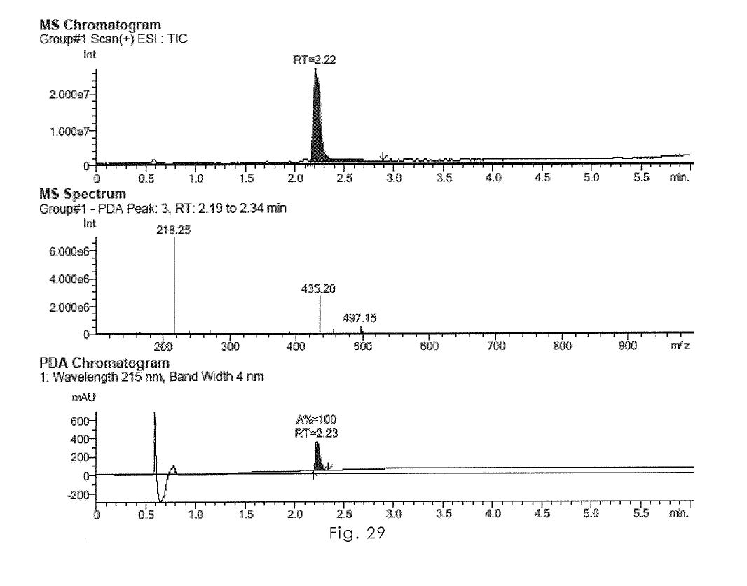

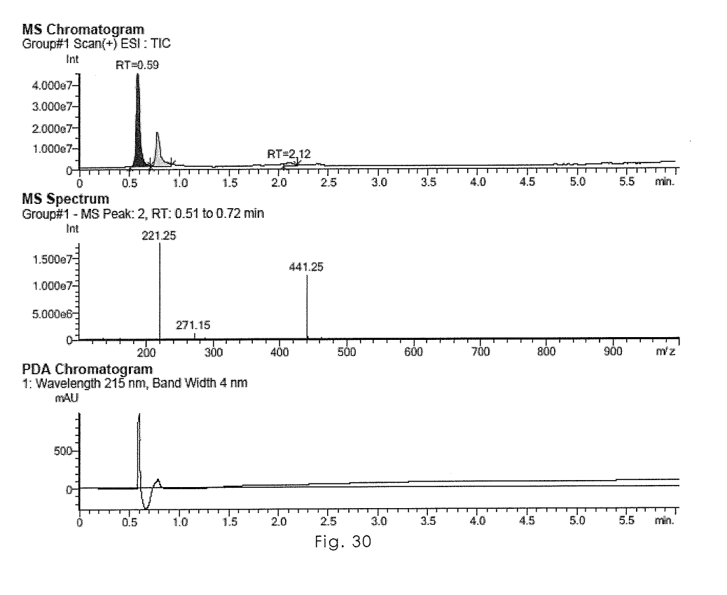

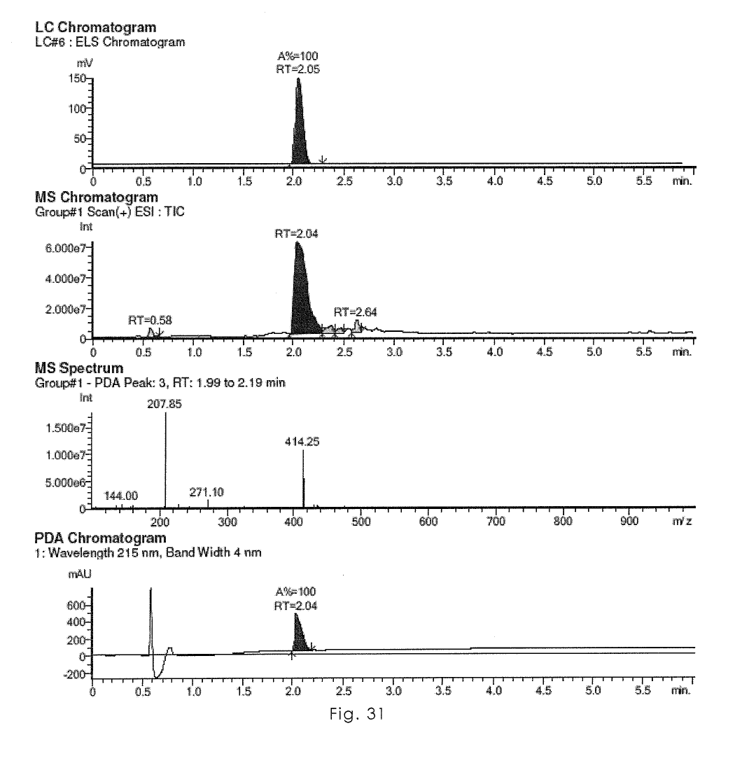

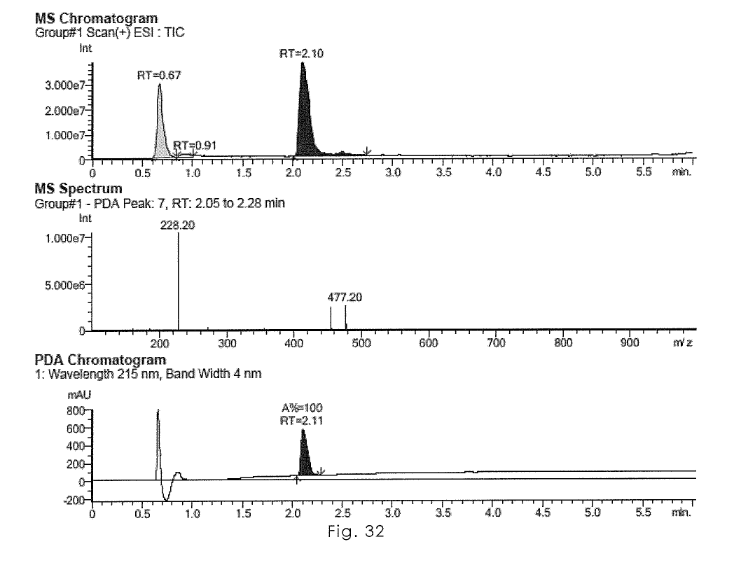

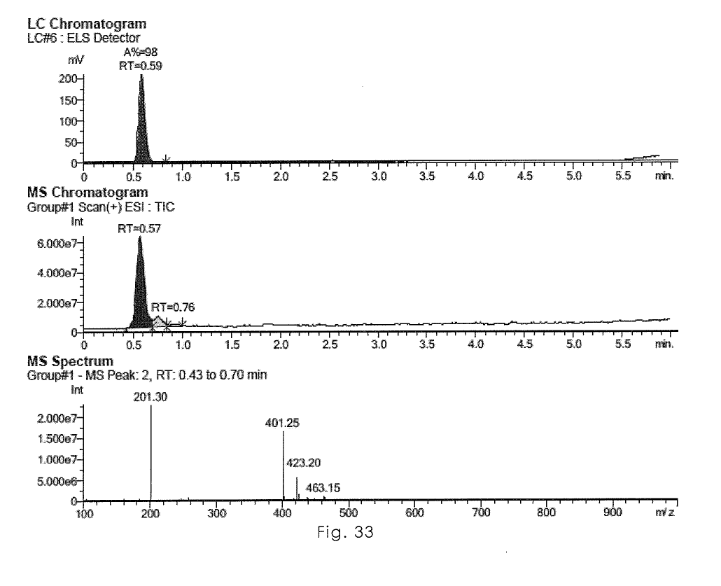

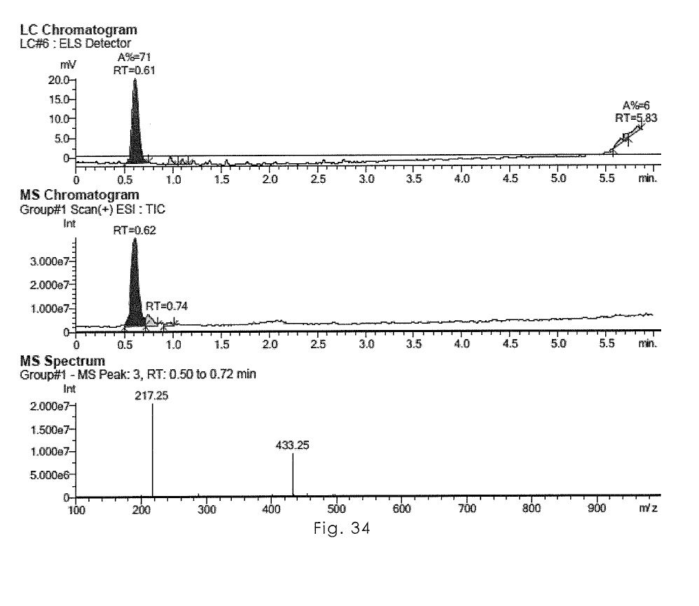

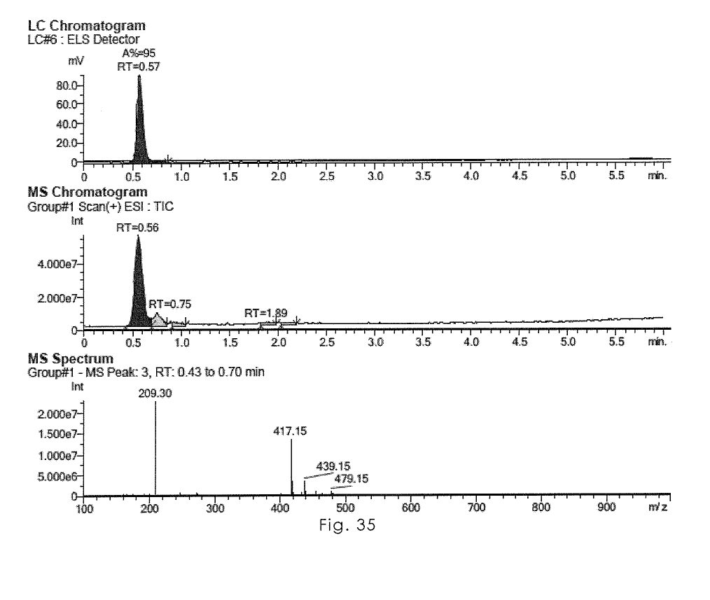

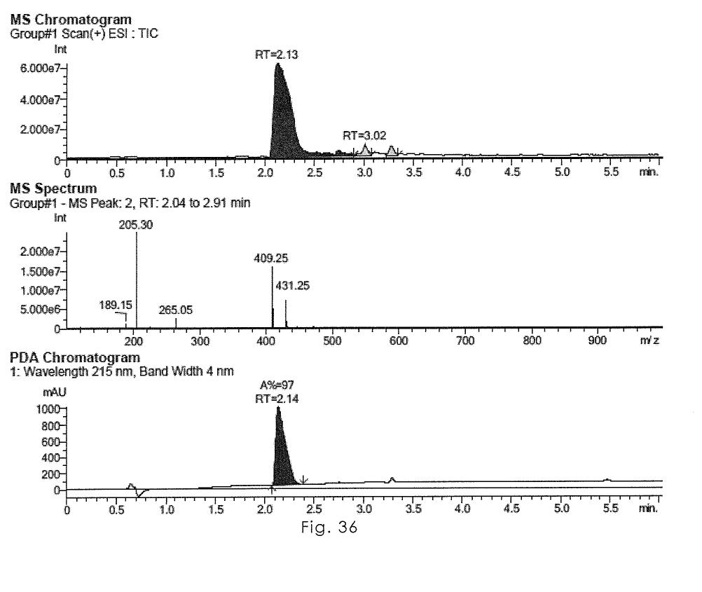

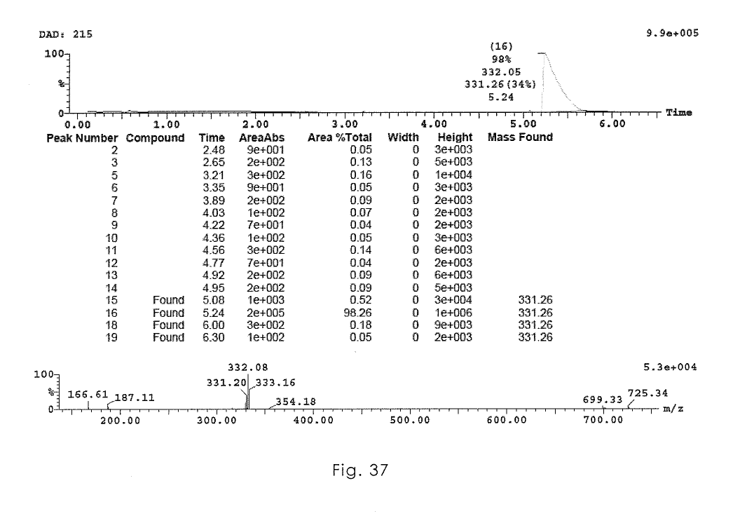

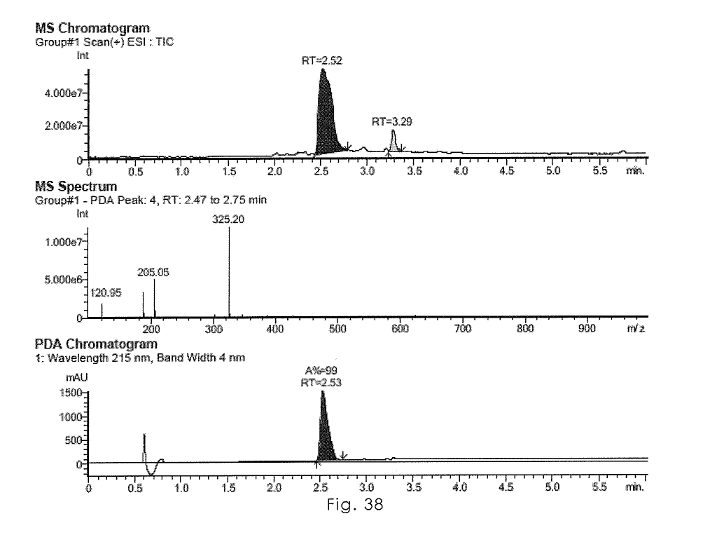

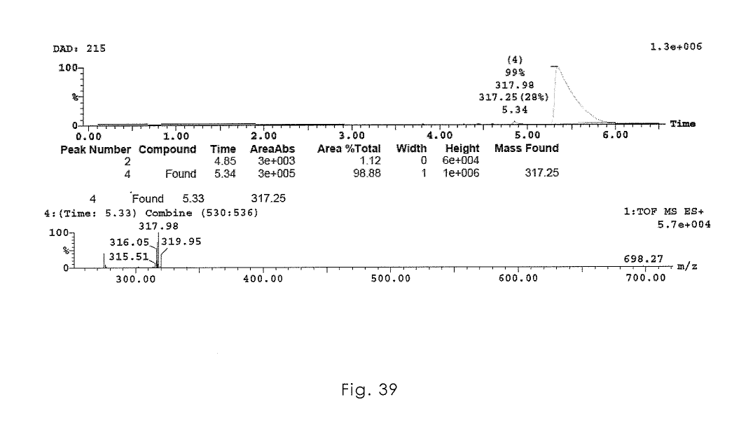

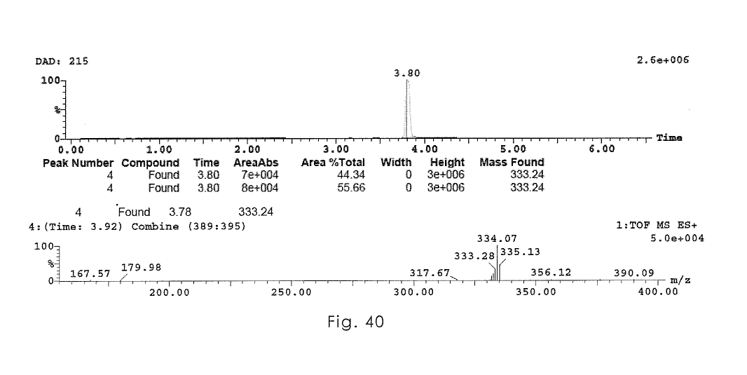

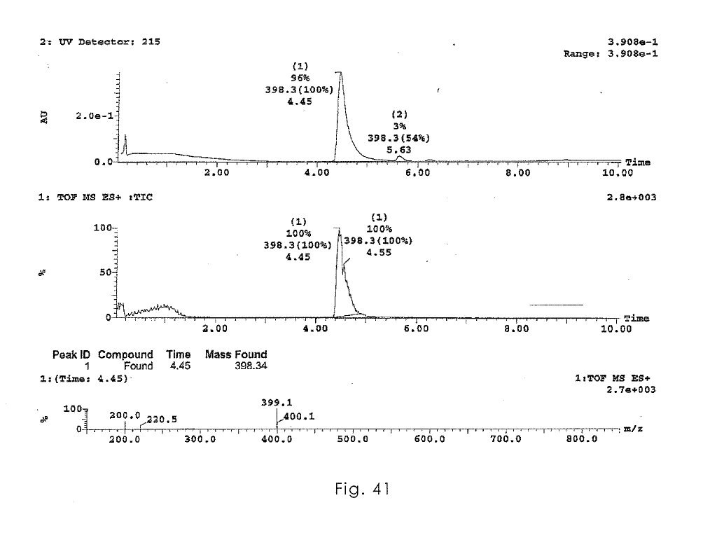

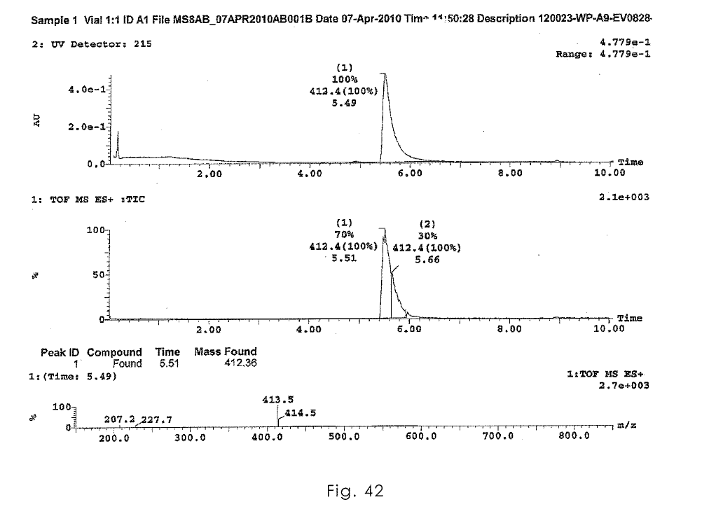

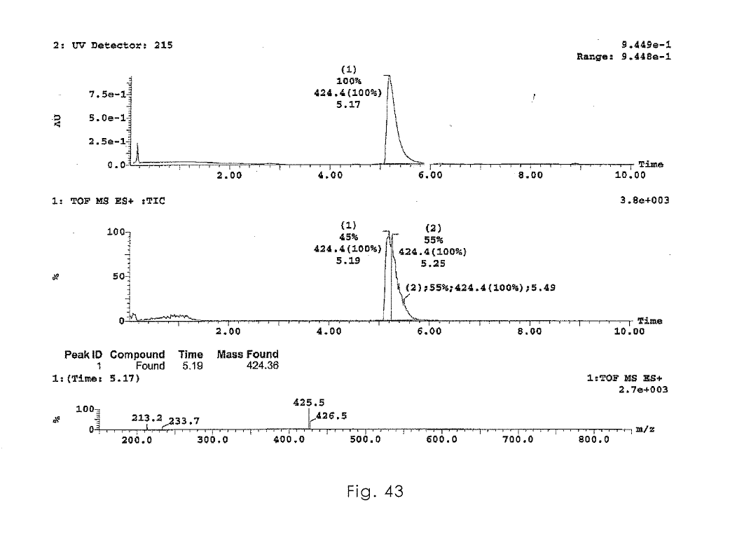

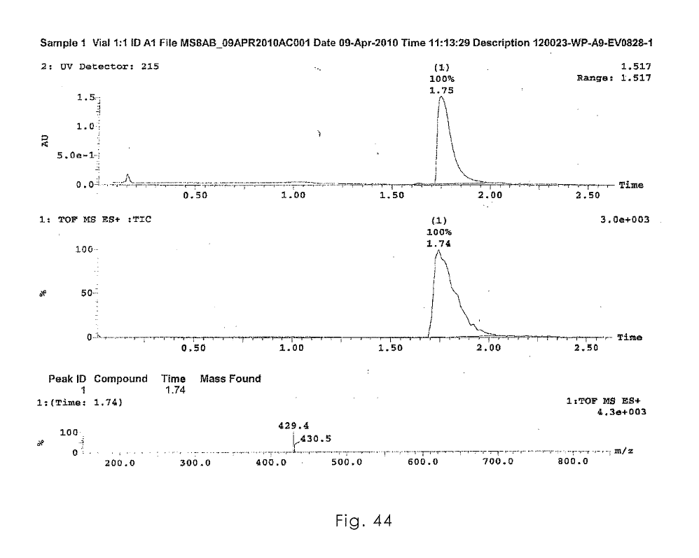

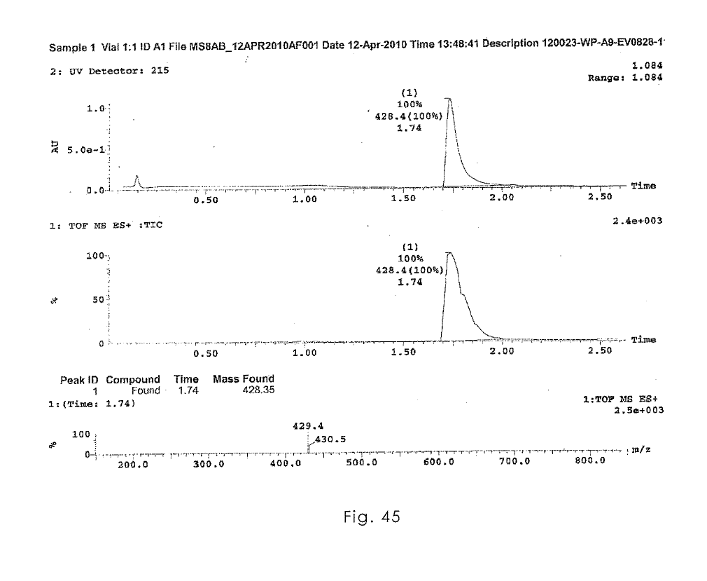

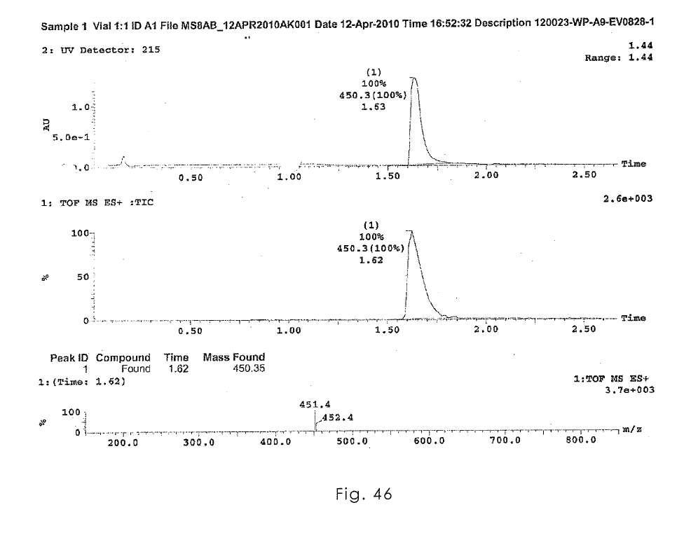

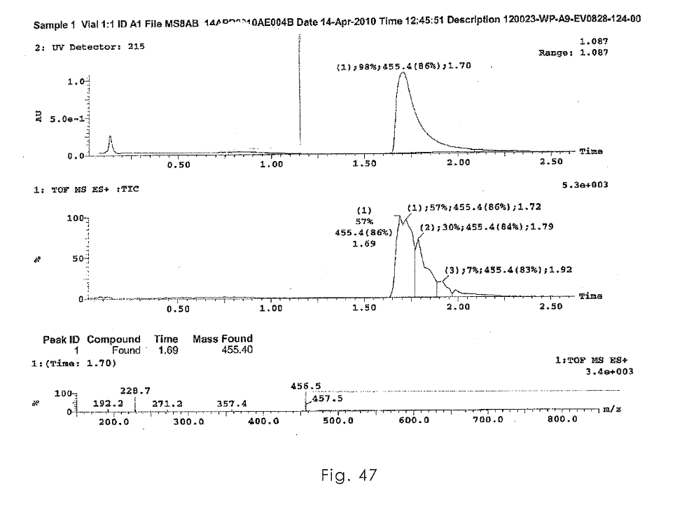

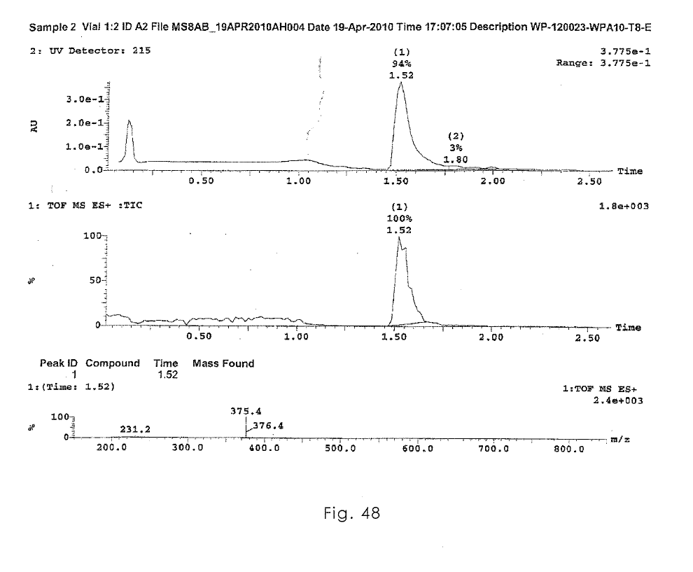

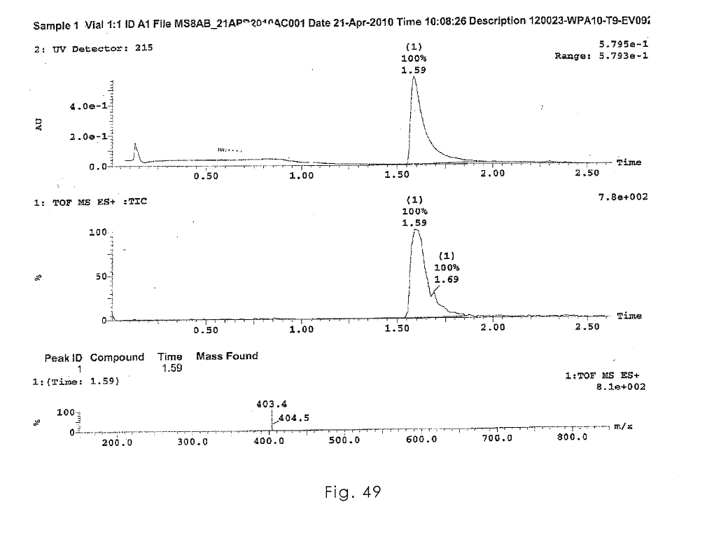

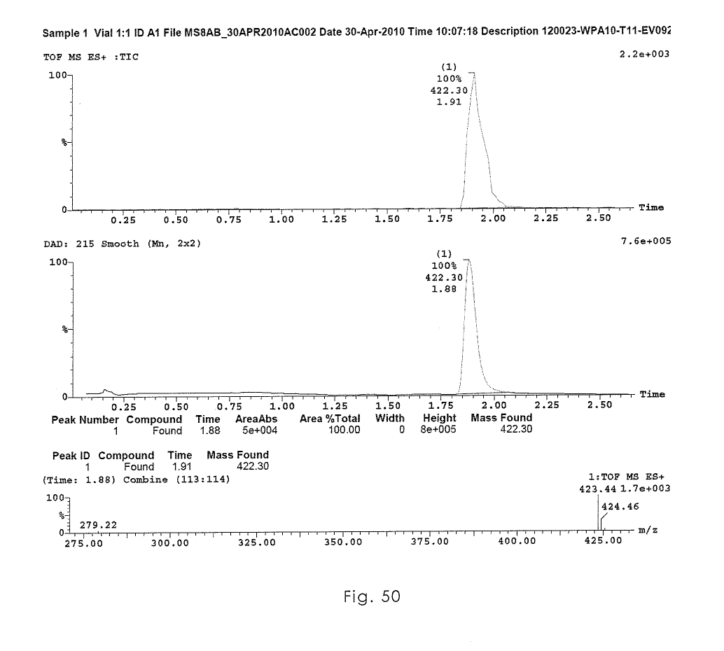

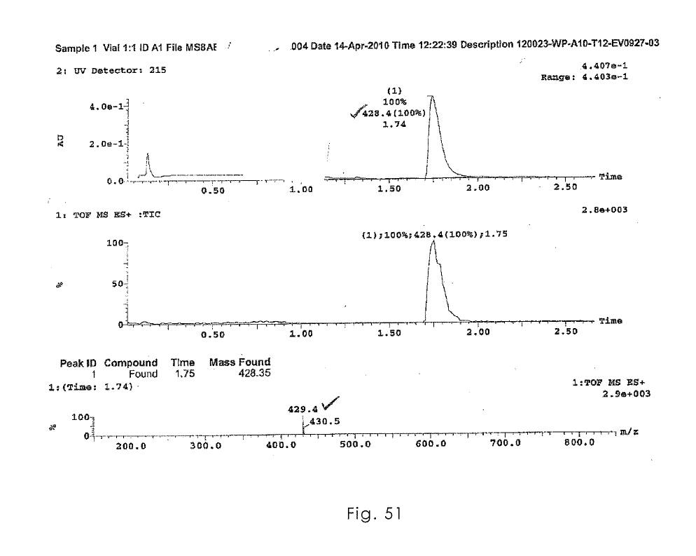

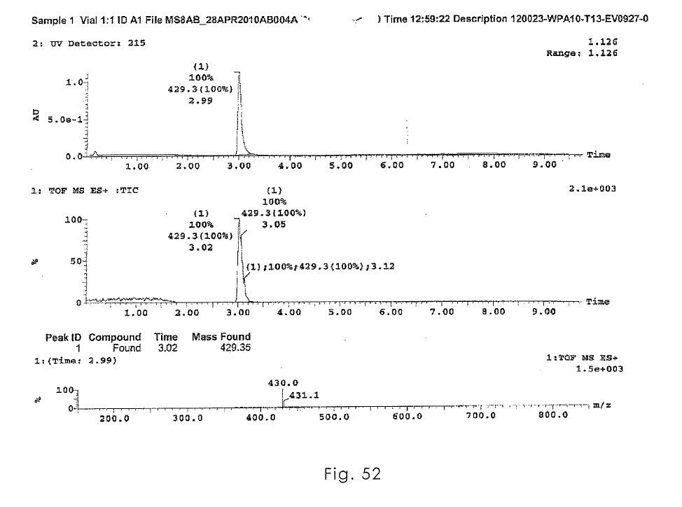

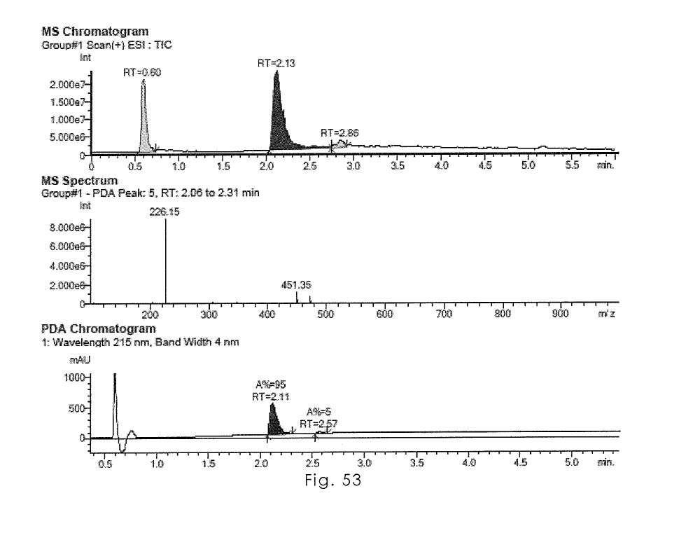

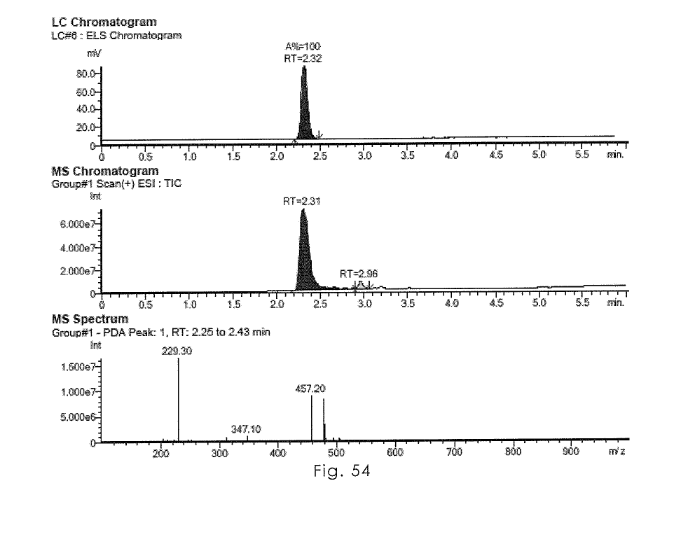

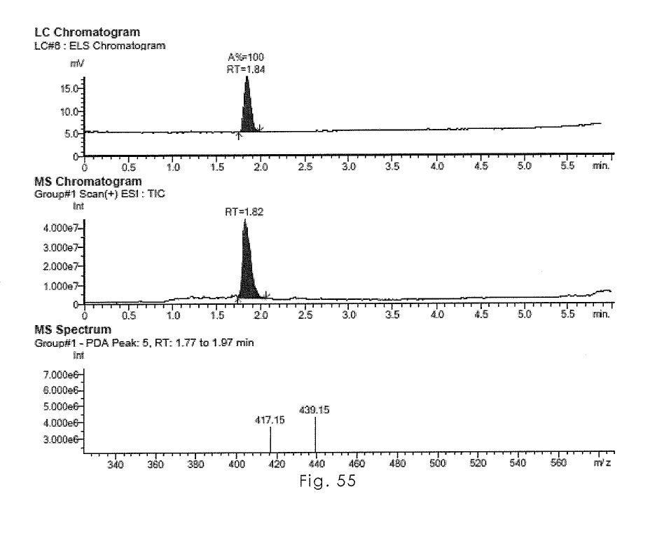

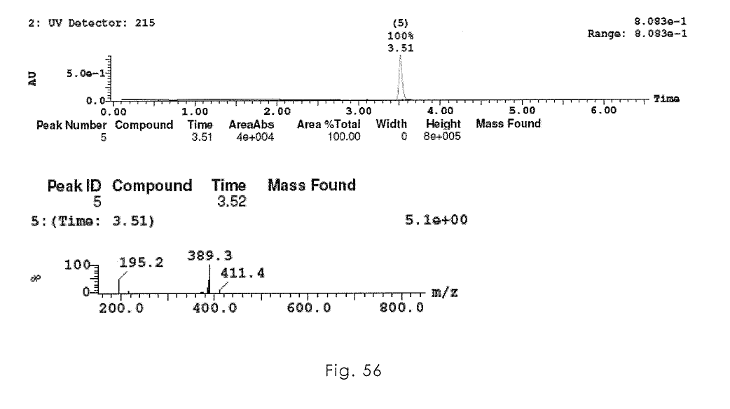

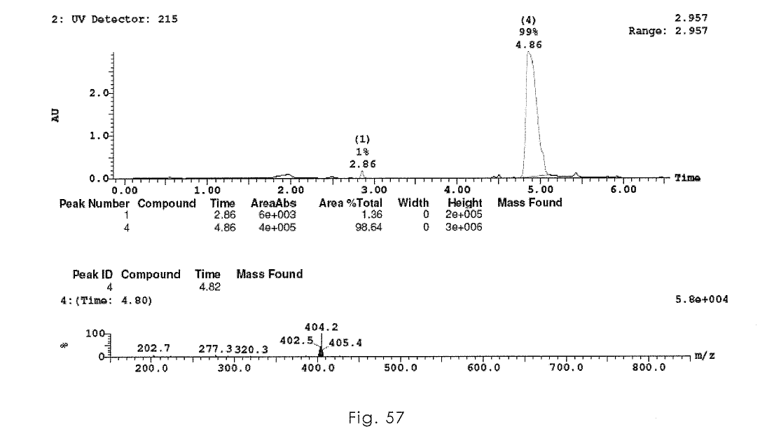

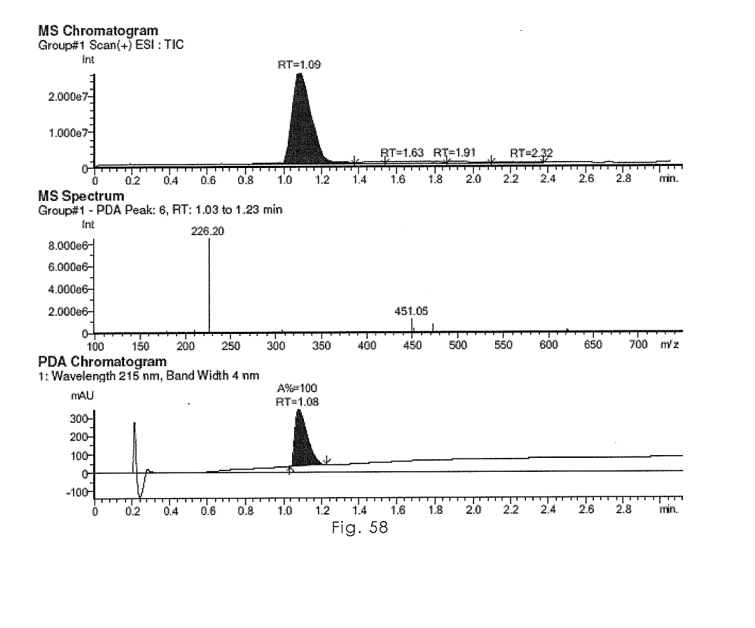

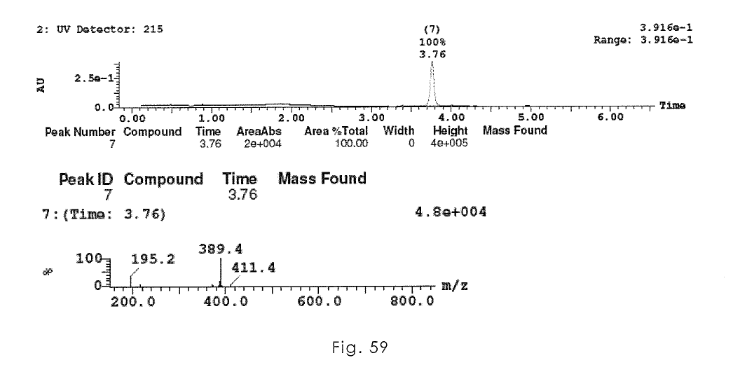

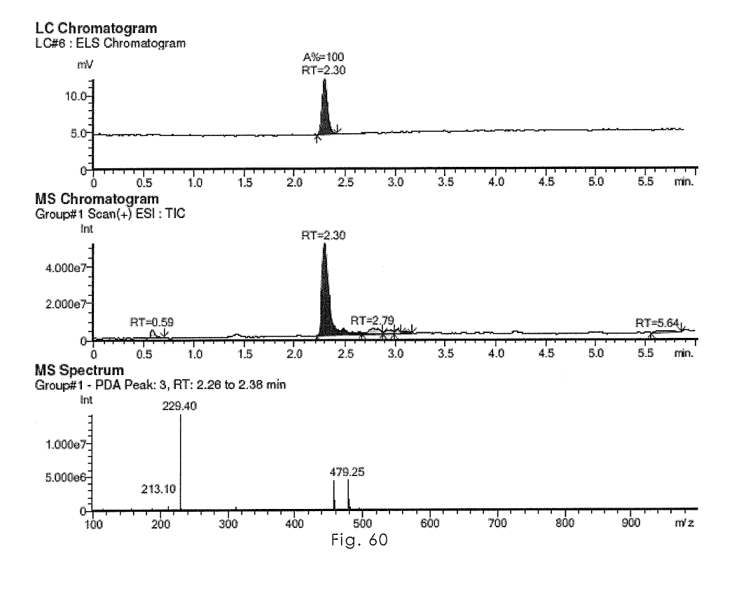

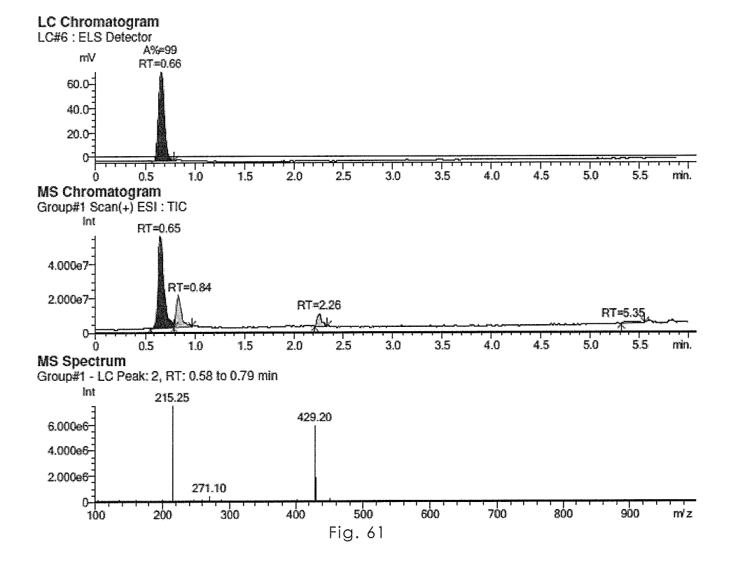

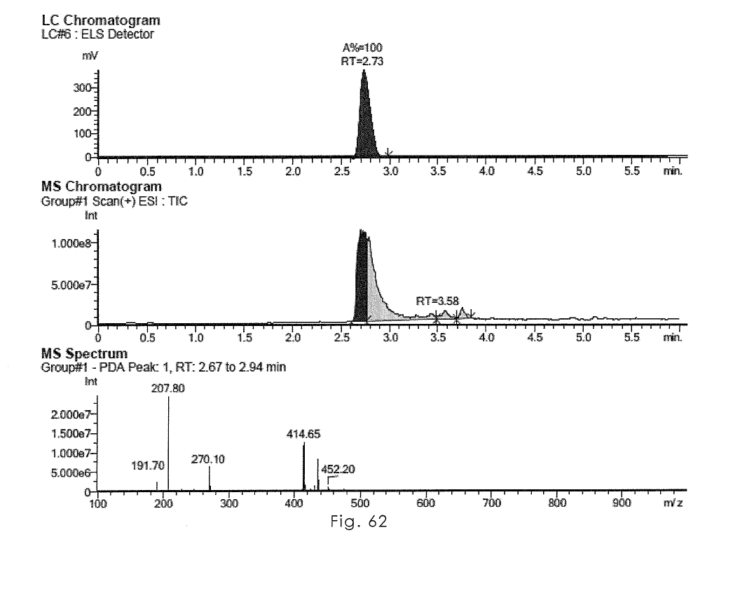

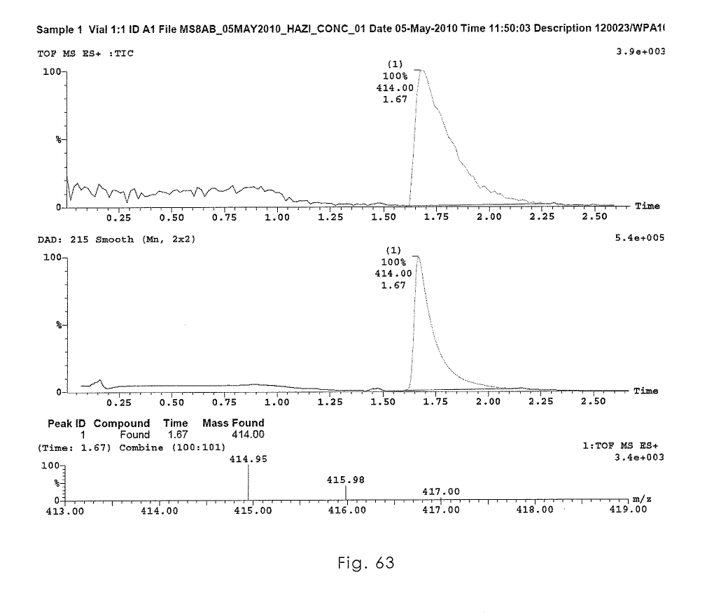

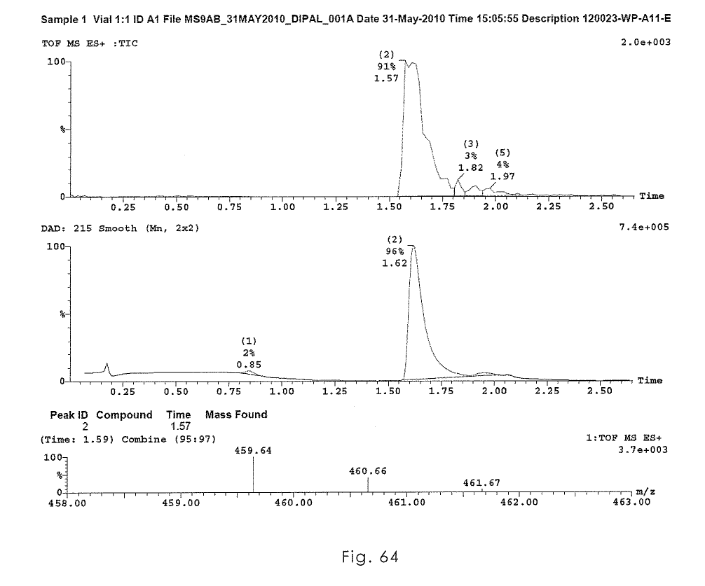

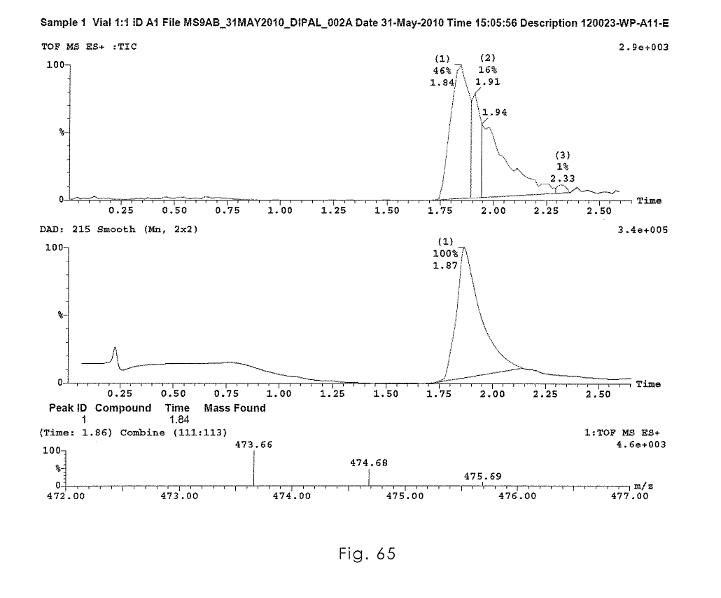

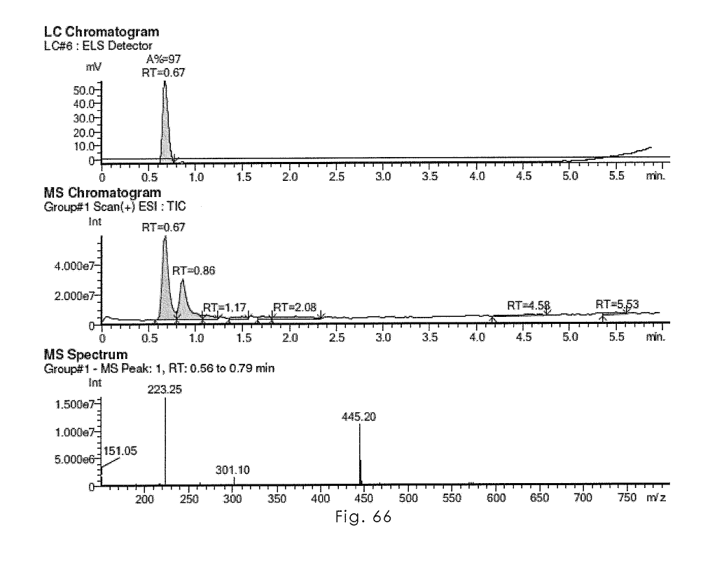

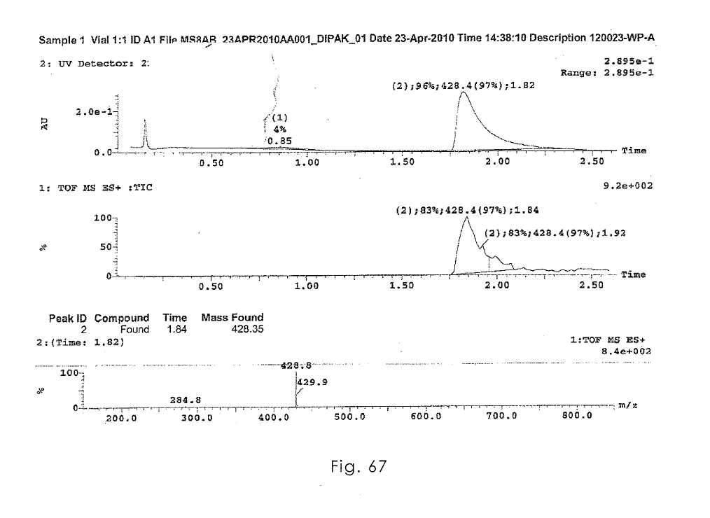

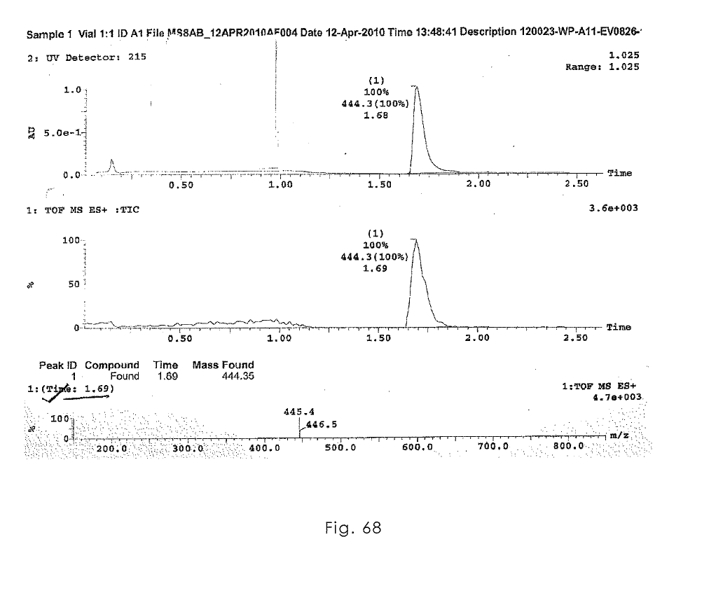

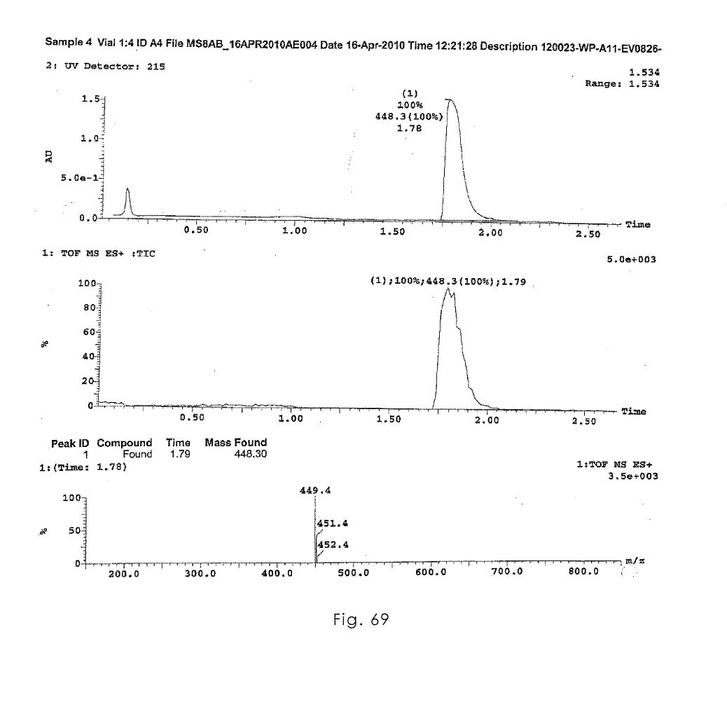

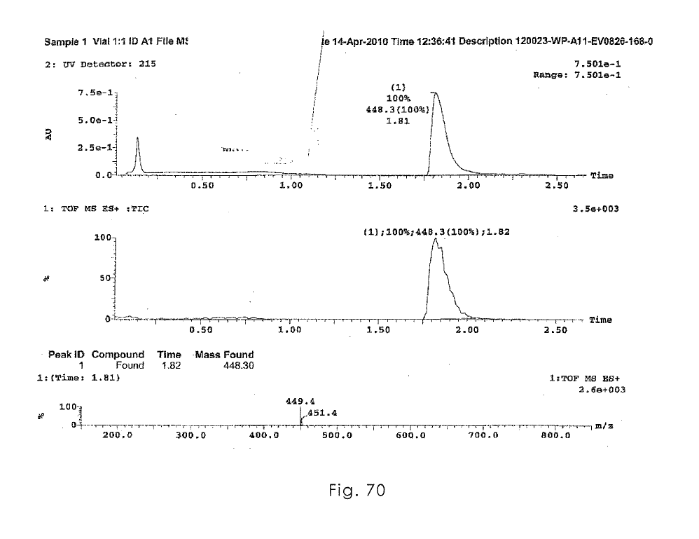

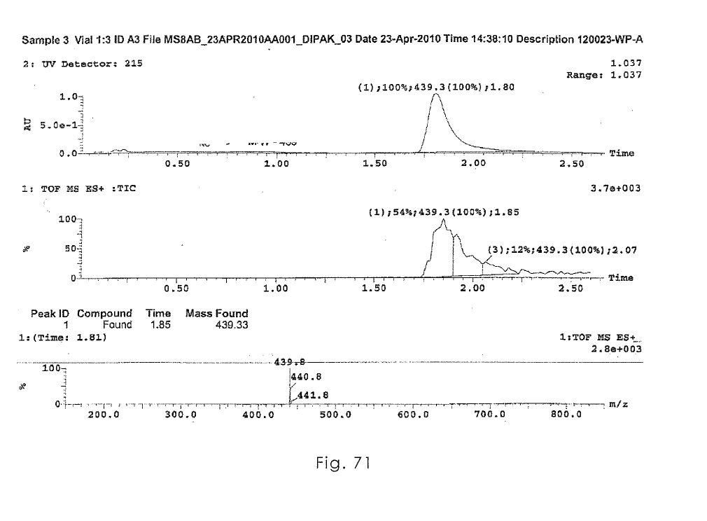

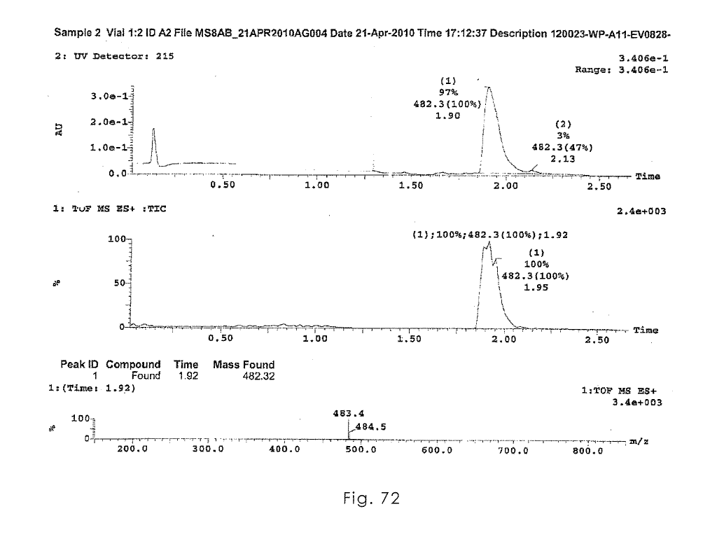

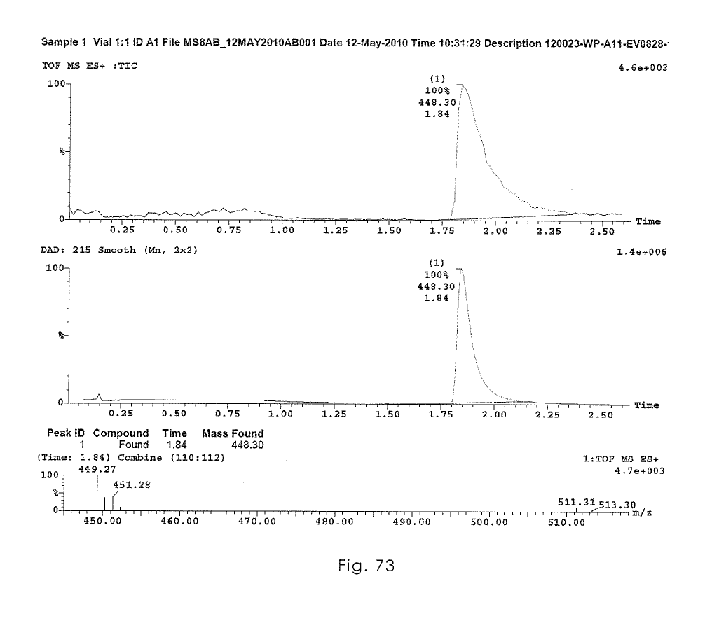

1-22. (canceled)

23. A method of treating iron metabolism disorders comprising administering to a patient in need a preparation including compounds of the formula (I) 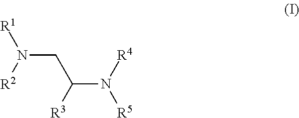
wherein
R¹ and R² are identical or different and are each chosen from the group consisting of:
hydrogen,
optionally substituted acyl,
optionally substituted alkyl,
optionally substituted aryl, and
optionally substituted heterocyclyl; or
R¹ and R² together with the nitrogen atom to which they are bonded form a saturated or unsaturated, optionally substituted 5- to 8-membered ring which can optionally contain further hetero atoms;
R³ is chosen from the group consisting of:
optionally substituted aryl, and
optionally substituted heterocyclyl;
R⁴ and R⁵ are identical or different and are each chosen from the group consisting of:
hydrogen,
optionally substituted alkyl-, aryl- or heterocyclylsulfonyl,
optionally substituted acyl,
optionally substituted alkyl,
optionally substituted alkenyl,
optionally substituted alkynyl,
optionally substituted aryl, and
optionally substituted heterocyclyl; or
R⁴ and R⁵ together with the nitrogen atom to which they are bonded form a saturated or unsaturated, optionally substituted 5- to 8-membered ring which can optionally contain further hetero atoms;
or pharmaceutically acceptable salts thereof
for use in the treatment of iron metabolism disorders.

24. The method of claim 23, wherein in the compound of formula (I), R¹ and R² are identical or different and are each chosen from the group consisting of:
hydrogen,
optionally substituted alkyl,
optionally substituted aryl, and
optionally substituted heterocyclyl; or
R¹ and R² together with the nitrogen atom to which they are bonded form a saturated or unsaturated, optionally substituted 5- to 6-membered ring which can optionally contain further hetero atoms;
R³ is chosen from the group consisting of:
optionally substituted aryl, and
optionally substituted heterocyclyl;
R⁴ and R⁵ are identical or different and are each chosen from the group consisting of hydrogen,
optionally substituted alkyl,
optionally substituted aryl, and
optionally substituted heterocyclyl; or
R⁴ and R⁵ together with the nitrogen atom to which they are bonded form a saturated or unsaturated, optionally substituted 5- to 6-membered ring which can optionally contain further hetero atoms;
or pharmaceutically acceptable salts thereof.

25. The method of claim 23, wherein the compound of formula (I) has the general formula (Ia) 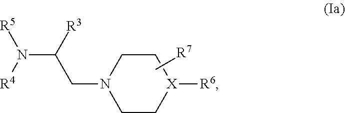
and wherein
X is chosen from: O, N or CH;
R⁶ is chosen from the group consisting of:
hydrogen,
optionally substituted alkyl,
optionally substituted alkenyl,
optionally substituted alkynyl,
optionally substituted acyl,
optionally substituted alkoxycarbonyl,
optionally substituted amino,
optionally substituted aminocarbonyl,
optionally substituted alkyl-, aryl- or heterocyclylsulfonyl,
optionally substituted aryl, and
optionally substituted heterocyclyl;
R⁷ is chosen from the group consisting of:
hydrogen,
hydroxyl,
halogen,
cyano,
nitro,
carboxyl,
sulfonic acid radical (—SO₃H),
optionally substituted amino,
optionally substituted aminocarbonyl,
optionally substituted aminosulfonyl,
optionally substituted acyl,
optionally substituted acyloxy,
optionally substituted alkoxy,
optionally substituted alkoxycarbonyl,
optionally substituted alkyl,
optionally substituted alkenyl,
optionally substituted alkynyl,
optionally substituted aryl, and
optionally substituted heterocyclyl,
or pharmaceutically acceptable salts thereof.

26. The method of claim 25, wherein in the compound of formula (I),
X is chosen from: N or CH;
R⁶ is chosen from the group consisting of:
hydrogen,
optionally substituted alkyl,
optionally substituted alkenyl,
optionally substituted alkynyl,
optionally substituted acyl,
optionally substituted alkoxycarbonyl,
optionally substituted aryl, and
optionally substituted heterocyclyl;
R⁷ is chosen from the group consisting of:
hydrogen,
hydroxyl,
halogen,
cyano,
nitro,
carboxyl,
sulfonic acid radical (—SO₃H),
optionally substituted amino,
optionally substituted aminocarbonyl,
optionally substituted aminosulfonyl,
optionally substituted acyl,
optionally substituted acyloxy,
optionally substituted alkoxy,
optionally substituted alkoxycarbonyl,
optionally substituted alkyl,
optionally substituted alkenyl,
optionally substituted alkynyl, optionally substituted aryl, and
optionally substituted heterocyclyl;
or pharmaceutically acceptable salts thereof.

27. The method of claim 25, wherein in the compound of formula (I),
X is chosen from: N or CH;
R⁶ is chosen from the group consisting of:
hydrogen,
optionally substituted alkyl,
optionally substituted acyl,
optionally substituted alkoxycarbonyl,
optionally substituted amino,
optionally substituted aminocarbonyl,
optionally substituted alkyl-, aryl- or heterocyclylsulfonyl,
optionally substituted aryl, and
optionally substituted heterocyclyl;
R⁷ is chosen from the group consisting of:
hydrogen,
halogen,
optionally substituted amino,
optionally substituted acyl,
optionally substituted alkoxy,
optionally substituted alkyl,
optionally substituted aryl, and
optionally substituted heterocyclyl;
R³ is chosen from the group consisting of:
optionally substituted aryl, and
optionally substituted heterocyclyl;
R⁴ and R⁵ are identical or different and are each chosen from the group consisting of:
hydrogen,
optionally substituted alkyl,
optionally substituted aryl, and
optionally substituted heterocyclyl; or
R⁴ and R⁵ together with the nitrogen atom to which they are bonded form a saturated or unsaturated, optionally substituted 5- to 7-membered ring which can optionally contain further hetero atoms;
or pharmaceutically acceptable salts thereof.

28. The method of claim 25, wherein in the compound of formula (I),
X has the meaning N;
R⁶ is chosen from the group consisting of:
optionally substituted acyl,
optionally substituted alkyl-, aryl- or heterocyclylsulfonyl,
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of
hydrogen or
optionally substituted alkyl, or
R⁴ and R⁵ together with the nitrogen atom to which they are bonded form a saturated or unsaturated, optionally substituted 6-membered ring which can optionally contain further hetero atoms.
or pharmaceutically acceptable salts thereof.

29. The method of claim 25, wherein in the compound of formula (I),
X has the meaning N;
R⁶ is chosen from the group consisting of:
optionally substituted acyl,
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of:
hydrogen or
optionally substituted alkyl,
or pharmaceutically acceptable salts thereof.

30. The method of claim 23, wherein in the compound of formula (I),
X has the meaning CH; and
R⁶ is chosen from the group consisting of:
hydrogen,
optionally substituted alkyl,
optionally substituted alkoxycarbonyl,
optionally substituted amino,
optionally substituted aminocarbonyl,
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of:
hydrogen,
optionally substituted alkyl; or
R⁴ and R⁵ together with the nitrogen atom to which they are bonded form a saturated or unsaturated, optionally substituted 6- or 7-membered ring which can optionally contain further hetero atoms;
or pharmaceutically acceptable salts thereof.

31. The method of claim 23, wherein in the compound of formula (I),
X has the meaning CH; and
R⁶ is chosen from the group consisting of:
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of:
hydrogen,
optionally substituted alkyl; or
R⁴ and R⁵ together with the nitrogen atom to which they are bonded form a saturated or unsaturated, optionally substituted 6-membered ring which can optionally contain further hetero atoms;
or pharmaceutically acceptable salts thereof.

32. The method of claim 23, wherein in the compound of formula (I), R¹ and R² together with the nitrogen atom to which they are bonded form an optionally substituted, saturated or unsaturated 6-membered ring which can optionally contain one to 3 further hetero atoms and/or wherein R³ is optionally substituted aryl or optionally substituted heterocyclyl.

33. The method of claim 23, wherein in the compound of formula (I), R⁴ and R⁵ are identical and denote hydrogen, or one of the radicals R⁴ or R⁵ is hydrogen, and the other radical of the radicals R⁴ or R⁵ is optionally substituted alkyl, or wherein R⁴ and R⁵ together with the nitrogen atom to which they are bonded faun an optionally substituted, saturated or unsaturated 6- or 7-membered ring which can optionally contain one to 3 further hetero atoms.

34. The method of claim 23, wherein the compound of formula (I) is selected from the group consisting of: 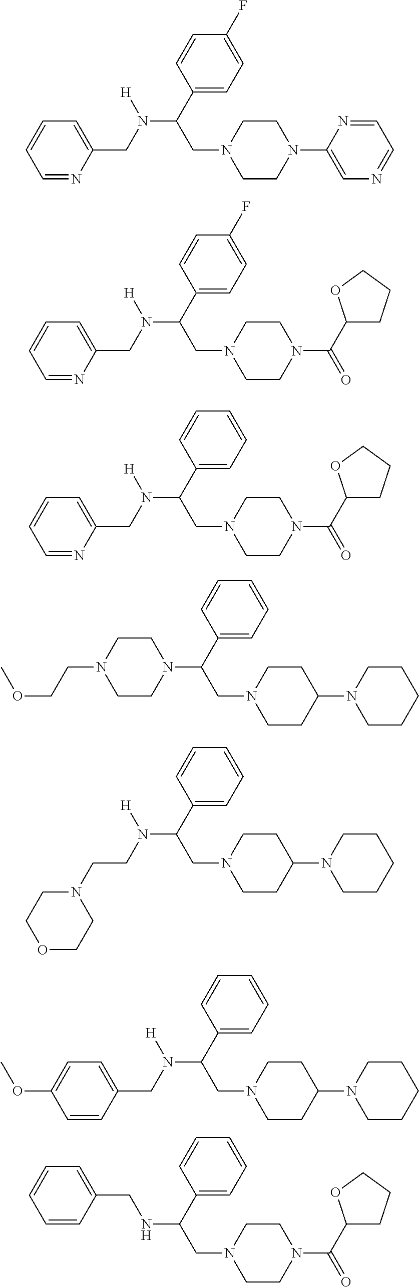 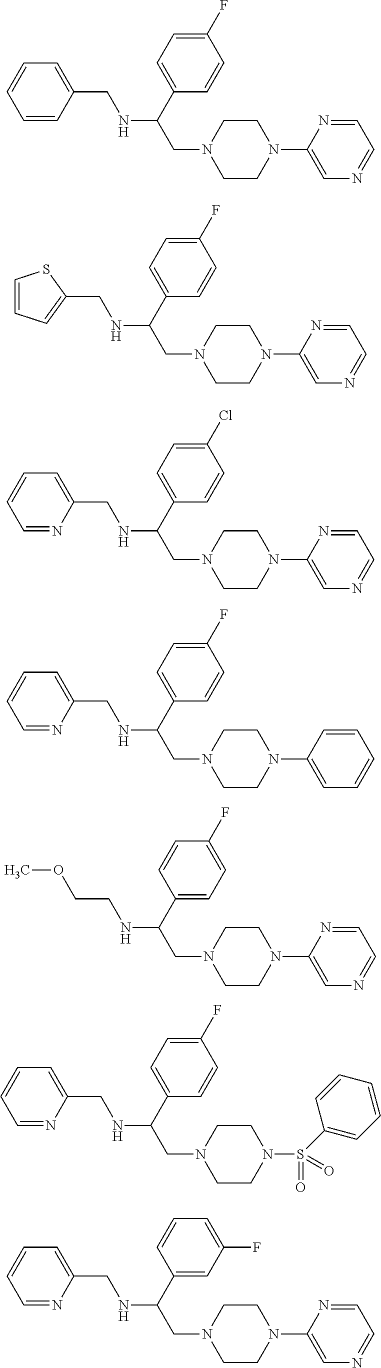 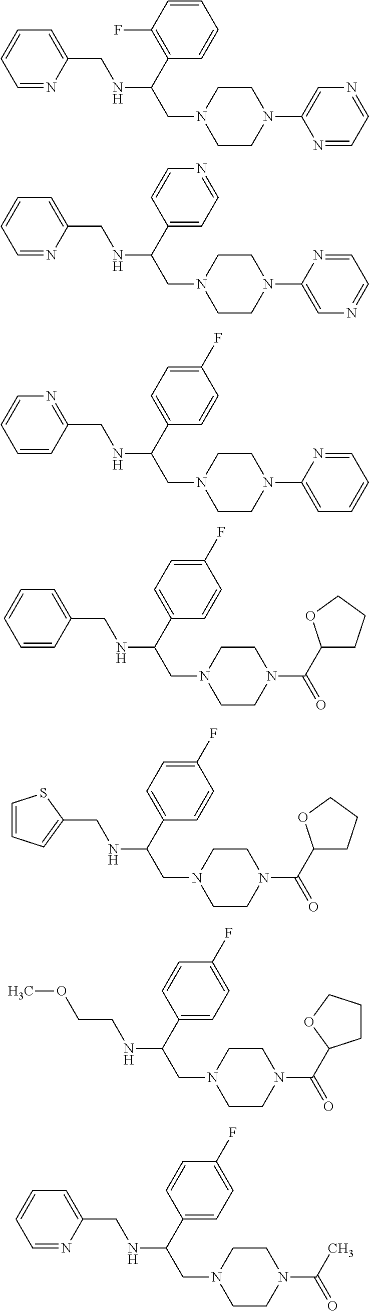 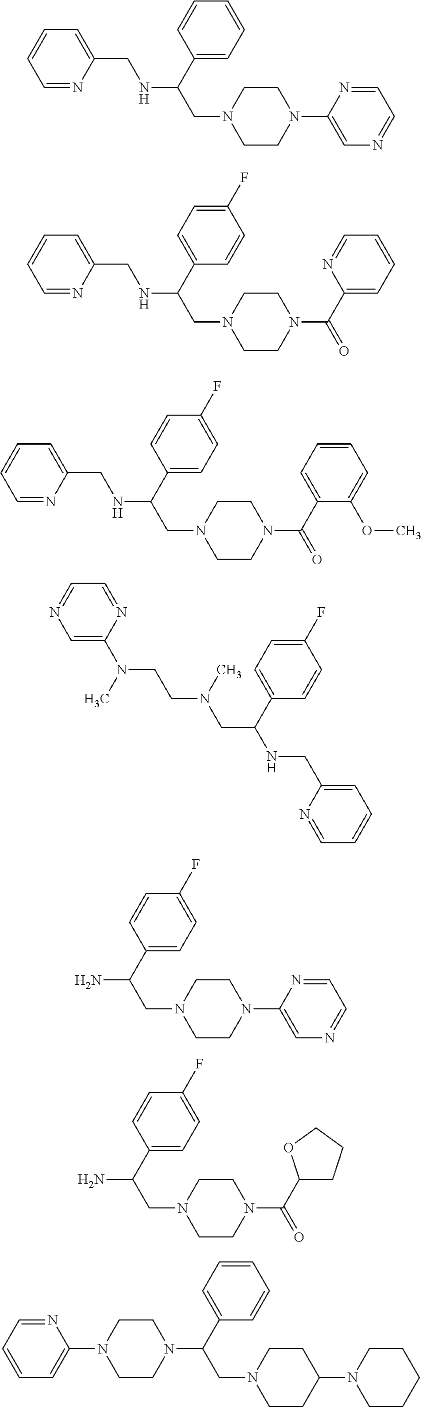 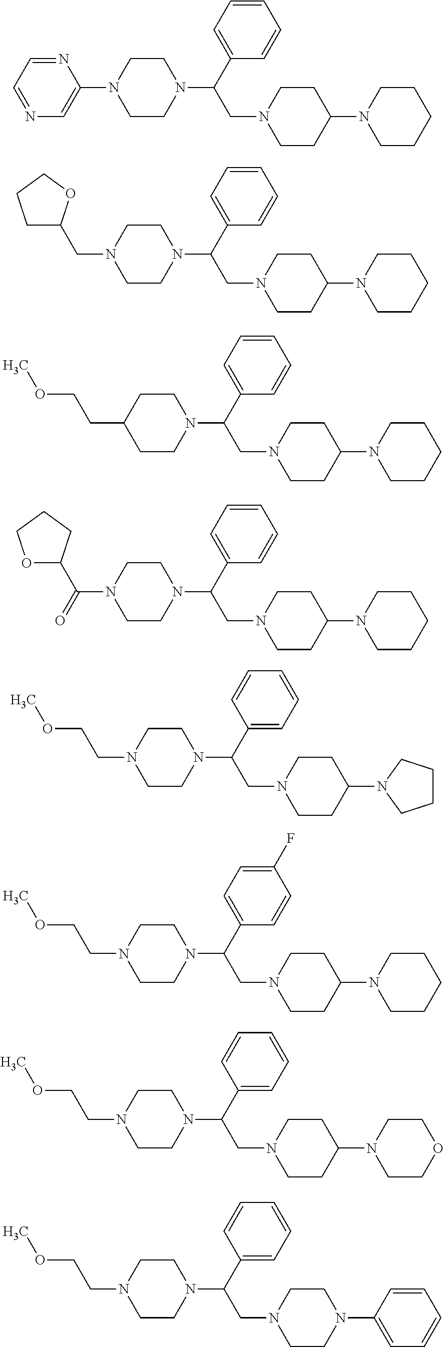 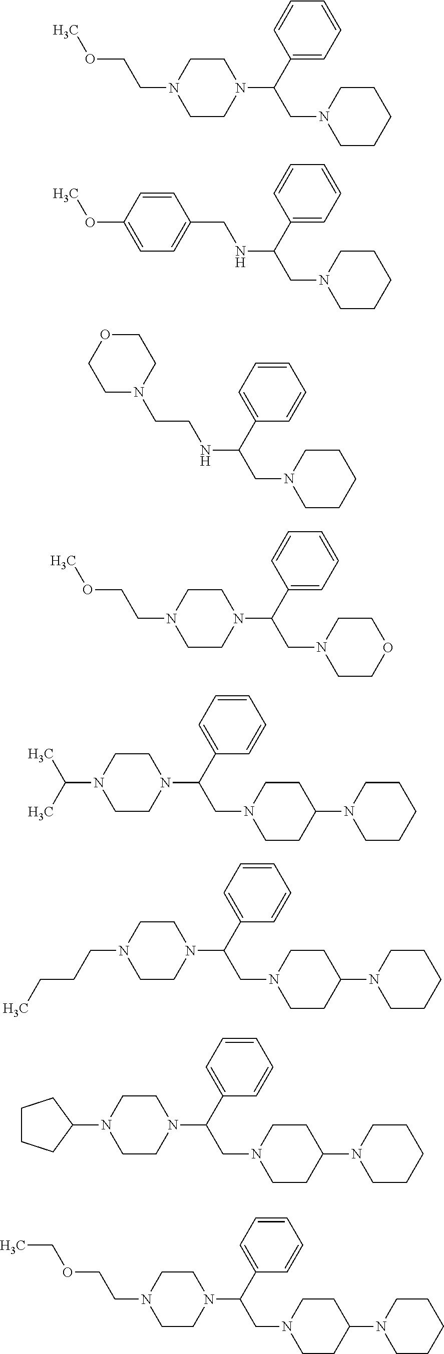 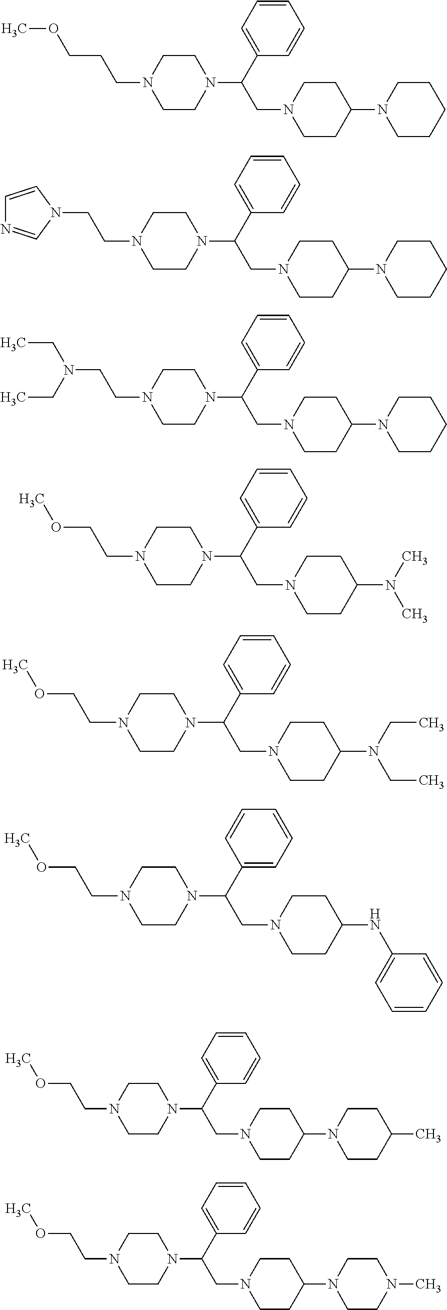 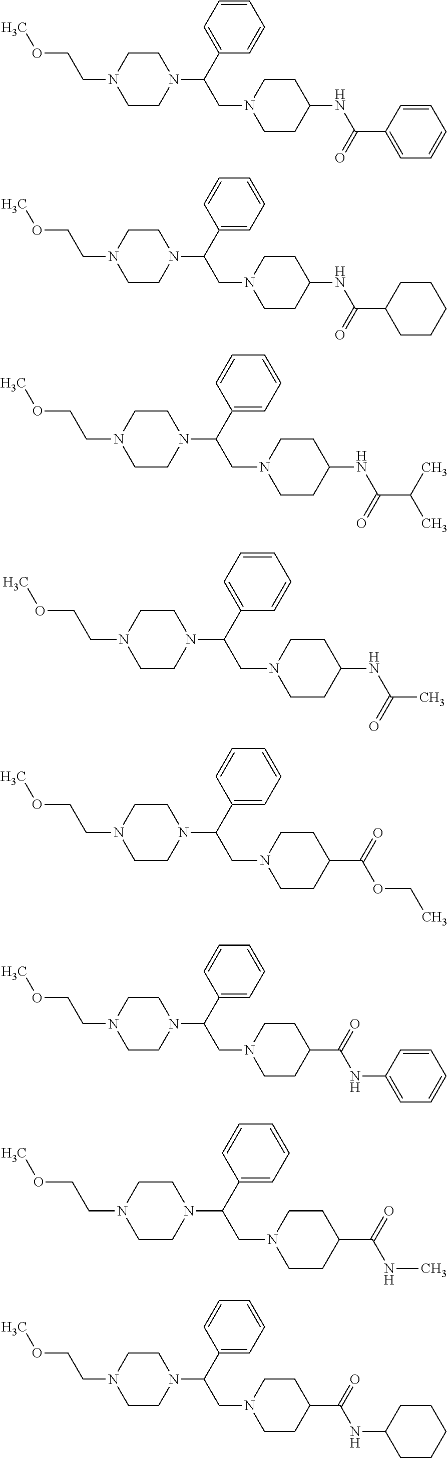 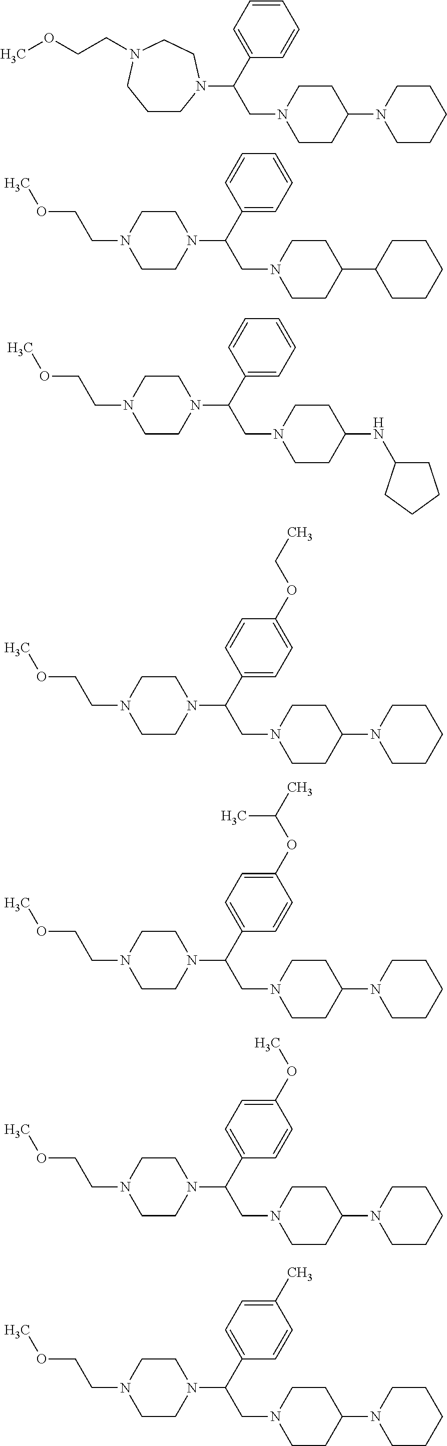 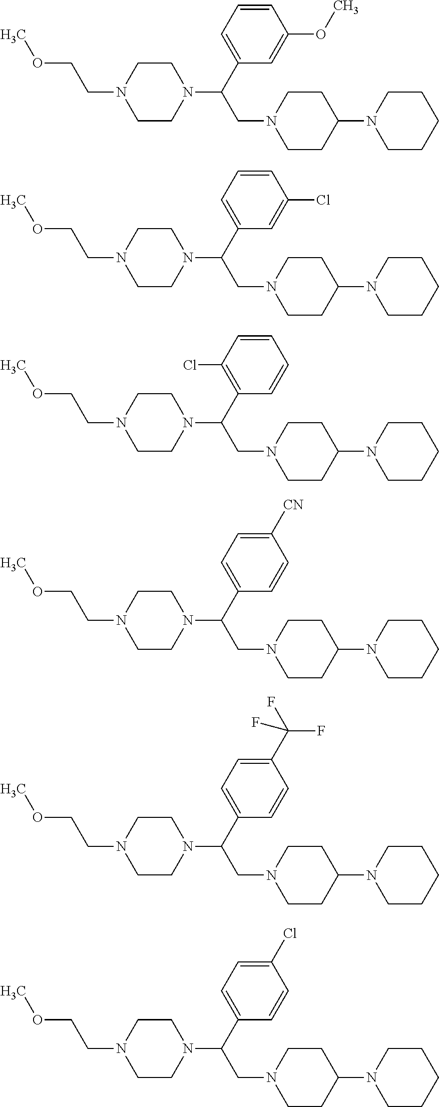
or pharmaceutically acceptable salts thereof.

35. The method of claim 23, wherein the disorders in iron metabolism are selected from the group consisting of: iron deficiency diseases, anaemias, anaemias with cancer, anaemia induced by chemotherapy, anaemia induced by inflammation, anaemias with congestive cardiac insufficiency, anaemia with chronic renal insufficiency stage 3-5, anaemia induced by chronic inflammation, anaemia with rheumatic arthritis, anaemia with systemic lupus erythematosus and anaemia with inflammatory intestinal diseases.

36. The method of claim 23, wherein the preparation further comprises at least one of pharmaceutical carriers, auxiliary substances, and solvents for use in the treatment of iron metabolism disorders.

37. The method of claim 23, wherein the preparation further comprises at least one further pharmaceutically active compound, which is a compound for treatment of disorders in iron metabolism and the accompanying symptoms.

38. The method of claim 23, wherein the compound for treatment of disorders in iron metabolism and the accompanying symptoms is an iron-containing compound for the use in the treatment of iron metabolism disorders.

39. The method of claim 24, wherein the compound of formula (I) has the general formula (Ia) 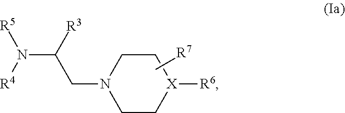
and wherein
X is chosen from: O, N or CH;
R⁶ is chosen from the group consisting of
hydrogen,
optionally substituted alkyl,
optionally substituted alkenyl,
optionally substituted alkynyl,
optionally substituted acyl,
optionally substituted alkoxycarbonyl,
optionally substituted amino,
optionally substituted aminocarbonyl,
optionally substituted alkyl-, aryl- or heterocyclylsulfonyl,
optionally substituted aryl, and
optionally substituted heterocyclyl;
R⁷ is chosen from the group consisting of:
hydrogen,
hydroxyl,
halogen,
cyano,
nitro,
carboxyl,
sulfonic acid radical (—SO₃H),
optionally substituted amino,
optionally substituted aminocarbonyl,
optionally substituted aminosulfonyl,
optionally substituted acyl,
optionally substituted acyloxy,
optionally substituted alkoxy,
optionally substituted alkoxycarbonyl,
optionally substituted alkyl,
optionally substituted alkenyl,
optionally substituted alkynyl,
optionally substituted aryl, and
optionally substituted heterocyclyl,
or pharmaceutically acceptable salts thereof.

40. The method of claim 26, wherein in the compound of formula (I),
X is chosen from: N or CH;
R⁶ is chosen from the group consisting of:
hydrogen,
optionally substituted alkyl,
optionally substituted acyl,
optionally substituted alkoxycarbonyl,
optionally substituted amino,
optionally substituted aminocarbonyl,
optionally substituted alkyl-, aryl- or heterocyclylsulfonyl,
optionally substituted aryl, and
optionally substituted heterocyclyl;
R⁷ is chosen from the group consisting of:
hydrogen,
halogen,
optionally substituted amino,
optionally substituted acyl,
optionally substituted alkoxy,
optionally substituted alkyl,
optionally substituted aryl, and
optionally substituted heterocyclyl;
R³ is chosen from the group consisting of:
optionally substituted aryl, and
optionally substituted heterocyclyl;
R⁴ and R⁵ are identical or different and are each chosen from the group consisting of:
hydrogen,
optionally substituted alkyl,
optionally substituted aryl, and
optionally substituted heterocyclyl; or
R⁴ and R⁵ together with the nitrogen atom to which they are bonded from a saturated or unsaturated, optionally substituted 5- to 7-membered ring which can optionally contain further hetero atoms;
or pharmaceutically acceptable salts thereof.

41. The method of claim 26, wherein in the compound having formula (I),
X has the meaning N;
R⁶ is chosen from the group consisting of:
optionally substituted acyl,
optionally substituted alkyl-, aryl- or heterocyclylsulfonyl,
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of:
hydrogen or
optionally substituted alkyl, or
R⁴ and R⁵ together with the nitrogen atom to which they are bonded form a saturated or unsaturated, optionally substituted 6-membered ring which can optionally contain further hetero atoms.
or pharmaceutically acceptable salts thereof.

42. The method of claim 26, wherein in the compounds having the formula (I),
X has the meaning N;
R⁶ is chosen from the group consisting of:
optionally substituted acyl,
optionally substituted aryl, and
optionally substituted heterocyclyl;
R⁷ is hydrogen;
R³ is chosen from the group consisting of:
optionally substituted aryl, and
optionally substituted heterocyclyl;
R⁴ and R⁵ are identical or different and are each chosen from the group consisting of:
hydrogen or
optionally substituted alkyl,
or pharmaceutically acceptable salts thereof.
